(19) United States
(12) Patent Application Publication
(10) Pub. No.: US 2022/0259254 A1
(43) Pub. Date: Aug. 18, 2022

(54) TRANS-CYCLOOCTENE BIOORTHOGONAL AGENTS AND USES IN CANCER AND IMMUNOTHERAPY

(71) Applicant: Tambo, Inc., San Francisco, CA (US)

(72) Inventors: Jose Manuel Mejia Oneto, San Francisco, CA (US); Nathan A. Yee, San Francisco, CA (US); Sangeetha Srinivasan, San Francisco, CA (US); Michael Zakharian, San Francisco, CA (US)

(21) Appl. No.: 17/624,625

(22) PCT Filed: Jul. 6, 2020
(86) PCT No.: PCT/US2020/040891

§ 371 Date: Jan. 4, 2022

Related U.S. Application Data
(60) Provisional application No. 62/981,401, filed on Feb. 25, 2020.
(60) Provisional application No. 62/971,196, filed on Feb. 6, 2020.
(60) Provisional application No. 62/871,051, filed on Jul. 5, 2019.

(51) Int. Cl. C07H 21/02; A61P 35/04; A61K 47/61; A61K 47/54

(52) U.S. Cl. CPC C07H 21/02; A61K 47/555; A61K 47/61; A61P 35/04

(57) ABSTRACT

Trans-cyclooctene conjugates of therapeutic agents may be used for bioorthogonal delivery to a targeted location in a subject. The compositions and methods have applications in the treatment of various diseases or conditions including cancer, tumor growths, and bacterial infections.

### RELATED APPLICATIONS

This application claims priority to U.S. Provisional Patent Application No. 62/871,051, filed on Jul. 5, 2019, U.S. Provisional Patent Application No. 62/971,196, filed on Feb. 6, 2020, and U.S. Provisional Patent Application No. 62/981,401, filed on Feb. 25, 2020, each of which is incorporated herein by reference in its entirety.

### TECHNICAL FIELD

The present disclosure provides trans-cyclooctene derivatives and use for bioorthogonal delivery in a subject for cancer and/or immunotherapy.

### BACKGROUND

Immunotherapy to boost the immune system against tumor growth and dissemination of cancer has been clinically validated. Immunotherapy strategies harness immune cells and include monoclonal antibodies against tumor antigens, immune checkpoint inhibitors, vaccination, adoptive cell therapies (e.g., CAR-T cells) and cytokine administration.

TLR agonists play a fundamental role in activating innate and adaptive immune responses. In mouse models, treatment with TLR agonists has been shown to reduce tumor growth and in some cases, destroy established tumors when used in combination with other therapeutic agents, such chemotherapy drugs, mAb, and various tumor antigen vaccines in the form of proteins, peptides, or plasmid DNA. TLR agonists activate professional antigen-presenting cells (APCs), namely dendritic cells (DCs). TLRs can induce preferable anti-tumor effect by eliciting inflammatory cytokines expression and cytotoxic T lymphocytes (CTLs) response. As adjuvant, TLRs agonists can launch a strong immune response to assist cancer radiotherapy and biochemotherapy. The engagement of TLRs on various T cell subsets has more recently been demonstrated to augment their responses and thus represents a novel and promising strategy to enhance the efficacy of cancer immunotherapies.

The central role of STING in controlling anticancer immune responses was exemplified by observations that spontaneous and radiation-induced adaptive anticancer immunity was reduced in the absence of STING, illustrating the potential of STING-targeting for cancer immunotherapy.

Bioorthogonal conjugation or click reactions are selective and orthogonal (non-interacting with) functionalities found in biological systems, and have found use in various applications in the fields of chemistry, chemical biology, molecular diagnostics, and medicine, where they can be used to facilitate the selective manipulation of molecules, cells, particles and surfaces, and the tagging and tracking of biomolecules in vitro and in vivo. These reactions include the Staudinger ligation, the azide-cyclooctyne cycloaddition, and the inverse-electron-demand Diels-Alder reaction.

WO2017/044983 describes anti-tumor effects of a trans-cyclooctene conjugate of doxorubicin through release of doxorubicin at a tumor site by bioorthogonal reaction with a tetrazine-functionalized alginate implanted at the tumor site.

### SUMMARY OF THE INVENTION

The present disclosure provides trans-cyclooctene derivatives for delivering payload molecules in a subject using bioorthogonal chemistry. The disclosure also provides methods of producing the compositions, as well as methods of using the same.

In one aspect, the invention provides compounds of formula (I), or a pharmaceutically acceptable salt thereof,

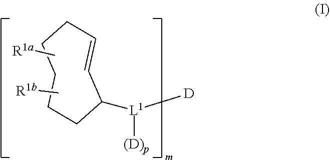

wherein
R^{1a}, at each occurrence, is independently selected from the group consisting of hydrogen, C₁₋₄alkyl, and C₁₋₄haloalkyl;
R^{1b}, at each occurrence, is independently selected from the group consisting of hydrogen, C₁₋₄alkyl, C₁₋₄haloalkyl, C(O)OH, C(O)OC₁₋₄alkyl, C(O)N(R^{1c})CHR^{1e}CO₂H, C(O)N(R^{1c})CHR^{1e}C(O)OC₁₋₄alkyl, C(O)N(R^{1c})—C₁₋₆alkylene-CO₂H, and C(O)N(R^{1c})—C₁₋₆alkylene-C(O)OC₁₋₄ alkyl;
R^{1c}, at each occurrence, is independently hydrogen or C₁₋₄alkyl;
R^{1e}, at each occurrence, is independently —C₁₋₄alkylene-CO₂H, —C₁₋₄alkylene-CONH₂, or —C₁₋₄alkylene-OH;
D, at each occurrence, is independently a payload selected from the group consisting of a toll-like receptor (TLR) agonist and a stimulator of interferon genes (STING) agonist;
L¹, at each occurrence, is independently a linker;
m, at each occurrence, is independently 1, 2, or 3; and
p, at each occurrence, is independently 0, 1, or 2.

In another aspect, the invention provides a pharmaceutical composition comprising a compound of formula (I), or a pharmaceutically acceptable salt thereof, and a pharmaceutically acceptable carrier.

In another aspect, the invention provides a method of treating or preventing a condition or disorder or enhancing or eliciting an immune response, the method comprising administering to a subject in need thereof, a therapeutically effective amount of a compound of formula (I), or a pharmaceutically acceptable salt or composition thereof; and a therapeutic support composition, the therapeutic support composition comprising a biocompatible support and a tetrazine-containing group of formula

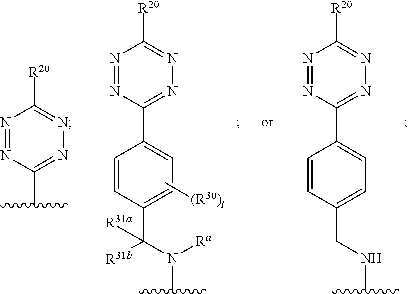

wherein
R²⁰ is selected from the group consisting of hydrogen, halogen, cyano, nitro, alkyl, alkenyl, alkynyl, heteroalkyl, aryl, heteroaryl, heterocycle, cycloalkyl, cycloalkenyl, CF₃, CF₂—R′, NO₂, OR′, SR′, C(═O)R′, C(═S)R′, OC(═O)R′″, SC(═O)R′″, OC(═S)R″, SC(═S)R′″, S(═O)R′, S(═O)₂R′″, S(═O)₂NR′R″, C(═O)O—R′, C(═O)S—R′, C(═S)O—R′, C(═S)S—R′, C(═O)NR′R″, C(═S)NR′ R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S) R′R′″, SC(═S)R′R″, NR′C(═O)NR″R″, and NR′C(═S)NR″R″;
R′ and R″ at each occurrence are independently selected from hydrogen, aryl and alkyl;
R′″ at each occurrence is independently selected from aryl and alkyl;
R³⁰ is halogen, cyano, nitro, hydroxy, alkyl, haloalkyl; alkenyl, alkynyl, alkoxy; halalkoxy; heteroalkyl, aryl, heteroaryl, heterocycle, cycloalkyl, or cycloalkenyl;
R^{a}, R^{31a} and R^{31b} are each independently hydrogen, C₁-C₆-alkyl, or C1-C₆-haloalkyl; and
t is 0, 1, 2, 3, or 4.

In another aspect, the invention provides a pharmaceutical combination comprising a compound of formula (I), or a pharmaceutically acceptable salt, or composition thereof; and a therapeutic support composition, the therapeutic support composition comprising a biocompatible support and a tetrazine-containing group of formula

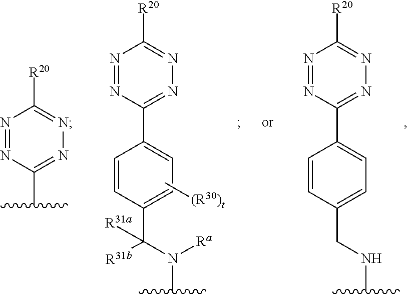

as defined herein
for use in the treatment or prevention of a disease or disorder, such as cancer, infections, tissue injury, stenosis, ischemia, re-vascularization, myocardial infarction, arrhythmias, vascular occlusion, inflammation, autoimmune disorders, transplant rejection, macular degeneration, rheumatoid arthritis, osteoarthritis, peri-prosthetic infections, and pigmented villonodular synovitis; or for use in enhancing or eliciting an immune response.

In another aspect, the invention provides the use of a combination comprising a compound of formula (I), or a pharmaceutically acceptable salt, or composition thereof; and a therapeutic support composition, the therapeutic support composition comprising a biocompatible support and a tetrazine-containing group of formula

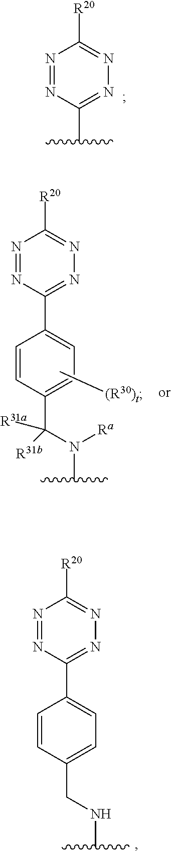

as defined herein
in the manufacture of a medicament for the treatment or prevention of a condition or disorder such as cancer, infections, tissue injury, stenosis, ischemia, re-vascularization, myocardial infarction, arrhythmias, vascular occlusion, inflammation, autoimmune disorders, transplant rejection, macular degeneration, rheumatoid arthritis, osteoarthritis, peri-prosthetic infections, and pigmented villonodular synovitis; or for use in enhancing or eliciting an immune response.

Aspects of the present disclosure include a method for delivering an effective amount of a payload to a target location in a subject, where the method includes administering to the subject a therapeutic support composition, as defined herein.

Aspects of the present disclosure also include a kit comprising a compound of formula (I), a therapeutic support composition as defined herein, and optionally a compound of formula (I-B), as defined herein.

Another aspect of the invention provides a method of treating cancer or enhancing or eliciting an immune response comprising administering to a subject in need thereof:
a) a therapeutically effective amount of a compound of formula (II-A) or (III-A), or a pharmaceutically acceptable salt thereof,

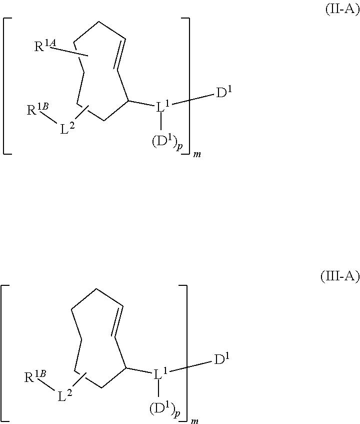

wherein
R^{1A}, at each occurrence, is independently selected from the group consisting of C₁₋₄alkyl, C₁₋₄ haloalkyl, and C₁₋₄alkoxy;
R^{1B}, at each occurrence, is independently selected from the group consisting of G′, OH, —NR^{1c}—C₁₋₄alkylene-G¹, —NR^{1c}—C₁₋₄alkylene-N(R^{1d})₂, —N(R^{1c})CHR^{1e}CO₂H, —N(R^{1c})—C₁₋₆alkylene-CO₂H, —N(R^{1f})—C₂₋₄alkylene-(N(C₁₋₄alkylene-CO₂H)—C₂₋₄alkylene)ₙ-N(C₁₋₄alkylene-CO₂H)₂, —N(R^{1c})CHR^{1e}(O)OC₁₋₆alkyl, —N(R^{1c})—C₁₋₆alkylene-C(O)OC₁₋₆alkyl, and —N(R^{1f})—C₂₋₄alkylene-(N(C₁₋₄alkylene-C(O)OC₁₋₆alkyl)-C₂₋₄alkylene)ₙ-N(C₁₋₄alkylene-C(O)OC₁₋₆alkyl)₂;
R^{1c} and R^{1d}, at each occurrence, are independently hydrogen or C₁₋₄alkyl;
R^{1e}, at each occurrence, is independently —C₁₋₄alkylene-CO₂H, —C₁₋₄alkylene-CONH₂, or —C₁₋₄alkylene-OH;
R^{1f}, at each occurrence, is independently hydrogen, C₁₋₆alkyl, or C₁₋₄alkylene-CO₂H;
D¹, at each occurrence, is independently an anticancer agent payload;
L¹, at each occurrence, is independently a linker;
L², at each occurrence, is independently selected from the group consisting of —C(O)— and C₁₋₃ alkylene;
G¹, at each occurrence, is independently an optionally substituted heterocyclyl;
m is 1, 2, or 3
n, at each occurrence, is independently 0, 1, 2, or 3; and
p, at each occurrence, is independently 0, 1, or 2;
b) a therapeutic support composition comprising a support and a tetrazine-containing group of formula

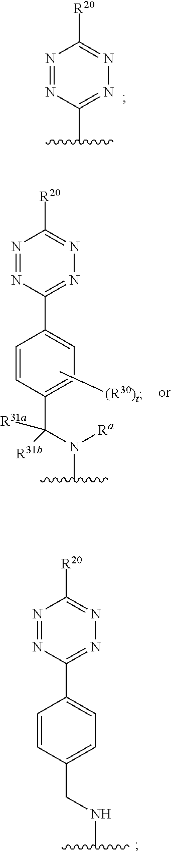

wherein R²⁰ is selected from the group consisting of hydrogen, halogen, cyano, nitro, alkyl, alkenyl, alkynyl, heteroalkyl, aryl, heteroaryl, heterocycle, cycloalkyl, cycloalkenyl, CF₃, CF₂—R′, NO₂, OR′, SR′, C(═O)R′, C(═S)R′, OC(═O)R′″, SC(═O)R′″, OC(═S)R′″, SC(═S)R′″, S(═O)R′, S(═O)₂R, S(═O)₂NR′ R″, C(═O)O—R′, C(═O)S—R′, C(═S)O—R′, C(═S)S—R′, C(═O)NR′R″, C(═S)NR′ R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S) R′R′″, SC(═S)R′R″, NR′C(═O)NR″R″, and NR′C(═S)NR″R″; R′ and R″ at each occurrence are independently selected from hydrogen, aryl and alkyl; and R′″ at each occurrence is independently selected from aryl and alkyl; R³⁰ is halogen, cyano, nitro, hydroxy, alkyl, haloalkyl; alkenyl, alkynyl, alkoxy; halalkoxy; heteroalkyl, aryl, heteroaryl, heterocycle, cycloalkyl, or cycloalkenyl; R^{a}, R^{31a} and R^{31b} are each independently hydrogen, C₁-C₆-alkyl, or C₁-C₆-haloalkyl; and t is 0, 1, 2, 3, or 4;
wherein the tetrazine-containing group is linked or directly bonded to the support;
and
c) a therapeutically effective amount one or more immunomodulatory agents, or a pharmaceutically acceptable salt thereof.

In another aspect, the invention provides a pharmaceutical combination of a compound of formula (II-A) or (III-A), or a pharmaceutically acceptable salt, or composition thereof; a therapeutic support composition; and immunomodulatory agents for use in the treatment or prevention of a disease or disorder, such as cancer, infections, tissue injury, stenosis, ischemia, re-vascularization, myocardial infarction, arrhythmias, vascular occlusion, inflammation, autoimmune disorders, transplant rejection, macular degeneration, rheumatoid arthritis, osteoarthritis, peri-prosthetic infections, and pigmented villonodular synovitis; or for use in enhancing or eliciting an immune response.

In another aspect, the invention provides the use of a combination of a compound of formula (II-A) or (III-A), or a pharmaceutically acceptable salt, or composition thereof; a therapeutic support composition; and immunomodulatory agents in the manufacture of a medicament for the treatment or prevention of a condition or disorder such as cancer, infections, tissue injury, stenosis, ischemia, re-vascularization, myocardial infarction, arrhythmias, vascular occlusion, inflammation, autoimmune disorders, transplant rejection, macular degeneration, rheumatoid arthritis, osteoarthritis, peri-prosthetic infections, and pigmented villonodular synovitis; or for use in enhancing or eliciting an immune response.

Another aspect of the invention provides a kit comprising a) the compound of formula (II-A) or (III-A), or a pharmaceutically acceptable salt or composition thereof; b) immunomodulatory agents, or a pharmaceutically acceptable salt or composition thereof; and c) instructions for use.

Another aspect of the invention provides a kit comprising a) the therapeutic support composition; b) immunomodulatory agents, or a pharmaceutically acceptable salt or composition thereof; and c) instructions for use.

Another aspect of the invention provides a pharmaceutical composition comprising a) the compound of formula (II-A) or (III-A), or a pharmaceutically acceptable salt thereof; b) immunomodulatory agents, or a pharmaceutically acceptable salt thereof; and c) a pharmaceutically acceptable carrier.

Another aspect of the invention provides a pharmaceutical composition comprising a) the therapeutic support composition; b) immunomodulatory agents, or a pharmaceutically acceptable salt thereof; and c) a pharmaceutically acceptable carrier.

Aspects of the present disclosure include a method for delivering an effective amount of a payload to a target location in a subject, where the method includes administering to the subject a therapeutic support composition, as defined herein.

Another aspect of the invention provides a method of treating cancer comprising:
a) administering to a subject in need thereof, a therapeutically effective amount of a compound of formula (II-A), or a pharmaceutically acceptable salt thereof,
   wherein

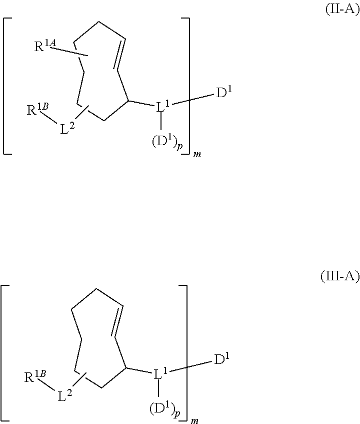

R^{1A}, at each occurrence, is independently selected from the group consisting of C₁₋₄alkyl, C₁₋₄haloalkyl, and C₁₋₄ alkoxy;
R^{1B}, at each occurrence, is independently selected from the group consisting of G¹, OH, —NR^{1c}—C₁₋₄alkylene-G¹, —NR^{1c}—C₁₋₄alkylene-N(R^{1d})₂, —N(R^{1c})CHR^{1e}CO₂H, —N(R^{1c})—C₁₋₆alkylene-CO₂H, —N(R^{1f})—C₂₋₄alkylene-(N(C₁₋₄alkylene-CO₂H)—C₂₋₄alkylene)ₙ-N(C₁₋₄alkylene-CO₂H)₂, —N(R^{1c})CHR^{1e}C(O)OC₁₋₆alkyl, —N(R^{1c})—C₁₋₆alkylene-C(O)OC₁₋₆alkyl, and —N(R^{1f})—C₂₋₄alkylene-(N(C₁₋₄alkylene-C(O)OC₁₋₆alkyl)-C₂₋₄alkylene)ₙ-N(C₁₋₄alkylene-C(O)OC₁₋₆alkyl)₂;
R^{1c} and R^{1d}, at each occurrence, are independently hydrogen or C₁₋₄alkyl;
R^{1e}, at each occurrence, is independently —C₁₋₄alkylene-CO₂H, —C₁₋₄alkylene-CONH₂, or —C₁₋₄ alkylene-OH;
R^{1f}, at each occurrence, is independently hydrogen, C₁₋₆ alkyl, or C₁₋₄ alkylene-CO₂H;
D¹, at each occurrence, is independently an anticancer agent payload;
L¹, at each occurrence, is independently a linker;
L², at each occurrence, is independently selected from the group consisting of —C(O)— and C₁₋₃ alkylene;
G¹, at each occurrence, is independently an optionally substituted heterocyclyl;
m is 1, 2, or 3
n, at each occurrence, is independently 0, 1, 2, or 3; and
p, at each occurrence, is independently 0, 1, or 2; and
b) locally administering at a first tumor in the subject, a therapeutic support composition comprising a support and a tetrazine-containing group of formula

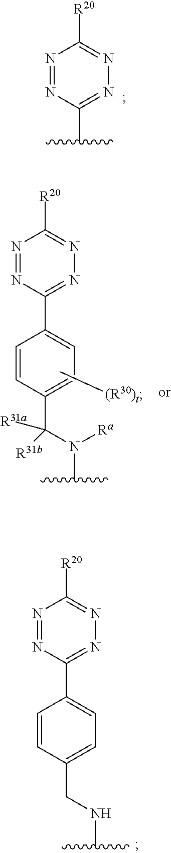

wherein R²⁰ is selected from the group consisting of hydrogen, halogen, cyano, nitro, alkyl, alkenyl, alkynyl, heteroalkyl, aryl, heteroaryl, heterocycle, cycloalkyl, cycloalkenyl, CF₃, CF₂—R′, NO₂, OR′, SR′, C(═O)R′, C(═S)R′, OC(═O)R′″, SC(═O)R′″, OC(═S)R′″, SC(═S)R′″, S(═O)R′, S(═O)₂R, S(═O)₂NR′ R″, C(═O)O—R′, C(═O)S—R′, C(═S)O—R′, C(═S)S—R′, C(═O)NR′R″, C(═S)NR′ R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S) R′R′″, SC(═S)R′R″, NR′C(═O)NR″R″, and NR′C(═S)NR″R″; R′ and R″ at each occurrence are independently selected from hydrogen, aryl and alkyl; and R′″ at each occurrence is independently selected from aryl and alkyl; R³⁰ is halogen, cyano, nitro, hydroxy, alkyl, haloalkyl; alkenyl, alkynyl, alkoxy; halalkoxy; heteroalkyl, aryl, heteroaryl, heterocycle, cycloalkyl, or cycloalkenyl; R^{a}, R^{31a} and R^{31b} are each independently hydrogen, C₁-C₆-alkyl, or C₁-C₆-haloalkyl; and t is 0, 1, 2, 3, or 4;
wherein the tetrazine-containing group is linked or directly bonded to the support;
wherein the subject has a second tumor and the administration of a) and the administration of b) inhibits growth of a second tumor in the patient.

In another aspect, the invention provides a method of enhancing or eliciting an immune response against a second tumor in a subject comprising a) administering a compound of formula (II-A) or (III-A), or a pharmaceutically acceptable salt thereof to the subject; b) locally administering a therapeutic support composition to the subject at a first tumor; wherein the compound of formula (II-A) or (III-A) and the therapeutic support composition are as defined herein, and the administration of a) and the administration of b) enhances or elicits an immune response against the second tumor.

In another aspect, the invention provides a method of inhibiting tumor metastasis in a subject at risk of tumor metastasis comprising a) administering a compound of formula (II-A) or (III-A), or a pharmaceutically acceptable salt thereof to the subject; and b) locally administering a therapeutic support composition to the subject at a first tumor; wherein the compound of formula (II-A) or (III-A) and the therapeutic support composition are as defined herein.

In another aspect, the invention provides a pharmaceutical combination comprising a) a compound of formula (II-A) or (III-A), or a pharmaceutically acceptable salt, or composition thereof; and b) a therapeutic support composition; for use in a method of inhibiting growth of a second tumor in a subject, wherein the therapeutic support composition is locally administered at a first tumor in the subject and the compound of formula (II-A) or (III-A), or a pharmaceutically acceptable salt, or composition thereof is administered to the subject.

In another aspect, the invention provides a pharmaceutical combination comprising a) a compound of formula (II-A) or (III-A), or a pharmaceutically acceptable salt, or composition thereof; and b) a therapeutic support composition; for use in a method of enhancing or eliciting an immune response against a second tumor in a subject, wherein the therapeutic support composition is locally administered at a first tumor in the subject and the compound of formula (II-A) or (III-A), or a pharmaceutically acceptable salt, or composition thereof is administered to the subject.

In another aspect, the invention provides a pharmaceutical combination comprising a) a compound of formula (II-A) or (III-A), or a pharmaceutically acceptable salt, or composition thereof; and b) a therapeutic support composition; for use in a method of inhibiting tumor metastasis in a subject at risk of tumor metastasis, wherein the therapeutic support composition is locally administered at a first tumor in the subject and the compound of formula (II-A) or (III-A), or a pharmaceutically acceptable salt, or composition thereof is administered to the subject.

In another aspect, the invention provides the use of a combination comprising a) a compound of formula (II-A) or (III-A), or a pharmaceutically acceptable salt, or composition thereof; and b) a therapeutic support composition; in the manufacture of a medicament for inhibiting growth of a second tumor, wherein the therapeutic support composition is locally administered at a first tumor in the subject and the compound of formula (II-A) or (III-A), or a pharmaceutically acceptable salt, or composition thereof is administered to the subject.

In another aspect, the invention provides use of a combination comprising a) a compound of formula (II-A) or (III-A), or a pharmaceutically acceptable salt, or composition thereof; and b) a therapeutic support composition; in the manufacture of a medicament for enhancing or eliciting an immune response against a second tumor, wherein the therapeutic support composition is locally administered at a first tumor in the subject and the compound of formula (I-A), or a pharmaceutically acceptable salt, or composition thereof is administered to the subject.

In another aspect, the invention provides use of a combination comprising a) a compound of formula (II-A) or (III-A), or a pharmaceutically acceptable salt, or composition thereof; and b) a therapeutic support composition; in the manufacture of a medicament for inhibiting tumor metastasis in a subject at risk of tumor metastasis, wherein the therapeutic support composition is locally administered at a first tumor in the subject and the compound of formula (II-A) or (III-A), or a pharmaceutically acceptable salt, or composition thereof is administered to the subject.

### BRIEF DESCRIPTION OF THE DRAWINGS

FIG. 1A shows body weight effects in mice injected with MC38 colorectal tumor cells in the right flank and treated with a modified sodium hyaluronate biomaterial at the tumor injection site in combination with systemic treatment with saline (G1), Prodrug 1 (G2), or Prodrug 1 and a TLR9a agonist (G3), as described in Examples B1 and C1. Data points represent group mean body weight. Error bars represent standard error of the mean (SEM).

FIG. 1B shows body weight effects in mice injected with MC38 colorectal tumor cells in both the right flank and left flank and treated with a modified sodium hyaluronate biomaterial at the tumor injection site in the right flank, in combination with systemic treatment with saline (G4), doxorubicin (G5), doxorubicin and a TLR9a agonist (G6), Prodrug 1 and a TLR9a agonist (G7), or Prodrug 1 (G8), as described in Example C1. Data points represent group mean body weight. Error bars represent standard error of the mean (SEM).

FIG. 2A shows effects on tumor size in mice injected with MC38 colorectal tumor cells in the right flank and treated with a modified sodium hyaluronate biomaterial at the tumor injection site in combination with systemic treatment with saline (G1), Prodrug 1 (G2), or Prodrug 1 and a TLR9a agonist (G3), as described in Examples B1 and C1. Data points represent group mean body weight. Error bars represent standard error of the mean (SEM).

FIG. 2B shows effects on right flank tumor size in mice injected with MC38 colorectal tumor cells in both the right flank and left flank and treated with a modified sodium hyaluronate biomaterial at the tumor injection site in the right flank, in combination with systemic treatment with saline (G4), doxorubicin (G5), doxorubicin and a TLR9a agonist (G6), Prodrug 1 and a TLR9a agonist (G7), or Prodrug 1 (G8), as described in Example C1. Data points represent group mean body weight. Error bars represent standard error of the mean (SEM).

FIG. 3A shows effects on tumor size in mice injected with MC38 colorectal tumor cells in the right flank and treated with a modified sodium hyaluronate biomaterial at the tumor injection site in combination with systemic treatment with saline (G1), Prodrug 1 (G2), or Prodrug 1 and a TLR9a agonist (G3), as described in Examples B1 and C1. Data points represent group mean body weight. Error bars represent standard error of the mean (SEM).

FIG. 3B shows effects on right flank tumor size in mice injected with MC38 colorectal tumor cells in both the right flank and left flank and treated with a modified sodium hyaluronate biomaterial at the tumor injection site in the right flank, in combination with systemic treatment with saline (G4), doxorubicin (G5), doxorubicin and a TLR9a agonist (G6), Prodrug 1 and a TLR9a agonist (G7), or Prodrug 1 (G8), as described in Example C1. Data points represent group mean body weight. Error bars represent standard error of the mean (SEM).

FIG. 4 shows effects on left flank tumor size in mice injected with MC38 colorectal tumor cells in both the right flank and left flank and treated with a modified sodium hyaluronate biomaterial at the tumor injection site in the right flank, in combination with systemic treatment with saline (G4), doxorubicin (G5), doxorubicin and a TLR9a agonist (G6), Prodrug 1 and a TLR9a agonist (G7), or Prodrug 1 (G8), as described in Example C1. Data points represent group mean body weight. Error bars represent standard error of the mean (SEM).

FIG. 5 shows effects on left flank tumor size in mice injected with MC38 colorectal tumor cells in both the right flank and left flank and treated with a modified sodium hyaluronate biomaterial at the tumor injection site in the right flank, in combination with systemic treatment with saline (G4), doxorubicin (G5), doxorubicin and a TLR9a agonist (G6), Prodrug 1 and a TLR9a agonist (G7), or Prodrug 1 (G8), as described in Example C1. Data points represent group mean body weight. Error bars represent standard error of the mean (SEM).

FIG. 6A shows the treatment schedule for treatment groups G4, G5, and G8.

FIG. 6B shows a comparison of the effects on growth in volume of the right flank tumor injected with biomaterial followed by treatment groups G4, G5, and G8. The shaded region represents the BIOMATERIAL 1/PRODRUG 1 treatment duration.

FIG. 6C shows a comparison of the effects on growth in volume of the left flank tumor not injected with biomaterial followed by treatment groups G4, G5, and G8. The shaded region represents the BIOMATERIAL 1/PRODRUG 1 treatment duration.

FIG. 6D shows Kaplan-Meier survival curves for the mice in treatment groups G4, G5, and G8.

FIG. 7A shows the tumor growth curves for individual mice in treatment group G8.

FIG. 7B shows the tumor growth curves for individual mice in treatment group G5.

FIG. 7C shows the tumor growth curves for individual mice in treatment group G4.

FIG. 8 shows the tumor-infiltrating immune cell profile at 2 weeks post-treatment for treatment group G8 in the right flank tumor injected with biomaterial.

FIG. 9 shows the tumor-infiltrating immune cell profile at 2 weeks post-treatment for treatment group G8 in the left flank tumor not injected with biomaterial.

FIG. 10A shows the effect on tumor size in one mouse injected with MC38 colorectal tumor cells in the right flank (day 0) and treated with a modified sodium hyaluronate biomaterial at the tumor injection site in combination with systemic treatment with Prodrug 1 (G2), followed by a second injection of MC38 colorectal tumor cells in the left flank at day 70 (shown as arrow in FIG. 10A), as described in Example C1.

FIG. 10B shows a comparison of the effects for the treatment group of FIG. 10A with five naive mice injected with the MC38 colorectal tumor cells on the same day.

FIG. 11 shows Kaplan-Meier survival curves for the mice in treatment groups G4, G6, and G7. *** Statistical significance in survival was determined by log-rank (Mantel-Cox) test.

### DETAILED DESCRIPTION

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

The conjunctive term “or” includes any and all combinations of one or more listed elements associated by the conjunctive term. For example, the phrase “an apparatus comprising A or B” may refer to an apparatus including A where B is not present, an apparatus including B where A is not present, or an apparatus where both A and B are present. The phrases “at least one of A, B, . . . and N” or “at least one of A, B, . . . N, or combinations thereof” are defined in the broadest sense to mean one or more elements selected from the group comprising A, B, . . . and N, that is to say, any combination of one or more of the elements A, B, . . . or N including any one element alone or in combination with one or more of the other elements which may also include, in combination, additional elements not listed.

Definitions of specific functional groups and chemical terms are described in more detail below. For purposes of this disclosure, the chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

The term “alkoxy” as used herein, refers to an alkyl group, as defined herein, appended to the parent molecular moiety through an oxygen atom. Representative examples of alkoxy include, but are not limited to, methoxy, ethoxy, propoxy, 2-propoxy, butoxy and tert-butoxy.

The term “alkyl” as used herein, means a straight or branched, saturated hydrocarbon chain containing from 1 to 30 carbon atoms. The term “lower alkyl” or “C₁-C₆-alkyl” means a straight or branched chain hydrocarbon containing from 1 to 6 carbon atoms. The term “C₁-C₃-alkyl” means a straight or branched chain hydrocarbon containing from 1 to 3 carbon atoms. Representative examples of alkyl include, but are not limited to, methyl, ethyl, n-propyl, isopropyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkenyl” as used herein, means a hydrocarbon chain containing from 2 to 30 carbon atoms with at least one carbon-carbon double bond. The alkenyl group may be substituted or unsubstituted. For example, the alkenyl group may be substituted with an aryl group, such as a phenyl.

The term “alkynyl,” as used herein, refers to straight or branched monovalent hydrocarbyl groups having from 2 to 30 carbon atoms, such as 2 to 20, or 2 to 10 carbon atoms and having at least 1 site of triple bond unsaturation. The term “alkyne” also includes non-aromatic cycloalkyl groups of from 5 to 20 carbon atoms, such as from 5 to 10 carbon atoms, having single or multiple rings and having at least one triple bond. Examples of such alkynyl groups include, but are not limited to acetylenyl (—C≡CH), and propargyl (—CH₂C≡CH), and cycloalkynyl moieties, such as, but not limited to, substituted or unsubstituted cyclooctyne moieties.

The term “alkoxyalkyl” as used herein, refers to an alkoxy group, as defined herein, appended to the parent molecular moiety through an alkyl group, as defined herein.

The term “alkylene”, as used herein, refers to a divalent group derived from a straight or branched chain hydrocarbon of 1 to 30 carbon atoms, for example, of 2 to 10 carbon atoms. Representative examples of alkylene include, but are not limited to, —CH₂CH₂—, —CH₂CH₂CH₂—, —CH₂CH₂CH₂CH₂—, and —CH₂CH₂CH₂CH₂CH₂—.

The term “amino acid” refers to both natural and unnatural amino acids. It also includes protected natural and unnatural amino acids.

The term “aryl” as used herein, refers to a phenyl group, or bicyclic aryl or tricyclic aryl fused ring systems. Bicyclic fused ring systems are exemplified by a phenyl group appended to the parent molecular moiety and fused to a phenyl group. Tricyclic fused ring systems are exemplified by a phenyl group appended to the parent molecular moiety and fused to two other phenyl groups. Representative examples of bicyclic aryls include, but are not limited to, naphthyl. Representative examples of tricyclic aryls include, but are not limited to, anthracenyl. The monocyclic, bicyclic, and tricyclic aryls are connected to the parent molecular moiety through any carbon atom contained within the rings, and can be unsubstituted or substituted.

The term “azide” as used herein, refers to the functional group —N₃.

The term “cycloalkyl” as used herein, refers to a carbocyclic ring system containing three to ten carbon atoms, zero heteroatoms and zero double bonds. Representative examples of cycloalkyl include, but are not limited to, cyclopropyl, cyclobutyl, cyclopentyl, cyclohexyl, cycloheptyl, cyclooctyl, cyclononyl and cyclodecyl. “Cycloalkyl” also includes carbocyclic ring systems in which a cycloalkyl group is appended to the parent molecular moiety and is fused to an aryl group as defined herein, a heteroaryl group as defined herein, or a heterocycle as defined herein.

The term “cycloalkenyl” as used herein, means a non-aromatic monocyclic or multicyclic ring system containing at least one carbon-carbon double bond and preferably having from 5-10 carbon atoms per ring. Exemplary monocyclic cycloalkenyl rings include cyclopentenyl, cyclohexenyl or cycloheptenyl.

The term “cyclooctene” as used herein, refers to a substituted or unsubstituted non-aromatic cyclic alkyl group of 8 carbon atoms, having a single ring with a double bond. Examples of such cyclooctene groups include, but are not limited to, substituted or unsubstituted trans-cyclooctene (TCO).

The term “fluoroalkyl” as used herein, means an alkyl group, as defined herein, in which one, two, three, four, five, six, seven or eight hydrogen atoms are replaced by fluorine. Representative examples of fluoroalkyl include, but are not limited to, 2-fluoroethyl, 2,2,2-trifluoroethyl, trifluoromethyl, difluoromethyl, pentafluoroethyl, and trifluoropropyl such as 3,3,3-trifluoropropyl.

The term “alkoxyfluoroalkyl” as used herein, refers to an alkoxy group, as defined herein, appended to the parent molecular moiety through a fluoroalkyl group, as defined herein.

The term “fluoroalkoxy” as used herein, means at least one fluoroalkyl group, as defined herein, is appended to the parent molecular moiety through an oxygen atom. Representative examples of fluoroalkyloxy include, but are not limited to, difluoromethoxy, trifluoromethoxy and 2,2,2-trifluoroethoxy.

The term “halogen” or “halo” as used herein, means Cl, Br, I, or F.

The term “haloalkyl” as used herein, means an alkyl group, as defined herein, in which one, two, three, four, five, six, seven or eight hydrogen atoms are replaced by a halogen.

The term “haloalkoxy” as used herein, means at least one haloalkyl group, as defined herein, is appended to the parent molecular moiety through an oxygen atom.

The term “heteroalkyl” as used herein, means an alkyl group, as defined herein, in which one or more of the carbon atoms has been replaced by a heteroatom selected from S, Si, O, P and N. The heteroatom may be oxidized. Representative examples of heteroalkyls include, but are not limited to, alkyl ethers, secondary and tertiary alkyl amines, and alkyl sulfides.

The term “heteroaryl” as used herein, refers to an aromatic monocyclic ring or an aromatic bicyclic ring system or an aromatic tricyclic ring system. The aromatic monocyclic rings are five or six membered rings containing at least one heteroatom independently selected from the group consisting of N, O and S (e.g. 1, 2, 3, or 4 heteroatoms independently selected from O, S, and N). The five membered aromatic monocyclic rings have two double bonds and the six membered six membered aromatic monocyclic rings have three double bonds. The bicyclic heteroaryl groups are exemplified by a monocyclic heteroaryl ring appended to the parent molecular moiety and fused to a monocyclic cycloalkyl group, as defined herein, a monocyclic aryl group, as defined herein, a monocyclic heteroaryl group, as defined herein, or a monocyclic heterocycle, as defined herein. The tricyclic heteroaryl groups are exemplified by a monocyclic heteroaryl ring appended to the parent molecular moiety and fused to two of a monocyclic cycloalkyl group, as defined herein, a monocyclic aryl group, as defined herein, a monocyclic heteroaryl group, as defined herein, or a monocyclic heterocycle, as defined herein. Representative examples of monocyclic heteroaryl include, but are not limited to, pyridinyl (including pyridin-2-yl, pyridin-3-yl, pyridin-4-yl), pyrimidinyl, pyrazinyl, thienyl, furyl, thiazolyl, thiadiazolyl, isoxazolyl, pyrazolyl, and 2-oxo-1,2-dihydropyridinyl. Representative examples of bicyclic heteroaryl include, but are not limited to, chromenyl, benzothienyl, benzodioxolyl, benzotriazolyl, quinolinyl, thienopyrrolyl, thienothienyl, imidazothiazolyl, benzothiazolyl, benzofuranyl, indolyl, quinolinyl, imidazopyridine, benzooxadiazolyl, and benzopyrazolyl. Representative examples of tricyclic heteroaryl include, but are not limited to, dibenzofuranyl and dibenzothienyl. The monocyclic, bicyclic, and tricyclic heteroaryls are connected to the parent molecular moiety through any carbon atom or any nitrogen atom contained within the rings, and can be unsubstituted or substituted.

The term “heterocycle” or “heterocyclic” as used herein, means a monocyclic heterocycle, a bicyclic heterocycle, or a tricyclic heterocycle. The monocyclic heterocycle is a three-, four-, five-, six-, seven-, or eight-membered ring containing at least one heteroatom independently selected from the group consisting of O, N, and S. The three- or four-membered ring contains zero or one double bond, and one heteroatom selected from the group consisting of O, N, and S. The five-membered ring contains zero or one double bond and one, two or three heteroatoms selected from the group consisting of O, N and S. The six-membered ring contains zero, one or two double bonds and one, two, or three heteroatoms selected from the group consisting of O, N, and S. The seven- and eight-membered rings contains zero, one, two, or three double bonds and one, two, or three heteroatoms selected from the group consisting of O, N, and S. Representative examples of monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, 1,3-dimethylpyrimidine-2,4(1H,3H)-dione, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, oxetanyl, piperazinyl, piperidinyl, pyranyl, pyrazolinyl, pyrazolidinyl, pyrrolinyl, pyrrolidinyl, tetrahydrofuranyl, tetrahydropyranyl, tetrahydropyridinyl, tetrahydrothienyl, thiadiazolinyl, thiadiazolidinyl, 1,2-thiazinanyl, 1,3-thiazinanyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to a phenyl group, or a monocyclic heterocycle fused to a monocyclic cycloalkyl, or a monocyclic heterocycle fused to a monocyclic cycloalkenyl, or a monocyclic heterocycle fused to a monocyclic heterocycle, or a spiro heterocycle group, or a bridged monocyclic heterocycle ring system in which two non-adjacent atoms of the ring are linked by an alkylene bridge of 1, 2, 3, or 4 carbon atoms, or an alkenylene bridge of two, three, or four carbon atoms. Representative examples of bicyclic heterocycles include, but are not limited to, benzopyranyl, benzothiopyranyl, chromanyl, 2,3-dihydrobenzofuranyl, 2,3-dihydrobenzothienyl, 2,3-dihydroisoquinoline, 2-azaspiro[3.3]heptan-2-yl, azabicyclo[2.2.1]heptyl (including 2-azabicyclo[2.2.1]hept-2-yl), 2,3-dihydro-1H-indolyl, isoindolinyl, octahydrocyclopenta[c]pyrrolyl, octahydropyrrolopyridinyl, and tetrahydroisoquinolinyl. Tricyclic heterocycles are exemplified by a bicyclic heterocycle fused to a phenyl group, or a bicyclic heterocycle fused to a monocyclic cycloalkyl, or a bicyclic heterocycle fused to a monocyclic cycloalkenyl, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms. Examples of tricyclic heterocycles include, but are not limited to, octahydro-2,5-epoxypentalene, hexahydro-2H-2,5-methanocyclopenta[b]furan, hexahydro-1H-1,4-methanocyclopenta[c]furan, aza-adamantane (1-azatricyclo[3.3.1.1^{3,7}]decane), and oxa-adamantane (2-oxatricyclo[3.3.1.1^{3,7}]decane). The monocyclic, bicyclic, and tricyclic heterocycles are connected to the parent molecular moiety through any carbon atom or any nitrogen atom contained within the rings, and can be unsubstituted or substituted.

The term “hydroxyl” as used herein, means an —OH group.

The term “hydroxyalkyl” as used herein, means an alkyl group, as defined herein, in which one, two, three, four, five, six, seven or eight hydrogen atoms are replaced by a hydroxyl group.

In some instances, the number of carbon atoms in a hydrocarbyl substituent (e.g., alkyl or cycloalkyl) is indicated by the prefix “Cₓ-C_{y}-” or “C_{x-y},” wherein x is the minimum and y is the maximum number of carbon atoms in the substituent. Thus, for example, “C₁-C₃-alkyl” and “C₁₋₃alkyl” refer to an alkyl substituent containing from 1 to 3 carbon atoms. The two conventions “Cₓ-C_{y}-” and “C_{x-y}” are used interchangeably and have the same meaning.

In some instances, the number of carbon atoms in a hydrocarbyl substituent (e.g., alkyl or cycloalkyl) is indicated by the prefix “Cₓ-C_{y}”, wherein x is the minimum and y is the maximum number of carbon atoms in the substituent. Thus, for example, “C₁-C₃-alkyl” refers to an alkyl substituent containing from 1 to 3 carbon atoms.

The term “substituted” refers to a group that may be further substituted with one or more non-hydrogen substituent groups. Substituent groups include, but are not limited to, halogen, ═O, ═S, cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycle, cycloalkylalkyl, heteroarylalkyl, arylalkyl, hydroxy, hydroxyalkyl, alkoxy, alkoxyalkyl, alkylene, aryloxy, phenoxy, benzyloxy, amino, alkylamino, acylamino, aminoalkyl, arylamino, sulfonyl amino, sulfinylamino, sulfonyl, alkyl sulfonyl, aryl sulfonyl, aminosulfonyl, sulfinyl, —COOH, ketone, amide, carbamate, and acyl.

The term “tetrazine” refers to a substituted or unsubstituted aromatic cyclic group of 2 carbon atoms and 4 nitrogen atoms, having a single ring with three double bonds. Examples of tetrazine groups include 1,2,3,4-tetrazine and 1,2,4,5-tetrazine. As used herein, 1,2,4,5-tetrazine is referred to as a “Tz” group.

The term “selectively delivering” refers to delivering an agent (e.g., a payload) to an organ or tissue (or portion thereof) in need of treatment or diagnosis, without significant binding to other non-target organs or tissues (or portions thereof).

The term “payload” refers to an agent for delivery to a target site in a subject. Payloads include therapeutic agents.

The term “therapeutic agent” refers to an agent capable of treating and/or ameliorating a condition or disease, or one or more symptoms thereof, in a subject. Therapeutic agents of the present disclosure also include prodrug forms of therapeutic agents.

The term “diagnostic agent” refers to agents that assist in diagnosing conditions or diseases. Representative diagnostic agents include imaging agents such as paramagnetic agents, optical probes, radionuclides, and the like. Paramagnetic agents are imaging agents that are magnetic under an externally applied field. Examples of paramagnetic agents include, but are not limited to, iron particles including iron nanoparticles and iron microparticles. Optical probes are fluorescent compounds that can be detected by excitation at one wavelength of radiation and detection at a second, different, wavelength of radiation. Optical probes of the present disclosure include, but are not limited to, Cy5.5, Alexa 680, Cy5, DiD (1,1′-dioctadecyl-3,3,3′,3′-tetramethylindodicarbocyanine perchlorate) and DiR (1,1′-dioctadecyl-3,3,3′,3′-tetramethylindotricarbocyanine iodide). Other optical probes include quantum dots. Radionuclides are elements that undergo detectable radioactive decay. Radionuclides useful in embodiments of the present disclosure include, but are not limited to, ³H, ¹¹C, ¹³N, ¹⁸F, ¹⁹F, ⁶⁰Co, ⁶⁴Cu, ⁶⁷Cu, ⁶⁸Ga, ⁸²Rb, ⁹⁰Sr, ⁹⁰Y, ⁹⁹Tc, ^{99m}Tc, ¹¹¹In, ¹²³I, ¹²⁴I, ¹²⁵I, ¹²⁹I, ¹³¹I, ¹³⁷Cs, ¹⁷⁷Lu, ¹⁸⁶Re, ¹⁸⁸Re, ²¹¹At, Rn, R^{a}, Th, U, Pu and ²⁴¹Am.

The term “targeting agent” refers to a chemical or biological agent that specifically binds to a target (e.g., a targeted organ or tissue), thereby forming a stable association between the targeting agent and the specific target. By “stably associated” or “stable association” is meant that a moiety is bound to or otherwise associated with another moiety or structure under standard physiological conditions. Bonds may include covalent bonds and non-covalent interactions, such as, but not limited to, ionic bonds, hydrophobic interactions, hydrogen bonds, van der Waals forces (e.g., London dispersion forces), dipole-dipole interactions, and the like. A targeting agent may be a member of a specific binding pair, such as, but are not limited to: a member of a receptor/ligand pair; a ligand-binding portion of a receptor; a member of an antibody/antigen pair; an antigen-binding fragment of an antibody; a hapten; a member of a lectin/carbohydrate pair; a member of an enzyme/substrate pair; biotin/avidin; biotin/streptavidin; digoxin/antidigoxin; a member of a DNA or RNA aptamer binding pair; a member of a peptide aptamer binding pair; and the like. Targeting agents include ligands that specifically bind (or substantially specifically bind) a particular clinically-relevant target receptor or cell surface target. The ligand can be an antibody, peptide, nucleic acid, phage, bacteria, virus, or other molecule with a specific affinity for a target receptor or cell surface target. Examples of receptors and cell surface targets include, but are not limited to, PD-1, CTLA-4, HER2/neu, HER1/EGFR, VEGFR, BCR-ABL, SRC, JAK2, MAP2K, EML4-ALK, BRAF V600E, 4-1BB, GITR, GSK3beta, LT4—human mAb directed against the inhibitory immune checkpoint receptor immunoglobulin-like transcript 4 (ILT4; leukocyte immunoglobulin-like receptor subfamily B member 2, LILRB2, lymphocyte immunoglobulin-like receptor 2, LIR2, monocyte/macrophage immunoglobulin-like receptor 10, MIR-10, CD85d, or other cellular receptors or cell surface targets.

The term “targeted organ or tissue” refers to an organ or tissue that is being targeted for delivery of the payload. Representative organs and tissues for targeting include those that can be targeted by chemical or biological targeting agents, as well as those organs and tissues that cannot be targeted by chemical or biological targeting agents.

The term “implanting” refers to surgical implantation into a subject's body.

The term “contacting” or “contact” refers to the process of bringing into contact at least two distinct species such that they can interact with each other, such as in a non-covalent or covalent binding interaction or binding reaction. It should be appreciated, however, the resulting complex or reaction product can be produced directly from an interaction or a reaction between the added reagents or from an intermediate from one or more of the added reagents or moieties, which can be produced in the contacting mixture.

The term “binding agent” refers to an agent having a functional group capable of forming a covalent bond to a complementary functional group of another binding agent in a biological environment. Binding between binding agents in a biological environment may also be referred to as bioconjugation. Binding agents include bioorthogonal binding agents, which are binding agents having bioorthogonal functional groups. Bioorthogonal functional groups of bioorthogonal binding agents selectively react with a complementary bioorthogonal functional group of another bioorthogonal binding partner. Selective reaction between bioorthogonal binding partners can minimize side reactions with other binding agents, biological compounds, or other non-complementary bioorthogonal binding agents or non-complementary bioorthogonal functional groups. Bioorthogonal functional groups of bioorthogonal binding agents include, but are not limited to, an azide and alkyne for formation of a triazole via Click-chemistry reactions, trans-cyclooctene (TCO) and tetrazine (Tz) (e.g., 1,2,4,5-tetrazine), and others. The binding agents useful in the present disclosure may have a high reactivity with the corresponding binding agent so that the reaction is rapid.

The term “functionalized” refers to a moiety having a functional group attached to the moiety, such as for example a moiety having a binding agent functional group (e.g., a bioorthogonal functional group) attached thereto.

The term “administering” refers to any suitable route of administration to a subject, such as, but not limited to, oral administration, administration as a suppository, topical contact, parenteral, intravenous, intraperitoneal, intramuscular, intralesional, intranasal or subcutaneous administration, intrathecal administration, or the implantation of a slow-release device e.g., a mini-osmotic pump, to the subject.

The term “parenterally,” as used herein, refers to modes of administration which include intravenous, intramuscular, intraperitoneal, intrasternal, subcutaneous and intraarticular injection and infusion.

The term “leaving group” refers to an atom (or a group of atoms) with electron withdrawing ability that can be displaced as a stable species, taking with it the bonding electrons. Examples of suitable leaving groups include halides (e.g., Br, Cl, I), sulfonate esters (e.g., triflate, mesylate, tosylate, and brosylate), and nitrophenols.

The term “pharmaceutically effective amount” and “therapeutically effective amount” refer to an amount of a compound sufficient to treat a specified disorder or disease or one or more of its symptoms and/or to prevent or reduce the risk of the occurrence or reoccurrence of the disease or disorder or symptom(s) thereof. In reference to tumorigenic proliferative disorders, a pharmaceutically or therapeutically effective amount comprises an amount sufficient to, among other things, cause the tumor to shrink or decrease the growth rate of the tumor.

As used herein, the term “subject,” “patient,” or “organism” includes humans and mammals (e.g., mice, rats, pigs, cats, dogs, and horses). Typical subjects to which an agent(s) of the present disclosure may be administered may include mammals, particularly primates, especially humans. For veterinary applications, suitable subjects may include, for example, livestock such as cattle, sheep, goats, cows, swine, and the like; poultry such as chickens, ducks, geese, turkeys, and the like; and domesticated animals particularly pets such as dogs and cats. For diagnostic or research applications, suitable subjects may include mammals, such as rodents (e.g., mice, rats, hamsters), rabbits, primates, and swine such as inbred pigs and the like.

The term “treating” or “treatment” as used herein means the treating or treatment of a disease or medical condition or symptom(s) thereof in a patient, such as a mammal (particularly a human) that includes: (a) ameliorating the disease or medical condition or symptom(s) thereof, such as, eliminating or causing regression of the disease or medical condition or symptom(s) thereof in a patient; (b) suppressing the disease or medical condition or symptom(s) thereof, for example by, slowing or arresting the development of the disease or medical condition or symptom(s) thereof in a patient; or (c) alleviating a symptom of the disease or medical condition or symptom(s) thereof in a patient.

The term “physiological conditions” is meant to encompass those conditions compatible with living cells, e.g., predominantly aqueous conditions of a temperature, pH, salinity, etc. that are compatible with living cells.

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed, to the extent that such combinations embrace subject matter that are, for example, compounds that are stable compounds (i.e., compounds that can be made, isolated, characterized, and tested for biological activity). In addition, all sub-combinations of the various embodiments and elements thereof (e.g., elements of the chemical groups listed in the embodiments describing such variables) are also specifically embraced by the present invention and are disclosed herein just as if each and every such sub-combination was individually and explicitly disclosed herein.

### 2. Compositions

A. Trans-Cyclooctene Functionalized Payloads

Trans-cyclooctene functionalized payloads of the present disclosure include a compound of formula (I), wherein D, R^{1a}, R^{1b} L¹ m and p are as defined herein.

Compounds of formula (I) may have formula (I-A), wherein D, R^{1a}, R^{1b}, L¹, m and p are as defined herein.

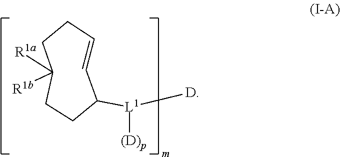

In the compounds described herein, R^{1a} and R^{1b} may be hydrogen.

In the compounds described herein, R^{1a} is C₁₋₄alkyl; and R^{1b} may be selected from the group consisting of C(O)OH, C(O)OC₁₋₄alkyl, C(O)N(R^{1c})CHR^{1e}CO₂H, C(O)N(R^{1c})CHR^{1e}C(O)OC₁₋₄alkyl, C(O)N(R^{1c})—C₁₋₆alkylene-CO₂H, and C(O)N(R^{1c})—C₁₋₆alkylene-C(O)OC₁₋₄alkyl. R^{1b} may be further selected from the group consisting of C(O)OH, C(O)N(R^{1c})CHR^{1e}CO₂H, and C(O)N(R^{1c})CH₂CO₂H.

In the compounds described herein, R^{1e} may be —CH₂CO₂H, —CH₂CH₂CO₂H, —CH₂CONH₂, —CH₂CH₂CONH₂, —CH₂OH, or —CH(CH₃)OH; or R^{1e} may be —C₁₋₄alkylene-CO₂H; or R^{1e} is —CH₂CO₂H.

In the compounds described herein, R^{1a} may be hydrogen.

In the compounds described herein, R^{1a} may be C₁₋₄alkyl.

In the compounds described herein, R^{1a} may be CH₃.

In the compounds described herein, R^{1b} may be hydrogen.

In the compounds described herein, R^{1b} may be C(O)N(R^{1c})—C₁₋₆alkylene-CO₂H.

In the compounds described herein, R^{1b} may be C(O)N(R¹⁰)CH₂CO₂H.

In the compounds described herein, R^{1b} may be C(O)OH.

In the compounds described herein, R^{1c} may be hydrogen.

Trans-cyclooctene functionalized payloads of the present disclosure include a compound of formula (I-B), or a pharmaceutically acceptable salt thereof,

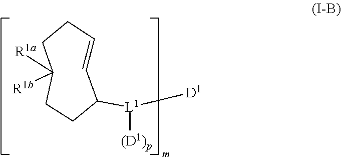

wherein D¹, at each occurrence, is independently a payload selected from an anticancer drug payload, a microbial immunosuppressive drug payload, an anti-restenosis drug payload, antibiotic drug payload, antifungal drug payload, antiviral drug payload, anti-inflammatory/anti-arthritic drug payload, a corticosteroid drug payload, and an immunosuppressant drug payload; and R^{1a}, R^{1b}, L¹, and m are as defined herein for formula (I-B). For example, p may be 0; with m being 1; and L¹ being

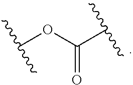 [SMILES: *OC(*)=O]

In some embodiments, the anticancer drug is doxorubicin.

Trans-cyclooctene functionalized payloads of the present disclosure include a compound of formula (II-A), wherein D¹, R^{1A}, R^{1B}, L², m and p are as defined herein.

Compounds of formula (II-A) may have formula (II-A′), wherein D¹, R^{1A}, R^{1B}, L¹, m and p are as defined herein.

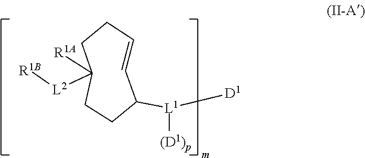

In some embodiments, R^{1B} is selected from the group consisting of G¹, OH, —NR_{1c}—C₁₋₄alkylene-G¹, NR^{1c}C₁₋₄alkylene-N(R^{1d})₂, —N(R^{1c})CHR^{1e}CO₂H, —N(R^{1c})CH₂CO₂H, and —N(R^{1f})—CH₂CH₂—(N(CH₂CO₂H)CH₂CH₂)ₙ—N(CH₂CO₂H)₂; R^{1e} is —CH₂CO₂H, —CH₂CH₂CO₂H, —CH₂CONH₂, —CH₂CH₂CONH₂, —CH₂OH, or —CH(CH₃)OH; and R^{1f} is hydrogen or CH₂CO₂H, wherein n, G¹ and R^{1c} are as defined herein.

In some embodiments, R^{1A} is C₁₋₄ alkyl; R^{1B} is selected from the group consisting of G¹, OH, —NR^{1c}—C₁₋₄alkylene-G¹, —NR^{1c}—C₁₋₄alkylene-N(R^{1d})₂, —N(R^{1c})CHR^{1e}CO₂H, —N(R^{1c})CH₂CO₂H, and —N(R^{1f})—CH₂CH₂—(N(CH₂CO₂H)CH₂CH₂)ₙ—N(CH₂CO₂H)₂; R^{1e} is —C₁₋₄alkylene-CO₂H; R^{1f} is hydrogen or C₁₋₄alkylene-CO₂H; G¹ is a 4- to 8-membered monocyclic heterocyclyl containing a first nitrogen and optionally one additional heteroatom selected from nitrogen, oxygen, and sulfur, G¹ being attached at the first nitrogen and optionally substituted with 1-4 substituents independently selected from the group consisting of C₁₋₄alkyl, C₁₋₄haloalkyl, halo, cyano, OH, —OC₁₋₄alkyl, and oxo; and n is 0, 1, or 2, wherein R^{1c} and R^{ed} are as defined herein.

In some embodiments, R^{1A} is CH₃; R^{1e} is —CH₂CO₂H; R^{1f} is hydrogen or CH₂CO₂H; and G¹ is a piperazinyl (e.g., piperazin-1-yl), morpholinyl (e.g., morpholin-4-yl), piperidinyl (e.g., piperidin-1-yl), azepanyl (e.g., azepan-1-yl), or pyrrolidinyl (e.g., pyrrolidin-1-yl), attached through a ring nitrogen atom and optionally substituted with 1-4 substituents independently selected from the group consisting of C₁₋₄ alkyl, C₁₋₄ haloalkyl, halo, cyano, OH, —OC₁₋₄alkyl, and oxo.

In some embodiments, L² is —C(O)—.

In some embodiments, R^{1B} is selected from the group consisting of OH, N(H)CH₂CO₂H, —N(H)CHR^{1e}CO₂H, —N(H)—CH₂CH₂—(N(CH₂CO₂H)CH₂CH₂)ₙ—N(CH₂CO₂H)₂, and —N(CH₂CO₂H)—CH₂CH₂—N(CH₂CO₂H)₂; and R^{1e} is —CH₂CO₂H.

In some embodiments, L² is —C(O)—; R^{1A} is C₁₋₄alkyl; R^{1B} is OH, —N(R^{1c})CHR^{1e}CO₂H, —N(R^{1c})—C₁₋₆alkylene-CO₂H, or —N(R^{1f})—C₂₋₄alkylene-(N(C₁₋₄alkylene-CO₂H)—C₂₋₄alkylene)ₙ-N(C₁₋₄alkylene-CO₂H)₂; R^{1c} is hydrogen or C₁₋₄alkyl; R^{1e} is —C₁₋₄alkylene-CO₂H; R^{1f} is hydrogen or C₁₋₄alkylene-CO₂H; and m, n, p, D¹, and L¹ are as defined herein.

In some embodiments, L² is —C(O)—; R^{1A} is C₁₋₄alkyl; R^{1B} is OH, —N(R^{1c})CHR^{1e}CO₂H, —N(R^{1c})CH₂CO₂H, or —N(R^{1f})—CH₂CH₂—(N(CH₂CO₂H)CH₂CH₂)ₙ—N(CH₂CO₂H)₂; R^{1c} is hydrogen or C₁₋₄alkyl; R^{1e} is —C₁₋₄alkylene-CO₂H; R^{1f} is hydrogen or C₁₋₄alkylene-CO₂H; and m, n, p, D¹, and L¹ are as defined herein.

In further embodiments, L² is —C(O)—; R^{1A} is CH₃; R^{1B} is OH, —N(R^{1c})CHR^{1e}CO₂H, —N(R^{1c})CH₂CO₂H, or —N(R^{1f})—CH₂CH₂—(N(CH₂CO₂H)CH₂CH₂)ₙ—N(CH₂CO₂H)₂; R^{1e} is —CH₂CO₂H, —CH₂CH₂CO₂H, —CH₂CONH₂, —CH₂CH₂CONH₂, —CH₂OH, or —CH(CH₃)OH; R^{1f} is hydrogen or CH₂CO₂H, R^{1c} is hydrogen or CH₃; and m, n, p, D¹, and L¹ are as defined herein.

In still further embodiments, L² is —C(O)—; R^{1A} is CH₃; R^{1B} is OH, N(H)CH₂CO₂H, —N(H)CHR^{1e}CO₂H, —N(H)—CH₂CH₂—(N(CH₂CO₂H)CH₂CH₂)ₙ—N(CH₂CO₂H)₂, or —N(CH₂CO₂H)—CH₂CH₂—N(CH₂CO₂H)₂; lee is —CH₂CO₂H; and m, n, p, D¹, and L¹ are as defined herein.

Trans-cyclooctene functionalized payloads of the present disclosure include a compound of formula (III-A), wherein D¹, R^{1B} L¹, L² m and p are as defined herein for formula (II-A) and (II-A′).

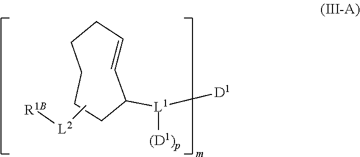

In the compounds described herein, linker L¹ may have 1 to 100 linking atoms, and may include ethylene-oxy groups, amines, esters, amides, carbamates, carbonates, and ketone functional groups. For example, linkers may have from 1 to 50 linking atoms, or from 5 to 50 linking atoms, or from 10 to 50 linking atoms.

The linker may be a non-releasable linker. A non-releasable linker is a linker that forms an attachment between at least two moieties, where the attachment is not significantly disrupted under the conditions that compositions using the non-releasable linker are used (e.g., covalent bonds in the linker remain intact and are not cleaved).

The linker may be a releasable linker. A releasable linker is a linker that forms an attachment between at least two moieties, where the attachment may be disrupted under releasing conditions such that the moieties are no longer attached to each other (e.g., one or more covalent bonds in the linker may be cleaved). Releasable linkers may have the attachment between the moieties disrupted by exposure of the releasable linker to releasing conditions, such as, but not limited to, light, heat, sound, a releasing agent (e.g., chemical releasing agent (e.g., an acid, a base, an oxidizing agent, a reducing agent), a solvent, an enzyme, etc.), combinations thereof, and the like. In some embodiments, the releasable linker may not require the application of an external stimulus or contact with releasing conditions to disrupt the attachment between the moieties. For example, a releasable linker may include one or more unstable bonds or functional groups in the linker that can be cleaved spontaneously without contact with an external stimulus or releasing conditions, thereby releasing the payload from the support composition. Examples of bonds or functional groups that can be spontaneously cleaved as described above include, but are not limited to, carbamates, which release carbon dioxide upon spontaneous cleavage. Functionalized payloads of the present disclosure that include a releasable linker may facilitate delivery of a payload to a target location in a subject.

In some cases, the payload may be released as described above by contacting the releasable linker to releasing conditions. The releasing conditions can be target specific, such as releasing conditions that are directly applied to a desired target location in a subject (e.g., a target location where the therapeutic support composition is present). In some embodiments, the releasing conditions may be non-specific, such as by exposure of the releasable linker to an extracellular mechanism (e.g., low pH in tumor tissue, hypoxia, enzymes, and the like). In other instances, release of the payload can be achieved through intracellular, such as lysosomal, release mechanisms (e.g., glutathione, proteases (e.g., cathepsin), catabolism, and the like). In these cases, the therapeutic support composition may be internalized within a cell and subsequently exposed to releasing conditions present within the cell. Intracellular releasing conditions (e.g., glutathione, cathepsin, and the like) may result in release of the payload from the therapeutic support composition such that the payload can be dispersed from the cell and provide a therapeutic effect on neighboring cells. Examples of these types of releasable linkers include, but are not limited to, hydrazones (acid labile), peptide linkers (cathepsin B cleavable), disulfide moieties (thiol cleavable), and the like. This type of release mechanism of action may facilitate providing treatment to diseases or conditions, such as tumors (e.g., tumors with heterogeneous receptor expression, or with poor mAb penetration).

In certain embodiments, the linker between the payload and the trans-cyclooctene is an immolative linker.

In certain embodiments, the linker between the payload and the trans-cyclooctene is a pH tunable linker.

In some instances, the therapeutic agent is covalently attached to the linker through an amide bond; e.g., the therapeutic agent may be an amine-containing therapeutic agent for attachment of the therapeutic agent to a carbonyl group of the linker, or, in other cases, the therapeutic agent may be a carboxyl-containing therapeutic agent for attachment of the therapeutic agent to an amine group of the linker. In some instances, the therapeutic agent and linker, together form a carbamate group; e.g., the therapeutic agent may be an amine-containing therapeutic agent for attachment of the therapeutic agent to an acyloxy group of the linker. In some instances, the therapeutic agent and linker, together form a carbonate group; e.g., the therapeutic agent may be a hydroxy-containing therapeutic agent for attachment of the therapeutic agent to an acyloxy group of the linker.

For example, in the compounds described herein, L¹ may be or

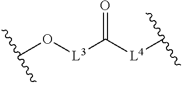

—O—; wherein L³ is a bond or C₁₋₆alkylene; L⁴ is a bond, —NHN; —N(R¹⁰)—C₂₋₆ alkylene-N(R¹¹)—, —N(R¹²)—C₂₋₃ alkylene-N(R¹³)C(O)—, —N(R¹⁰)—C₁₋₆alkylene-C(O)NHN; —NHNHC(O)C₁₋₆ alkylene-C(O)NHN; —CH(NHC(O)R¹⁴)C₁₋₄alkylene-S—S—C₁₋₄alkylene-OC(O)—, —NHNHC(O)CH(NHC(O)R¹⁵)CH₂C(O)—, —C₁₋₆alkylene-CH(G^{x})OC(O)—,

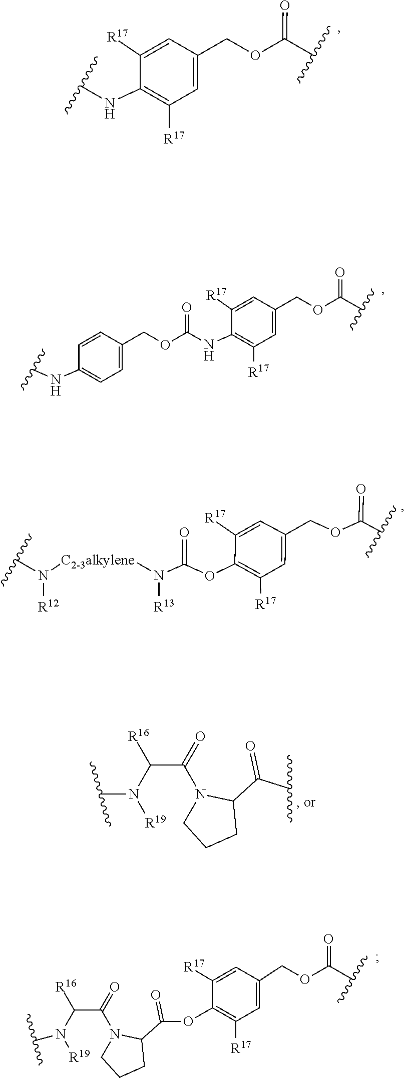

R¹⁰, R¹¹, R¹², R¹³, R¹⁴, R¹⁵ and R¹⁹ are each independently hydrogen or C₁₋₄alkyl; R¹⁶ is hydrogen, C₁₋₄alkyl, —C₁₋₄alkylene-OH, —C₁₋₄ alkylene-OC₁₋₄ alkyl, —C₁₋₄alkylene-CO₂H, or —C₁₋₄ alkylene-CONH₂; R¹⁷, at each occurrence, is independently hydrogen or —CH₂OC(O)—; and G^{x} is phenyl optionally substituted with 1-5 substituents independently selected from the group consisting of halogen, C₁₋₄alkyl, C₁₋₄haloalkyl, C₁₋₄ alkoxy, cyano, and nitro.

In the compounds described herein, m may be 1. Where m is 1,

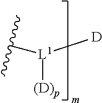

may be

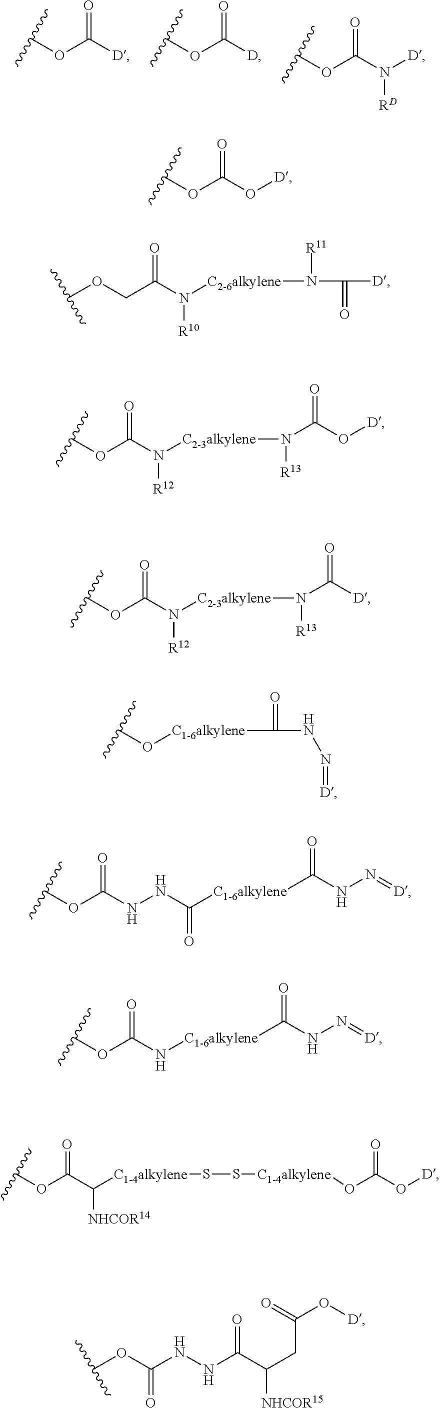

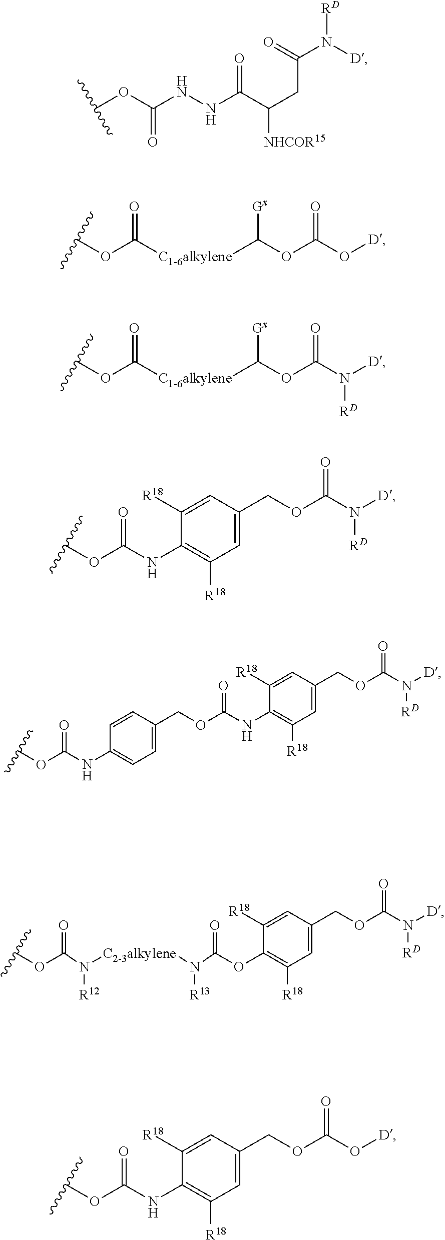

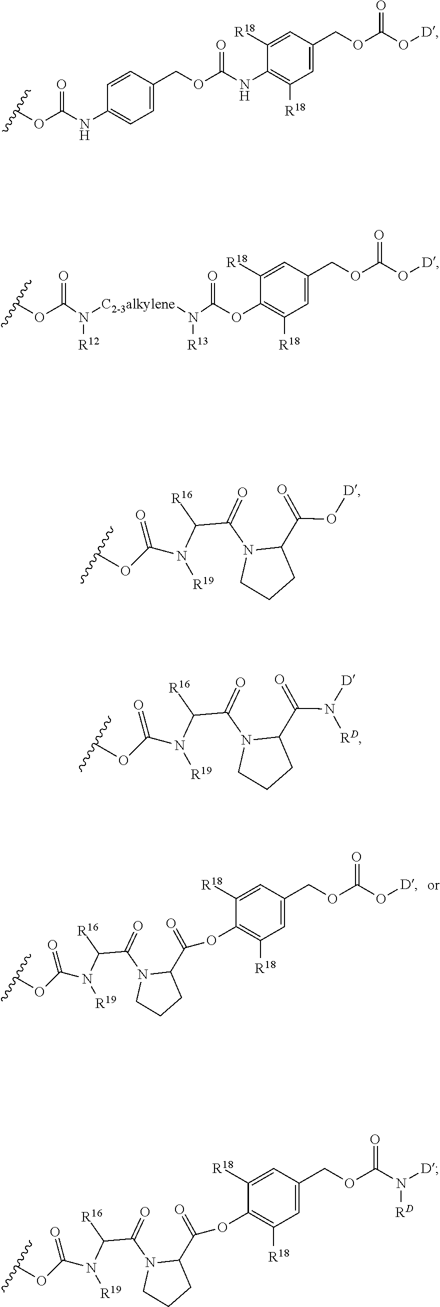

wherein R¹⁸, at each occurrence, is independently hydrogen or —CH₂OC(O)NHD′; R^{D} is hydrogen or C₁₋₄ alkyl on a nitrogen atom of the payload; and D′ is a payload moiety (e.g., cyclic dinucleotide payload moiety, imidazo[4,5-c]quinolin-4-amine payload moiety, TLR agonist payload moiety, STING agonist payload moiety).

In the compounds described herein,

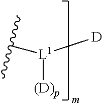

may be

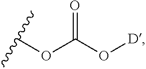

wherein D′ is a cyclic dinucleotide payload moiety.

Where m is 1,

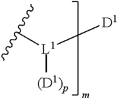

may be

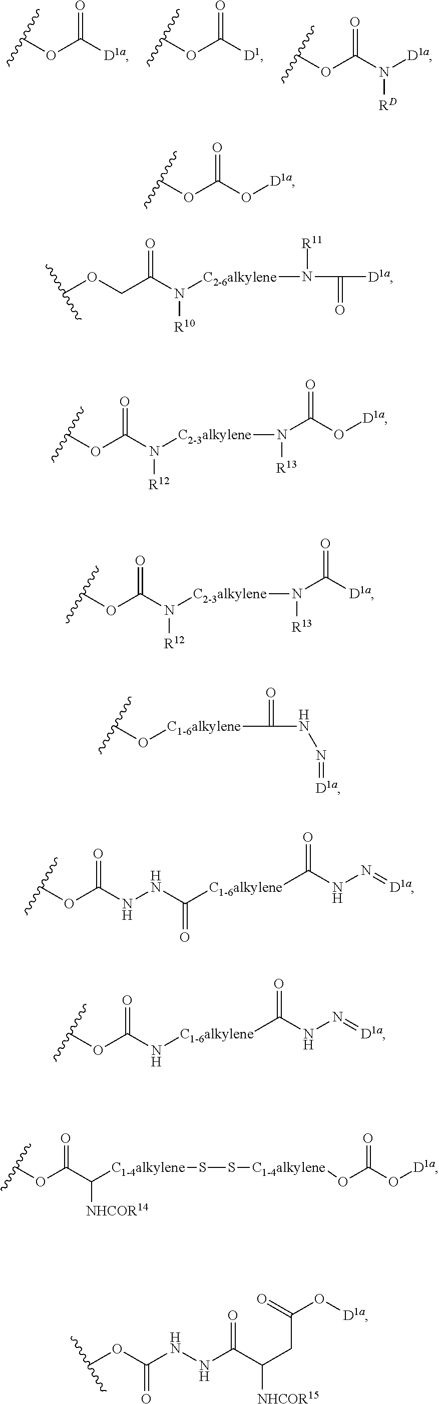

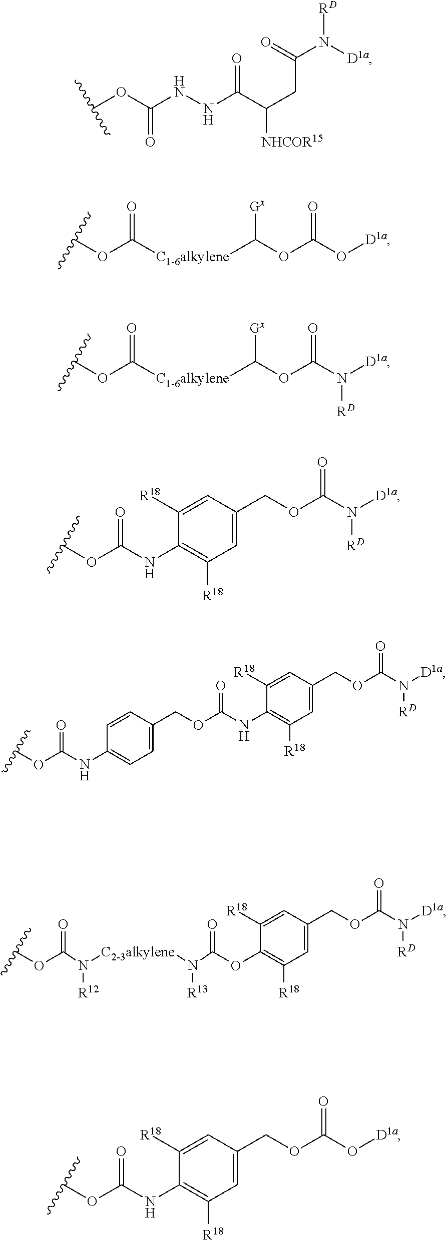

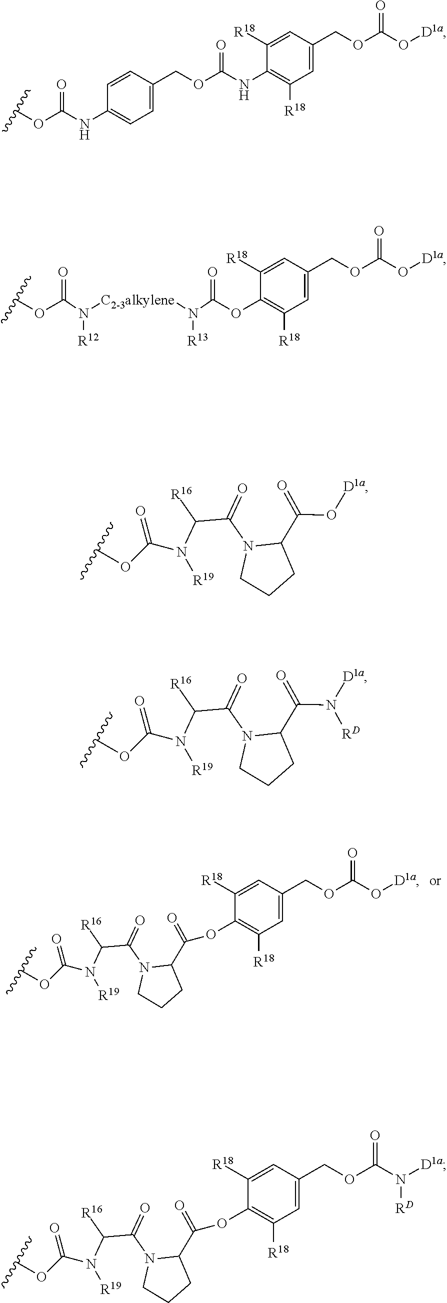

wherein R¹⁸, at each occurrence, is independently hydrogen or —CH₂OC(O)NHD^{1a}; R^{D} is hydrogen or C₁₋₄alkyl on a nitrogen atom of the payload; and D^{1a} is a payload moiety (e.g., an anti-cancer payload moiety).

The person skilled in the art will recognize that a payload D/D¹ bonded to a linker does not refer to a payload molecule per se, but refers to the portion of the payload molecule bonded to the linker. Release of the payload D/D¹ from a compound herein, releases the payload per se.

A “payload moiety” as used herein refers to a payload DID′ minus its nucleophilic group such as NH, NC₁₋₄alkyl, O, or S that attaches to a linker or minus its electrophilic group such as C(O) that attaches to a linker, i.e., the remainder of the payload. For example, a compound of formula

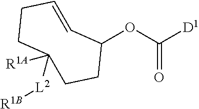

includes a compound such as

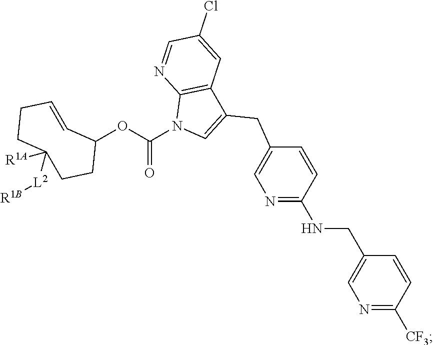

a compound

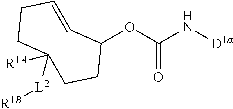

includes a compound such as

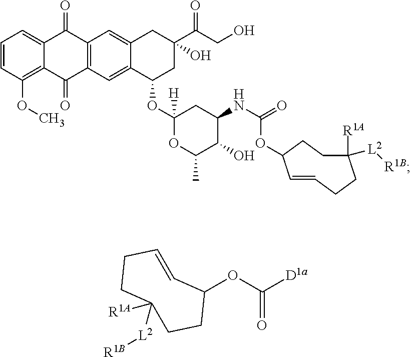

includes a compound such as a

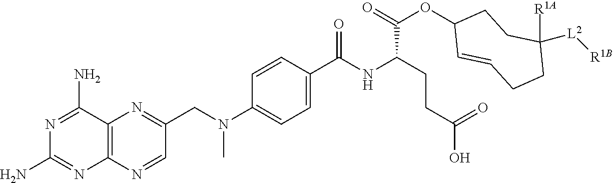

compound of formula

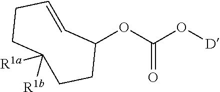

includes a compound such as

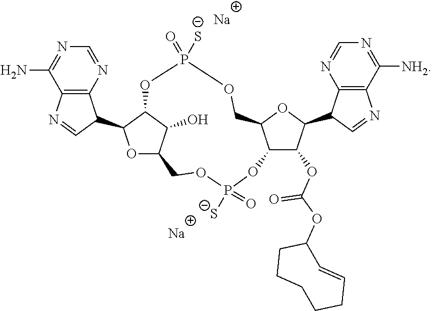 [SMILES: [Na+] ; Nc1ncnc2c1N=CC2[C@@H]1O[C@@H]2COP(=O)([S-])O[C@H]3[C@@H](OC(=O)OC4/C=C/CCCCC4)[C@H](C4C=Nc5c(N)ncnc54)O[C@@H]3COP(=O)([S-])O[C@@H]1[C@@H]2O ; [Na+]]

Release of D′H, NH₂-D¹a, HOOC-D^{1a}, or HO-D′ releases the payload molecule per se.

In the compounds described herein, p may be 0.

In the compounds described herein, m may be 2 or 3. In some embodiments, m is 2 and

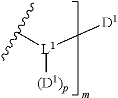

is

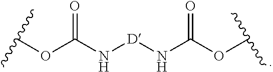

In some embodiments, m is 2 and

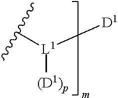

is

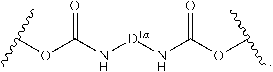

D¹ may be a drug payload selected from an anticancer drug payload, a microbial immunosuppressive drug payload, or an anti-restenosis drug payload. The anticancer drug may be one or more selected from methotrexate, purines, pyrimidines, plant alkaloids, epothilones, triptolide compounds, antibiotics (notably actinomycin D), hormones and antibodies. From among the plant alkaloids, mention may notably be made of paclitaxel, doxorubicin, maytansin, auristatin, calicheamycin, duocarmycin, tubulysin and camptothecin. The microbial immunosuppressive drug may be one or more selected from cyclosporin A, tacrolimus and its analogues, despergualin, mycophenolate esters, rapamycin and its derivatives, FR-900520 substance from Streptomyces strains, FR-900523 substance from Streptomyces strains, daclizumab, pentanamide, kanglemycin C, spergualin, prodigiosin-25C, tranilast, myriocin, cyclosporin C, bredinin, mycophenolic acid, brefeldin A and ketosteroids. The anti-restenosis drug may be one or more selected from batimastat, metalloproteinase inhibitors, 17β-estradiol, NO donors, 2-chlorodeoxyadeno sine, 2-deoxycoformycin, fingolimod, mycophenolate sodium, ISA_{TX}247 (a cyclosporin A derivative), elsibucol, daclizumab, basiliximab, anti-thymocyte globulin, everolimus, methotrexate, neoral, cyclophosphamide, brequinar sodium, leflunomide and mizoribine.

Exemplary anti-cancer drugs include, but are not limited to, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Adrucil (Fluorouracil), Afatinib Dimaleate, Afinitor (Everolimus), Aldara (Imiquimod), Aldesleukin, Alemtuzumab, Alimta (Pemetrexed Disodium), Aloxi (Palonosetron Hydrochloride), Ambochlorin (Chlorambucil), Amboclorin (Chlorambucil), Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Avastin (Bevacizumab), Axitinib, Azacitidine, BEACOPP, Bendamustine Hydrochloride, BEP, Bevacizumab, Bexarotene, Bexxar (Tositumomab and I 131 Iodine Tositumomab), Bicalutamide, Bleomycin, Bortezomib, Bosulif (Bosutinib), Bosutinib, Brentuximab Vedotin, Busulfan, Busulfex (Busulfan), Cabazitaxel, Cabozantinib-S-Malate, CAF, Campath (Alemtuzumab), Camptosar (Irinotecan Hydrochloride), Capecitabine, CAPDX, Carboplatin, Carboplatin-Taxol, Carfilzomib, Casodex (Bicalutamide), CeeNU (Lomustine), Cerubidine (Daunorubicin Hydrochloride), Cervarix (Recombinant HPV Bivalent Vaccine), Cetuximab, Chlorambucil, Chlorambucil-Prednisone, CHOP, Cisplatin, Clafen (Cyclophosphamide), Clofarabine, Clofarex (Clofarabine), Clolar (Clofarabine), CMF, Cometriq (Cabozantinib-S-Malate), COPP, COPP-ABV, Cosmegen (Dactinomycin), Crizotinib, CVP, Cyclophosphamide, Cyfos (Ifosfamide), Cytarabine, Cytarabine, Liposomal, Cytosar-U (Cytarabine), Cytoxan (Cyclophosphamide), Dabrafenib, Dacarbazine, Dacogen (Decitabine), Dactinomycin, Dasatinib, Daunorubicin Hydrochloride, Decitabine, Degarelix, Denileukin Diftitox, Denosumab, DepoCyt (Liposomal Cytarabine), DepoFoam (Liposomal Cytarabine), Dexrazoxane Hydrochloride, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Efudex (Fluorouracil), Elitek (Rasburicase), Ellence (Epirubicin Hydrochloride), Eloxatin (Oxaliplatin), Eltrombopag Olamine, Emend (Aprepitant), Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Etopophos (Etoposide Phosphate), Etoposide, Etoposide Phosphate, Evacet (Doxorubicin Hydrochloride Liposome), Everolimus, Evista (Raloxifene Hydrochloride), Exemestane, Fareston (Toremifene), Faslodex (Fulvestrant), FEC, Femara (Letrozole), Filgrastim, Fludara (Fludarabine Phosphate), Fludarabine Phosphate, Fluoroplex (Fluorouracil), Fluorouracil, Folex (Methotrexate), Folex PFS (Methotrexate), Folfiri, Folfiri-Bevacizumab, Folfiri-Cetuximab, Folfirinox, Folfox (Leucovorin, Fluorouracil, Oxaliplatin), Folotyn (Pralatrexate), FU-LV, Fulvestrant, Gardasil (Recombinant HPV Quadrivalent Vaccine), Gazyva (Obinutuzumab), Gefitinib, Gemcitabine Hydrochloride, Gemcitabine-Cisplatin, Gemcitabine-Oxaliplatin, Gemtuzumab Ozogamicin, Gemzar (Gemcitabine Hydrochloride), Gilotrif (Afatinib Dimaleate), Gleevec (Imatinib Mesylate), Glucarpidase, Goserelin Acetate, Halaven (Eribulin Mesylate), Herceptin (Trastuzumab), HPV Bivalent Vaccine, Recombinant, HPV Quadrivalent Vaccine, Recombinant, Hycamtin (Topotecan Hydrochloride), Hyper-CVAD, Ibritumomab Tiuxetan, Ibrutinib, ICE, Iclusig (Ponatinib Hydrochloride), Ifex (Ifosfamide), Ifosf amide, Ifosfamidum (Ifosfamide), Imatinib Mesylate, Imbruvica (Ibrutinib), Imiquimod, Inlyta (Axitinib), Intron A (Recombinant Interferon Alfa-2b), Iodine 131 Tositumomab and Tositumomab, Ipilimumab, Iressa (Gefitinib), Irinotecan Hydrochloride, Istodax (Romidepsin), Ixabepilone, Ixempra (Ixabepilone), Jakafi (Ruxolitinib Phosphate), Jevtana (Cabazitaxel), Kadcyla (Ado-Trastuzumab Emtansine), Keoxifene (Raloxifene Hydrochloride), Kepivance (Palifermin), Kyprolis (Carfilzomib), Lapatinib Ditosylate, Lenalidomide, Letrozole, Leucovorin Calcium, Leukeran (Chlorambucil), Leuprolide Acetate, Levulan (Aminolevulinic Acid), Linfolizin (Chlorambucil), LipoDox (Doxorubicin Hydrochloride Liposome), Liposomal Cytarabine, Lomustine, Lupron (Leuprolide Acetate), Lupron Depot (Leuprolide Acetate), Lupron Depot-Ped (Leuprolide Acetate), Lupron Depot-3 Month (Leuprolide Acetate), Lupron Depot-4 Month (Leuprolide Acetate), Marqibo (Vincristine Sulfate Liposome), Matulane (Procarbazine Hydrochloride), Mechlorethamine Hydrochloride, Megace (Megestrol Acetate), Megestrol Acetate, Mekinist (Trametinib), Mercaptopurine, Mesna, Mesnex (Mesna), Methazolastone (Temozolomide), Methotrexate, Methotrexate LPF (Methotrexate), Mexate (Methotrexate), Mexate-AQ (Methotrexate), Mitomycin C, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Nelarabine, Neosar (Cyclophosphamide), Neupogen (Filgrastim), Nexavar (Sorafenib Tosylate), Nilotinib, Nolvadex (Tamoxifen Citrate), Nplate (Romiplostim), Obinutuzumab, Ofatumumab, Omacetaxine Mepesuccinate, Oncaspar (Pegaspargase), Ontak (Denileukin Diftitox), OEPA, OPPA, Oxaliplatin, Paclitaxel, Paclitaxel Albumin-stabilized Nanoparticle Formulation, Palifermin, Palonosetron Hydrochloride, Pamidronate Disodium, Panitumumab, Paraplat (Carboplatin), Paraplatin (Carboplatin), Pazopanib Hydrochloride, Pegaspargase, Peginterferon Alfa-2b, PEG-Intron (Peginterferon Alfa-2b), Pemetrexed Disodium, Perj eta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Pralatrexate, Prednisone, Procarbazine Hydrochloride, Proleukin (Aldesleukin), Prolia (Denosumab), Promacta (Eltrombopag Olamine), Provenge (Sipuleucel-T), Purinethol (Mercaptopurine), Radium 223 Dichloride, Raloxifene Hydrochloride, Rasburicase, R-CHOP, R-CVP, Recombinant HPV Bivalent Vaccine, Recombinant HPV Quadrivalent Vaccine, Recombinant Interferon Alfa-2b, Regorafenib, Revlimid (Lenalidomide), Rheumatrex (Methotrexate), Rituxan (Rituximab), Rituximab, Romidepsin, Romiplostim, Rubidomycin (Daunorubicin Hydrochloride), Ruxolitinib Phosphate, Sclerosol Intrapleural Aerosol (Talc), Sipuleucel-T, Sorafenib Tosylate, Sprycel (Dasatinib), Stanford V, Sterile Talc Powder (Talc), Steritalc (Talc), Stivarga (Regorafenib), Sunitinib Malate, Sutent (Sunitinib Malate), Sylatron (Peginterferon Alfa-2b), Synovir (Thalidomide), Synribo (Omacetaxine Mepesuccinate), Tafinlar (Dabrafenib), Talc, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Temodar (Temozolomide), Temozolomide, Temsirolimus, Thalidomide, Thalomid (Thalidomide), Toposar (Etoposide), Topotecan Hydrochloride, Toremifene, Torisel (Temsirolimus), Tositumomab and 1 131 Iodine Tositumomab, Totect (Dexrazoxane Hydrochloride), Trametinib, Trastuzumab, Treanda (Bendamustine Hydrochloride), Trisenox (Arsenic Trioxide), Tykerb (Lapatinib Ditosylate), Vandetanib, VAMP, Vectibix (Panitumumab), VelP, Velban (Vinblastine Sulfate), Velcade (Bortezomib), Velsar (Vinblastine Sulfate), Vemurafenib, VePesid (Etoposide), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, Vismodegib, Voraxaze (Glucarpidase), Vorinostat, Votrient (Pazopanib Hydrochloride), Wellcovorin (Leucovorin Calcium), Xalkori (Crizotinib), Xeloda (Capecitabine), Xelox, Xgeva (Denosumab), Xofigo (Radium 223 Dichloride), Xtandi (Enzalutamide), Yervoy (Ipilimumab), Zaltrap (Ziv-Aflibercept), Zelboraf (Vemurafenib), Zevalin (Ibritumomab Tiuxetan), Zinecard (Dexrazoxane Hydrochloride), Ziv-Aflibercept, Zoladex (Goserelin Acetate), Zoledronic Acid, Zolinza (Vorinostat), Zometa (Zoledronic Acid), and Zytiga (Abiraterone Acetate).

In certain embodiments, the drug payload of D¹ is a PBD dimer, calicheamicin, speromycin, tubulysin B, rhizoxin, dolastatin, didemnin B, camptothecin, CBI, temsirolimus, actinomycin D, epothilone B, taxol, cryptophycin, SN38, velcade, bruceantin, DAVLBH, DM1, Phyllanthoside, Alimta, T2 Toxin, MMC, vantalanib, vinorelbine, brefeldin, sunitinib, daunomycin, semaxanib, tarceva, iressa, irinotecan, LY-541503, geldanomycin, gemcitabine, methotrexate, gleevec, topotecan, bleomycin, doxorubicin, cisplatin, N-mustards, etoposide, or 5-FU.

In certain embodiments, an anticancer drug is an anthracycline. In certain embodiments, anticancer drug is a taxane. In certain embodiments, anticancer drug is gemcitabine. In certain embodiments, anticancer drug is doxorubicin. In certain embodiments, anticancer drug is docetaxel. In certain embodiments, anticancer drug is SN38. In certain embodiments, anticancer drug is monomethyl auristatin E. In certain embodiments, the drug payload of D¹ is dexamethasone. In certain embodiments, the drug payload of D¹ is celecoxib. In certain embodiments, the drug payload of D¹ is gentamicin. In some embodiments, the drug payload of D¹ is vancomycin. In some embodiments, the drug payload of D¹ is daptomycin. In some embodiments, the drug payload of D¹ is doxorubicin. In some embodiments, the drug payload of D¹ is gemcitabine. In some embodiments, the drug payload of D¹ is docetaxel. In some embodiments, the drug payload of D¹ is cyclic-adenosine monophosphatidyl (c-AMP).

Particular

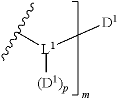

include

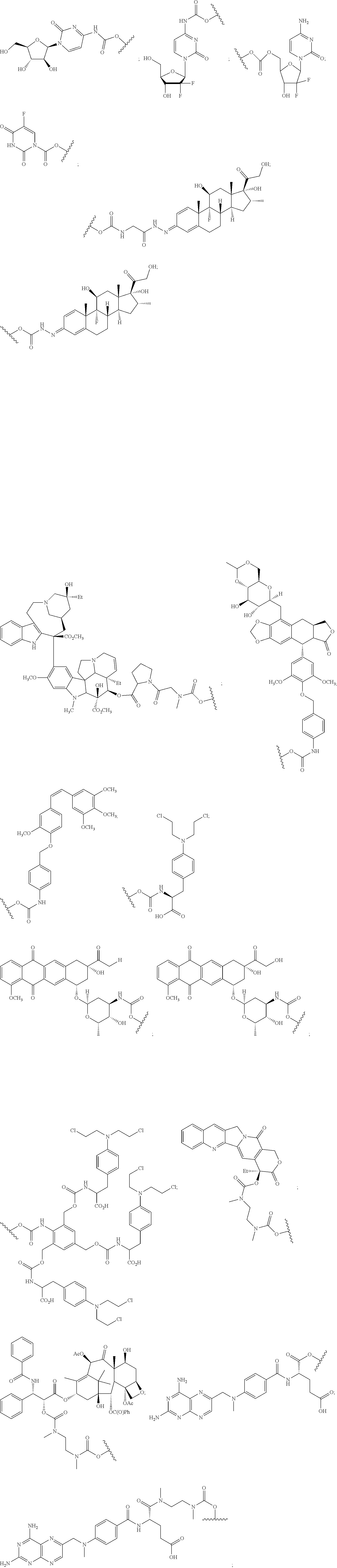

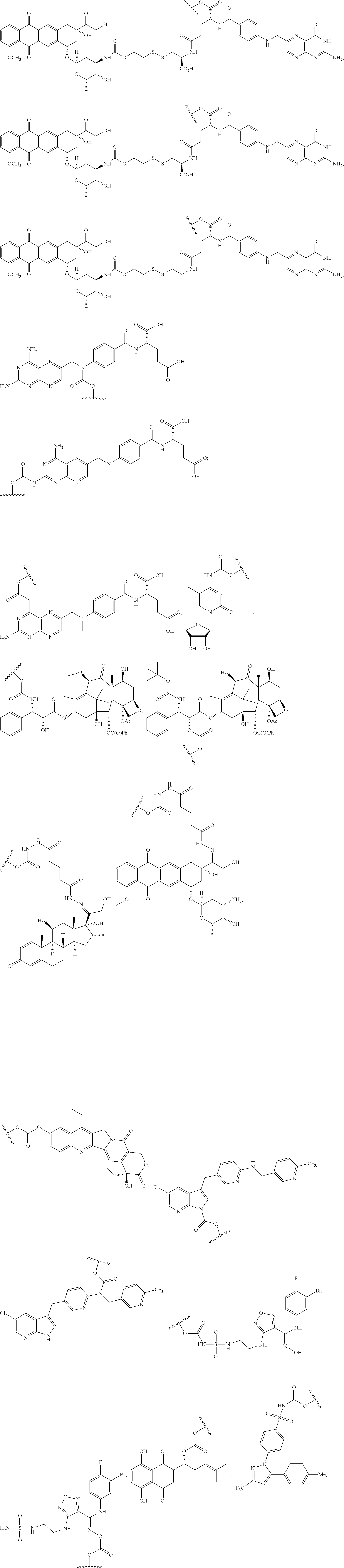

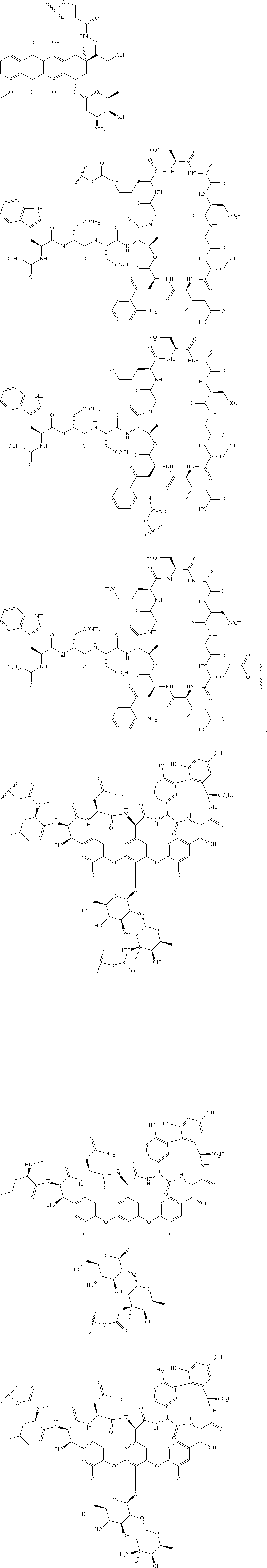

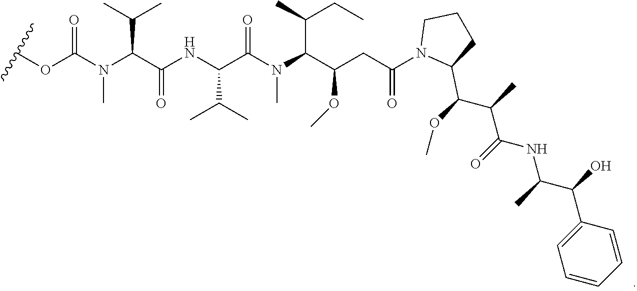 [SMILES: *OC(=O)N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

In any of the embodiments described herein are further embodiments wherein

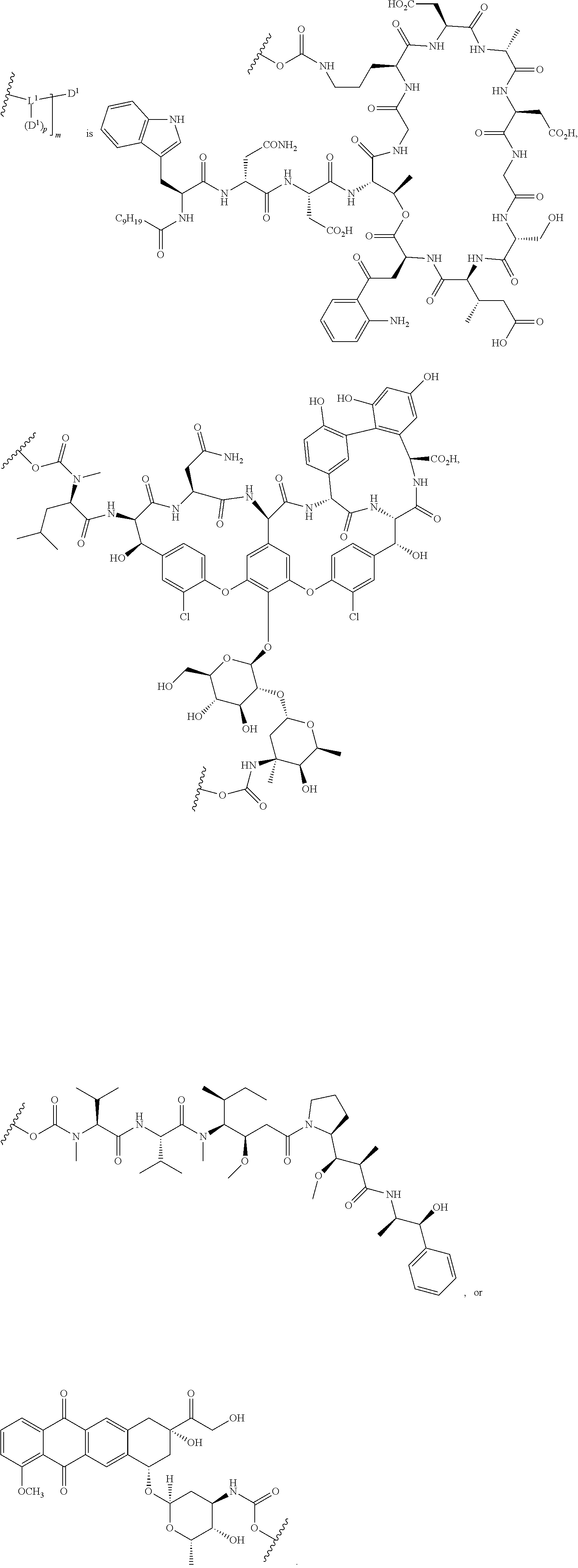

Preferred compounds of formula (I-B) and (II-A) include compounds of formula

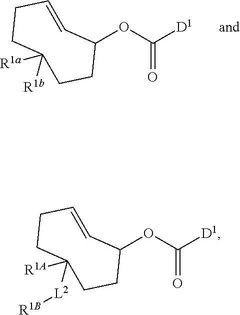

such as

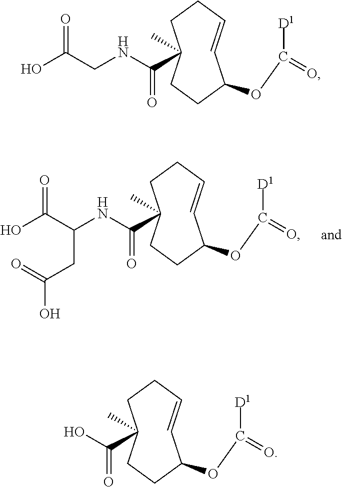

Preferred compounds of formula (I-B) and (II-A) include compounds of formula

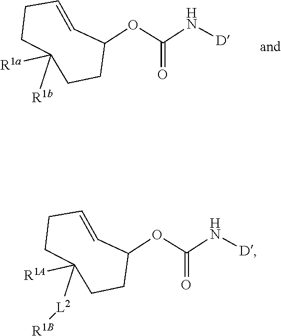

such as

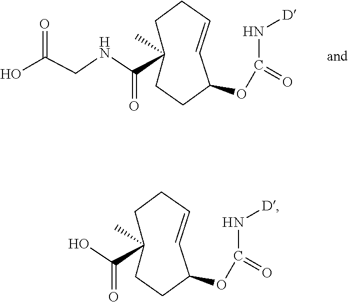

such as

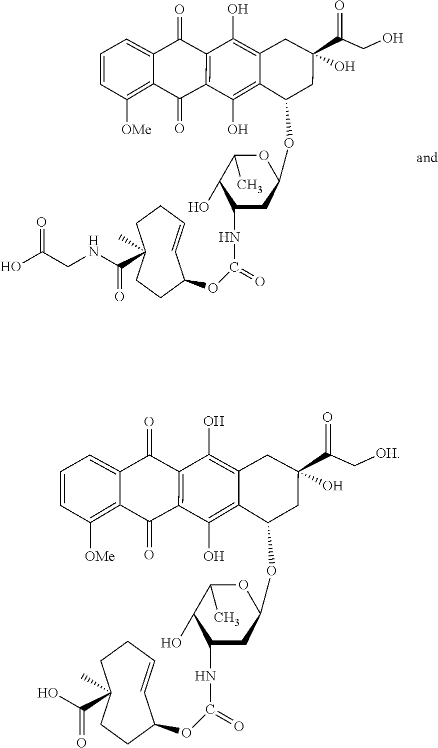

Preferred compounds of formula (I-A) include compounds of formula

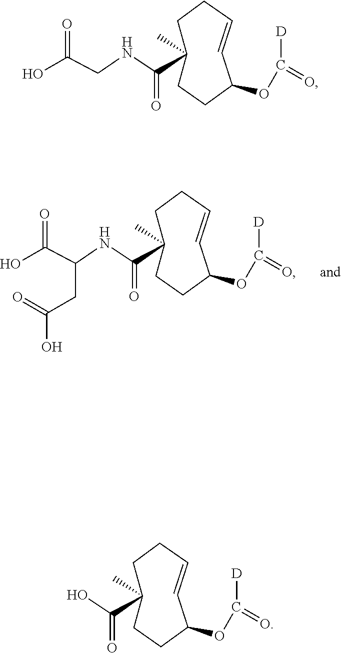

Payload D is a TLR agonist or STING agonist.

TLR agonists are immunomodulatory agents. TLR-mediated signaling in response to pathogen-associated molecular patterns (PAMPs) is a sequential cascade of transcriptional regulatory events that vary depending on the TLR agonists, cell types involved and pathogenicity of the antigen. Individual genes (notably proinflammatory cytokines, e.g., IL-1 (alpha and beta), IL-6, IL-18, TNF-C.) are induced transiently reflecting the ability that the innate immune system has to interpret the infection and orchestrate appropriate responses while promoting resolution (T. Ravasi, C. A. Wells, D. A. Hume, Bioessays 29, 1215 (Nov. 15, 2007); J. C. Roachet al., Proc Natl Acad Sci USA 104, 16245 (Oct. 9, 2007); M. Gilchrist et al., Nature 441, 173 (May 11, 2006)).

TLR agonists include but are not limited to agonists of TLR1/2 heterodimer (e.g., Pam3CSK4, i.e., TrispalmitoylCysSerLysLysLysLys), TLR3 (e.g., Poly I:C, Poly ICLC), TLR4 (e.g., monophosphoryl lipid A, lipopolysaccharide, GLA-SE, G100), TLR5 (e.g. flagellin), TLR2/6 heterodimer (e.g., diacyl lipopetides of gram positive bacteria, mycoplasma and fungi), TLR7 (e.g., imidazo[4,5-c]quinolin-4-amines such as imiquimod and as described in U.S. Pat. No. 4,689,338, which is incorporated herein by reference and polyriboinosinic-polyribocytidylic acid (Poly I:C)) TLR3 (Polyadenylic-polyuridylic acid (Poly A:U)), TLR2 (peptidoglycan), TLR2 and TLR4 (e.g. is Bacillus Calmette-Guerin (BCG)),

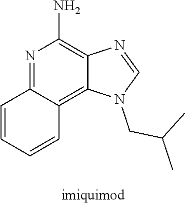 [SMILES: CC(C)Cn1cnc2c(N)nc3ccccc3c21]

TLR7/8 (e.g., loxoribine; imidazo[4,5-c]quinolin-4-amines such as resiquimod (R848) and MEDI9197),

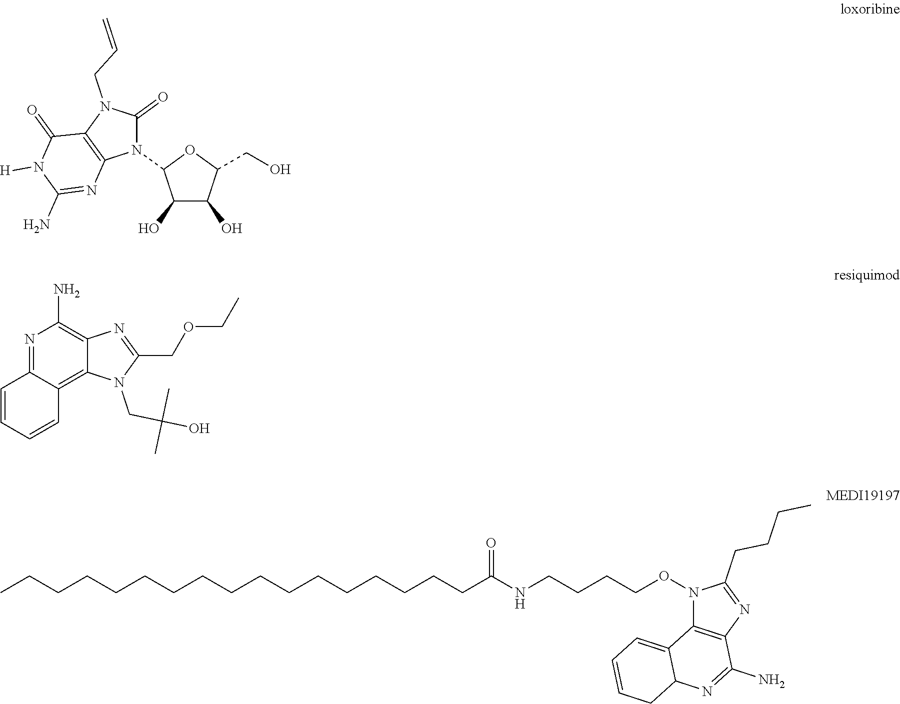

### TLR8 (e.g., VTX-2337)

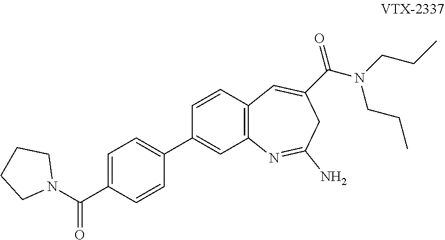 [SMILES: CCCN(CCC)C(=O)C1=Cc2ccc(-c3ccc(C(=O)N4CCCC4)cc3)cc2N=C(N)C1]

and TLR9 (e.g., CpG ODNs such as ODN D-SL01, MGN1703, CPG7909, SD-101, EMD 1201081). CpG ODNs are short synthetic single-stranded DNA molecules containing unmethylated CpG dinucleotides in particular sequence contexts (CpG motifs). CpG ODNs possess a partially or completely phosphorothioated (PS) backbone, as opposed to the natural phosphodiester (PO) backbone found in genomic bacterial DNA. Three major classes of stimulatory CpG ODNs have been identified based on structural characteristics and activity on human peripheral blood mononuclear cells (PBMCs), in particular B cells and plasmacytoid dendritic cells (pDCs). CpG-A ODNs are characterized by a PO central CpG-containing palindromic motif and a PS-modified 3′ poly-G string. They induce high IFN-α production from pDCs but are weak stimulators of TLR9-dependent NF-κB signaling and pro-inflammatory cytokine (e.g. IL-6) production. CpG-B ODNs contain a full PS backbone with one or more CpG dinucleotides. They strongly activate B cells and TLR9-dependent NF-κB signaling but weakly stimulate IFN-α secretion. CpG-C ODNs combine features of both classes A and B. They contain a complete PS backbone and a CpG-containing palindromic motif. C-Class CpG ODNs induce strong IFN-α production from pDC as well as B cell stimulation.

Further TLR9 agonists are described in WO2019/115402, EP2017281, US2019/0160173, US2019/0151345, US2011/0311518, US2011/0293565, each of which is incorporated herein by reference.

Single and double-stranded RNA may function as a TLR agonist, as described by Roers et al. in Immunity (2016) 44, 739-754, which is incorporated herein by reference.

STING agonists are immunomodulatory agents responsible for controlling numerous pro-inflammatory host defense genes, including type I inteferons, and pro-inflammatory cytokines, following the recognition of cyclic dinucleotides in the cytosol of a cell. These signals can then stimulate the adaptive immune system through cross presentation of antigen and T-cell priming, along with other mechanisms (Barber GN. STING: infection, inflammation and cancer. Nat Rev Immunol. 2015; 15(12):760-70) TLR and STING agonists are also capable of promoting anti-tumor immune responses in solid cancers and cancers being treated with immunotherapy (Berger G, Marloye M, Lawler S E. Immunotherapy. Trends Mol Med. 2019; 25(5):412-427).

STING agonists include ADU-S100 and 2′3′-cG^{S}A^{S}MP. STING agonists include cyclic dinucleotides and analogs thereof, such as

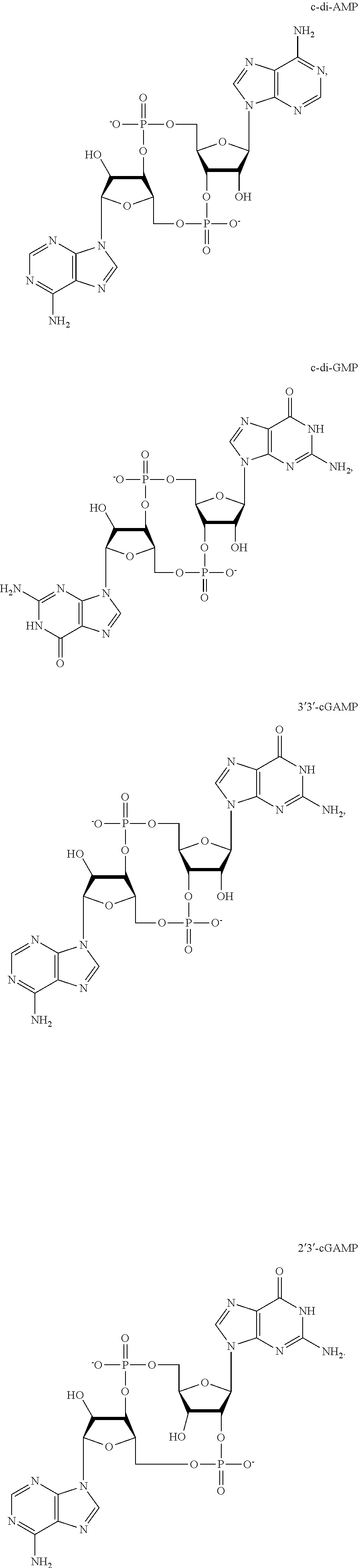

STING agonists further include modified cyclic dinucleotides. In some embodiments, the modified cyclic dinucleotide may not occur in nature or may be chemically synthesized. In some embodiments, the modified cyclic dinucleotide is a compound of the formula:

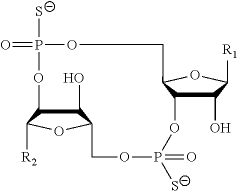

In some embodiments, R₁ and R₂ may each independently be 9-purine, 9-adenine, 9-guanine, 9-hypoxan thine, 9-Xanthine, 9-uric acid, or 9-isoguanine, the structures of which are shown below, the structures of which are:

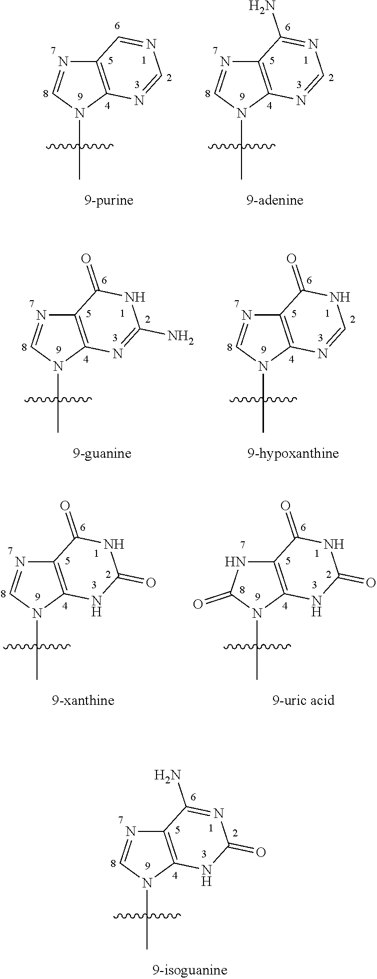

R₁ and R₂ may be identical or different. In some embodiments, the compound may be provided in the form of predominantly Rp.Rp or Rp.Sp. stereoisomers, or prodrugs, or pharmaceutically acceptable salts thereof, as described in US 2016/0287623, which is incorporated herein by reference. In some embodiments, the compound may be provided in the form of predominantly Rp.Rp stereoisomers. In particular embodiments, the compound may be a compound of the formula below or in the form of predominantly Rp.Rp stereoisomers thereof:

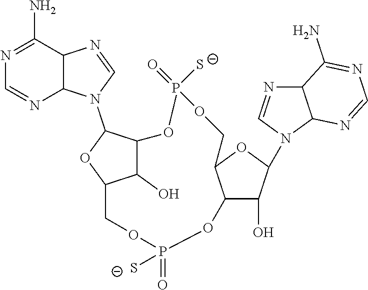 [SMILES: NC1=NC=NC2C1N=CN2C1OC2COP(=O)([S-])OC3C(O)C(COP(=O)([S-])OC2C1O)OC3N1C=NC2C(N)=NC=NC21]

STING agonists may include compounds of formula

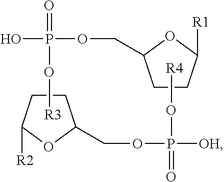

as described in US2017/0333552, which is incorporated herein by reference.

STING agonists may include compounds of formula

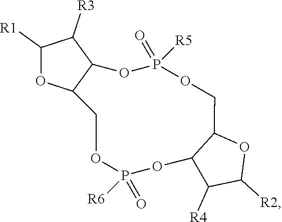

as described in US2018/0064745, which is incorporated herein by reference.

STING agonists may include compounds of formula

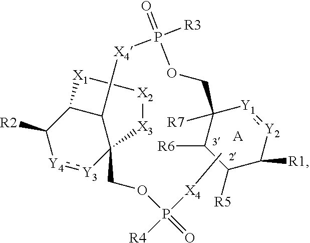

as described in US2019/0185511, which is incorporated herein by reference.

STING agonists may include compounds of formula

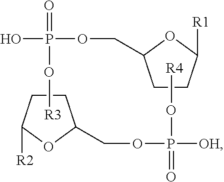

as described in WO2014/189806, which is incorporated herein by reference.

STING agonists may include compounds of formula

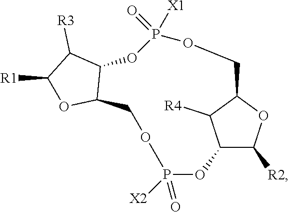

as described in US2019/0062365, which is incorporated herein by reference.

STING agonists may include compounds of formula

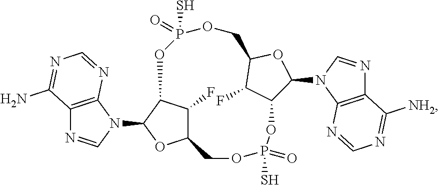 [SMILES: Nc1ncnc2c1ncn2[C@@H]1O[C@@H]2CO[P@@](=O)(S)O[C@@H]3[C@H](F)[C@@H](CO[P@@](=O)(S)O[C@@H]1[C@@H]2F)O[C@H]3n1cnc2c(N)ncnc21]

as described in WO2018/198076, which is incorporated herein by reference.

STING agonists may include compounds of formula

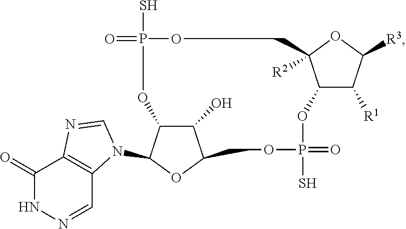

as described in US2018/0092937, which is incorporated herein by reference.

STING agonists may include compounds of formula

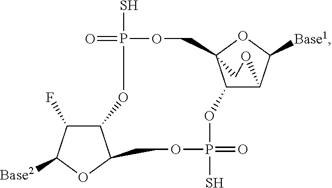

as described in US2018/0273578, which is incorporated herein by reference.

STING agonists may include compounds of formula

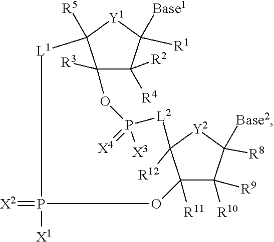

as described in US2019/0183917, which is incorporated herein by reference.

STING agonists may include compounds of formula

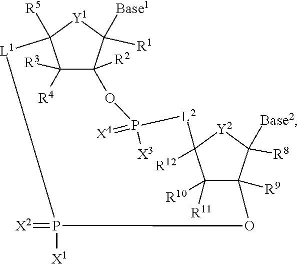

as described in US2019/0185509, which is incorporated herein by reference.

STING agonists may include compounds of formula

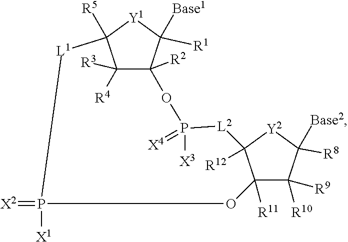

as described in US2019/0185510, which is incorporated herein by reference.

STING agonists may include compounds of formula

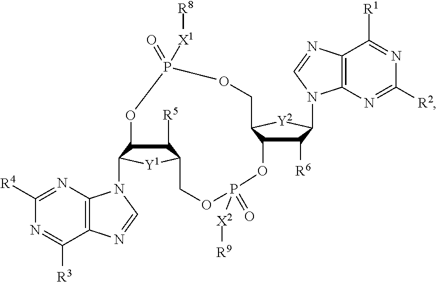

as described in US2017/0233430, which is incorporated herein by reference.

STING agonists may include compounds of formula

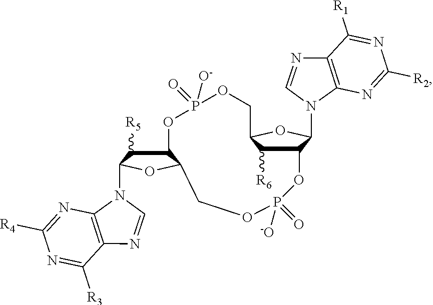

as described in US2018/0002369, which is incorporated herein by reference.

STING agonists may include compounds of formula

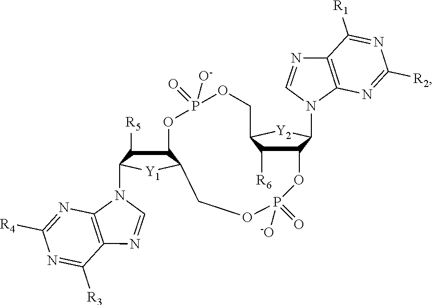

as described in US2018/0186828, which is incorporated herein by reference.

STING agonists may include compounds of formula

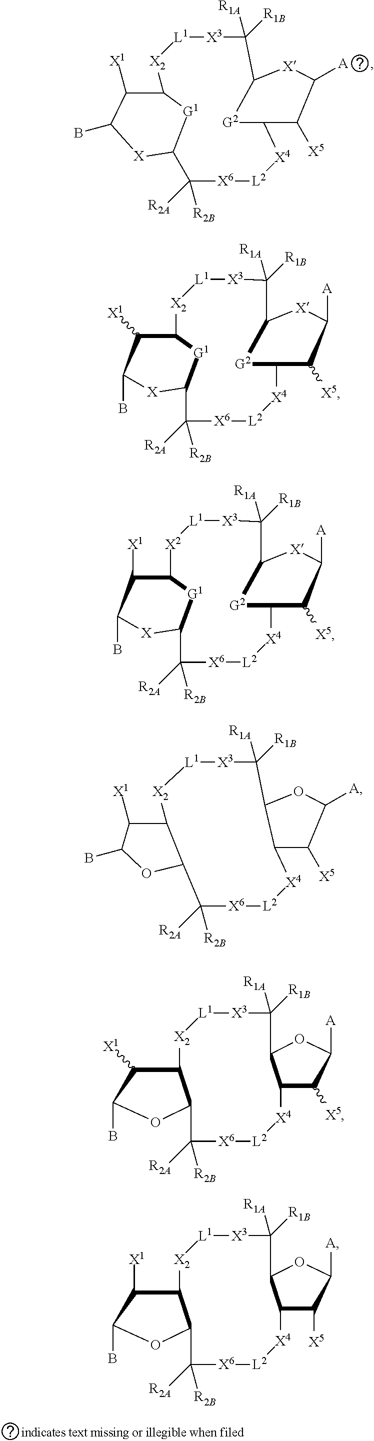

as described in US2019/0016750, which is incorporated herein by reference.

STING agonists may include compounds of formula

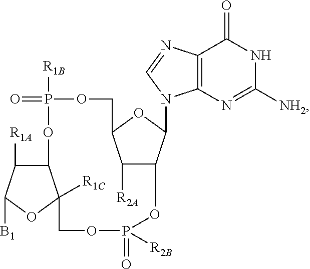

as described in US2018/0162899, which is incorporated herein by reference.

STING agonists may include compounds of formula

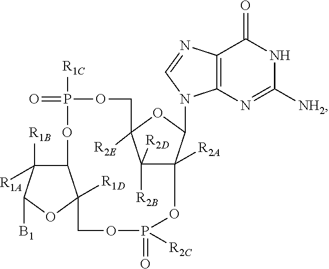

as described in WO2018/138684, which is incorporated herein by reference.

STING agonists may include compounds of formula

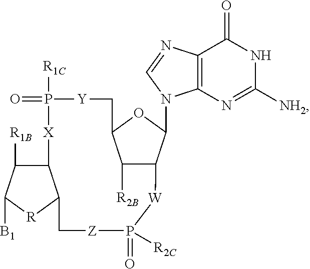

as described in WO2018/138685, which is incorporated herein by reference.

STING agonists may include compounds of formula

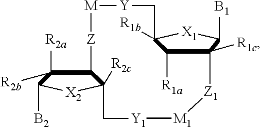

as described in WO2019/118839, which is incorporated herein by reference.

STING agonists may include compounds of formula

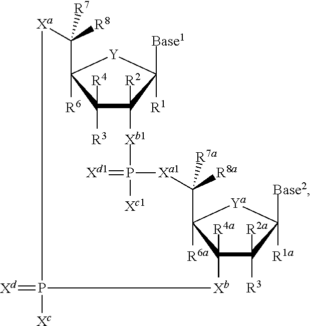

as described in US2017/0044206, which is incorporated herein by reference.

STING agonists may include compounds of formula

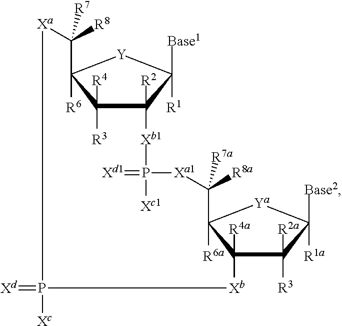

as described in WO2018/118665, which is incorporated herein by reference.

STING agonists may include compounds of formula

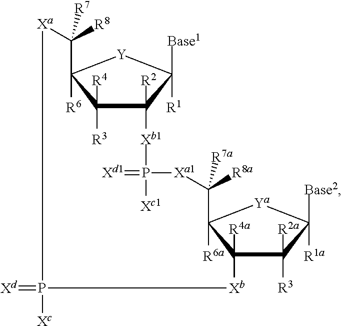

as described in WO2018/208667, which is incorporated herein by reference.

STING agonists may include compounds of formula

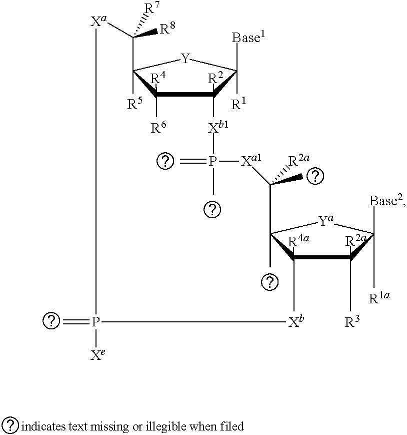

as described in WO2019/125974, which is incorporated herein by reference.

STING agonists may include compounds of formula

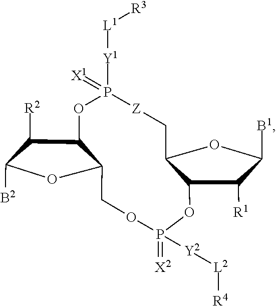

as described in WO2018/009648, which is incorporated herein by reference.

STING agonists may include compounds of formula

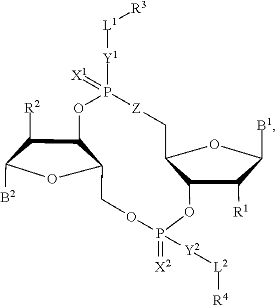

as described in WO2018/009652, which is incorporated herein by reference.

STING agonists may include compounds of formula

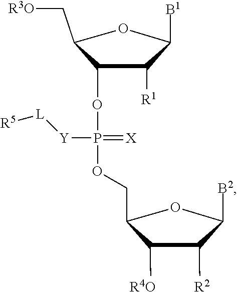

as described in WO2018/013887, which is incorporated herein by reference.

STING agonists may include compounds of formula

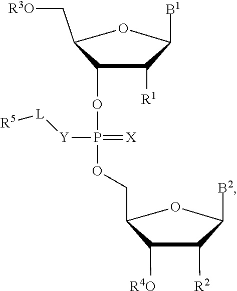

as described in WO2018/013908, which is incorporated herein by reference.

STING agonists may include compounds of formula

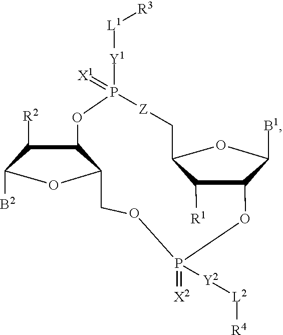

as described in WO2019/046511, which is incorporated herein by reference.

STING agonists may include compounds of formula

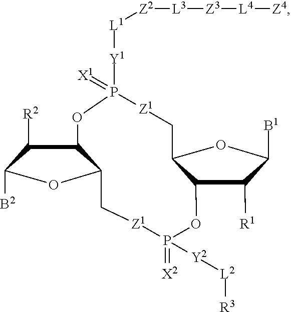

as described in WO2019/051488, which is incorporated herein by reference.

STING agonists may include compounds of formula

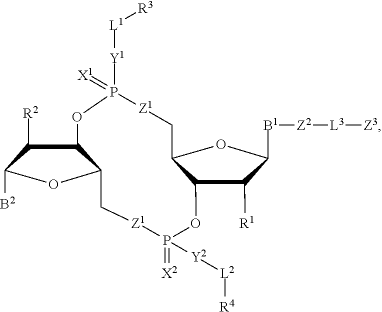

as described in WO2019/051489, which is incorporated herein by reference.

STING agonists may include compounds of formula

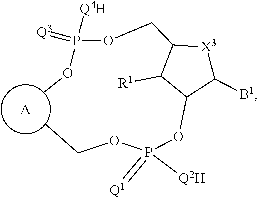

as described in US2019/0192549, which is incorporated herein by reference.

STING agonists may include compounds of formula

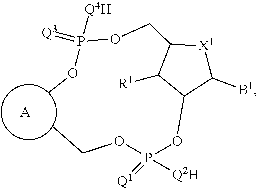

as described in WO2018/100558, which is incorporated herein by reference.

STING agonists may include compounds of formula

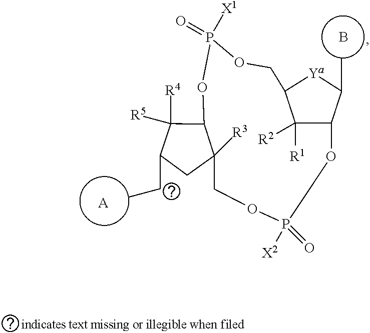

as described in WO2019/092660, which is incorporated herein by reference.

STING agonists may include compounds of formula

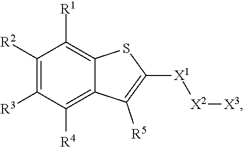

as described in WO2019/027858, which is incorporated herein by reference.

STING agonists may include compounds of formula

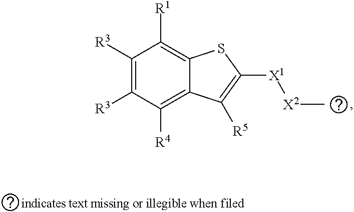

as described in US2018/0093964, which is incorporated herein by reference.

STING agonists may include compounds of formula

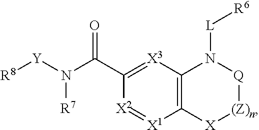

wherein X, X¹-X³, L, Q, Z, Y, n, and R⁶—R⁸ are as described in WO2018/234805, which is incorporated herein by reference.

STING agonists may include compounds of formula

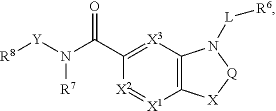

wherein X, X¹-X³, L, Q, Y, and R⁶—R⁸ are as described in WO2018/234807, which is incorporated herein by reference.

STING agonists may include compounds of formula

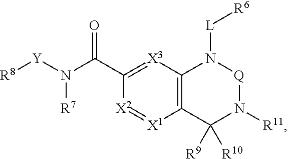

wherein X¹—X³, L, Q, Y, and R⁶—R″ are as described in WO2018/234808, which is incorporated herein by reference.

STING agonists include, for example, the compound DMXAA:

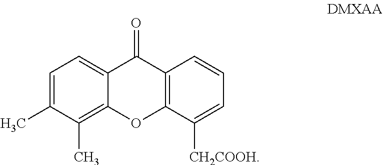

STING agonists include di-amidobenimidazoles, such as

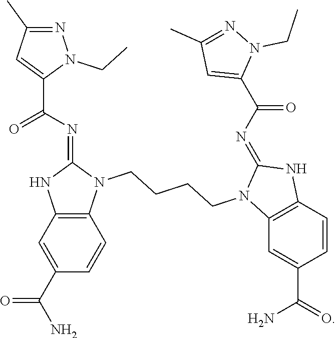 [SMILES: CCn1nc(C)cc1C(=O)/N=c1\[nH]c2cc(C(N)=O)ccc2n1CCCCn1/c(=N/C(=O)c2cc(C)nn2CC)[nH]c2ccc(C(N)=O)cc21]

Preferably, D is a cyclic dinucleotide, such as

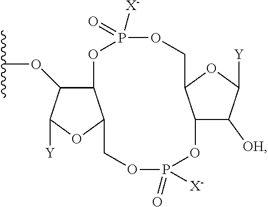

wherein Y is a nucleobase and X is O or S, and as illustrated below. A nucleobase includes naturally-occurring purine and pyrimidine bases, as well as modified purine and pyrimidine bases and other heterocyclic bases which have been modified. Such modifications include methylated purines or pyrimidines, acylated purines or pyrimidines, and the like. Nucleobase modifications may include, for example, deazapurines, N-1-methylguanosine, isoguanine, 2-aminopurine, 1,3-diaza-2-oxophenothiazine, 1,3-diaza-2-oxophenoxazine, 7-nitro-1,3-diaza-2-oxophenothiazine, 2,6-diaminopurine, purine, 6-thioguanine, hypoxanthine, 2-pyrimidinone, 2-pyridone, 4-thiouridine, imidazole-4-carboxamide, N-substituted 5-(carboxyamide)uridines such as 5-(N-benzylcarboxyamide)-uridine, or 5-fluoro-deoxyuridine.

In accordance with the foregoing definition of a payload moiety, a “cyclic dinucleotide payload moiety” is a cyclic dinucleotide minus its nucleophilic group (typically O) that attaches to a linker. For example, when

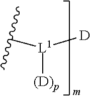

is

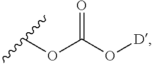

a cyclic dinucleotide payload moiety may be

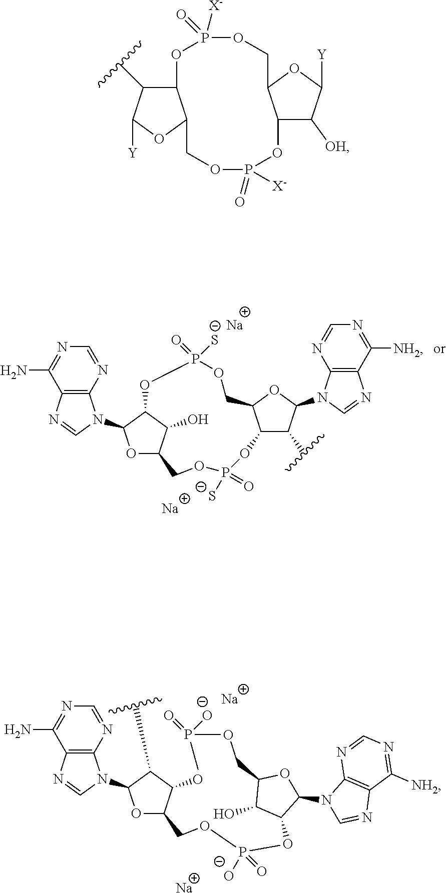

In some embodiments, the payload D is

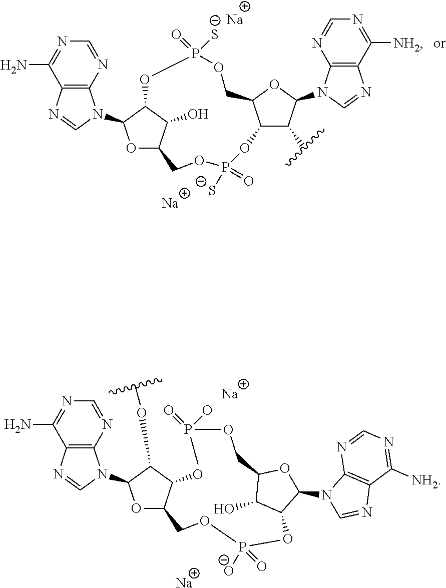 [SMILES: *[C@@H]1[C@@H]2OP(=O)([S-])OC[C@H]3O[C@@H](n4cnc5c(N)ncnc54)[C@H](OP(=O)([S-])OC[C@H]2O[C@H]1n1cnc2c(N)ncnc21)[C@@H]3O ; [Na+] ; [Na+] ; *O[C@@H]1[C@H](O)[C@@H](COP(=O)([O-])O[C@@H]2[C@H](O)[C@@H](COP(C)(=O)O)O[C@H]2n2cnc3c(N)ncnc32)O[C@H]1n1cnc2c(N)ncnc21 ; [Na+] ; [Na+]]

Compounds of formula (I)/(I-A) include

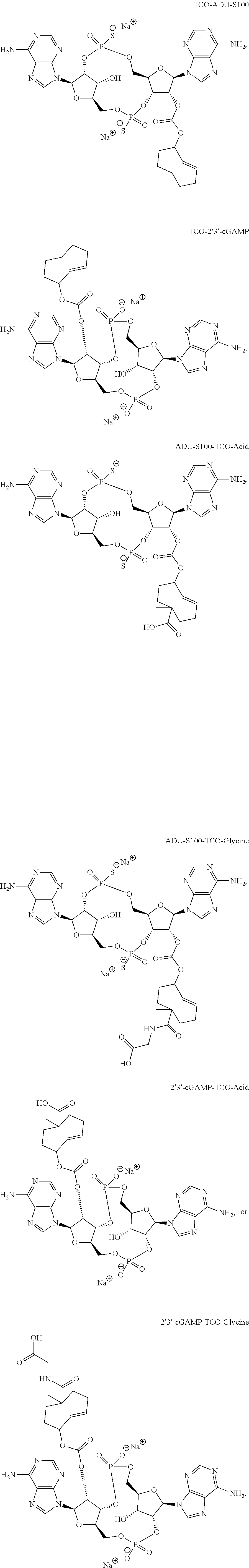

Preferably, D is an imidazo[4,5-c]quinolin-4-amine, such as

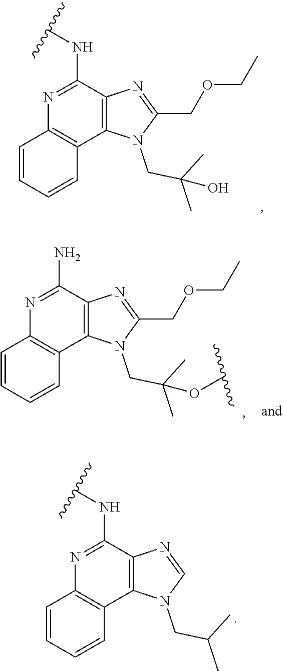 [SMILES: *Nc1nc2ccccc2c2c1nc(COCC)n2CC(C)(C)O ; *OC(C)(C)Cn1c(COCC)nc2c(N)nc3ccccc3c21 ; *Nc1nc2ccccc2c2c1ncn2CC(C)C]

In accordance with the foregoing definition of a payload moiety, an “imidazo[4,5-c]quinolin-4-amine payload moiety” is an imidazo[4,5-c]quinolin-4-amine minus its nucleophilic group (typically 0 or N) that attaches to a linker. For example, when

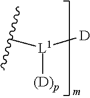

is

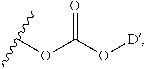

an imidazo[4,5-c]quinolin-4-amine payload moiety D′ may be

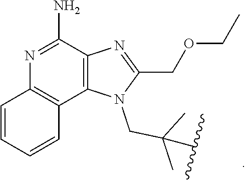 [SMILES: *C(C)(C)Cn1c(COCC)nc2c(N)nc3ccccc3c21]

For example, when

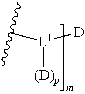

is

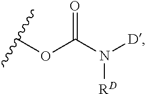

an imidazo[4,5-c]quinolin-4-amine payload moiety D′ may be

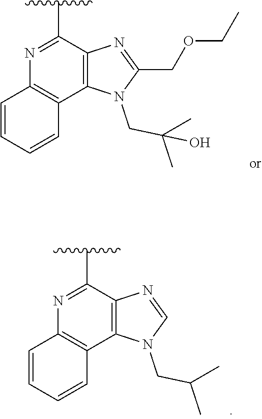 [SMILES: *c1nc2ccccc2c2c1nc(COCC)n2CC(C)(C)O ; *c1nc2ccccc2c2c1ncn2CC(C)C]

Compounds of formula (I)/(I-A) include

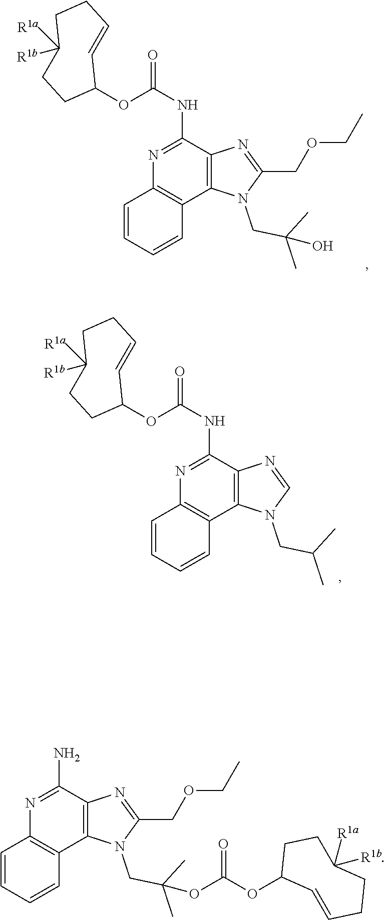

Synthetic methods of preparation of trans-cyclooctene modified payloads are described in detail in WO2018/187740, WO2014/205126, WO2015/139025, WO2017/044983, which are incorporated herein by reference.

The compounds may exist as stereoisomers wherein asymmetric or chiral centers are present. The stereoisomers are “R” or “S” depending on the configuration of substituents around the chiral carbon atom. The terms “R” and “S” used herein are configurations as defined in IUPAC 1974 Recommendations for Section E, Fundamental Stereochemistry, in Pure Appl. Chem., 1976, 45: 13-30. The disclosure contemplates various stereoisomers and mixtures thereof, and these are specifically included within the scope of this invention. Stereoisomers include enantiomers and diastereomers and mixtures of enantiomers or diastereomers. Individual stereoisomers of the compounds may be prepared synthetically from commercially available starting materials, which contain asymmetric or chiral centers or by preparation of racemic mixtures followed by methods of resolution well-known to those of ordinary skill in the art. These methods of resolution are exemplified by (1) attachment of a mixture of enantiomers to a chiral auxiliary, separation of the resulting mixture of diastereomers by recrystallization or chromatography, and optional liberation of the optically pure product from the auxiliary as described in Furniss, Hannaford, Smith, and Tatchell, “Vogel's Textbook of Practical Organic Chemistry”, 5^{th} edition (1989), Longman Scientific & Technical, Essex CM20 2JE, England, or (2) direct separation of the mixture of optical enantiomers on chiral chromatographic columns, or (3) fractional recrystallization methods.

It should be understood that the compounds may possess tautomeric forms as well as geometric isomers, and that these also constitute an aspect of the invention.

The present disclosure also includes isotopically-labeled compounds, which are identical to those recited herein, but for the fact that one or more atoms are replaced by an atom having an atomic mass or mass number different from the atomic mass or mass number usually found in nature. Examples of isotopes suitable for inclusion in the compounds of the invention are hydrogen, carbon, nitrogen, oxygen, phosphorus, sulfur, fluorine, and chlorine, such as, but not limited to, ²H, ³H, ¹³C, ¹⁴C, ¹⁵N, ¹⁸O, ¹⁷O, ³¹P, ³²P, ³⁵S, and ³⁶Cl, respectively. Substitution with heavier isotopes such as deuterium, i.e., ²H, can afford certain therapeutic advantages resulting from greater metabolic stability, for example increased in vivo half-life or reduced dosage requirements, and, hence, may be preferred in some circumstances. The compound may incorporate positron-emitting isotopes for medical imaging and positron-emitting tomography (PET) studies for determining the distribution of receptors. Suitable positron-emitting isotopes that can be incorporated in compounds of formula (I), (II-A), or (III-A) are ¹¹C, ¹³N, ¹⁵O, and ¹⁸F. Isotopically-labeled compounds disclosed herein can generally be prepared by conventional techniques known to those skilled in the art or by processes analogous to those described in the accompanying Examples using appropriate isotopically-labeled reagent in place of non-isotopically-labeled reagent.

B. Therapeutic Support Compositions

The therapeutic support composition comprises a support. Supports may be biocompatible supports compositions, i.e., compatible with the subject's body. In some instances, a support is non-toxic to the subject and does not substantially react with tissue or biological compounds in the subject. For example, the support can be a hydrogel, among others. A support is capable of implantation into a subject's body and supporting binding agents (e.g., tetrazine-containing group), as well as payloads after the binding agents conjugate. Representative supports include, but are not limited to polymers, viscous or non-viscous liquid materials, gels, hydrogels, polysaccharide hydrogels, a cross-linked polymer matrix, a metal, a ceramic, a plastic, a bone graft material, alginate, cellulose, chitosan, hyaluronic acid, chondroitin sulfate, heparin, and the like. Supports also include particles, such as nanoparticles, microparticles, and the like.

Hydrogels may be polysaccharide hydrogels, alginate, cellulose, hyaluronic acid, chitosan, chitosin, chitin, hyaluronic acid, chondroitin sulfate, heparin, and the like. Other suitable sugar-based biomaterials include those described in Polymer Advanced Technology, 2014, 25, 448-460. Polymers that may be used as the support can include, but are not limited to, polyphosphazenes, polyanhydrides, polyacetals, poly(ortho esters), polyphosphoesters, polycaprolactones, polyurethanes, polylactides, polycarbonates, polyamides, and polyethers, and blends/composites/co-polymers thereof. Representative polyethers include, but are not limited to, poly(ethylene glycol) (PEG), polypropylene glycol) (PPG), triblock Pluronic ([PEG]ₙ-[PPG]ₘ-[PEG]n), PEG diacrylate (PEGDA), and PEG dimethacrylate (PEGDMA). The support can also include proteins and other poly(amino acids), such as collagen, gelatin, elastin and elastin-like polypeptides, albumin, fibrin, poly(gamma-glutamic acid), poly(L-lysine), poly(L-glutamic acid), poly(aspartic acid), and the like.

In some embodiments, the support is a hydrogel. In some embodiments, the support is an alginate. In some embodiments, the support is chitin. In some embodiments, the support is a hyaluronic acid (e.g., a non-hydrogel hyaluronic acid substantially without crosslinks). In some embodiments, the support is chitosin.

In certain embodiments, the support is a particle. Particles of the present disclosure can have a diameter that is 2 cm or less, such as 1.5 cm or less, or 1 cm or less, or 0.5 cm or less. For example, the particles can be nanoparticles or microparticles. Nanoparticles include particles having average dimensions in the nanometer scale (e.g., 1000 nm or less). Microparticles are particles having average dimensions in the micrometer scale (e.g., 1000 μm or less). By “average” is meant the arithmetic mean. In some embodiments, the nanoparticles have a diameter ranging from 1 nm to 1 such as from 10 nm to 1 or 25 nm to 1 or 50 nm to or 75 nm to 1 or 100 nm to 1 or 150 nm to 1 or 200 nm to 1 or 250 nm to 1 or 300 nm to 1 or 350 nm to 1 or 400 nm to 1 or 450 nm to 1 or 500 nm to 1 In other embodiments, the microparticles have a diameter ranging from 1 μm to 1 mm, such as from 10 μm to 1 mm, or 25 μm to 1 mm, or 50 μm to 1 mm, or 75 μm to 1 mm, or 100 μm to 1 mm, or 150 μm to 1 mm, or 200 μm to 1 mm, or 250 μm to 1 mm, or 300 μm to 1 mm, or 350 μm to 1 mm, or 400 μm to 1 mm, or 450 μm to 1 mm, or 500 μm to 1 mm. In further embodiments, small particles on the order of 10-100 nm in diameter may be assembled to form larger complexes, such as clusters or assemblies on the order of 1-10 Particles of the present disclosure may be substantially spherical, such that the particles have a substantially circular cross-section. Other particle shapes may also be used, such as, but not limited to, ellipsoid, cubic, cylindrical, conical, needle, or other irregular shapes.

A “particle” may take the form of any fabricated material, a molecule, cryptophan, a virus, a phage, etc. The particle may be composed of a material, such as, but not limited to, a metal, a ceramic, a plastic, a glass, a composite, a polymer, a hydrogel, and the like. For example, the particles may be made of an inert material, such as alginate or iron oxide. In some examples, the particles may be magnetic and can be formed from a paramagnetic, super-paramagnetic or ferromagnetic material, or other material that responds to a magnetic field. Further, a particle may be of any shape, for example, spheres, rods, non-symmetrical shapes, etc. The particles, or a group of several particles in a complex, may be functionalized with a receptor that has a specific affinity to bind to or interact with a clinically relevant substrate. The receptor may be inherent to the particle itself. For example, the particle itself may be a virus or a phage with an inherent affinity for certain substrates. Additionally or alternatively, the particles can be functionalized by covalently or otherwise attaching or associating a receptor that specifically binds or otherwise recognizes a particular clinically relevant substrate. The functionalized receptor can be an antibody, peptide, nucleic acid, phage, bacteria, virus, or any other molecule with a defined affinity for a target substrate. Examples of material that may be used for the “particles” and/or “carrier” include polylactic acid, polyglycolic acid, PLGA polymers, alginates and alginate derivatives, gelatin, collagen, fibrin, hyaluronic acid, laminin rich gels, agarose, natural and synthetic polysaccharides, polyamino acids, polypeptides, polyesters, poly anhydrides, polyphosphazines, poly(vinyl alcohols), poly(alkylene oxides), poly(allylamines)(PAM), poly(acrylates), modified styrene polymers, pluronic polyols, polyoxamers, poly(uronic acids), poly(vinylpyrrolidone) and copolymers or graft copolymers of any of the above. These examples do not limit their concentration, their cross-linking with different agents, their method of administration, their tailored degradation profiles and other characteristics known to those skilled in the art.

The particles, or a group of several particles in a complex, may be functionalized with a targeting agent (e.g., a ligand or antibody) that specifically binds (or substantially specifically binds) to a target (e.g., a target receptor or a cell surface target, such as a clinically relevant receptor or cell surface target (e.g., antigen)). The targeting agent may be attached directly to the particle itself. The targeting agent can be an antibody, peptide, nucleic acid, phage, bacteria, virus, or any other molecule with a specific affinity for a target receptor or cell surface target. In some instances, the receptor or cell surface target is PD-1, CTLA-4, HER2/neu, HER1/EGFR, VEGFR, BCR-ABL, SRC, JAK2, MAP2K, EML4-ALK, BRAF V600E, 4-1BB, GITR, GSK3beta, or other cellular receptors or cell surface targets. Other compounds or molecules, such as fluorophores or autofluorescent or luminescent markers, which may assist in detecting the particles (e.g., in vivo detection), may also be attached to the particles. The ligands and/or detectable labels may be attached directly to the particle or attached to the particle through bioorthogonal functional groups as described herein.

In certain embodiments, the support is a bone graft material, such as a bone graft substitute material. A bone graft substitute material is a material structurally similar to bone. In some instances, a bone graft substitute material is bioresorbable such that the bone graft substitute material can dissolve or be absorbed in the body over time. A bone graft substitute material can be osteoconductive, such that it facilitates blood vessel and new bone formation into the bone graft substitute material. In some instances, the bone graft substitute material is osteoinductive, such that facilitates the formation of new bone through active recruitment of mesenchymal stem cells from the surrounding tissue. For example, growth factors, such as bone morphogenetic proteins, may be included in the bone graft substitute material. Bone graft substitute materials include, but are not limited to, hydroxyapatite, tricalcium phosphate, demineralized bone matrix, bovine collagen, calcium sulfate, calcium phosphate, cancellous bone chips, and the like, and combinations thereof.

Therapeutic support compositions of the present disclosure include a support and a first binding agent covalently linked to the support. The binding agent may be attached to the support on a surface of the support, such as a solvent-accessible surface of the support (e.g., a surface of the support that is in contact with the surrounding solvent). In some cases, the binding agent is attached directly to the support. For example, the binding agent may be covalently attached to the surface of the support, e.g., through a covalent bond, such as an amide, amine, ester, carbamate, urea, thioether, thiocarbamate, thiocarbonate, thiourea, etc. In some instances, the binding agent is covalently attached to the support through an amide bond. In other instances, the binding agent may be linked to the support via a linker. Any suitable linker can be used to link the binding agent to the support. Representative linkers can have from 1 to 100 linking atoms, and can include ethylene-oxy groups, amines, esters, amides, carbamates, carbonates, and ketone functional groups. For example, linkers may have from 1 to 50 linking atoms, or from 5 to 50 linking atoms, or from 10 to 50 linking atoms. Representative linkers include, but are not limited to, those shown below:

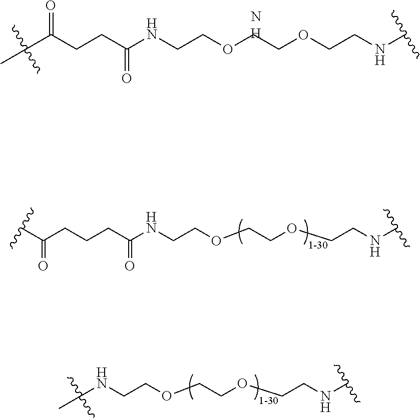

In certain embodiments, the therapeutic support compositions comprise a support and a tetrazine-containing group of formula:

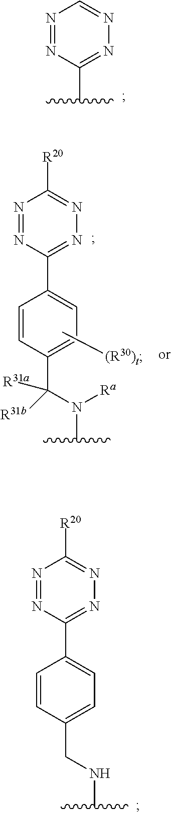

wherein R²⁰ is selected from the group consisting of hydrogen, halogen, cyano, nitro, alkyl, alkenyl, alkynyl, heteroalkyl, aryl, heteroaryl, heterocycle, cycloalkyl, cycloalkenyl, CF₃, CF₂—R′, NO₂, OR′, SR′, C(═O)R′, C(═S)R′, OC(═O)R′″, SC(═O)R′″, OC(═S)R′″, SC(═S)R′″, S(═O)R′, S(═O)₂R, S(═O)₂NR′ R″, C(═O)O—R′, C(═O)S—R′, C(═S)O—R′, C(═S)S—R′, C(═O)NR′R″, C(═S)NR′ R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S) R′R′″, SC(═S)R′R″, NR′C(═O)NR″R″, and NR′C(═S)NR″R″; R′ and R″ at each occurrence are independently selected from hydrogen, aryl and alkyl; and R′″ at each occurrence is independently selected from aryl and alkyl; R³⁰ is halogen, cyano, nitro, hydroxy, alkyl, haloalkyl; alkenyl, alkynyl, alkoxy; halalkoxy; heteroalkyl, aryl, heteroaryl, heterocycle, cycloalkyl, or cycloalkenyl; R^{a}, R^{31a} and R^{31b} are each independently hydrogen, C₁-C₆-alkyl, or C₁-C₆-haloalkyl; and t is 0, 1, 2, 3, or 4.

In certain embodiments, the therapeutic support compositions have formula:

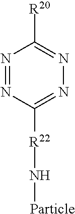

wherein

R²⁰ is selected from the group consisting of hydrogen, halogen, cyano, nitro, alkyl, alkenyl, alkynyl, heteroalkyl, aryl, heteroaryl, heterocycle, cycloalkyl, cycloalkenyl, CF₃, CF₂—R′, NO₂, OR′, SR′, C(═O)R′, C(═S)R′, OC(═O)R″, SC(═O)R′″, OC(═S)R′″, SC(═S)R′″, S(═O)R′, S(═O)₂R, S(═O)₂NR′ R″, C(═O)O—R′, C(═O)S—R′, C(═S)O—R′, C(═S)S—R′, C(═O)NR′R″, C(═S)NR′ R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S) R′R′″, SC(═S)R′R″, NR′C(═O)NR″R″, and NR′C(═S)NR″R″; R′ and R″ at each occurrence are independently selected from hydrogen, aryl and alkyl; R′″ at each occurrence is independently selected from aryl and alkyl; and R²² is a linker of 1 to 100 linking atoms, and can include ethylene-oxy groups, amines, esters, amides, carbamates, carbonates, and ketone functional groups. For example, linkers may have from 1 to 50 linking atoms, or from 5 to 50 linking atoms, or from 10 to 50 linking atoms.

In certain embodiments, the therapeutic support compositions have formula:

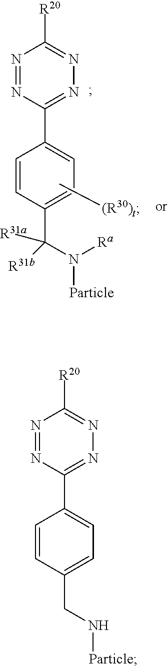

wherein

R²⁰ is selected from the group consisting of hydrogen, halogen, cyano, nitro, alkyl, alkenyl, alkynyl, heteroalkyl, aryl, heteroaryl, heterocycle, cycloalkyl, cycloalkenyl, CF₃, CF₂—R′, NO₂, OR′, SR′, C(═O)R′, C(═S)R′, OC(═O)R′″, SC(═O)R′″, OC(═S)R″, SC(═S)R′″, S(═O)R′, S(═O)₂R, S(═O)₂NR′ R″, C(═O)O—R′, C(═O)S—R′, C(═S)O—R′, C(═S)S—R′, C(═O)NR′R″, C(═S)NR′ R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S) R′R′″, SC(═S)R′R″, NR′C(═O)NR″R″, and NR′C(═S)NR″R″; R′ and R″ at each occurrence are independently selected from hydrogen, aryl and alkyl; R′″ at each occurrence is independently selected from aryl and alkyl; R³⁰ is halogen, cyano, nitro, hydroxy, alkyl, haloalkyl; alkenyl, alkynyl, alkoxy; halalkoxy; heteroalkyl, aryl, heteroaryl, heterocycle, cycloalkyl, or cycloalkenyl; R^{a}, R^{31a} and R^{31b} are each independently hydrogen, C₁-C₆-alkyl, or C₁-C₆-haloalkyl; and t is 0, 1, 2, 3, or 4.

In certain embodiments, the therapeutic support compositions comprise substituted alginate having units of formula:

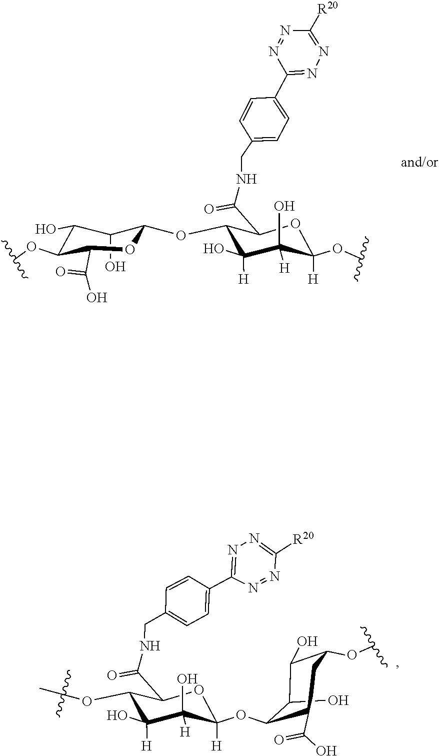

or a salt thereof,

wherein R²⁰ is selected from the group consisting of hydrogen, halogen, cyano, nitro, alkyl, alkenyl, alkynyl, heteroalkyl, aryl, heteroaryl, heterocycle, cycloalkyl, cycloalkenyl, CF₃, CF₂—R′, NO₂, OR′, SR′, C(═O)R′, C(═S)R′, OC(═O)R′″, SC(═O)R′″, OC(═S)R′″, SC(═S)R′″, S(═O)R′, S(═O)₂R, S(═O)₂NR′R″, C(═O)O—R′, C(═O)S—R′, C(═S)O—R′, C(═S)S—R′, C(═O)NR′R″, C(═S)NR′ R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S) R′R′″, SC(═S)R′R″, NR′C(═O)NR″R″, and NR′C(═S)NR″R″; R′ and R″ at each occurrence are independently selected from hydrogen, aryl and alkyl; and R′″ at each occurrence is independently selected from aryl and alkyl.

In certain embodiments, the therapeutic support compositions comprise units of formula:

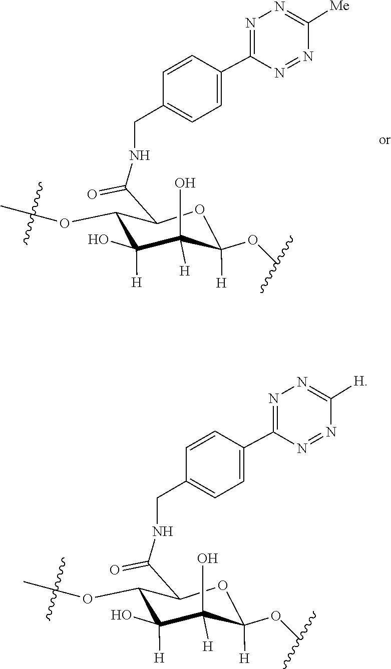

In some embodiments, the therapeutic support compositions comprise units of formula:

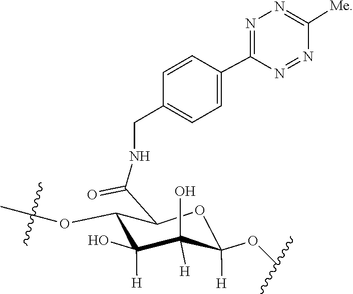

In some embodiments, the therapeutic support compositions comprise units of formula:

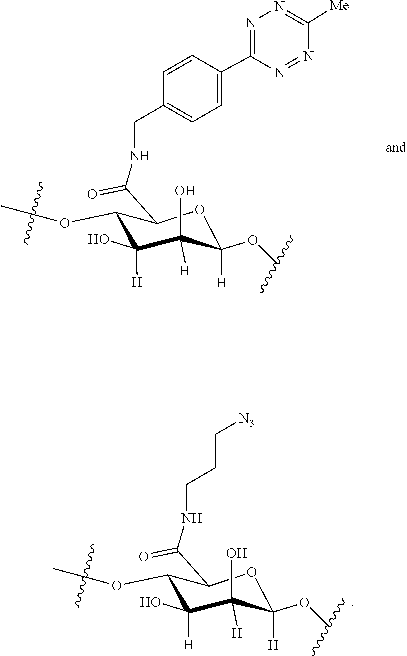

In some embodiments, the therapeutic support compositions comprise substituted hyaluronic acid having units of formula (II):

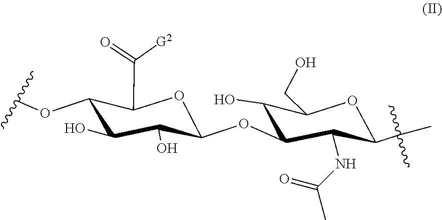

wherein G² is

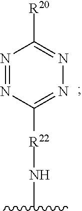

R²² is a linker of 1 to 100 linking atoms; and R²⁰ is as defined herein.

In further embodiments, G² is

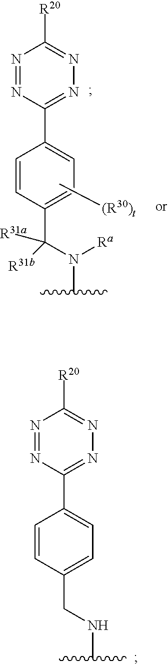

In still further embodiments, G² is

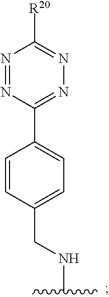

and R²⁰ is hydrogen or C₁₋₄ alkyl.

Compounds of formula (II) include compounds of formula (III):

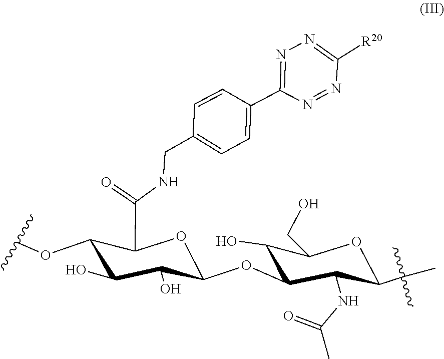

wherein R²⁰ is selected from the group consisting of hydrogen, halogen, cyano, nitro, alkyl, alkenyl, alkynyl, heteroalkyl, aryl, heteroaryl, heterocycle, cycloalkyl, cycloalkenyl, CF₃, CF₂—R′, NO₂, OR′, SR′, C(═O)R′, C(═S)R′, OC(═O)R′″, SC(═O)R′″, OC(═S)R′″, SC(═S)R′″, S(═O)R′, S(═O)₂R, S(═O)₂NR′ R″, C(═O)O—R′, C(═O)S—R′, C(═S)O—R′, C(═S)S—R′, C(═O)NR′R″, C(═S)NR′ R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S) R′R′″, SC(═S)R′R″, NR′C(═O)NR″R″, and NR′C(═S)NR″R″; R′ and R″ at each occurrence are independently selected from hydrogen, aryl and alkyl; and R′″ at each occurrence is independently selected from aryl and alkyl. In further embodiments according to formula (III), R²⁰ is hydrogen or C₁₋₄alkyl.

In some embodiments, the therapeutic support compositions comprise units of formula:

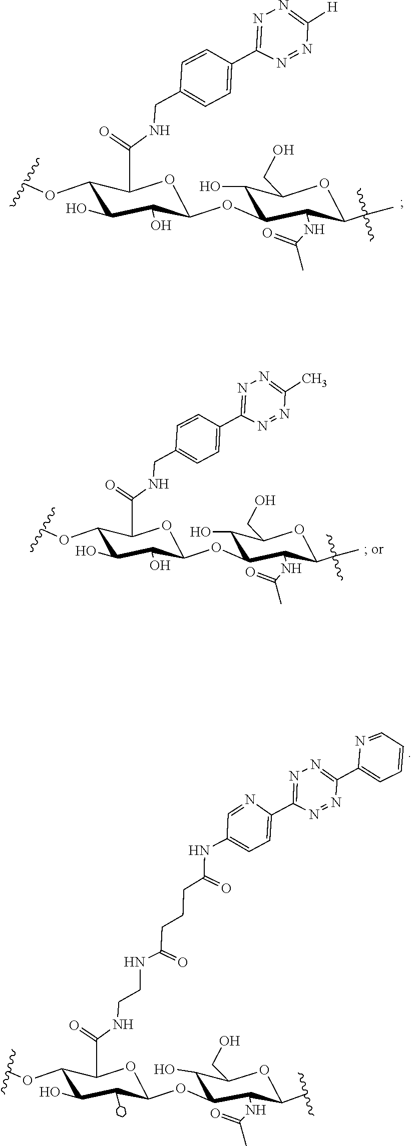

Additional therapeutic support compositions are exemplified in WO2017/044983, WO/2015/139025A1, and WO/2014/205126A1, the entire contents of each of which is incorporated herein by reference in their entirety.

The hyaluronic acid derivative includes a hyaluronic acid having a plurality of glucuronic acid units and a tetrazine-containing group linked or directly bonded to a glucuronic acid unit of the hyaluronic acid. The hyaluronic acid may also have a plurality of N-acetylglucosamine units. In certain embodiments, the N-acetylglucosamine units of the hyaluronic acid are not linked or conjugated to the tetrazine-containing group.

The tetrazine-containing group can be linked or directly bonded through a carboxylic acid of a glucuronic acid unit. The tetrazine-containing group can be incorporated into the hyaluronic acid from about 0.1% to about 80% as measured by the % of carboxylic acids being linked or conjugated to the tetrazine-containing group, such as about 1% to about 75%, about 5% to about 75%, about 10% to about 50%, or about 40% to about 75% as measured by the % of carboxylic acids being linked or conjugated to the tetrazine-containing group.

### 3. Synthetic Methods

The compounds of the present disclosure can be better understood in connection with the following synthetic schemes and methods, which illustrate means by which the compounds may be prepared.

In general, compounds of formula (I-A)/(I-B)/(II-A)/(III-A) may be prepared by reacting a payload having a primary amine, secondary amine, or a hydroxyl group with a suitably activated linker either before or after the linker is attached to the cyclooctene portion. It is to be understood that a reactive group on a linker (e.g., ester, carbonate, acyl chloride, carboxylic acid) can be located on any selected position of the linker group. Conversely, the linker may have a nucleophilic amine or hydroxyl group that may be reacted with a suitable group on the payload such as an aldehyde, ketone, ester, carbonate, carboxylic acid, or acyl chloride.

In certain embodiments, as shown below, a trans-cyclooctene activated for nucleophilic addition can be reacted with a suitable payload (D/D¹), or a payload attached to a linker L⁴-H, in the presence of a base to provide a functionalized payload. The payload or linker can include a primary amine, secondary amine, or hydroxyl group that reacts with the activated TCO. In certain embodiments, the leaving group (LG) is a chloro leaving group, a p-nitrophenol leaving group, or an N-hydroxysuccinimide leaving group. Exemplary bases for use in the reaction include organic and inorganic bases, such as for example, triethylamine, pyridine, sodium hydroxide, and sodium bicarbonate.

As shown in Scheme 1, a trans-cyclooctene having an activated carbonate ester may be coupled with (D/D¹)-L⁴-H to provide an intermediate 4, which may be further hydrolyzed to an acid 5 or coupled with an amine G¹-N(R^{1c})H under basic conditions to provide 7. Suitable G¹-N(R^{1c})H for the method of Scheme 1 include, for example, HN(R^{1c})CHR^{1e}CO₂H, HN(R^{1c})—C₁₋₆alkylene-CO₂H, HN(R^{1c})CHR^{1e}C(O)OC₁₋₄alkyl, and HN(R^{1c})—C₁₋₆alkylene-C(O)OC₁₋₄alkyl.

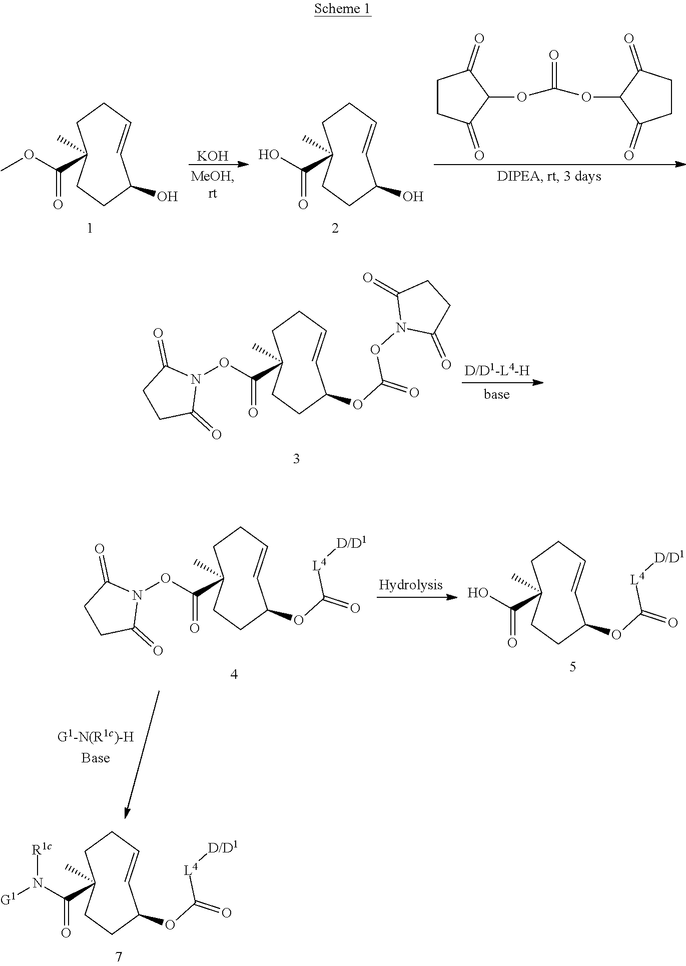

As shown in Scheme 2 below, a trans-cyclooctene having an activated carbonate ester may be coupled with a payload (e.g., doxorubicin, abbrev. as doxo) having an amine. The intermediate 4 may be hydrolyzed to the acid to provide functionalized payloads of the invention.

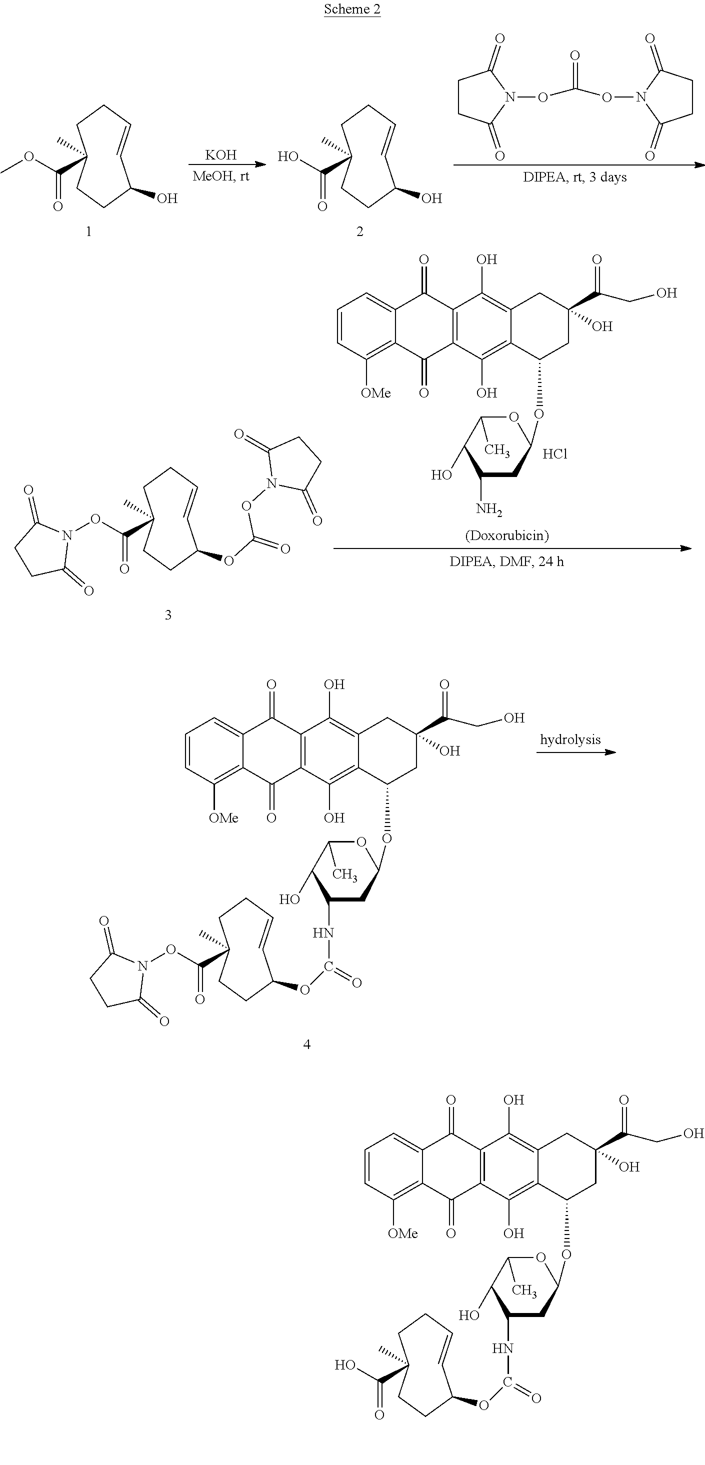

Scheme 3 illustrates further applications of the foregoing chemistry where the intermediate carbonate ester may be reacted with the ornithine side chain of daptomycin and further coupled with an amino-containing groups G¹-N(R^{1c})H under basic conditions.

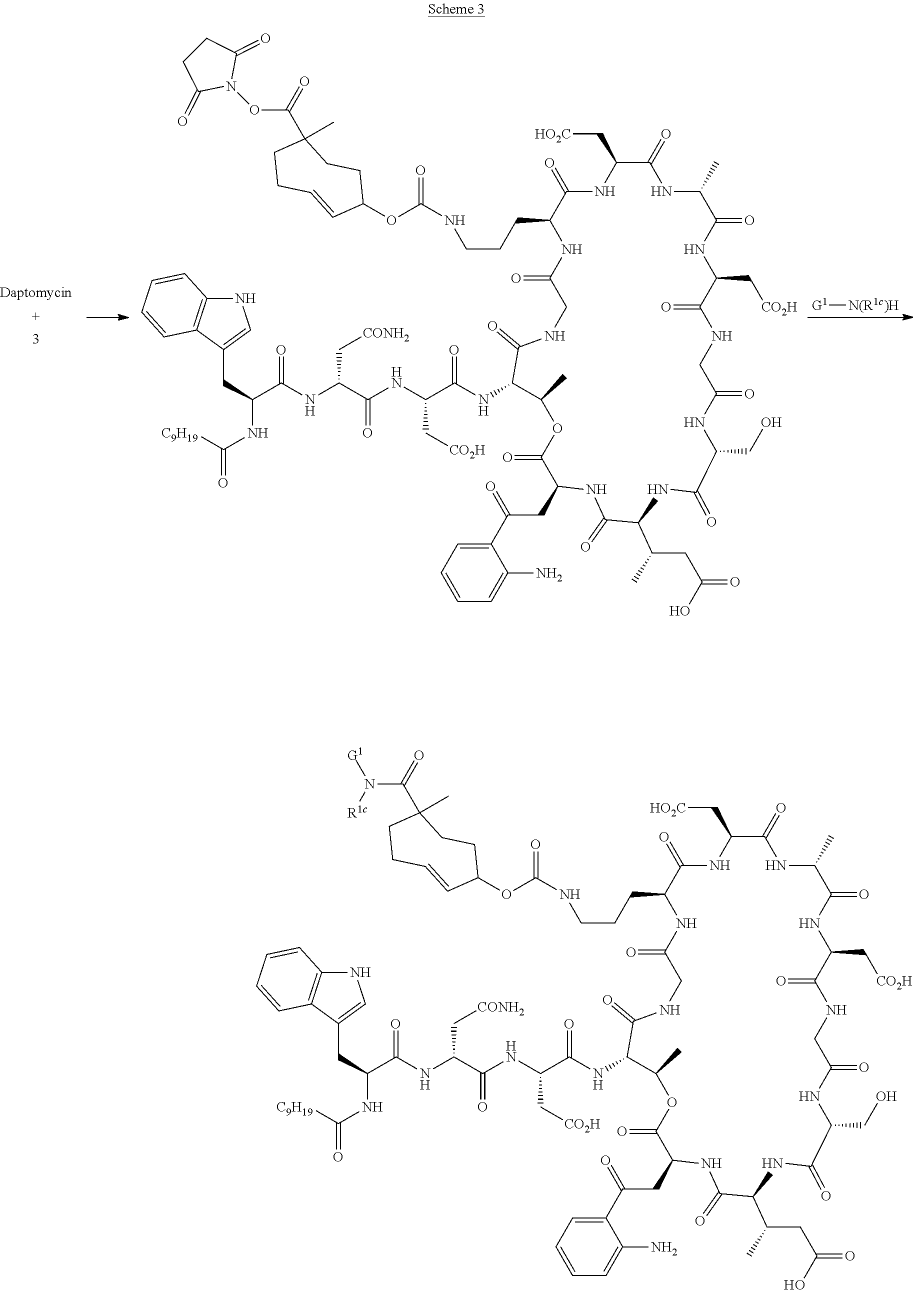

Scheme 4 shows a synthetic sequence to convert an intermediate 10 to an intermediate 11. Either 10 or 11 may be used to elaborate a linker, a protected linker, or a linker attached to a payload using general synthetic methods disclosed in WO2017/044983. The trimethylsilylethyl group may be removed at an appropriate point in the synthetic sequence to provide the carboxylic acid. The skilled artisan would be able to adapt the synthetic routes and protecting group strategies to arrive at compounds of the invention.

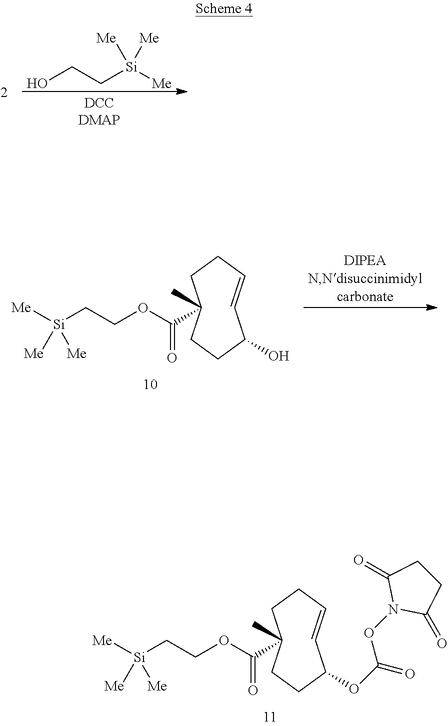

For example, Scheme 5 illustrated conversion of 11 to a carboxylic acid intermediate that may be further converted to payload-bearing products 13 and 14.

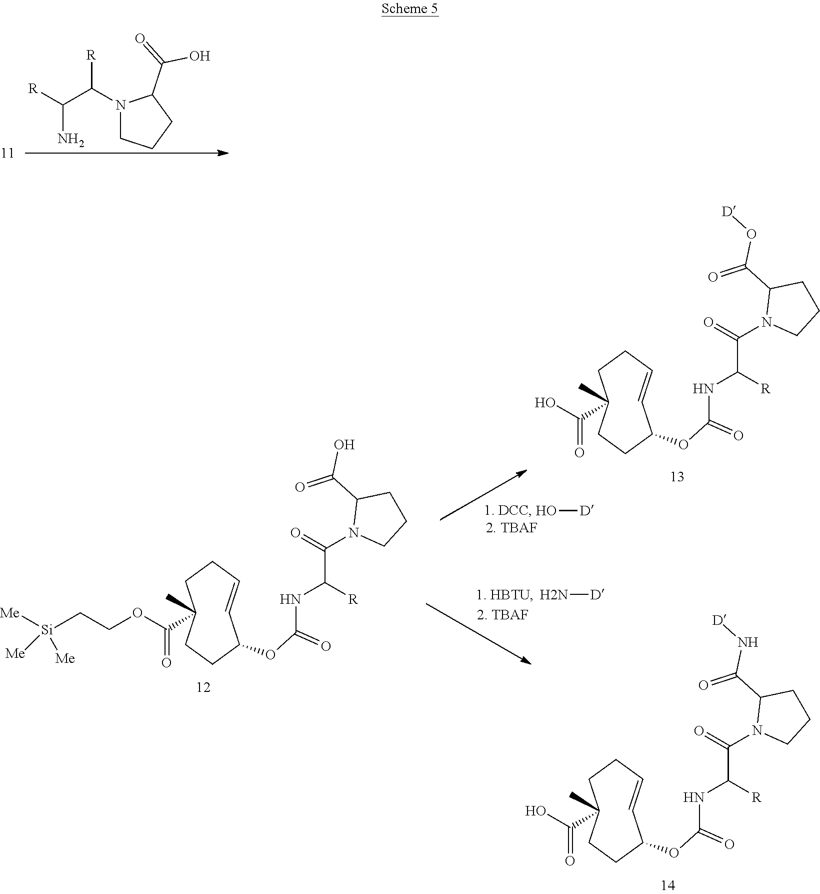

Other carboxylic acids that may be prepared using 11 include those shown in Scheme 6. The payload moiety D′ in Schemes 5 and 6 is a payload moiety of either payload D or D¹.

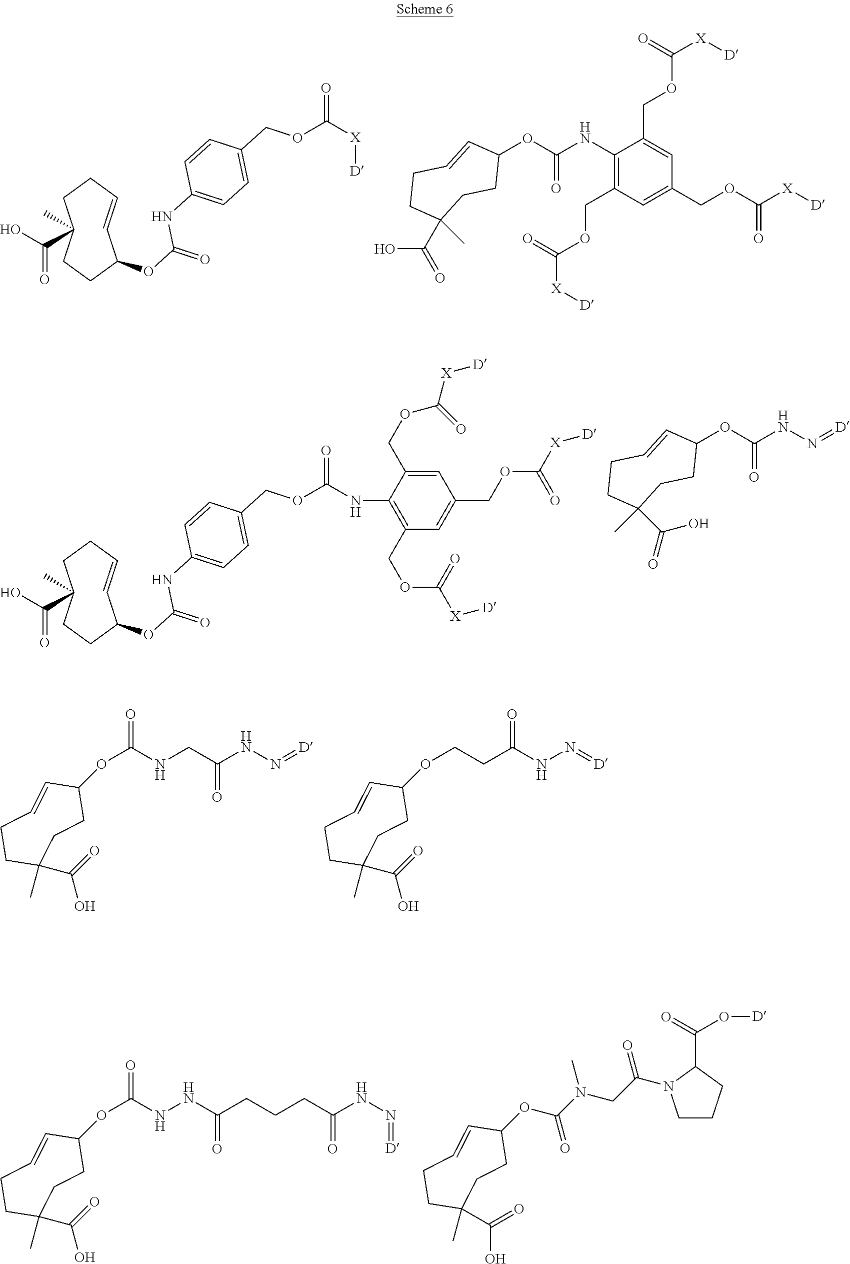

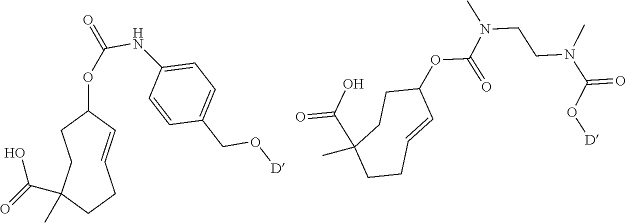

Scheme 7 illustrates general methods to prepare TCO conjugates with amide substitution on the TCO.

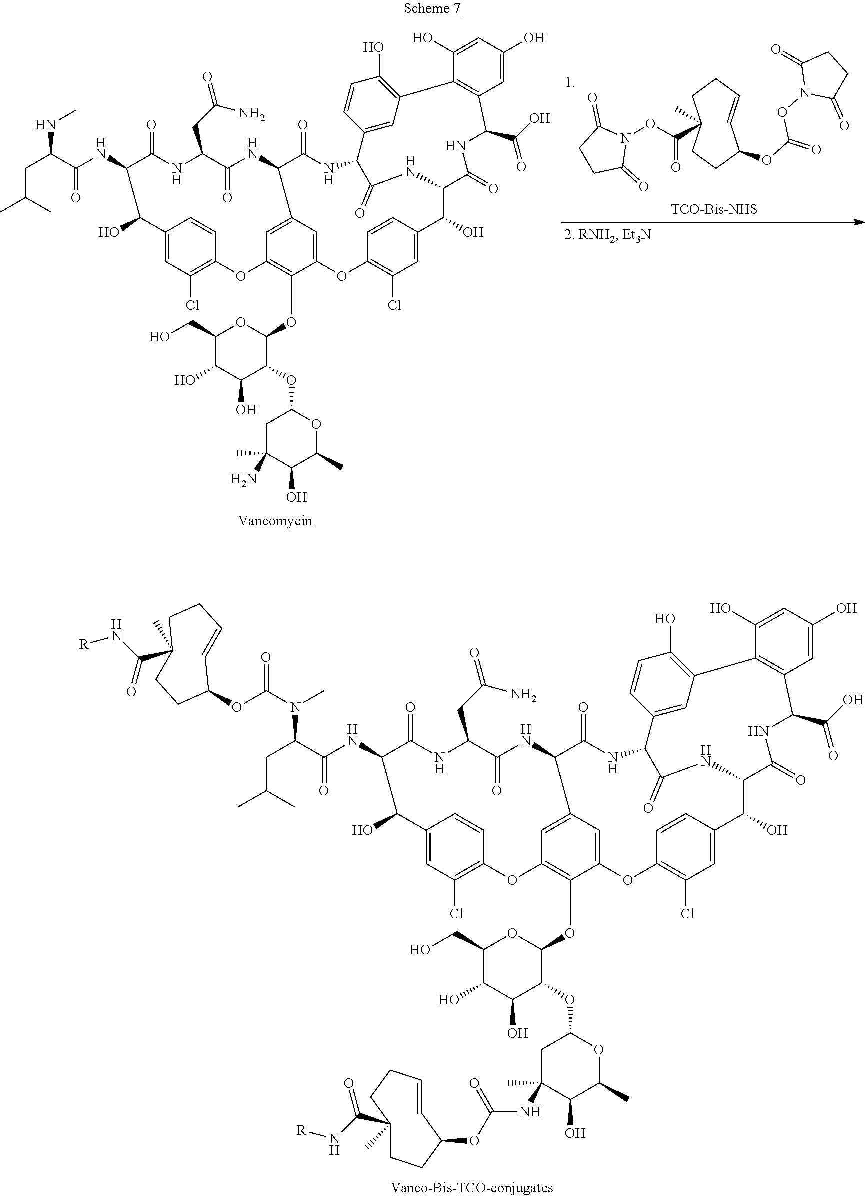

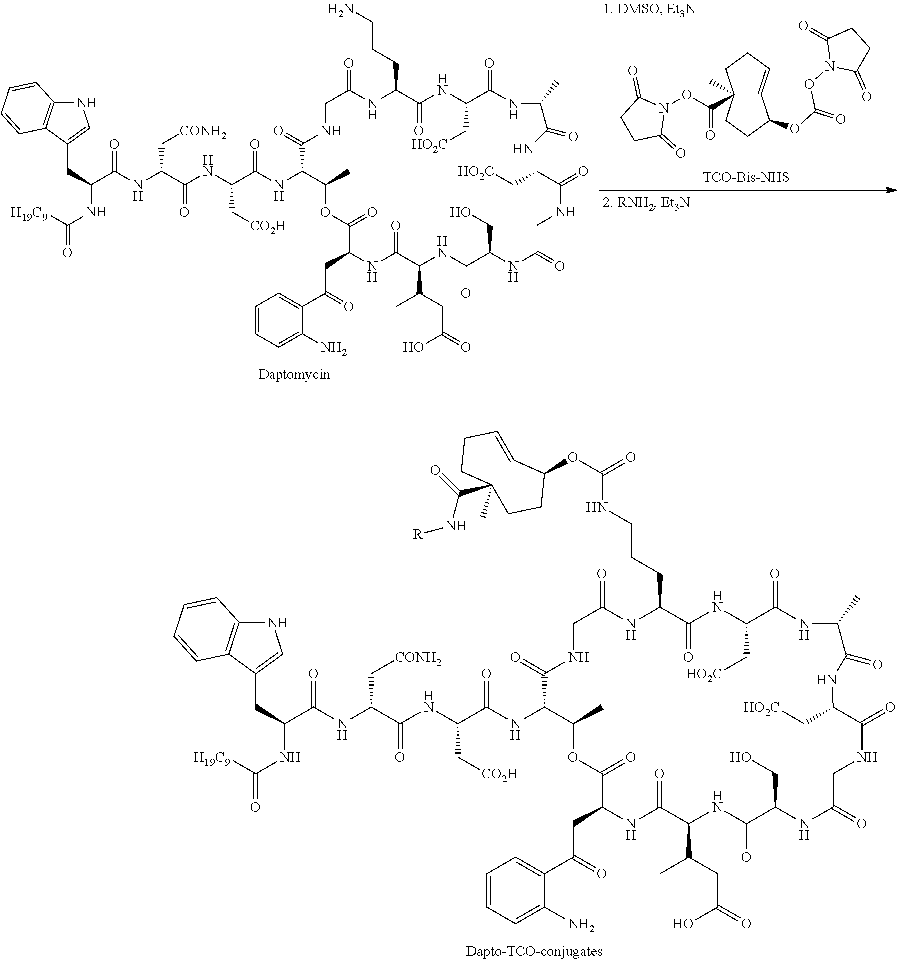

Synthetic methods to prepare representative STING agonist TCO conjugates are shown in Scheme 8.

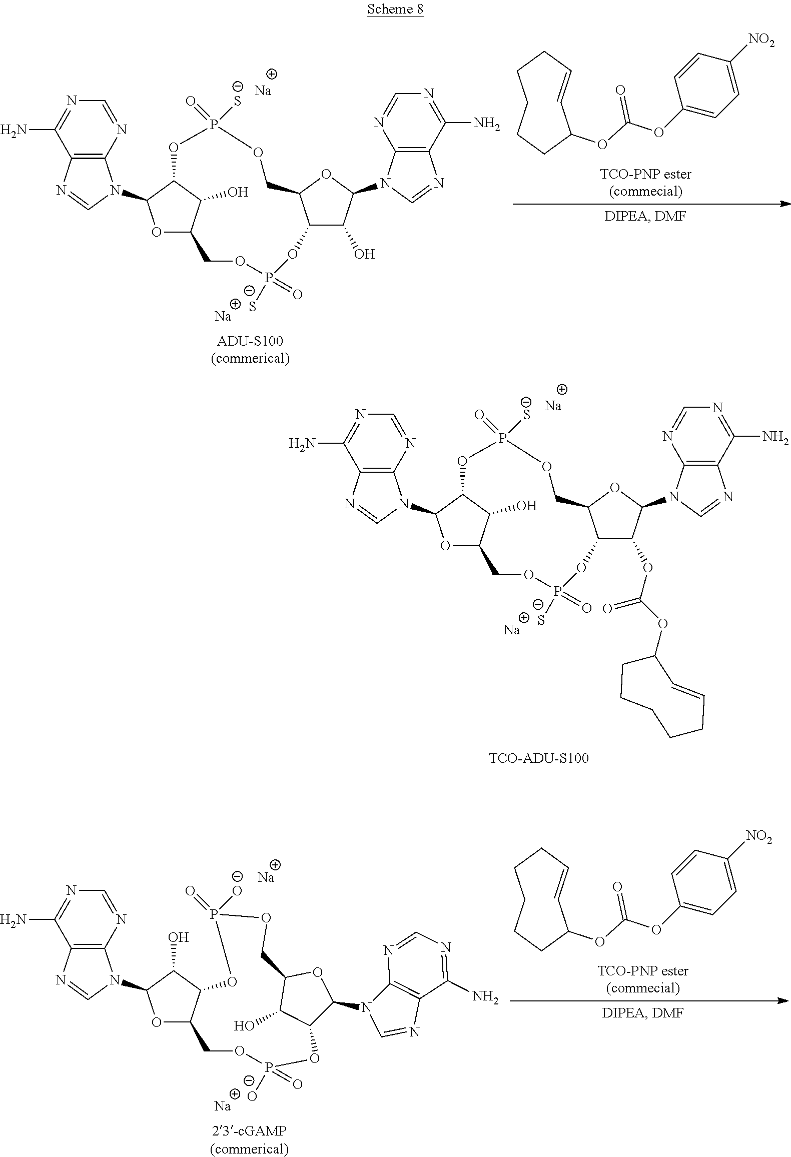

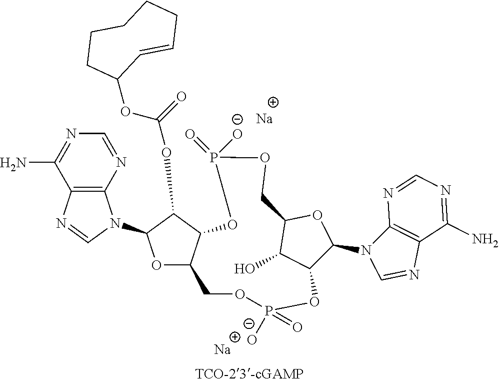 [SMILES: Nc1ncnc2c1ncn2[C@@H]1O[C@@H]2COP(=O)([O-])O[C@H]3[C@@H](OC(=O)OC4/C=C/CCCCC4)[C@H](n4cnc5c(N)ncnc54)O[C@@H]3COP(=O)([O-])O[C@@H]1[C@@H]2O ; [Na+] ; [Na+]]

Synthetic methods to prepare representative STING agonist TCO conjugates are shown in Scheme 9.

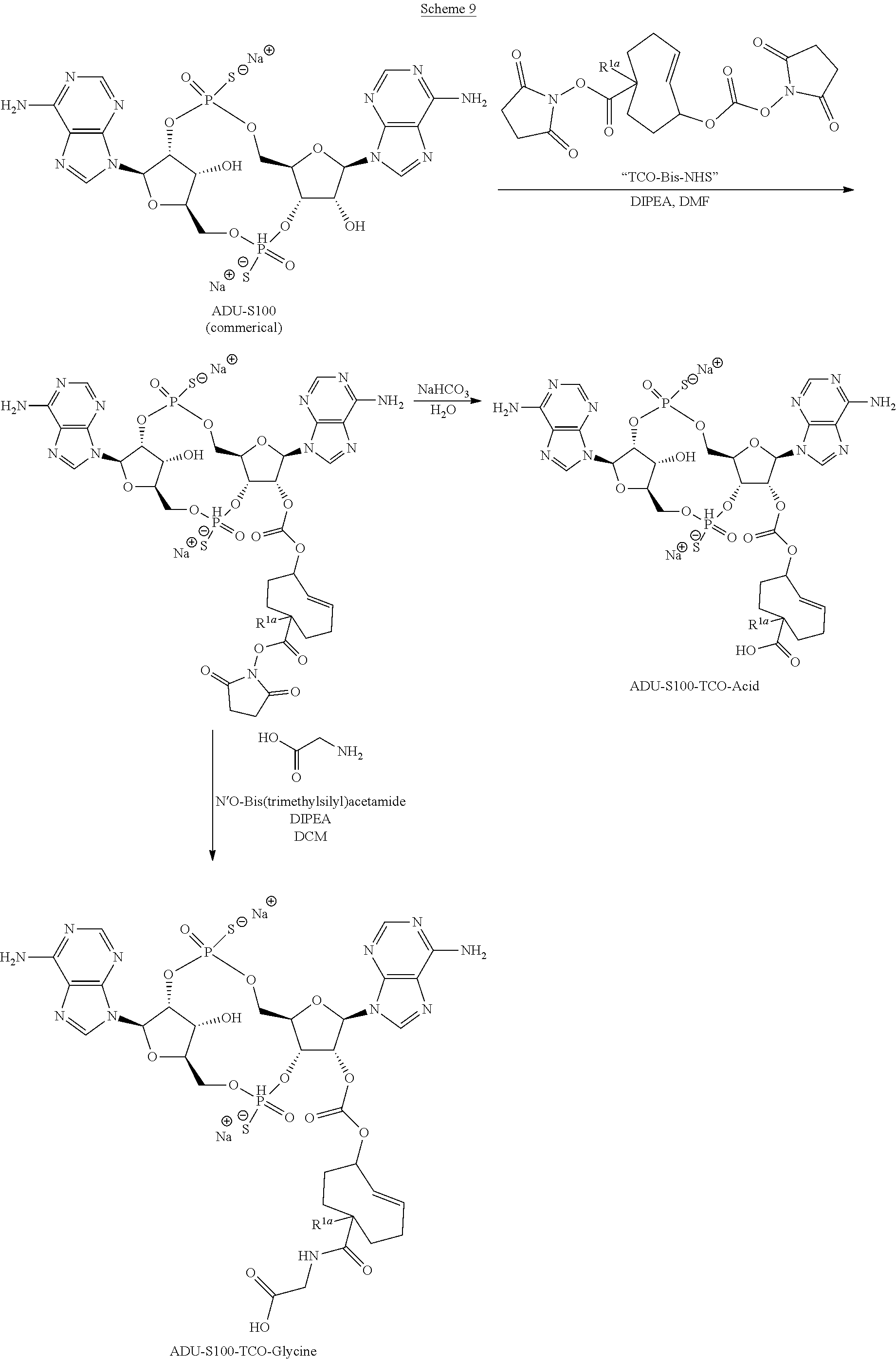

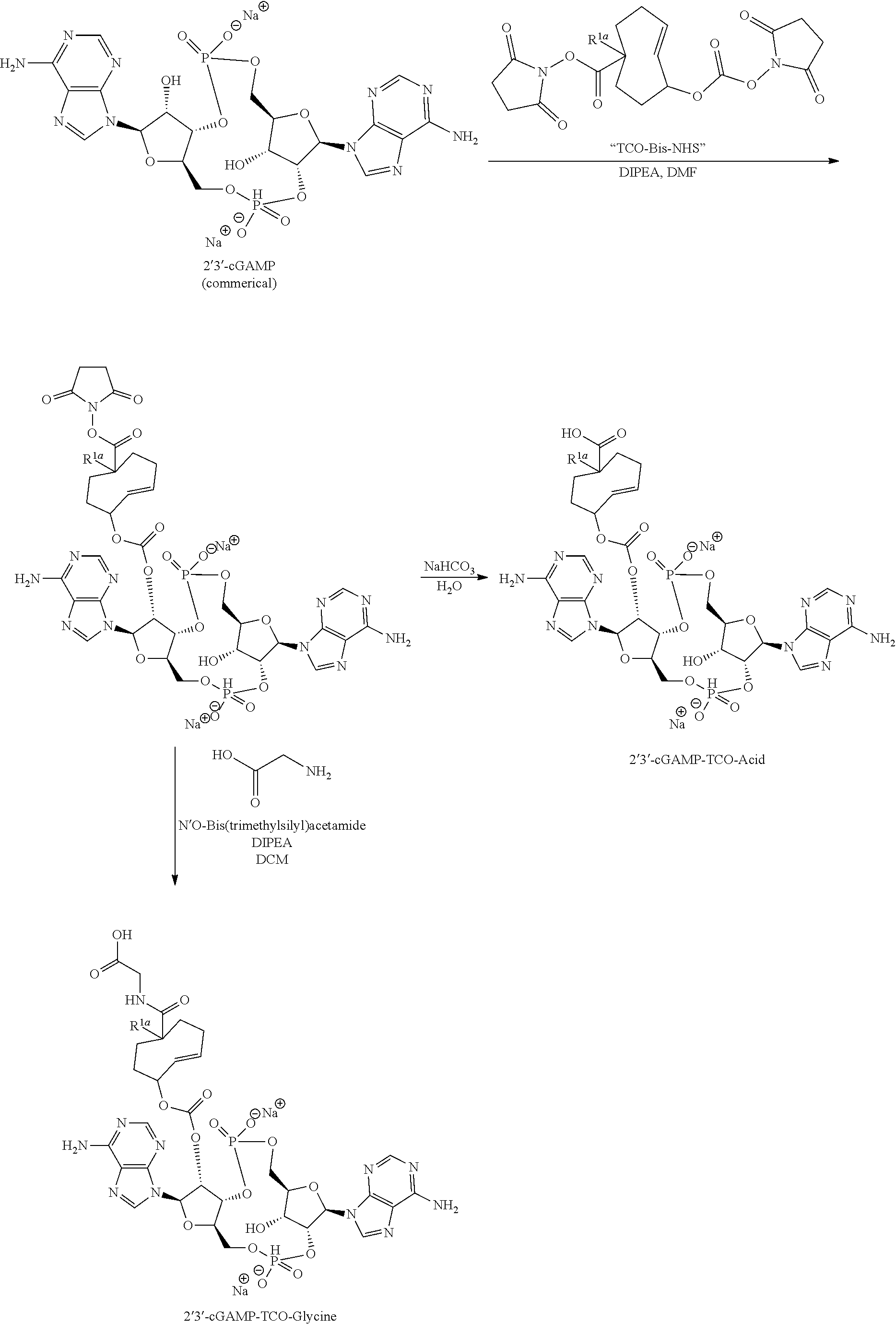

Scheme 10 illustrates a general method of conjugating a cyclic dinucleotide to a trans-cyclooctene, as in formula (I). The illustrated method proceeds by reaction of a cyclic dinucleotide molecule with a nitrophenyl carbonate substituted trans-cyclooctene in the presence of a base to form a mono- or bis-substituted cyclic dinucleotide, depending on the amount of trans-cyclooctene reagent.

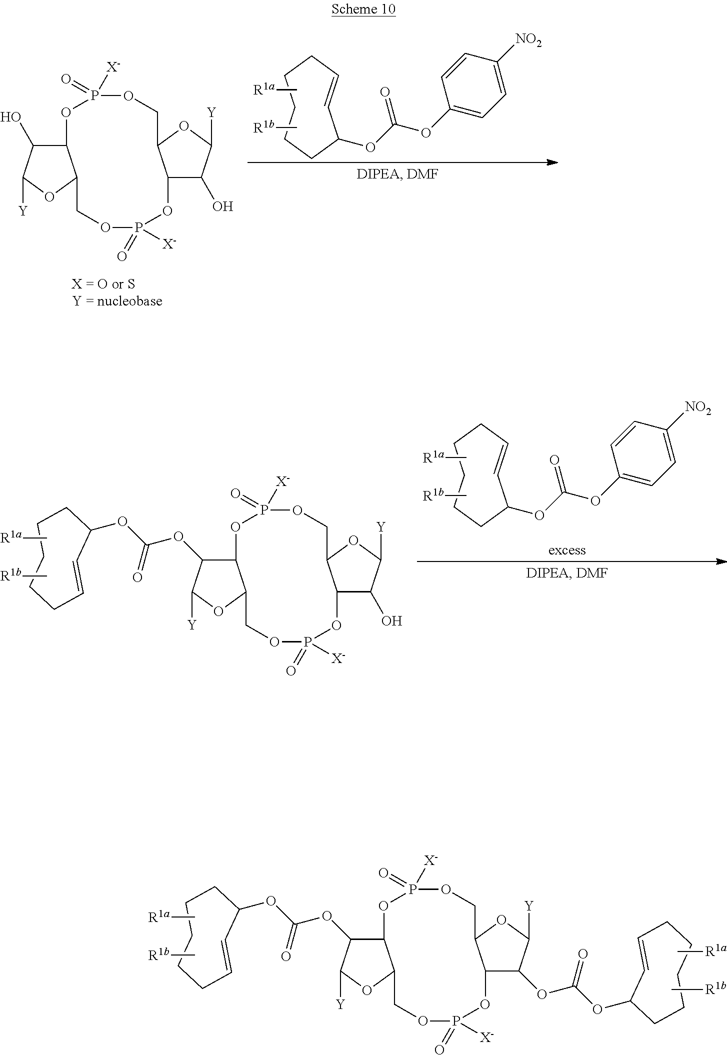

Scheme 11 illustrates a general method of conjugating a cyclic dinucleotide to a transcyclooctene wherein R² is —C₁₋₆alkylene-CO₂H, —CHR^{1e}CO₂H, —C₁₋₆alkylene-C(O)OC₁-4alkyl, C(O)OC₁₋₄alkyl, or —CHR^{1e}C(O)OC₁₋₄alkyl, which corresponds with R^{1b} in formula (I) being one of C(O)N(R^{1c})—C₁₋₆alkylene-CO₂H, C(O)OH, C(O)N(R^{1c})CHR^{1e}CO₂H, C(O)N(R^{1c})—C₁₋₆alkylene-C(O)OC₁₋₄alkyl, C(O)OC₁₋₄alkyl, or C(O)N(R^{1c})CHR^{1e}C(O)OC₁₋₄alkyl. The processes in Scheme 11 proceed analogously to those in Schemes 1-3, 7, and 9. The processes illustrated in Scheme 11 may be modified to provide bis-conjugated cyclic dinucleotides using excess trans-cyclooctene reagent, analogous to Scheme 10.

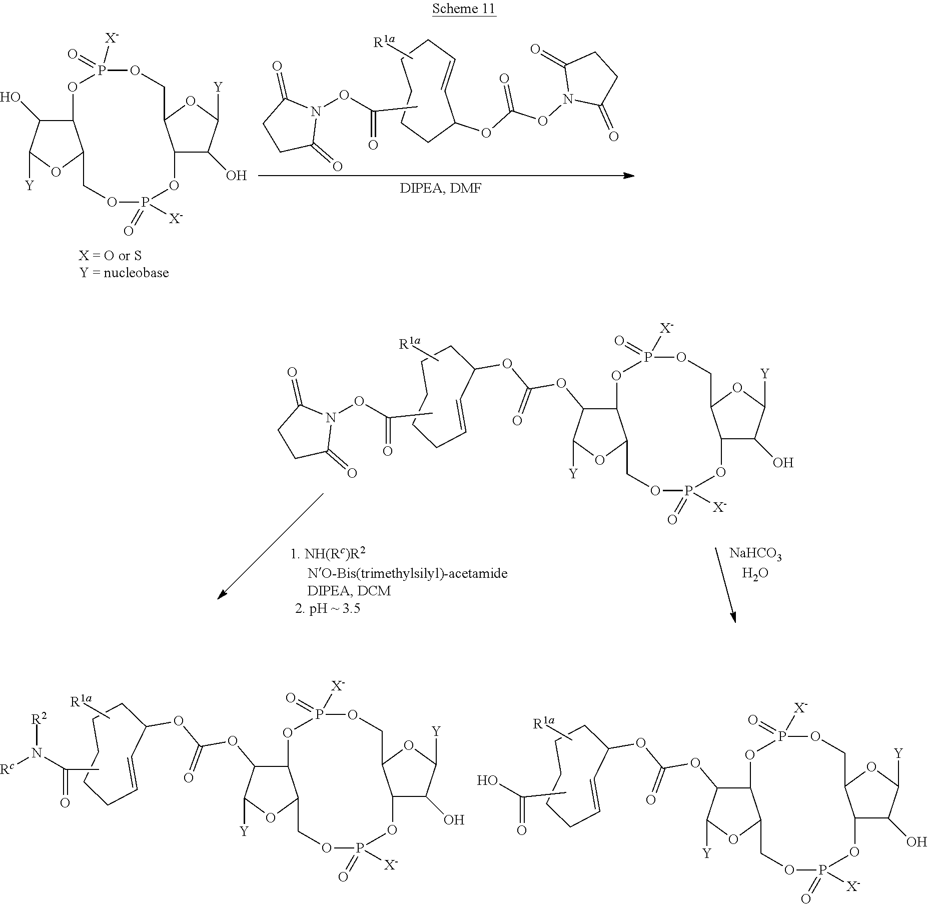

Schemes 12 and 13 illustrate representative synthetic methods of conjugating an imidazo[4,5-c]quinolin-4-amine to a trans-cyclooctene, as in formula (I), following analogous procedures to Schemes 10 and 11.

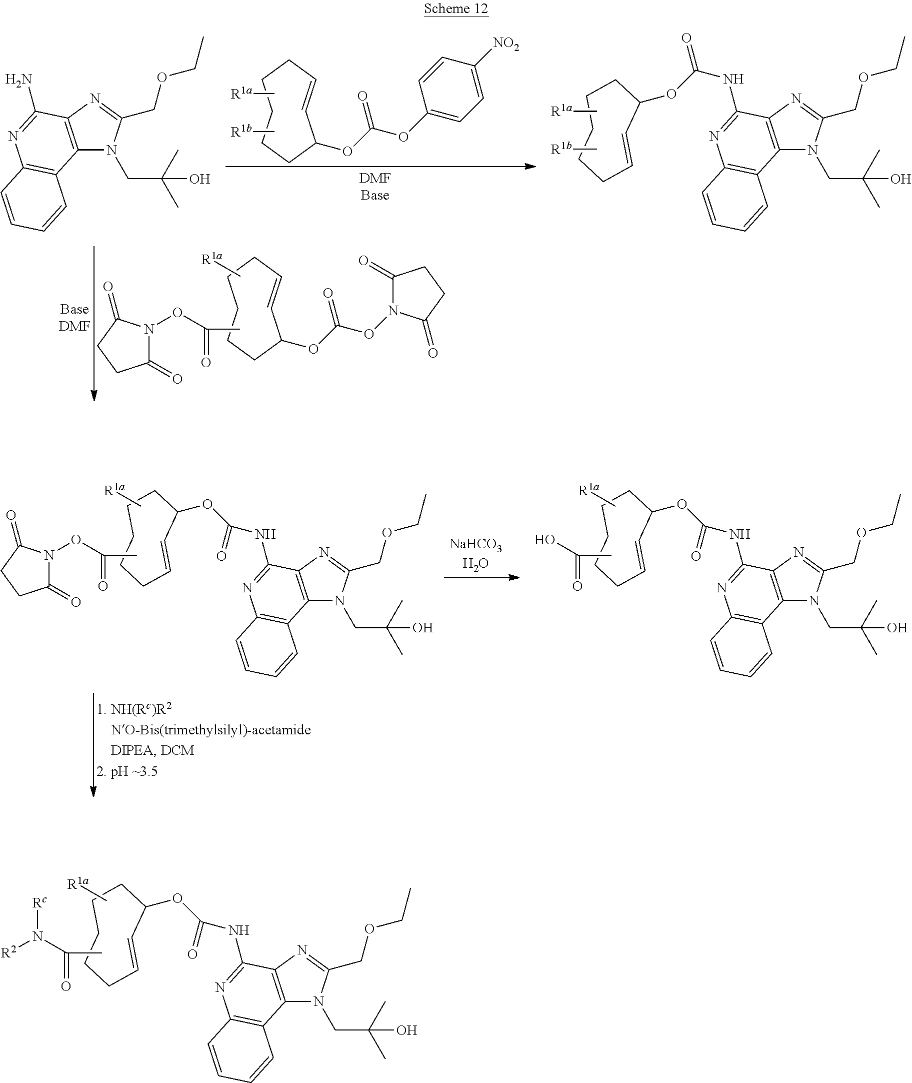

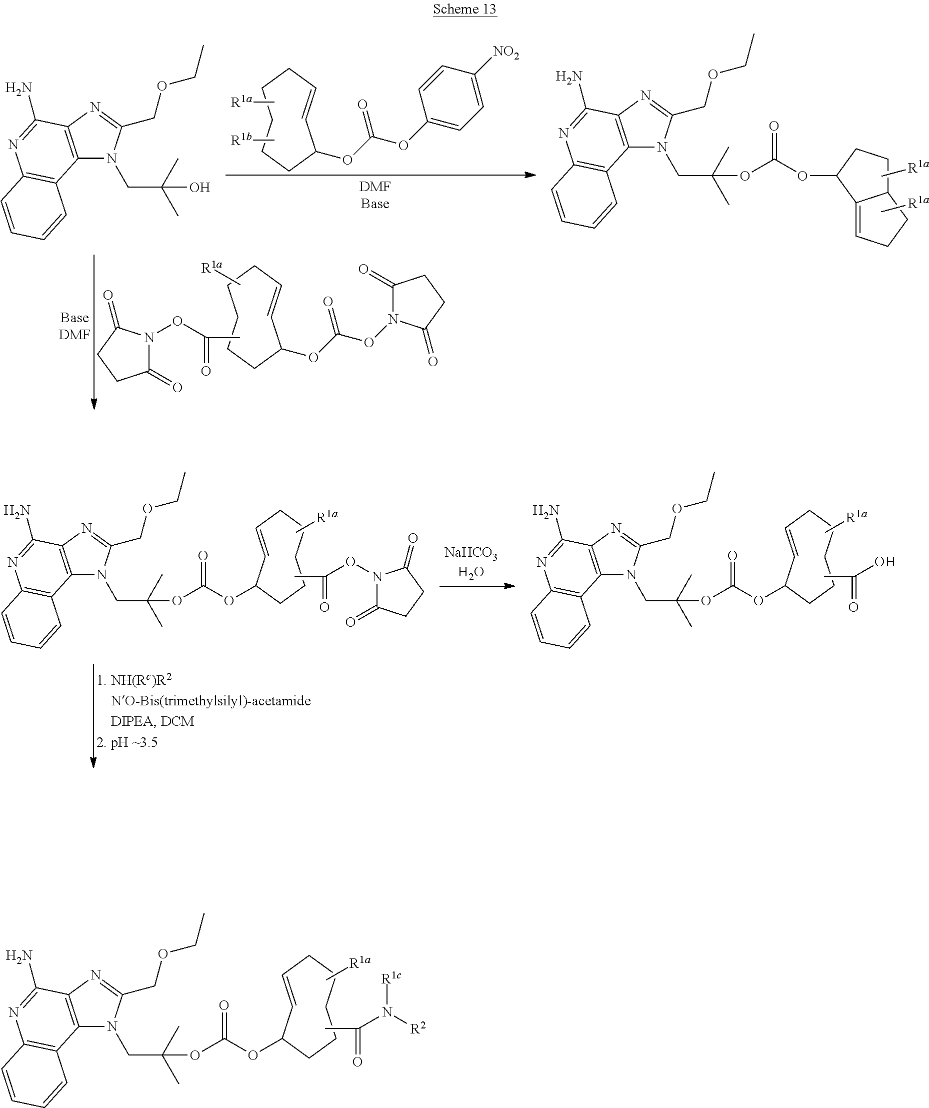

The disclosed compounds may be prepared in racemic form or as individual enantiomers or diastereomers by either stereospecific synthesis or by resolution. The compounds may, for example, be resolved into their component enantiomers or diastereomers by standard techniques, such as the formation of stereoisomeric pairs by salt formation with an optically active base, followed by fractional crystallization and regeneration of the free acid. The compounds may also be resolved by formation of stereoisomeric esters or amides, followed by chromatographic separation and removal of the chiral auxiliary. Alternatively, the compounds may be resolved using a chiral HPLC column. The enantiomers also may be obtained from kinetic resolution of the racemate of corresponding esters using lipase enzymes.

A compound described herein can be in the form of a salt, e.g., a pharmaceutically acceptable salt. The term “pharmaceutically acceptable salt” includes salts of the active compounds that are prepared with relatively nontoxic acids or bases, depending on the particular substituents found on the compounds described herein. Neutral forms of the compounds may be regenerated by contacting the salt with a base or acid and isolating the parent compound in a conventional manner. The parent form of the compound differs from the various salt forms in certain physical properties, such as solubility in polar solvents, but otherwise the salts are equivalent to the parent form of the compound for the purposes of this disclosure. Examples of pharmaceutically acceptable salts are discussed in Berge et al, 1977, “Pharmaceutically Acceptable Salts.” J. Pharm. Sci. Vol. 66, pp. 1-19.

For example, if the compound is anionic, or has a functional group which may be anionic (e.g., —COOH may be —COO), then a salt may be formed with a suitable cation. Examples of suitable inorganic cations include, but are not limited to, alkali metal ions such as Na⁺ and K⁺, alkaline earth cations such as Ca′ and Mg′, and other cations. Examples of suitable organic cations include, but are not limited to, ammonium ion (i.e., NH₄⁺) and substituted ammonium ions (e.g., NH₃R₁⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine.

If the compound is cationic, or has a functional group that may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous.

Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, gluchep tonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric. Examples of suitable polymeric organic anions include, but are not limited to, those derived from the following polymeric acids: tannic acid, carboxymethyl cellulose.

Unless otherwise specified, a reference to a particular compound also includes salt forms thereof.

It may be convenient or desirable to prepare, purify, and/or handle an active compound in a chemically protected form. The term “chemically protected form” is used herein in the conventional chemical sense and pertains to a compound in which one or more reactive functional groups are protected from undesirable chemical reactions under specified conditions (e.g., pH, temperature, radiation, solvent, and the like). In practice, well known chemical methods are employed to reversibly render unreactive a functional group, which otherwise would be reactive, under specified conditions. In a chemically protected form, one or more reactive functional groups are in the form of a protected or protecting group (also known as a masked or masking group or a blocked or blocking group). By protecting a reactive functional group, reactions involving other unprotected reactive functional groups can be performed, without affecting the protected group; the protecting group may be removed, usually in a subsequent step, without substantially affecting the remainder of the molecule. See, for example, Protective Groups in Organic Synthesis (T. Green and P. Wuts; 3rd Edition; John Wiley and Sons, 1999). Unless otherwise specified, a reference to a particular compound also includes chemically protected forms thereof.

A wide variety of such “protecting,” “blocking,” or “masking” methods are widely used and well known in organic synthesis. For example, a compound which has two nonequivalent reactive functional groups, both of which would be reactive under specified conditions, may be derivatized to render one of the functional groups “protected,” and therefore unreactive, under the specified conditions; so protected, the compound may be used as a reactant which has effectively only one reactive functional group. After the desired reaction (involving the other functional group) is complete, the protected group may be “deprotected” to return it to its original functionality.

A hydroxy group may be protected as an ether (—OR) or an ester (—OC(O)R), for example, as: a t-butyl ether; a benzyl, benzhydryl (diphenylmethyl), or trityl (triphenylmethyl) ether; a trimethylsilyl or t-butyldimethylsilyl ether; or an acetyl ester (—OC(O)CH₃, —OAc).

An aldehyde or ketone group may be protected as an acetal (RCH(OR)₂) or ketal (R₂C(OR)₂), respectively, in which the carbonyl group (R₂C═O) is converted to a diether (R₂C(OR)₂), by reaction with, for example, a primary alcohol. The aldehyde or ketone group is readily regenerated by hydrolysis using a large excess of water in the presence of acid.

An amine group may be protected, for example, as an amide (—NRC(O)R) or a urethane (—NRC(O)OR), for example, as: a methyl amide (—NHC(O)CH₃); a benzyloxy amide (—NHC(O)OCH₂C₆H5, —NH-Cbz); as a t-butoxy amide (—NHC(O)OC(CH₃)₃, —NH-Boc); a 2-biphenyl-2-propoxy amide (—NHCO(O)C(CH₃)₂C₆H₄C₆H₅, —NH-Bpoc), as a 9-fluorenylmethoxy amide (—NH—Fmoc), as a 6-nitroveratryloxy amide (—NH—Nvoc), as a 2-trimethylsilylethyloxy amide (—NH-Teoc), as a 2,2,2-trichloroethyloxy amide (—NH-Troc), as an allyloxy amide (—NH-Alloc), as a 2(-phenylsulphonyl)ethyloxy amide (—NH—Psec); or, in suitable cases (e.g., cyclic amines), as a nitroxide radical (>N-0«).

A carboxylic acid group may be protected as an ester, for example, as: an alkyl ester (e.g., a methyl ester; a t-butyl ester); a haloalkyl ester (e.g., a haloalkyl ester); a trialkylsilylalkyl ester; or an arylalkyl ester (e.g., a benzyl ester; a nitrobenzyl ester); or as an amide, for example, as a methyl amide.

A thiol group may be protected as a thioether (—SR), for example, as: a benzyl thioether; an acetamidomethyl ether (—S—CH₂NHC(O)CH₃).

A compound described herein can also be modified by appending appropriate functionalities to enhance selective biological properties. Such modifications are known in the art and include those that increase biological penetration into a given biological system (e.g., blood, lymphatic system, central nervous system), increase oral availability, increase solubility to allow administration by injection, alter metabolism, and/or alter rate of excretion. Examples of these modifications include, but are not limited to, esterification with polyethylene glycols, derivatization with pivolates or fatty acid substituents, conversion to carbamates, hydroxylation of aromatic rings, and heteroatom substitution in aromatic rings.

In certain embodiments, the products may be further modified, for example, by manipulation of substituents. These manipulations may include, but are not limited to, reduction, oxidation, organometallic cross-coupling, alkylation, acylation, and hydrolysis reactions which are commonly known to those skilled in the art. In some cases, the order of carrying out the foregoing reaction schemes may be varied to facilitate the reaction or to avoid unwanted reaction products.

### 4. Formulations

Another aspect of the invention provides a pharmaceutical composition comprising a) the compound of formula (II-A), or a pharmaceutically acceptable salt thereof; b) a one or more immunomodulatory agents, or a pharmaceutically acceptable salt thereof; and c) a pharmaceutically acceptable carrier.

Another aspect of the invention provides a pharmaceutical composition comprising a) a therapeutic support composition; b) one or more immunomodulatory agents, or a pharmaceutically acceptable salt thereof; and c) a pharmaceutically acceptable carrier.

Compositions (e.g., support composition, one or more immunomodulatory agents, and/or functionalized payload) can be provided in any suitable form, e.g., in the form of a pharmaceutically acceptable formulation, and can be formulated for any suitable route of administration, e.g., oral, topical or parenteral administration. Where the composition is provided as a liquid injectable (such as in those embodiments where they are administered intravenously or directly into a tissue), the composition can be provided as a ready-to-use dosage form, or as a reconstitutable storage-stable powder or liquid that may include pharmaceutically acceptable carriers and excipients.

A “pharmaceutically acceptable excipient,” “pharmaceutically acceptable diluent,” “pharmaceutically acceptable carrier,” or “pharmaceutically acceptable adjuvant” means an excipient, diluent, carrier, and/or adjuvant that are useful in preparing a pharmaceutical composition that are generally safe, non-toxic and neither biologically nor otherwise undesirable, and includes an excipient, diluent, carrier, and adjuvant that are acceptable for veterinary use and/or human pharmaceutical use. “A pharmaceutically acceptable excipient, diluent, carrier and/or adjuvant” as used herein includes one or more such excipients, diluents, carriers, and adjuvants.

Methods for formulating compositions can be adapted from those readily available. For example, compositions can be provided in a pharmaceutical formulation that includes a therapeutically effective amount of a composition and a pharmaceutically acceptable carrier (e.g., saline). The pharmaceutical formulation may optionally include other additives (e.g., buffers, stabilizers, preservatives, and the like). In some embodiments, the formulations are suitable for administration to a mammal, such as those that are suitable for administration to a human.

The compositions of the present disclosure can be prepared in a wide variety of oral, parenteral and topical dosage forms. Oral preparations include tablets, pills, powder, dragees, capsules, liquids, lozenges, cachets, gels, syrups, slurries, suspensions, etc., suitable for ingestion by the subject. The compositions of the present disclosure can also be administered by injection, that is, intravenously, intramuscularly, intracutaneously, subcutaneously, intraduodenally, or intraperitoneally. In some instances, the compositions described herein can be administered by inhalation, for example, intranasally. In some instances, the compositions of the present disclosure can be administered transdermally. In some instances, the compositions can be administered by intraocular, intravaginal, and intrarectal routes including suppositories, insufflation, powders and aerosol formulations (for examples of steroid inhalants, see Rohatagi, J. Clin. Pharmacol. 35: 1187-1193, 1995; Tjwa, Ann. Allergy Asthma Immunol. 75: 107-111, 1995). Accordingly, the present disclosure also provides pharmaceutical formulations including a composition as described herein and a pharmaceutically acceptable carrier or excipient.

For preparing pharmaceutical formulations from the compositions of the present disclosure, pharmaceutically acceptable carriers can be solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are found, for example in Remington's Pharmaceutical Sciences, Maack Publishing Co, Easton Pa. (“Remington's”).

In some embodiments, the pharmaceutical composition of the invention is a vaccine that comprises a compound of formula (I-A), or a pharmaceutically acceptable salt thereof, a pharmaceutically acceptable carrier, and optionally an antigen. Antigens for use in the immunogenic compositions provided herein may be provided in an effective amount (e.g., an amount effective for use in therapeutic or prophylactic methods). For example, immunogenic compositions of the invention may be used to treat or prevent diseases or conditions such as infections and cancer. Exemplary antigens include, but are not limited to, tumor antigens and infectious disease antigens. Antigens for use in the immunogenic compositions provided herein are typically macromolecules (e.g., polypeptides, polysaccharides, polynucleotides) that are foreign to the host. An antigen may be any target epitope, molecule (including a biomolecule), molecular complex (including molecular complexes that contain biomolecules), subcellular assembly, cell or tissue against which elicitation or enhancement of immunoreactivity in a subject is desired. Frequently, the term antigen may refer to a polypeptide antigen of interest. However, antigen, as used herein, may also refer to a recombinant construct which encodes a polypeptide antigen of interest (e.g, an expression construct). In certain preferred embodiments the antigen may be, or may be derived from, or may be immunologically cross-reactive with, an infectious pathogen and/or an epitope, biomolecule, cell or tissue that is associated with infection, cancer, autoimmune disease, allergy, asthma, or any other condition where stimulation of an antigen-specific immune response would be desirable or beneficial.

In certain embodiments, a tumor antigen or cancer antigen is used in conjunction with the immunogenic compositions provided herein. In certain embodiments, the tumor antigen is a peptide-containing tumor antigens, such as a polypeptide tumor antigen or glycoprotein tumor antigens. In certain embodiments, the tumor antigen is a saccharide-containing tumor antigen, such as a glycolipid tumor antigen or a ganglioside tumor antigen. In certain embodiments, the tumor antigen is a polynucleotide-containing tumor antigen that expresses a polypeptide-containing tumor antigen, for instance, an RNA vector construct or a DNA vector construct, such as plasmid DNA. In certain embodiments, the tumor antigen is a whole, live or dead or permeabilized cancer cell. Tumor antigens appropriate for the use in conjunction with the immunogenic compositions provided herein encompass a wide variety of molecules, such as (a) polypeptide-containing tumor antigens, including polypeptides (which can range, for example, from 8-20 amino acids in length, although lengths outside this range are also common), lipopolypeptides and glycoproteins, (b) saccharide-containing tumor antigens, including polysaccharides, mucins, gangliosides, glycolipids and glycoproteins, and (c) polynucleotides that express antigenic polypeptides.

In certain embodiments, the tumor antigens are, for example, (a) full length molecules associated with cancer cells, (b) homologs and modified forms of the same, including molecules with deleted, added and/or substituted portions, and (c) fragments of the same. In certain embodiments, the tumor antigens are provided in recombinant form. In certain embodiments, the tumor antigens include, for example, class I-restricted antigens recognized by CD8+ lymphocytes or class II-restricted antigens recognized by CD4+ lymphocytes.

In certain embodiments, the tumor antigens include, but are not limited to, (a) cancer-testis antigens such as NYESO-1, SSX2, SCP1 as well as RAGE, BAGE, GAGE and MAGE family polypeptides, for example, GAGE-1, GAGE-2, MAGE-1, MAGE-2, MAGE-3, MAGE-4, MAGE-5, MAGE-6, and MAGE-12 (which can be used, for example, to address melanoma, lung, head and neck, NSCLC, breast, gastrointestinal, and bladder tumors), (b) mutated antigens, for example, p53 (associated with various solid tumors, e.g., colorectal, lung, head and neck cancer), p21/Ras (associated with, e.g., melanoma, pancreatic cancer and colorectal cancer), CDK4 (associated with, e.g., melanoma), MUM1 (associated with, e.g., melanoma), caspase-8 (associated with, e.g., head and neck cancer), CIA 0205 (associated with, e.g., bladder cancer), HLA-A2-R1 701, beta catenin (associated with, e.g., melanoma), TCR (associated with, e.g., T-cell non-Hodgkins lymphoma), BCR-ab1 (associated with, e.g., chronic myelogenous leukemia), triosephosphate isomerase, KIA 0205, CDC-27, and LDLR-FUT, (c) over-expressed antigens, for example, Galectin 4 (associated with, e.g., colorectal cancer), Galectin 9 (associated with, e.g., Hodgkin's disease), proteinase 3 (associated with, e.g., chronic myelogenous leukemia), WT 1 (associated with, e.g., various leukemias), carbonic anhydrase (associated with, e.g., renal cancer), aldolaseA (associated with, e.g., lung cancer), PRAME (associated with, e.g., melanoma), HER-2/neu (associated with, e.g., breast, colon, lung and ovarian cancer), alpha-fetoprotein (associated with, e.g., hepatoma), KSA (associated with, e.g., colorectal cancer), gastrin (associated with, e.g., pancreatic and gastric cancer), telomerase catalytic protein, MUC-1 (associated with, e.g., breast and ovarian cancer), G-250 (associated with, e.g., renal cell carcinoma), p53 (associated with, e.g., breast, colon cancer), and carcinoembryonic antigen (associated with, e.g., breast cancer, lung cancer, and cancers of the gastrointestinal tract such as colorectal cancer), (d) shared antigens, for example, melanomamelanocyte differentiation antigens such as MART-1/Melan A, gp 100, MCI R, melanocyte-stimulating hormone receptor, tyrosinase, tyrosinase related protein-1/TRP1 and tyrosinase related protein-2/TRP2 (associated with, e.g., melanoma), (e) prostate associated antigens such as PAP, PSA, PSMA, PSHP1, PSM-P1, PSM-P2, associated with e.g., prostate cancer, (f) immunoglobulin idiotypes (associated with myeloma and B cell lymphomas, for example), and (g) other tumor antigens, such as polypeptide- and saccharide-containing antigens including (i) glycoproteins such as sialyl Tn and sialyl Lex (associated with, e.g., breast and colorectal cancer) as well as various mucins; glycoproteins are coupled to a carrier protein (e.g., MUC-1 are coupled to KLH); (ii) lipopolypeptides (e.g., MUC-1 linked to a lipid moiety); (iii) polysaccharides (e.g., Globo H synthetic hexasaccharide), which are coupled to a carrier proteins (e.g., to KLH), (iv) gangliosides such as GM2, GM12, GD2, GD3 (associated with, e.g., brain, lung cancer, melanoma), which also are coupled to carrier proteins (e.g., KLH).

In certain embodiments, the tumor antigens include, but are not limited to, p15, Hom/MeI-40, H-Ras, E2A-PRL, H4-RET, IGH-IGK, MYL-RAR, Epstein Barr virus antigens, EBNA, human papillomavirus (HPV) antigens, including E6 and E7, hepatitis Band C virus antigens, human T-cell lymphotropic virus antigens, TSP-180, p185erbB2, p180erbB-3, c-met, nm-23H₁, TAG-72-4, CA 19-9, CA 72-4, CAM 17.1, NuMa, K-ras, p16, TAGE, PSCA, CT7, 43-9F, 5T4, 791 Tgp72, beta-HCG, BCA225, BTAA, CA 125, CA 15-3 (CA 27.29\BCAA), CA 195, CA 242, CA-50, CAM43, CD68\KP1, CO-029, FGF-5, Ga733 (EpCAM), HTgp-175, M344, MA-50, MG7-Ag, MOV18, NB/70K, NY-CO-1, RCAS1, SDCCAG16, TA-90 (Mac-2 binding protein\cyclophilin C-associated protein), TAAL6, TAG72, TLP, TPS, and the like.

### 5. Methods of Treatment

Aspects of the present disclosure include methods for delivering a payload to a target location in a subject. In certain embodiments, the method includes selectively delivering a payload to the target location in a subject. Selective delivery of the payload includes delivering the payload to the target location (e.g., an organ or tissue, or portion thereof), without targeting other locations in the subject (e.g., other organs or tissues, or portions thereof) that do not need administration of the payload. Selective delivery of the payload may be achieved through use of the support compositions and the functionalized payloads described herein.

In some instances, a support composition of the present disclosure may be localized to a desired target location in a subject. For example, methods of the present disclosure may include administering to a subject a support composition as described herein. The support composition may be administered to the subject at a desired target location in the subject. In some instances, the support composition may be implanted into the subject at the desired target location in the subject. In some embodiments, the support composition may be attached to a targeting agent as described herein, and the method may include administering the support composition to the subject (e.g., administered systemically). In these embodiments, the support composition that is attached to a targeting agent may localize at a desired target location in the subject through specific binding of the targeting agent to its target (e.g., antibody-antigen interaction, and the like), or may localize on the surface of a desired target (e.g., a cell surface) through specific binding of the targeting agent to its target (e.g., antibody-antigen interaction, and the like).

As described herein, selective binding between bioorthogonal binding partners (e.g., between a tetrazine binding agent of the support composition and its complementary trans-cyclooctene binding agent of a functionalized payload) may occur. Due to the localized administration of the support composition to a desired location in the subject as described above, the selective binding between the binding agent of the support composition and its complementary binding agent of the functionalized payload will localize the payload to the desired target location. Accordingly, in certain embodiments, the method includes administering to the subject a functionalized payload such that the functionalized payload binds to the support composition to form a support complex. For example, the functionalized payload may be administered systemically to the subject. Upon administration of the functionalized payload to the subject, contact between the binding agent of the support composition and the complementary binding agent of the functionalized payload may occur, such that the binding agent and its complementary binding agent bind to one another to form a support complex, thereby selectively delivering the payload to the target location in the subject. In some embodiments, selective delivery of the functionalized payload results in a concentration of the payload at the target location that is greater than the concentration of the payload elsewhere in the subject (e.g., at non-targeted areas in the subject).

Indications for this approach, include cancer, both hematological and solid cancers, infections, wound healing, stenosis, ischemia, re-vascularization, myocardial infarction, arrhythmias, vascular occlusion (thrombi, through anticoagulants), inflammation through anti-proliferative drugs, corticosteroids and derivatives, and/or NSAIDS, autoimmune disorders, transplants, macular degeneration, rheumatoid arthritis, osteoarthritis, peri-prosthetic infections, through coating of implants, paste, wax, polymethylmethacrylate (PMMA) constructs, and others. In certain embodiments, the approach can be used for the treatment and/or diagnosis of soft tissue sarcomas: rhabdomyosarcoma, fibrosarcoma, Ewing's sarcoma, and all the different subtypes of soft tissue sarcoma as well as osteosarcoma. The compositions can be for the treatment and/or diagnosis of pigmented vilonodular synovitis.

The compositions of the present disclosure find use in treatment and/or diagnosis of a condition or disease in a subject that is amenable to treatment or diagnosis by administration of the payload (e.g., the parent drug (i.e., the drug prior to conjugation to the composition)). By “treatment” is meant that at least an amelioration of the symptoms associated with the condition afflicting the subject is achieved, where amelioration is used in a broad sense to refer to at least a reduction in the magnitude of a parameter, e.g., symptom, associated with the condition being treated. As such, treatment also includes situations where the pathological condition, or at least symptoms associated therewith, are completely inhibited, e.g., prevented from happening, or stopped, e.g., terminated, such that the subject no longer suffers from the condition, or at least the symptoms that characterize the condition. Treatment may include inhibition, that is, arresting the development or further development of clinical symptoms, e.g., mitigating or completely inhibiting an active disease. Treatment may include relief, that is, causing the regression of clinical symptoms. For example, in the context of cancer, the term “treating” includes any or all of: reducing growth of a solid tumor, inhibiting replication of cancer cells, reducing overall tumor burden, prolonged survival and ameliorating one or more symptoms associated with a cancer.

The subject to be treated can be one that is in need of therapy, where the subject to be treated is one amenable to treatment using the parent drug. Accordingly, a variety of subjects may be amenable to treatment using the compositions disclosed herein. Generally, such subjects are “mammals”, with humans being of interest. Other subjects can include domestic pets (e.g., dogs and cats), livestock (e.g., cows, pigs, goats, horses, and the like), rodents (e.g., mice, guinea pigs, and rats, e.g., as in animal models of disease), as well as non-human primates (e.g., chimpanzees, and monkeys).

The functionalized payloads, therapeutic support compositions, and methods can be used for the treatment, prevention, and/or diagnosis of any targeted disease. Indications for this approach, include cancer, both hematological and solid cancers, infections, wound healing, stenosis, ischemia, re-vascularization, myocardial infarction, arrhythmias, vascular occlusion (thrombi, through anticoagulants), inflammation through anti-proliferative drugs, corticosteroids and derivatives, and/or NSAIDS, autoimmune disorders, transplants, macular degeneration, rheumatoid arthritis, osteoarthritis, peri-prosthetic infections, through coating of implants, paste, wax, polymethylmethacrylate (PMMA) constructs, and others. In certain embodiments, the functionalized payloads, therapeutics support compositions, and methods can be used for the treatment, prevention, and/or diagnosis of soft tissue sarcomas: rhabdomyosarcoma, fibrosarcoma, Ewing's sarcoma, and all the different subtypes of soft tissue sarcoma as well as osteosarcoma. The compositions can be for the treatment and/or diagnosis of pigmented vilonodular synovitis.

In certain embodiments, the functionalized payloads, therapeutic support compositions, additional therapeutic agents, one or more immunomodulatory agents, and methods can be used for the treatment, prevention, and/or diagnosis of solid tumors, including but not limited to, melanoma (e.g., unresectable, metastatic melanoma), renal cancer (e.g., renal cell carcinoma), prostate cancer (e.g., metastatic castration resistant prostate cancer), ovarian cancer (e.g., epithelial ovarian cancer, such as metastatic epithelial ovarian cancer), breast cancer (e.g., triple negative breast cancer), glioblastoma (e.g., glioblastoma multiforme), and lung cancer (e.g., non-small cell lung cancer), soft tissue sarcoma, fibrosarcoma, osteosarcoma, pancreatic cancer, among others. The disclosed approach lends itself well as an adjuvant/neoadjuvant system. For example, particles as disclosed herein could be placed during the biopsy, once the results from the study come back, the practitioner could deliver the appropriate cocktail to the desired site in the body. This would minimize the size of the tumor particularly in the context of a surgically resectable tumor. Then at the end of the surgery, the surgeon could place more particles around the surgical cavity and treat the patient with further doses of treatment (e.g. chemotherapy through the disclosed approach) to minimize the risk of any cancer cells that may have been missed in the surgical margins.

In certain embodiments, the disclosed methods provide the ability to place particles as disclosed herein at the time of the biopsy. When the results return, the practitioner can deliver through to the biopsy site immunomodulatory agents such as TLR agonists, STING agonists, chemokines (agents that attract cancerous cells and/or immune cells) and adjuvants to enhance the immune system with fewer side effects as well as the chemotherapeutics agents combined with immunotherapy agents. This combination approach would be beneficial to patients. The chemotherapy agent would treat the solid tumor or specific location, while the enhanced response of the immunotherapy would help with distant metastatic sites. For example, in certain embodiments, the disclosed compositions and methods could employ or be used with anthracyclines, taxanes, gemcitabine and other agents to enhance the efficacy of one or more immunomodulatory agents such as ipilimumab, nivolumab, pembrolizumab, avelumab (also known as MSB0010718C; Pfizer).

The disclosed compounds and compositions may be used in methods of treatment. The methods of treatment disclosed herein may be used to treat bacterial infections. The methods of treatment disclosed herein may be used to treat or prevent MRSA infections. The methods of treatment disclosed herein may be used to treat cancer. The methods of treatment disclosed herein may be used to treat pigmented villonodular synovitis. The methods of treatment disclosed herein may be used to treat diseases or disorders related to inflammation. The methods of treatment disclosed herein may be used to treat arthritis.

a. Bacterial Infections

The disclosed methods may be used to treat or prevent bacterial infections. Although bacteria may not be harmful, and in some cases may be beneficial, bacteria may also lead to infection. Bacterial infections can affect multiple organs and body systems including, but not limited to, skin, mucous membranes, blood, lungs, kidneys, urinary tract, eyes, heart, intestines, meninges, respiratory tract, genitals, stomach, bone, connective tissue, and tissue surrounding organs. Bacterial infections may affect more than one organ or body system. Bacterial infections may be systemic. Bacterial infections may be asymptomatic. Bacterial infections may cause a variety of symptoms including, but not limited to, fever, inflammation, wounds that do not heal, weeping wounds, skin rash, red bumps on the skin, abscesses, swollen lymph nodes, nausea, diarrhea, headaches, earaches, sore throat, fatigue, low blood pressure, hyperventilation, weak and rapid pulse, local or systemic pain, and muscle aches. Bacterial infections may cause death. Subjects with co-morbidities or a compromised immune system may be more susceptible to bacterial infections. Bacterial infections may occur at surgical sites. Bacterial infections may be related to catheter placement.

The diagnosis of a bacterial infection may include, but are not limited to, symptomatic diagnostics, microbial culture, microscopy, biochemical tests, PCR based diagnostics, and metagenomics sequencing. A microbial examination may include sample collection, microbial cultivation, identification, and test of antibiotic susceptibility. The diagnosis may include gram staining of the bacterial culture. The diagnosis may include a coagulase test of the bacterial culture. The diagnosis may include a catalase test of the bacterial culture. The diagnosis may include blood tests. The blood tests may include, but are not limited to, a full blood count, measurement of C-reactive protein, measurement of procalcitonin, and measurement of rapid plasma reagin. The diagnosis may include ELISA. The diagnosis may include PCR. A rapid latex agglutination test that detects the PBP2a protein may be conducted to identify MRSA. The sample may be grown on an agar plate. The sample may be grown in nutrient broth. The growth conditions may include varying factors (e.g., type of growth medium, nutrients, selective compounds, antibiotics, temperature, pH level, oxygen level) to determine the type of bacteria growing. The determination of bacteria growing on an agar plate or in a nutrient broth may determine the bacteria responsible for the subject's infection. Discs containing antibiotic compounds may be placed on the agar plates. The antibiotic compounds may kill the bacteria growing on the plate. The greater the zone of dead bacteria around the disc (zone of inhibition) may indicate a more effective antibiotic.

Samples for diagnosing a bacterial infection may be obtained from the subject in need of treatment. The sample for testing may be from the site of the infection. A sample for testing may be obtained from the subject by swabbing of the skin, throat, or nose. A sample for testing may be obtained from the subject by collecting pus or fluids from wounds, abscesses, or other skin infections. A sample for testing may be obtained from the subject by collecting body fluids. The body fluids may include blood, sputum, urine, and/or other body fluids. Multiple samples may be taken from the subject. Multiple samples may be taken around the site of a prosthesis or medical device.

Bacterial infections may be treated with the compounds and compositions disclosed herein. Bacterial infections that may be treated by the compounds and compositions disclosed herein include, but are not limited to, Staphylococcus aureus, methicillin-resistant Staphylococcus aureus (MRSA), methicillin-sensitive Staphylococcus aureus (MSSA), Enterococcus faecalis, Enterococcus faecium, Escherichia coli, Salmonella, Neisseria, Bacillus, Brucella, Nocardia, Listeria monocytogenes, Lactobacillus plantarum, Lactococcus lactis, Francisella, Legionella, Yersinia pestis, Pseudomonas aeruginosa, Burkholderia cenocepacia, Mycobacterium avium, vancomycin-resistant Enterococci (VRE), and vancomycin-resistant Staphylococcus aureus (VRSA). The bacterial infection to be treated may be resistant to one or many antibiotics. Bacterial infections treated herein may be caused by Gram-positive bacteria. Bacterial infections treated herein may be caused by Gram-positive bacterial strains that are resistant to vancomyocin. Bacterial infections treated herein may be caused by multi-drug-resistant Gram-positive bacteria.

i. MRSA Infections

The disclosed methods may be used to treat MRSA. MRSA is any strain of Staphylococcus aureus that has developed multi-resistance to beta-lactam antibiotics, which include the penicillins (methicillin, dicloxacillin, nafcillin, oxacillin, etc.) and the cephalosporins. MRSA evolved from horizontal gene transfer of the mecA gene to at least five distinct S. aureus lineages. MRSA infections can quickly cause serious and life threatening internal infections including, but not limited to, sepsis, endocarditis, MRSA pneumonia bone infections, and infections of implants. MRSA may cause infections of the skin. The MRSA skin infections may lead to boils or abscesses. MRSA may cause systemic or internal infections. Some MRSA infections are untreatable with currently available antibiotics, usually resulting in severe, debilitating infection, or death. The MRSA infection may occur in subjects who have been hospitalized, which is known as health care-associated MRSA (HA-MRSA). The MRSA infection may be spread by skin-to-skin contact, which is known as community-associated MRSA (CA-MRSA). Cases of MRSA have increased in livestock animals. CC398, a variant of MRSA, has emerged in animals and is found in intensively reared production animals (e.g., pigs, cattle, and poultry), where it can be transmitted to humans as LA-MRSA (livestock-associated MRSA).

The strains of MRSA to be treated by the compounds and compositions disclosed herein may include, but are not limited to, CBD-635, ST250 MRSA-1, ST2470-MRSA-I, ST239-MRSA-III, ST5-MRSA-II, ST5-MRSA-IV, ST239-MRSA-III, EMRSA15, EMRSA16, MRSA252, ST5:USA100, EMRSA 1, ST8:USA300, ST1:USA400, ST8:USA500, ST59:USA1000, USA1100, USA600, USA800, USA300, ST30, ST93, ST80, ST59, CC22, CC8, CC425, and CC398.

ii. Catheter-Related Bloodstream Infections

The disclosed methods may be used to treat catheter-related bloodstream infections. Catheter-related bloodstream infection (CRBSI) is defined as the presence of bacteremia originating from an intravenous catheter. CRBSI may occur frequently, may be lethal, and may be a common cause of nosocomial bacteremia. Intravascular catheters are integral to the modern practices and are inserted in critically-ill patients for the administration of fluids, blood products, medication, nutritional solutions, and for hemodynamic monitoring. Central venous catheters (CVCs) may pose a greater risk of device-related infections than any other types of medical device and may be major causes of morbidity and mortality. They may be a source of bacteremia and septicemia in hospitalized patients. CRBSIs may be associated with CVCs.

The disclosed methods may be used to deliver molecular payloads to an implanted biomaterial (e.g., polymer or hydrogel substituted with a bioorthogonal group). The material may be implanted at a desired location of the body during any local manipulation even if the specific pathogen or problem has not been determined yet such as a surgical implant or indwelling device insertion (“local injection”). For example, a suitably modified polymer or hydrogel such as hyaluronic acid modified with a tetrazine (HAT) may be used to coat catheter materials or other implanted medical device using known procedures for coating plastic materials with hyaluronic acid. Coating procedures can be optimized on small sections of polyurethane (PU) or polyvinyl chloride (PVC) tubing. PU or PVC tubing can be treated with 3-aminopropyltriethoxysilane in distilled water to incorporate amine groups for covalent functionalization with hyaluronic acid (HA). A base layer of HAT or unmodified HA can then be bonded to the surface using carbodiimide chemistry conditions as detailed in the literature. Additional layers of HAT or HA can be deposited through repeated manual dip coating procedures using similar carbodiimide chemistry conditions until a total of 10 additional layers have been applied. The final coated tubing can be characterized by scanning electron microscopy to examine surface morphology, confocal microscopy to determine coating thickness, and contact angle measurement to evaluate surface hydrophilicity.

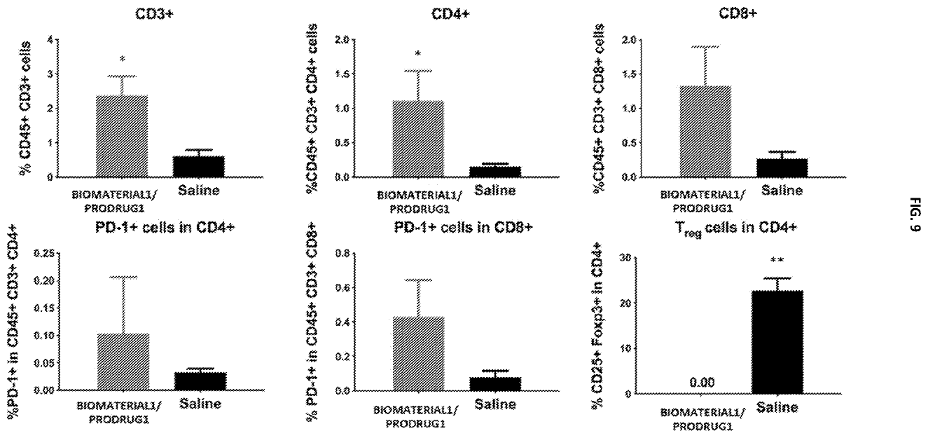

Following implantation of a biomaterial-coated device, an inactive prodrug, created by modifying a drug with the reaction partner, is injected into the blood stream whenever it is needed (“systemic exposure”). The inactive prodrugs spread throughout the body, but when they come near the biomaterial, whether in the form of a coating or gel, they quickly attach to it (“catch”), thus concentrating the prodrug at the desired location. Finally, the active drug is spontaneously released from the biomaterial to perform its function (“release”). This provides a system with the temporal control of systemic drug delivery, and effectively turns systemic drugs into localized medicines (FIG. 8).

Due to the limited systemic activity of the prodrug, problems related to the disruption of the body's natural microbiome, such as drug-resistant bacteria or the development of infections will be prevented. A supratherapeutic dose may be given, thus increasing the drug's therapeutic index and reducing the likelihood of bacteria at the site of infection developing resistance. Having the gel coat the surface of a CVC or other implanted device, the drug will be able to accumulate deep into tissues that systemic drugs in their usual doses cannot reach.

The disclosed methods may lead to “reloading” by a prodrug, ensuring local release and improved efficacy. This will lead to better utilization of antimicrobials and reduction of the emergence of resistant bacteria. If a bacterial or fungal infection turned out to be resistant to the first prodrug, then a second prodrug could be “caught and released” by the already-implanted gel or coated device. Standard technologies require implant removal and placement to achieve similar results. The disclosed biodegradable coating would not require an additional invasive procedure to implant or remove it.

b. Cancer

The disclosed methods may be used to treat or prevent cancer. Cancer is a group of related diseases that may include sustained proliferative signaling, evasion of growth suppressors, resistance to cell death, enablement of replicative immortality, induction of angiogenesis, and the activation of invasion and metastasis. The disclosed methods may enhance or elicits an immune response against a cancer in the subject. The immune response may lead to an increase in one or more of leukocytes, lymphocytes, monocytes, and eosinophils.

Cancer that may be treated by the disclosed methods, includes, but is not limited to, astrocytoma, adrenocortical carcinoma, appendix cancer, basal cell carcinoma, bile duct cancer, bladder cancer, bone cancer, brain cancer, brain stem cancer, brain stem glioma, breast cancer, cervical cancer, colon cancer, colorectal cancer, cutaneous T-cell lymphoma, diffuse intrinsic pontine glioma, ductal cancer, endometrial cancer, ependymoma, Ewing's sarcoma, esophageal cancer, eye cancer, fibrosarcoma, gallbladder cancer, gastric cancer, gastrointestinal cancer, germ cell tumor, glioma, hepatocellular cancer, histiocytosis, Hodgkin lymphoma, hypopharyngeal cancer, intraocular melanoma, Kaposi sarcoma, kidney cancer, laryngeal cancer, leukemia, liver cancer, lung cancer, lymphoma, macroglobulinemia, melanoma, mesothelioma, mouth cancer, multiple myeloma, nasopharyngeal cancer, neuroblastoma, non-Hodgkin lymphoma, osteosarcoma, ovarian cancer, pancreatic cancer, parathyroid cancer, penile cancer, pharyngeal cancer, pituitary cancer, prostate cancer, rectal cancer, renal cell cancer, retinoblastoma, rhabdomyosarcoma, sarcoma, skin cancer, small cell lung cancer, small intestine cancer, soft tissue carcinoma, soft tissue sarcoma, solid tumor, squamous cell carcinoma, stomach cancer, T-cell lymphoma, testicular cancer, throat cancer, thymoma, thyroid cancer, trophoblastic tumor, urethral cancer, uterine cancer, uterine sarcoma, vaginal cancer, vulvar cancer and Wilms tumor.

In some embodiments, the cancer that may be treated by the disclosed methods is melanoma, renal cancer, prostate cancer, ovarian cancer, breast cancer, glioma, lung cancer, soft tissue carcinoma, soft tissue sarcoma, osteosarcoma, or pancreatic cancer. In some embodiments, the cancer is a solid tumor. In some embodiments, the cancer is a soft tissue carcinoma. In some embodiments, the cancer is a fibrosarcoma. In some embodiments, the cancer is diffuse intrinsic pontine glioma.

Without being bound by a particular theory, local release of certain anti-cancer agents using the compounds and methods of the invention may produce or contribute to immunogenic cell death (ICD). For example, certain anti-cancer agents (e.g., anthracyclines, cyclophosphamide, oxaliplatin) have been reported to induce ICD. Kroemer et al. Annu. Rev. Immunol. 2013 (31), 51-72. Immunogenic apoptosis of cancer cells can induce an effective antitumour immune response through activation of dendritic cells (DCs) and consequent activation of specific T cell response. ICD is characterized by secretion of damage-associated molecular patterns (DAMPs). Three important DAMPs which are exposed to the cell surface during ICD. Calreticulin (CRT), one of the DAMP molecules, which is normally in the lumen of endoplasmic reticulum (ER), is translocated after the induction of immunogenic apoptosis to the surface of dying cell where it functions as an “eat me” signal for professional phagocytes. Other important surface exposed DAMPs are heat-shock proteins (HSPs), namely HSP70 and HSP90, which are under stress condition also translocated to the plasma membrane. On the cell surface they have an immunostimulatory effect, based on their interaction with number of antigen-presenting cell (APC) surface receptors like CD91 and CD40 and also facilitate crosspresentation of antigens derived from tumour cells on MHC class I molecule, which than leads to the CD8+ T cell response. Other important DAMPs, characteristic for ICD are secreted amphoterin (HMGB1) and ATP. HMGB1 is considered to be late apoptotic marker and its release to the extracellular space seems to be required for the optimal release and presentation of tumour antigens to dendritic cells. It binds to several pattern recognition receptors (PRRs) such as Toll-like receptor (TLR) 2 and 4, which are expressed on APCs. The most recently found DAMP released during immunogenic cell death is ATP, which functions as a “find-me” signal for monocytes when secreted and induces their attraction to the site of apoptosis. Kroemer et. al. Curr. Op. Immunol. 2008 (20), 504-511.

Thus, local release of ICD inducers using the compounds and methods of the invention may be beneficially combined with one or more immunomodulatory agents.

In one aspect, the invention provides a method of treating cancer comprising a) administering to a subject in need thereof a therapeutically effective amount of a compound of formula (II-A) or (III-A), or a pharmaceutically acceptable salt or composition thereof; and b) locally administering at a first tumor in the subject, a therapeutic support composition, as described herein; wherein the subject has a second tumor and the administration of a) and the administration of b) inhibits growth of the second tumor.

Another aspect provides a method of enhancing or eliciting an immune response against a second tumor in a subject comprising a) administering to the subject a therapeutically effective amount of a compound of formula (II-A) or (III-A), or a pharmaceutically acceptable salt or composition thereof; and b) locally administering at a first tumor in the subject, a therapeutic support composition, as described herein; wherein the administration of a) and the administration of b) enhances or elicits an immune response against the second tumor.

In another aspect, the invention provides a method of inhibiting tumor metastasis in a subject at risk of tumor metastasis comprising a) administering a compound of formula (II-A) or (III-A), or a pharmaceutically acceptable salt thereof to the subject; and b) locally administering a therapeutic support composition to the subject at a first tumor; wherein the compound of formula (II-A) or (III-A) and the therapeutic support composition are as defined herein.

In another aspect, the invention provides a pharmaceutical combination comprising a) a compound of formula (II-A), or a pharmaceutically acceptable salt, or composition thereof; and b) a therapeutic support composition; for use in a method of inhibiting growth of a second tumor in a subject, wherein the therapeutic support composition is locally administered at a first tumor in the subject and the compound of formula (II-A) or (III-A), or a pharmaceutically acceptable salt, or composition thereof is administered to the subject.

In another aspect, the invention provides a pharmaceutical combination comprising a) a compound of formula (II-A) or (III-A), or a pharmaceutically acceptable salt, or composition thereof; and b) a therapeutic support composition; for use in a method of enhancing or eliciting an immune response against a second tumor in a subject, wherein the therapeutic support composition is locally administered at a first tumor in the subject and the compound of formula (II-A) or (III-A), or a pharmaceutically acceptable salt, or composition thereof is administered to the subject.

In another aspect, the invention provides a pharmaceutical combination comprising a) a compound of formula (II-A) or (III-A), or a pharmaceutically acceptable salt, or composition thereof; and b) a therapeutic support composition; for use in a method of inhibiting tumor metastasis in a subject at risk of tumor metastasis, wherein the therapeutic support composition is locally administered at a first tumor in the subject and the compound of formula (II-A) or (III-A), or a pharmaceutically acceptable salt, or composition thereof is administered to the subject.

In another aspect, the invention provides the use of a combination comprising a) a compound of formula (II-A) or (III-A), or a pharmaceutically acceptable salt, or composition thereof; and b) a therapeutic support composition; in the manufacture of a medicament for inhibiting growth of a second tumor, wherein the therapeutic support composition is locally administered at a first tumor in the subject and the compound of formula (II-A) or (III-A), or a pharmaceutically acceptable salt, or composition thereof is administered to the subject.

In another aspect, the invention provides use of a combination comprising a) a compound of formula (II-A) or (III-A), or a pharmaceutically acceptable salt, or composition thereof; and b) a therapeutic support composition; in the manufacture of a medicament for enhancing or eliciting an immune response against a second tumor, wherein the therapeutic support composition is locally administered at a first tumor in the subject and the compound of formula (II-A) or (III-A), or a pharmaceutically acceptable salt, or composition thereof is administered to the subject.

In another aspect, the invention provides use of a combination comprising a) a compound of formula (II-A) or (III-A), or a pharmaceutically acceptable salt, or composition thereof; and b) a therapeutic support composition; in the manufacture of a medicament for inhibiting tumor metastasis in a subject at risk of tumor metastasis, wherein the therapeutic support composition is locally administered at a first tumor in the subject and the compound of formula (II-A) or (III-A), or a pharmaceutically acceptable salt, or composition thereof is administered to the subject.

The methods of inhibiting tumor metastasis in a subject at risk of tumor metastasis may further comprise a step of identifying and/or selecting the subject at risk of tumor metastasis. The subject at risk of tumor metastasis may be identified from a tumor biopsy to assess the pathological state of the tumor, through serum and/or tissue biomarkers, and/or through imaging techniques.

In the methods and uses disclosed herein, the second tumor may be present or absent in a subject at the time of administration of the compound of formula (II-A) or (III-A), or a pharmaceutically acceptable salt, or composition thereof; and the therapeutic support composition. In the methods and uses disclosed herein, the administration of the compound of formula (II-A) or (III-A), or a pharmaceutically acceptable salt, or composition thereof; and the therapeutic support composition may inhibit the formation or development of a second tumor (i.e., prevention). In some embodiments, the therapeutic support composition is not administered locally at the second tumor. The methods of treating cancer or enhancing or eliciting an immune response, and the disclosed pharmaceutical combination, may be further combined with use of an immunomodulatory agent. Alternatively, the optional immunomodulatory agent may be excluded.

Without wishing to be bound by a particular theory, the methods and uses disclosed herein may inhibit metastasis or formation or growth of a secondary tumor by eliciting or enhancing an immune response against the primary tumor (localized therapeutic support composition) and/or secondary tumor (no localized therapeutic support composition). The immune response may be an increase or decrease in one or more of innate and adaptive immune cells. For example, the immune response may be an increase or decrease of one or more of leukocytes, lymphocytes, monocytes, eosinophils, and antibodies. Further for example, the immune response may be an increase in CD3, CD4, CD8, and/or PD-1 positive tumor-infiltrating lymphocytes, in the first tumor and/or second tumor. The immune response may also be a decrease in regulatory T-cells in the first tumor and/or second tumor.

Without wishing to be bound by a particular theory, treatment of murine mammary carcinoma and fibrosarcoma tumors with doxorubicin results in IFN-g-producing CD8+ T cell proliferation and their recruitment to tumors As is similarly observed with radiation therapy, some types of cytotoxic compounds (e.g., anthracyclines, cyclophosphamide, and oxaliplatin) also activate immunogenic cell death pathways whereby cell surface expression of calreticulin is followed by release of ATP, HMGB1, and HSPs, thereby leading to DC-mediated crosspresentation of tumor antigens to CD8+ T cells. Corroborating in vitro evidence indicates that exposure of cancer cells to 5-fluoruracil or doxorubicin stimulates HSP release and promotes engulfment of cell debris by DCs, thereby promoting cross-presentation to CD8+ T cells. Similarly, when doxorubicin-treated cancer cells are injected into syngeneic mice, DCs phagocytose cell debris and generate a tumor-specific CD8+ T cell antitumor immune response (Medler T R, et. al. Trends Cancer. 2015; 1(1):66-75.).

Inhibition of metastasis or a secondary tumor untreated with a therapeutic support composition may be caused cell death in a treated tumor. Cell death may lead to release of stress molecules and antigens to the tumor microenvironment. These antigens can be presented to cytotoxic T-cells by antigen presenting cells which may elicit local and systemic immune responses to cells with similar antigens at the location of the second tumor. The treatment can recruit macrophages, NK cells, and cytotoxic T cells to a secondary tumor, leading to an overall increase in tumor infiltrating lymphocytes and subsequent immunologic anti-tumor response in secondary tumors.

The methods may be used to inhibit metastasis of solid malignant tumors in subjects at risk of tumor metastasis. Subjects at risk of tumor metastasis include those who are stage IV (metastatic disease) with multiple tumors, or stage II-III (local spread). Subjects at risk of tumor metastasis also include those who have high-grade solid tumors, those who show tissue and/or serum biomarkers that are indicative of metastasis. Tumors classified as either grade 3 or “high grade” have poor cell tissue differentiation and spread more quickly than grade 1 and 2 tumors. Biomarkers of metastasis include but are not limited to: CCR7, E-cadherin, CXCR4, VEGF, VEGFR, E-cadherin, EpCAM, VCAM, integrin-alpha10, N-cadherin, vimentin, and fibronectin. Further biomarkers include AGR2, AGR3, Alpha-enolase, CA125, CRP, SAA, IL6, IL8, CacyBP, CCR7, E-cadherin, CXCR4, CYFRA21-1, EGFR, EMP2, EphA2, Galectin-1, GDF15, H2K18ac, H3K4me2, H3K9me2, HE4, HER2-neu, HSP27, HSP60, IGFBP2, IGFBP3, IGFBP7, IL6, IL6sR, ILK, Integrin αᵥβ₆, LCN2, MSLN, Muc-1, PDX6, Plectin, SAA, SPARC, TFF3, TGF-β1, TGM2, TGM4, triosephosphate isomerase, USP9X, VCAM-1, VEGF-C, VEGF-D, VVEGFR-3, as described by Brinton et al., Cancer Genomics & Proteomics (2012) 9: 345-356, which is incorporated herein by reference.

Biomarkers may be protein biomarkers. Protein biomarkers may indicate a risk of tumor metastasis by either an increase or decrease in protein expression compared to a reference sample from a non-metastatic or non-cancer control.

In some embodiments, in the subject at risk of metastasis, first tumor cells are separated from the first tumor. In further embodiments, the first tumor cells are present in tissue surrounding the first tumor, present in tumor cell-platelet aggregates, present in systemic circulation of the subject, and/or present at a second tissue location in the subject.

In certain embodiments, the functionalized payloads, therapeutic support compositions, and methods can be used for the treatment, prevention, and/or diagnosis of solid tumors, including but not limited to, melanoma (e.g., unresectable, metastatic melanoma), renal cancer (e.g., renal cell carcinoma), prostate cancer (e.g., metastatic castration resistant prostate cancer), ovarian cancer (e.g., epithelial ovarian cancer, such as metastatic epithelial ovarian cancer), breast cancer (e.g., triple negative breast cancer), glioblastoma (e.g., glioblastoma multiforme), and lung cancer (e.g., non-small cell lung cancer), soft tissue sarcoma, fibrosarcoma, osteosarcoma, pancreatic cancer, among others.

The disclosed approach lends itself well as an adjuvant/neoadjuvant system. For example, therapeutic support compositions as disclosed herein could be placed during the biopsy, once the results from the study come back, the practitioner could administer the appropriate cocktail to deliver treatment to the desired site in the body (compound of formula (II-A) and optional additional therapeutic agent(s)). The results of the biopsy may indicate the amount and type of treatment to deliver to the site of a tumor. For example, chemokines (agents that attract cancerous cells and/or immune cells) and adjuvants to enhance the immune system with fewer side effects as well as the chemotherapeutics agents could be delivered and combined with immunotherapy agents.

The disclosed compounds and compositions may be administered prior to surgical resection. The disclosed methods may minimize the size of the tumor prior to surgical resection. This would minimize the size of the tumor particularly in the context of a surgically resectable tumor. The disclosed compounds and compositions may be administered during surgical resection. The disclosed compounds and compositions may be administered after surgical resection. Therapeutic support composition may be placed around the surgical cavity at the end of surgical resection and the subject may then be treated with further doses of a treatment (e.g., pro-doxorubicin) to minimize the risk of any cancer cells that may have been missed in the surgical margins.

The disclosed methods may include multiple systemic doses of functionalized payload that focus at one location. The disclosed methods may be used to deliver a second payload. The disclosed methods may be used to administer a second functionalized payload if the tumor is resistant to the first payload. A second payload may be a TCO-labeled payload of gemcitabine or docetaxel. The TCO-labeled payload of gemcitabine or docetaxel may be administered in combination with doxorubicin. The second functionalized payload may be activated by the therapeutic support composition used for the first prodrug.

The functionalized payloads disclosed herein may function as adjuvants. This combination approach would be beneficial to patients. The chemotherapy agent would treat the solid tumor or specific location and may enhance or elicit an immune response, while the enhanced response of the immunotherapy of the functionalized payload and/or separate agent may help with distant metastatic sites. For example, in certain embodiments, the disclosed compositions and methods could employ or be used with anthracyclines, taxanes, gemcitabine and other agents to enhance the efficacy of ipilimumab, nivolumab, pembrolizumab, avelumab (also known as MSB0010718C; Pfizer).

i. Diffuse Intrinsic Pontine Gliomas

The disclosed methods may be used to treat diffuse intrinsic pontine gliomas. Diffuse intrinsic pontine gliomas (DIPG) are pediatric brainstem tumors that may be highly malignant and may be difficult to treat. There is no known curative treatment for DIPG, and survival odds have remained dismal over the past four decades. DIPG patients have a median overall survival of just 11 months, with a two-year survival rate below 10%. DIPG account for 75-80% of brainstem tumors in children, affecting an estimated 200-300 children in the U.S. each year. The rarity of this devastating disease and previous lack of experimental model systems has impeded research, and over the past four decades survival odds have remained the same. Diagnosis of DIPG may begin with clinical symptoms and may be confirmed by MRI. The disease may begin with several months of generalized symptoms, including behavioral changes and difficulties in school, double vision, abnormal or limited eye movements, an asymmetric smile, loss of balance, and weakness. Alternately, severe neurologic deterioration may happen more quickly, with symptoms present for less than a month prior to diagnosis. Clinical examination may reveal the triad of multiple cranial neuropathies, long tract signs such as hyperreflexia and clonus, as well as ataxia. Expansion of the pons section of the brainstem may cause obstructive hydrocephalus and increased intracranial pressure.2

Nuclei critical for life-sustaining function such as breathing and heartbeat in are located in the pons and without treatment, breathing and heartbeat may be damaged by DIPG.

The disclosed methods may be used to deliver molecular payloads to the site of a DIPG (e.g., an HDAC inhibitor such as panobinostat). The disclosed methods may include delivering drugs systemically that are only activated at the tumor site. The disclosed methods may be used as a neoadjuvant or adjuvant therapy. The biomaterial may be placed during a biopsy. The results of the biopsy may indicate the amount and type of treatment to deliver to the site of a tumor. The disclosed compounds and compositions may be administered prior to surgical resection. The disclosed methods may minimize the size of the tumor prior to surgical resection. The disclosed compounds and compositions may be administered during surgical resection. The disclosed compounds and compositions may be administered after surgical resection. Biomaterial may be placed around the surgical cavity at the end of surgical resection and the subject may then be treated with further doses of a treatment (e.g., pro-doxorubicin). The disclosed biodegradable gel may be implanted at the time of biopsy or surgery. The disclosed methods may not require an additional invasive procedure to deliver additional doses of the disclosed compounds and compositions.

The disclosed methods may include multiple systemic doses of functionalized payload that focus at one location. The disclosed methods may be used to deliver a second payload. The disclosed methods may be used to administer a second functionalized payload if the tumor is resistant to the first payload. A second payload may be a TCO-labeled payload of gemcitabine or docetaxel. The TCO-labeled payload of gemcitabine or docetaxel may be administered in combination with doxorubicin. The second functionalized payload may be activated by the therapeutic support composition used for the first prodrug.

c. Inflammation Related Diseases or Disorders

The disclosed methods may be used to treat or prevent disease and disorders related to inflammation. Diseases and/or disorders which may be treated and/or prevented by the disclosed methods include, but are not limited to, asthma, arthritis, rheumatoid arthritis, osteoarthritis, autoimmune diseases, autoinflammatory diseases, celiac disease, chronic prostatis, diverticulitis, glomerulonephritis, otitis, necrotizing enterocolitis, inflammatory bowel disease, Crohn's disease, ulcerative colitis, colitis, Behcet's disease, vasculitis, transplant rejection, and autoimmune thyroid disease.

i. Pigmented Villonodular Synovitis

The disclosed methods may be used to treat pigmented villonodular synovitis. Pigmented villonodular synovitis (PVNS), also known as tenosynovial giant cell tumor (TGCT), is a chronic, progressive neoplastic process that causes the synovial lining of a joint, bursa, or tendon sheath to thicken and overgrow in an aggressive manner with a very low risk of metastasis. This condition affects approximately 1.8 people per million, or about 600, per year in the U.S and most commonly appears in those aged 20 to 45 years old. PVNS may be focal or diffuse. In the diffuse form, the disease process may accelerate tendon and joint wear and may have a 40-50% rate of local recurrence with traditional treatment strategies. The benign but aggressive behavior of PVNS makes treatment challenging as clinicians have to weigh the morbidity of treatment relative to the natural history of the disease process. Methods that locally deliver and activate therapeutics may be the solution to conditions such as diffuse PVNS. This limits systemic side effects of medications. Diffuse pigmented villonodular synovitis (PVNS) which synonymously goes by the name of tenosynovial giant cell tumor (TGCT) in extraarticular manifestations of the disease, is a primarily localized, aggressive neoplastic process affecting the synovial lining of a joint, bursa, or tendon sheath, causing it to thicken overgrow, and induce a destructive inflammatory process.

In both localized and diffuse types of PVNS, the majority of cases have a genetic rearrangement in chromosome 1p11-13, a site for the macrophage colony stimulating factor (CSF-1). The translocation leads to CSF-1 overexpression, attracting inflammatory cells expressing CSF-1 receptor (CSF1R) and driving the formation of PVNS.13 CSF-1, a secreted cytokine and hematopoietic growth factor, plays an essential role in the proliferation, differentiation, and survival of monocytes, macrophages, and related cells.

Within tissue affected by PVNS, only a small population of mononuclear stromal cells (2-16%) have been demonstrated to overexpress CSF-1, and these neoplastic cells constitute a minor component within the tumor. However, most of the cells are non-neoplastic, have high levels of receptor (CSFR1) expression and are recruited and activated by the CSF1 produced by the neoplastic cells. Because CSFR1 is a group III receptor tyrosine kinase, it has been theorized that a tyrosine kinase receptor inhibitor (TKI) targeting CSF1R (e.g., imatinib, nilotinib, emactuzumab, and PLX3397) might inhibit PVNS progression and reduce surgical morbidity and preserve patient quality of life.

There are at least two forms of the disease, which may be histologically identical. A first, focal PVNS/GCTTS may appear in joints or around tendon sheaths that support the joint. It may manifest as a localized extraarticular process, usually affecting the small joints of the hand or wrist (65%-89%) and the foot and ankle (5%-15%), or as localized intraarticular disease, usually affecting the knee, hip, or ankle. The disclosed methods may be used to treat the first form of PVNS/GCTTS. A second type of PVNS is the diffuse form that affects the entire synovial lining. This is most common in large joints usually the hip (4-16%) and knee (66-80%), but can occur in other joints as well (ankle, shoulder, elbow, spine, etc.). This form of the disease is more invasive and more difficult to successfully treat with surgical excision. The disclosed methods may be used to treat the second form of PVNS.

Patients with symptomatic, aggressive PVNS, especially the diffuse form, currently undergo treatments with long-term consequences. The contemporary approach of surgery and radiation is too morbid for a condition that is ultimately benign. The recent development of systemic medication with an effect on the CSF-1R pathway has created an exciting new approach to this frustrating condition. Use as an adjuvant to surgery has demonstrated promising early results, however, side effects continue to be a limitation. The disclosed methods that locally deliver and activate therapeutics will be readily beneficial to treat PVNS while avoiding the long-term sequelae of the treatment itself. The disclosed methods may eliminate the need for surgery in patients with PVNS. The disclosed methods may eliminate the need for surgery in the focal form of PVNS. The disclosed methods may reduce the recurrence of PVNS. The disclosed methods may reduce local recurrence in the diffuse form of PVNS.

ii. Arthritis

The disclosed methods of treatment may be used to treat arthritis. Arthritis is a term that may mean any disorder that affects joints. Symptoms may include joint pain and stiffness. Other symptoms may include redness, warmth, swelling, and decreased range of motion of the affected joints. In some types of arthritis, other organs may also be affected. Onset may be gradual or sudden. There may be over 100 types of arthritis. The most common forms are osteoarthritis and rheumatoid arthritis. Osteoarthritis may occur with aging and may affect the fingers, knees, and hips. Rheumatoid arthritis is an autoimmune disorder that may affect the hand joints, feet joints, skin, lungs, heart and blood vessels, blood, kidneys, eyes, liver, bones and neurological system.

In some embodiments, the disclosed compounds and compositions may be used to treat infections, tissue injury, stenosis, ischemia, re-vascularization, myocardial infarction, arrhythmias, vascular occlusion, inflammation, autoimmune disorders, transplant rejection, macular degeneration, rheumatoid arthritis, osteoarthritis, peri-prosthetic infections, and pigmented villonodular synovitis.

b. Modes of Administration

Methods of treatment may include any number of modes of administering a disclosed compound or composition. Modes of administration may include tablets, pills, dragees, hard and soft gel capsules, granules, pellets, skin patches, skin creams, skin gels, aqueous, lipid, oily or other solutions, emulsions such as oil-in-water emulsions, liposomes, aqueous or oily suspensions, syrups, elixirs, solid emulsions, solid dispersions or dispersible powders. For the preparation of pharmaceutical compositions for oral administration, the compound or compositions disclosed herein may be admixed with adjuvants and excipients, such as gum arabic, talcum, starch, sugars (such as, e.g., mannitose, methyl cellulose, lactose), gelatin, surface-active agents, magnesium stearate, aqueous or non-aqueous solvents, paraffin derivatives, cross-linking agents, dispersants, emulsifiers, lubricants, conserving agents, flavoring agents (e.g., ethereal oils), solubility enhancers (e.g., benzyl benzoate or benzyl alcohol) or bioavailability enhancers (e.g. Gelucire®). In the pharmaceutical composition, the compound or compositions disclosed herein may also be dispersed in a microparticle, e.g. a nanoparticulate composition.

For parenteral administration, the compounds or compositions disclosed herein may be dissolved or suspended in a physiologically acceptable diluent, such as water, buffer, oils with or without solubilizers, surface-active agents, dispersants or emulsifiers. Suitable oils may include, for example, olive oil, peanut oil, cottonseed oil, soybean oil, castor oil and sesame oil. For parenteral administration, the compound or compositions disclosed herein may be administered in the form of an aqueous, lipid, oily or other kind of solution or suspension, or even administered in the form of liposomes or nano-suspensions.

The term “parenterally,” as used herein, refers to modes of administration which include intravenous, intramuscular, intraperitoneal, intrasternal, subcutaneous and intraarticular injection and infusion.

The compounds and compositions disclosed herein may be administered topically. A topical composition disclosed herein may be applied to the skin of a subject in need thereof. The area of skin selected for treatment may be the site of a bacterial infection. The area of skin selected for treatment may be skin surrounding the infection site. The area of skin selected for treatment may be the site of a bacterial infection and the skin surrounding the infection site. The infection of the skin may be caused by MRSA. A topical composition disclosed herein may be applied to a mucous membrane of a subject in need thereof. The mucous membrane selected for treatment may be the site of a bacterial infection. The area of the mucous membrane selected for treatment may be the mucous membrane surrounding the bacterial infection. The mucous membrane selected for treatment may be the site of a bacterial infection and the mucous membrane surrounding the site of the infection. The infection of the mucous membrane may be caused by MRSA.

The topical administration may be with a patch containing the compounds and compositions disclosed herein. The topical administration may be with a dissolvable patch containing the compound and compositions disclosed herein. The topical administration may be with a cream containing the compound and compositions disclosed herein. The topical administration may be with foam containing the compound and compositions disclosed herein. The topical administration may be with lotion containing the compound and compositions disclosed herein. The topical administration may be with an ointment containing the compound and compositions disclosed herein. The topical administration may be with gel containing the compound and compositions disclosed herein. The topical administration may have fewer side effects than systemic administration of antibiotics.

In some embodiments, a topical composition comprising a therapeutically effective amount of the compounds and compositions disclosed herein may be applied to the infected skin and/or mucous membrane of a subject to reduce or eliminate the infection, and/or improve healing of the wounded skin and/or mucous membrane. In particular embodiments, a topical composition comprising a therapeutically effective amount of the compounds and compositions disclosed herein may be applied to an area of the skin and/or mucous membrane infected by MRSA, including infections caused by MRSA biofilm. In these embodiments, the compounds and compositions disclosed herein may be administered alone or in combination of one or more other active agents to reduce infection and/or promote skin and/or mucous membrane healing.

Therapeutic support compositions are preferably administered locally at the site of a tumor, such as by injection or implantation. Functionalized payloads, such as compounds of formula (I-A), (I-B), (II-A), or (III-A), may be administered by any convenient route, in view of a subject's condition and judgment of medical professionals. Parenteral administration is a suitable means of administering compounds of formula (I-A), (I-B), (II-A), or (III-A).

The amount of composition administered to a subject can be initially determined based on guidance of a dose and/or dosage regimen of the parent drug. In general, the compositions can provide for targeted delivery and/or enhanced serum half-life of the bound drug, thus providing for at least one of reduced dose or reduced administrations in a dosage regimen. Thus, the compositions can provide for reduced dose and/or reduced administration in a dosage regimen relative to the parent drug prior to being conjugated in a composition of the present disclosure.

The compositions of the present disclosure can be delivered by any suitable means, including oral, parenteral and topical methods. For example, transdermal administration methods, by a topical route, can be formulated as applicator sticks, solutions, suspensions, emulsions, gels, creams, ointments, pastes, jellies, paints, powders, and aerosols.

The pharmaceutical formulation may be provided in unit dosage form. In such form the pharmaceutical formulation may be subdivided into unit doses containing appropriate quantities of the compositions of the present disclosure. The unit dosage form can be a packaged preparation, the package containing discrete quantities of the preparation, such as packeted tablets, capsules, and powders in pouches, vials or ampoules. Also, the unit dosage form can be a capsule, tablet, dragee, cachet, or lozenge, or it can be the appropriate number of any of these in packaged form.

Compositions of the present disclosure can be present in any suitable amount, and can depend on various factors including, but not limited to, weight and age of the subject, state of the disease, etc. Suitable dosage ranges for the composition of the present disclosure include from 0.1 mg to 10,000 mg, or 1 mg to 1000 mg, or 10 mg to 750 mg, or 25 mg to 500 mg, or 50 mg to 250 mg. For instance, suitable dosages for the composition of the present disclosure include 1 mg, 5 mg, 10 mg, 20 mg, 30 mg, 40 mg, 50 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg or 1000 mg.

In some embodiments, multiple doses of a composition are administered. The frequency of administration of a composition can vary depending on any of a variety of factors, e.g., severity of the symptoms, condition of the subject, etc. For example, in some embodiments, a composition is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid).

The compositions of the present disclosure can be administered at any suitable frequency, interval and duration. For example, the composition of the present disclosure can be administered once an hour, or two, three or more times an hour, once a day, or two, three, or more times per day, or once every 2 days, 3 days, 4 days, 5 days, 6 days, or 7 days, so as to provide the desired dosage level to the subject. When the composition of the present disclosure is administered more than once a day, representative intervals include 5 min, 10 min, 15 min, 20 min, 30 min, 45 min and 60 minutes, as well as 1 hr, 2 hr, 4 hr, 6 hr, 8 hr, 10 hr, 12 hr, 16 hr, 20 hr, and 24 hours. The composition of the present disclosure can be administered once, twice, or three or more times, for an hour, for 1 to 6 hours, for 1 to 12 hours, for 1 to 24 hours, for 6 to 12 hours, for 12 to 24 hours, for a single day, for 1 to 7 days, for a single week, for 1 to 4 weeks, for a month, for 1 to 12 months, for a year or more, or even indefinitely.

The compositions of the present disclosure can be co-administered with another active agent. Co-administration includes administering the composition of the present disclosure and active agent within 0.5 hr, 1 hr, 2 hr, 4 hr, 6 hr, 8 hr, 10 hr, 12 hr, 16 hr, 20 hr, or 24 hours of each other. Co-administration also includes administering the composition of the present disclosure and active agent simultaneously or approximately simultaneously (e.g., within about 1 min, 5 min, 10 min, 15 min, 20 min, or 30 minutes of each other), or sequentially in any order. In addition, the composition of the present disclosure and the active agent can each be administered once a day, or two, three, or more times per day so as to provide the desired dosage level per day.

Co-administration can be accomplished by coimplantation or coinjection.

In some embodiments, co-administration can be accomplished by co-formulation, e.g., preparing a single pharmaceutical formulation including both the composition of the present disclosure and the active agent. In other embodiments, the composition of the present disclosure and the active agent can be formulated separately and co-administered to the subject.

The composition of the present disclosure and the active agent can be present in a formulation in any suitable weight ratio, such as from 1:100 to 100:1 (w/w), or 1:50 to 50:1, or 1:25 to 25:1, or 1:10 to 10:1, or 1:5 to 5:1 (w/w). The composition of the present disclosure and the other active agent can be present in any suitable weight ratio, such as 1:100 (w/w), 1:75, 1:50, 1:25, 1:10, 1:5, 1:4, 1:3, 1:2, 1:1, 2:1, 3:1, 4:1, 5:1, 10:1, 25:1, 50:1, 75:1, or 100:1 (w/w). Other dosages and dosage ratios of the composition of the present disclosure and the active agent are suitable in the formulations and methods described herein.

c. Combination Therapies

In one aspect, the invention provides a method of treating cancer or enhancing or eliciting an immune response comprising administering to a subject in need thereof: a therapeutically effective amount of a compound of formula (II-A) or (III-A), or a pharmaceutically acceptable salt or composition thereof a therapeutic support composition, as described herein; and a therapeutically effective amount of one or more immunomodulatory agents, or a pharmaceutically acceptable salt thereof.

The invention also provides a pharmaceutical combination comprising of a compound of formula (II-A) or (III-A), or a pharmaceutically acceptable salt, or composition thereof a therapeutic support composition, as described herein; and one or more immunomodulatory agents, for use in the treatment or prevention of a disease or disorder, such as cancer, infections, tissue injury, stenosis, ischemia, re-vascularization, myocardial infarction, arrhythmias, vascular occlusion, inflammation, autoimmune disorders, transplant rejection, macular degeneration, rheumatoid arthritis, osteoarthritis, peri-prosthetic infections, and pigmented villonodular synovitis; or for use in enhancing or eliciting an immune response. The invention also provides a pharmaceutical combination comprising of a compound of formula (II-A) or (III-A), or a pharmaceutically acceptable salt, or composition thereof a therapeutic support composition, as described herein; and one or more immunomodulatory agents, for use in a method of treating or preventing a disease or disorder, such as cancer, infections, tissue injury, stenosis, ischemia, re-vascularization, myocardial infarction, arrhythmias, vascular occlusion, inflammation, autoimmune disorders, transplant rejection, macular degeneration, rheumatoid arthritis, osteoarthritis, peri-prosthetic infections, and pigmented villonodular synovitis; or for use in a method of enhancing or eliciting an immune response.

The invention also provides the use of a pharmaceutical combination comprising a) a compound of formula (II-A) or (III-A), or a pharmaceutically acceptable salt, or composition thereof; a therapeutic support composition; and one or more immunomodulatory agents in the manufacture of a medicament for the treatment or prevention of a condition or disorder such as cancer, infections, tissue injury, stenosis, ischemia, re-vascularization, myocardial infarction, arrhythmias, vascular occlusion, inflammation, autoimmune disorders, transplant rejection, macular degeneration, rheumatoid arthritis, osteoarthritis, peri-prosthetic infections, and pigmented villonodular synovitis; or for use in enhancing or eliciting an immune response.

In the methods and uses described herein, the pharmaceutical combination of the compound of formula (II-A) or (III-A), or a pharmaceutically acceptable salt, or composition thereof; the therapeutic support composition; and the one or more immunomodulatory agents may be administered/used simultaneously, separately, or sequentially, and in any order, and the components may be administered separately or as a fixed combination. For example, the delay of progression or treatment of diseases according to the invention may comprise administration of the first active ingredient in free or pharmaceutically acceptable salt form and administration of the second active ingredient in free or pharmaceutically acceptable salt form, simultaneously or sequentially in any order, in jointly therapeutically effective amounts or effective amounts, e.g. in daily dosages corresponding to the amounts described herein. The individual active ingredients of the combination can be administered separately at different times during the course of therapy or concurrently in divided or single dosage forms. The instant invention is therefore to be understood as embracing all such regimes of simultaneous or alternating treatment and the term “administering” is to be interpreted accordingly. Thus, a pharmaceutical combination, as used herein, defines either a fixed combination in one dosage unit form or separate dosages forms for the combined administration where the combined administration may be independently at the same time or at different times. As a further example, the therapeutic support composition and the one or more immunomodulatory agents may be administered/used simultaneously (e.g., through coinjection or coimplantation), separately, or sequentially, followed by administration of the compound of formula (II-A) or (III-A).

The methods and uses in treating cancer include administering/localizing the therapeutic support composition at a tumor. In the methods and uses disclosed herein, the administration of the compound of formula (II-A) or (III-A), or a pharmaceutically acceptable salt, or composition thereof; the therapeutic support composition; and the one or more immunomodulatory agents may inhibit the growth of the tumor.

Any of the methods and uses, including the combination therapy disclosed herein using formulas (I), (I-A), (II-A), or (III-A) may be further combined with additional therapeutic agents, such as anticancer agents, antibacterial agents, immunomodulatory agents, and vaccines.

An additional therapeutic agent may be an anticancer agent, wherein the anticancer agent may be any anticancer agent described herein as an anticancer payload drug in formula (I-B) or (II-A).

An additional therapeutic agent may be a vaccine that comprises an adjuvant and/or an antigen.

An additional therapeutic agent may be a TLR or STING agonist as described herein for formula (I)/(I-A). Other immunomodulatory agents include cytokines, chemokines, chemokine antagonists, and immune checkpoint inhibitors.

Cytokines may limit tumour cell growth by a direct anti-proliferative or pro-apoptotic activity, or indirectly by stimulating the cytotoxic activity of immune cells against tumour cells. Cytokines that may be used as immunomodulatory agents include, but are not limited to, IFN-alpha, IFN-beta, and IFN-gamma, interleukins (e.g., IL-1 to IL-29, in particular, IL-7, IL-12, IL-15, IL-18, and IL-21), tumor necrosis factors (e.g., TNF-alpha and TNF-beta), erythropoietin (EPO), MIP3a, ICAM, macrophage colony stimulating factor (M-CSF), granulocyte colony stimulating factor (GCSF) and granulocyte-macrophage colony stimulating factor (GM-CSF) as described in US 2008/0014222. In embodiments of the invention, the cytokine is IL-2, IL-2 covalently bound to immunoglobulins (e.g., cergutuzumab amunaleukin, RO6874281) or PEG molecules (e.g., NKTR-214), IL-10, PEGylated IL-10 (e.g., pegilodecakin), IL-12, IL-15, recombinant aglycosylated IL-15, fusion protein of IL-15 with the binding domain of IL-15R^{a} (e.g., RLI), triple fusion protein comprising human IL-15, the binding domain of IL-15R^{a} and apolipoprotein A-I, ALT-803 (i1-15 fused to IgG1 Fc domain), IL-21, GM-CSF, talimogene laherparepvec, IFN-α, pegylated IFN-α, apolipoprotein A-I fusion protein with IFN-α.

The inhibitors of certain cytokines, their cognate receptor agonists and/or antagonists may also be used as cancer therapy. Cytokines are secreted or membrane-bound proteins that act as mediators of intercellular signaling to regulate homeostasis of the immune system. They are produced by cells of innate and adaptive immunity in response to microbes, self-antigens and tumor antigens. Inhibitors of TNF-α (e.g., infliximab, certolizumab) particularly in the context of PD-1 pathway blockade, TGF-β (e.g., galunisertib, fresolimumab, M7824), and CSF-1 (e.g., pexidartinib, cabiralizumab) may be used in the methods of the invention.

Immunotherapy with cytokines and cytokine inhibitors is described by Berraondo et al., British Journal of Cancer (2019) 120, 6-15, which is incorporated herein by reference.

Chemokines and/or chemokine receptor inhibitors may be used as immunomodulatory agents; they are chemotactic proteins that have the potential to attract macrophages, T-cells, eosinophils, basophils, and other cells to sites of inflammation, infection and/or tumor growth. These proteins are usually of low molecular mass (7-9 kD). Chemokines form four main structural subclasses (C, CC, CXC, and CX3C) categorized through their primary amino acid structure, which contain various combinations of conserved cysteine residues.

Immunomodulatory chemokines that may be suitable are CCL27 and CCL28, CC (CCL2, CCL3, CCL5) and CXC (CXCL1, CXCL2, CXCL5, CXCL6, CXCL8, CXCL9, CXCL10, CXL12).

ELRCXC chemokines, including IL-8, GROα, GROβ, GROγ, NAP-2, and ENA-78 (Strieter, 1995, J Biol Chem, 270:27348-57), have also been implicated in the induction of tumor angiogenesis (new blood vessel growth). Angiogenic activity is due to ELRCXC-chemokine binding to, and activation of CXCR2, and possibly CXCR1 for IL-8, expressed on the surface of vascular endothelial cells (ECs) in surrounding vessels. Many different types of tumors have been shown to produce ELRCXC chemokines. Chemokine production has been correlated with a more aggressive phenotype (Inoue, 2000, Clin Cancer Res, 6:2104-2119) and poor prognosis (Yoneda, 1998, J Nat Cancer Inst, 90:447-54). Chemokines are potent chemotactic factors and the ELRCXC chemokines, in particular, have been shown to induce EC chemotaxis. Thus, these chemokines are thought to induce chemotaxis of endothelial cells toward their site of production in the tumor. This may be a critical step in the induction of angiogenesis by the tumor. Inhibitors of CXCR2 or dual inhibitors of CXCR2 and CXCR1 will inhibit the angiogenic activity of the ELRCXC chemokines and therefore block the growth of the tumor. This anti-tumor activity has been demonstrated for antibodies to IL-8 (Arenberg, 1996, J Clin Invest, 97:2792-802), ENA-78 (Arenberg, 1998, J Clin Invest, 102:465-72), and GROα (Haghnegandar, 2000, J Leukoc Biology, 67:53-62). CXC chemokine inhibitors include

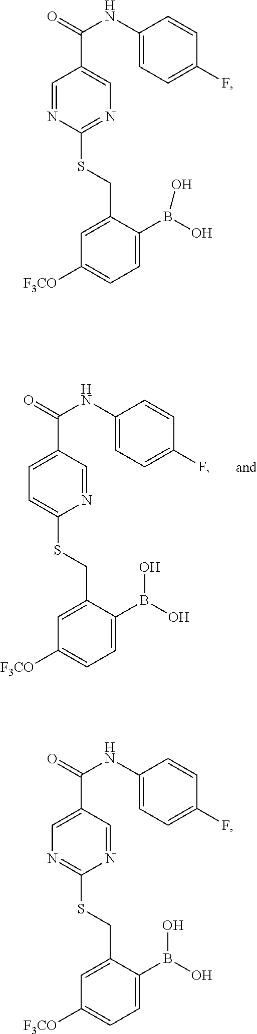 [SMILES: O=C(Nc1ccc(F)cc1)c1cnc(SCc2cc(OC(F)(F)F)ccc2B(O)O)nc1 ; O=C(Nc1ccc(F)cc1)c1ccc(SCc2cc(OC(F)(F)F)ccc2B(O)O)nc1 ; "and" ; O=C(Nc1ccc(F)cc1)c1cnc(SCc2cc(OC(F)(F)F)ccc2B(O)O)nc1]

as described in U.S. Pat. No. 10,046,002, which is incorporated herein by reference.

Immunomodulatory agents suitable for use with the methods of the present invention include chemokine or chemokine receptor antagonists that inhibit the recruitment of suppressive immune cells into the tumor microenvironment. For example, but not exclusively, the methods of the present invention may use a CCR1, CCR2 or CCR5 antagonist that reduces the infiltration of myeloid suppressor cells and regulatory T cells.

Suitable CCR, CXCR, and CCL inhibitors include inhibitors of CCR1 (e.g., CCX721, BL5923), CCR2 (e.g., CCX9588, PF-04136309, CCX872, RDC018, 747, iCCR2), CCL2 (e.g., CNTO 888), CCR4 (e.g., Affi 5, AF399/420/1802), CCR5 (e.g. Maraviroc), CCR7 (e.g., siRNA, MSM R707), CXCR2 (e.g., Navarixin, SB225002, Reparixin, SB265610, AZD5069), CXCR4 (e.g., AMD3100, AMD3465, LY2510924, BKT140, BMS-936564, PF-06747143, PRX177561, POL5551, USL311, CTCE-9908), as described by Poeta et al., Frontiers in Immunology (2019), Article 379, doi: 10.3389/fimmu.2019.00379; Yu et al., Cancer Biol. Ther. (2008) 7:1037-43; and Chi et al., Int. J Clin Exp Pathol. (2015) 8:12533-40.

Immune checkpoint inhibitors include but are not limited to PD-1 inhibitors (e.g. nivolumab, pembrolizumab, pidilizumab, sintilimab, AMP-224), PD-L1 inhibitors (e.g. atezolizumab, avelumab, durvalumab, BMS-936559), CTLA4 inhibitors (e.g. ipilimumab, tremelimumab) IDO inhibitors (e.g. indoximod, epacadostat), TIGIT inhibitors (e.g., LAG-3, such as an anti-LAG-3 antibody described in US2015/0259420, which is incorporated herein by reference; TIM-3, such as an anti-TIM-3 antibody described in US2015/0218274, which is incorporated herein by reference), and a BTLA pathway antagonist.

In some embodiments, the immune response is modulated using a xenobiotic agent, biologic agent, natural or artificially-derived adjuvants, cell-based therapy and/or checkpoint inhibitors including but not limited to the inhibitors of PD-1, PD-L1, CTLA-4, B7 molecules, TIGIT, Tim-3 and/or Lag-3, indoleamine 2,3-dioxygenase (IDO).

An additional therapeutic agent may be an immune checkpoint inhibitor. Immune checkpoint inhibitors include PD-1 inhibitors (e.g. nivolumab, pidilizumab, sintilimab), PD-L1 inhibitors (e.g. atezolizumab, avelumab, durvalumab, BMS-936559), CTLA4 inhibitors (e.g. ipilimumab, tremelimumab) or IDO inhibitors (e.g. indoximod, epacadostat).

An additional therapeutic agent may be a compound of formula (I-B), or a pharmaceutically acceptable salt thereof.

For the treatment of bacterial infection, the compounds and compositions may be combined with a variety of antibiotics. The antibiotics include, but are not limited to, vancomycin, linezolid, teicoplanin, ceftaorline, clindamycin, mupirocin, trimethoprim-sulfamethoxazole, tetracyclines, daptomycin, sulfa drugs, ceftobiprole, ceftaroline, dalbavancin, telavancin, torezolid, iclaprim, nemonoxacin, platensimycin, and oxadiazoles.

The compounds and compositions may be combined with agents that inhibit bacterial biofilm formation. The agents that inhibit bacterial biofilm formation include, but are not limited to, imidazole derivatives, indole derivatives, emodin, flavonoids, ginger extracts, Hypericum perforatum, 7-epiclusianone, isolimonic acid, tannic acid, chelerythrine, carvacrol, bgugaine, resveratrol, garlic extracts, natural halogenated furanones, brominated alkylidene lactams, and AHLs-based inhibitors.

The compounds and compositions may be combined with lysine-conjugated aliphatic norspermidine analogues. The compounds and compositions may be combined with phage therapy. In the case of infection involving a medical device or prosthesis, the compounds and compositions may be administered in combination with the removal of the medical device or prosthesis. A new, sterile medical device or prosthesis may be implanted in the subject.

The compounds and compositions may be combined with agents to modify potential side effects from antibacterial agents. Agents that may mediate or treat side effects include, but are not limited to, probiotics, anti-diarrheal agents, anti-emetic agents, and analgesics.

The subject may also be undergoing a variety of treatments for co-morbidities.

Additional therapeutic agent(s) may be administered simultaneously or sequentially with the disclosed compounds and compositions. Sequential administration includes administration before or after the disclosed compound and compositions. An additional therapeutic agent may be administered before the disclosed compounds and compositions. An additional therapeutic agent may be administered after the disclosed compounds and compositions. An additional therapeutic agent may be administered at the same time as the disclosed compounds and compositions. In some embodiments, the additional therapeutic agent or agents may be administered in the same composition as the disclosed compounds. In other embodiments, there may be an interval of time between administration of the additional therapeutic agent and the disclosed compounds or compositions. In some embodiments, administration of an additional therapeutic agent with a disclosed compound or composition may allow lower doses of the other therapeutic agents and/or administration at less frequent intervals. When used in combination with one or more other active ingredients, the compounds or compositions of the present invention and the other active ingredients may be used in lower doses than when each is used singly. Accordingly, the pharmaceutical compositions of the present invention include those that contain one or more other active ingredients, in addition to a compound of the present disclosure.

### 5. Kits

Aspects of the present disclosure include kits that have a composition as described herein.

A kit may include a compound of formula (I-A), or a pharmaceutically acceptable salt or composition thereof, and a therapeutic support composition. A kit may include a compound of formula (I-A), or a pharmaceutically acceptable salt or composition thereof, and a compound of formula (I-B), formula (II-A), or formula (III-A), or a pharmaceutically acceptable salt or composition thereof.

A kit may include a compound of formula (II-A) or formula (III-A), or a pharmaceutically acceptable salt or composition thereof, and one or more immunomodulatory agents, or a pharmaceutically acceptable salt or composition thereof, and optionally a therapeutic support composition. A kit may include a therapeutic support composition, as described herein, and one or more immunomodulatory agents, or a pharmaceutically acceptable salt or composition thereof.

The therapeutic support composition, one or more immunomodulatory agents, and the compound of formula (I-A), (I-B), (II-A), and/or (III-A) may be in separate containers in the packaging. One or more therapeutic support compositions may be provided in a kit.

The kits described herein may include a packaging configured to contain the composition (e.g., therapeutic support composition and/or one or more immunomodulatory agents). Similarly, one or more compounds of formula (I-A), (I-B), (II-A), and/or (III-A) may be provided in a kit. The packaging may be a sealed packaging, such as a sterile sealed packaging. By “sterile” is meant that there are substantially no microbes (such as fungi, bacteria, viruses, spore forms, etc.). In some instances, the packaging may be configured to be sealed, e.g., a water vapor-resistant packaging, optionally under an air-tight and/or vacuum seal.

In certain embodiments, the kit includes a reagent that may be used as the releasing agent for a releasable linker as described herein. The releasing reagent may be any one of the releasing agents described herein, such as, but not limited to, a chemical releasing agent (e.g., an acid, a base, an oxidizing agent, a reducing agent, etc.), a solvent, and the like. The releasing reagent in the kit may be provided in any convenient form, such as, but not limited to, a gas, a solution, a solid, granules, a powder, a suspension, and the like. The releasing reagent may be packaged in a separate container from the composition(s) in the kit.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Another form for the instructions would be a computer readable medium, e.g., CD, DVD, Blu-Ray, computer-readable memory (e.g., flash memory), etc., on which the information has been recorded or stored. Yet another form for the instructions that may be present is a website address which may be used via the Internet to access the information at a removed site. Any convenient means may be present in the kits.

### 6. Examples

The present disclosure has multiple aspects, illustrated by the following non-limiting examples. The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. By “average” is meant the arithmetic mean. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

Many general references providing commonly known chemical synthetic schemes and conditions useful for synthesizing the disclosed compounds are available (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978).

Compounds as described herein can be purified by any purification protocol known in the art, including chromatography, such as HPLC, preparative thin layer chromatography, flash column chromatography and ion exchange chromatography. Any suitable stationary phase can be used, including normal and reversed phases as well as ionic resins. In certain embodiments, the disclosed compounds are purified via silica gel and/or alumina chromatography. See, e.g., Introduction to Modern Liquid Chromatography, 2nd Edition, ed. L. R. Snyder and J. J. Kirkland, John Wiley and Sons, 1979; and Thin Layer Chromatography, ed E. Stahl, Springer-Verlag, New York, 1969.

During any of the processes for preparation of the subject compounds, it may be necessary and/or desirable to protect sensitive or reactive groups on any of the molecules concerned. This may be achieved by means of conventional protecting groups as described in standard works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie”, Houben-Weyl, 4^{th} edition, Vol. 15/1, Georg Thieme Verlag, Stuttgart 1974, in H.-D. Jakubke and H. Jescheit, “Aminosauren, Peptide, Proteine”, Verlag Chemie, Weinheim, Deerfield Beach, and Basel 1982, and/or in Jochen Lehmann, “Chemie der Kohlenhydrate: Monosaccharide and Derivate”, Georg Thieme Verlag, Stuttgart 1974. The protecting groups may be removed at a convenient subsequent stage using methods known from the art.

The subject compounds can be synthesized via a variety of different synthetic routes using commercially available starting materials and/or starting materials prepared by conventional synthetic methods. A variety of examples of synthetic routes that can be used to synthesize the compounds disclosed herein are described in the schemes below. Synthetic procedures to prepare compounds of formula (I-B) may be followed to prepare compounds of formula (I-A).

The following abbreviations are used herein:

ACN acetonitrile

Boc tert-butoxycarbonyl

Cy5 cyanine 5

Cy5.5 cyanine 5.5

daptodaptomycin

DCC N,N′-dicyclohexylcarbodiimide

DCM dichloromethane

dd doubly distilled

DIBAL diisobutylaluminum hydride

DIPEA diisopropylethylamine

DMAP 4-dimethylaminopyridine

DMFN,N-dimethylformamide

DMSO dimethylsulfoxide

doxo doxorubicin

EDCI N-(3-dimethylaminopropyl)-N′-ethylcarbodiimide hydrochloride

Et ethyl

EtOAc ethyl acetate

FCC flash column chromatography

Fmoc fluorenylmethyloxycarbonyl

h or hr hour

HA hyaluronic acid

HAT hyaluronic acid modified with tetrazine

HATU 1-[Bis(dimethylamino)methylene]-1H-1,2,3-triazolo[4,5-b]pyridinium 3oxide hexafluorophosphate

HBTU hexafluorophosphate benzotriazole tetramethyl uronium

HMThydrogel modified tetrazine

HOAt 1-hydroxy-7-azabenzotriazole

HOBt 1-hydroxybenzotriazole

HPLC high performance liquid chromatograph

iPrOH isopropyl alcohol

LCMS liquid chromatography-mass spectrometry

Me methyl

MeCN acetonitrile

MeOH methanol

MES 2-(N-morpholino)ethanesulfonic acid

MeTz methyltetrazine

min minutes

MTDmaximum tolerated dose

NHS N-hydroxysuccinimide

NMPN-methylpiperazine

PBS phosphate buffered saline

Ph phenyl

ppm parts per million

pyr pyridine

rt/RT room temperature

SEM standard error of the mean

sulfo-NHS N-hydroxysulfosuccinimide

TAG tetrazine-modified activating gel

TBAF tetrabutylammonium fluoride

TBME tert-butyl methyl ether

TCO trans-cyclooctene

TEA triethylamine

THF tetrahydrofuran

TLC thin-layer chromatography

TFA trifluoroacetic acid

TsCl tosyl chloride or toluenesulfonyl chloride

UV LVG ultrapure low viscosity guluronic acid

Vanco vancomycin

### Example A1

### Acid-TCO-Doxorubicin (axial isomer)

### rel-(1R,4E,6R,pS)-6-hydroxy-1-methylcyclooct-4-ene-1-carboxylic acid (axial isomer 2)

A solution of 5.34 g (95.2 mmol) potassium hydroxide in 16.7 mL water was added over a 5 min period to a water-cooled solution of the trans-cyclooctene ester 1 isomer mixture (Rossin et. al., Bioconjugate Chem., 2016, 27, 1697-1706) (1.64 g, 8.28 mmol, ratio of the axial/equatorial isomer ca. 1.2:1 for this particular batch) in 37 mL methanol. The solution was stirred for 18 h at room temperature. Water (51 mL) was added and the mixture was extracted with 3×150 mL TBME. The combined organic layers were washed with 100 mL water and then dried in vacuo to give the non-hydrolyzed equatorial ester 1b. The combined aqueous layers were treated with 300 mL TBME, and then with 15 g citric acid. The layers were separated and the aqueous layer was extracted with TBME (3×150 mL). The combined organic layers were dried and rotary evaporated at 25° C. to afford 873 mg (57%) of the pure axial isomer 2 of the trans-cyclooctene acid as a colorless oil. ¹H-NMR (CDCl₃): δ=6.15-5.95 (m, 1H), 5.6 (d, 1H), 4.45 (bs, 1H), 2.4-1.7 (m, 7H), 1.6 (dd, 1H), 1.18 (s, 3H). ¹³C-NMR (CDCl₃): δ=185.4 (C═O), 134.8 (═CH), 130.7 (═CH), 69.8 (CH), 44.8, 38.2, 31.0, 29.8 (CH₂), 18.1 (CH₃).

rel-(1R,4E,6R,pS)-2,5-dioxopyrrolidin-1-yl-6-((((2,5-dioxopyrrolidin-1-yl)oxy)carbonyl)oxy)-1-methylcyclooct-4-ene-1-carboxylate (axial isomer 3). To a solution of compound 2 (873 mg, 4.74 mmol) in 24.0 mL MeCN was added DIPEA (4.59 g, 35.6 mmol), followed by N,N′-disuccinimidyl carbonate (5.22 g, 20.4 mmol). The mixture was stirred for 3 days at RT, and subsequently evaporated in vacuo at 25° C. The residue was chromatographed on 40 g silica, with dichloromethane as eluent, followed by elution with dichloromethane containing an increasing amount of TBME (0-20%). The product fractions were combined and dried in vacuo. The resulting residue was stirred with TBME until a homogeneous suspension was obtained. Filtration and washing gave 761 mg of product 3 as a white solid (38%); ¹H-NMR (CDCl₃): δ=6.07 (ddd, J=16.8, 10.7, 3.5 Hz, 1H), 5.62 (dd, J=16.7, 2.5 Hz, 1H), 5.25 (s, 1H), 2.83 (2s, 8H), 2.5-2.25 (m, 4H), 2.2-1.9 (m, 4H), 1.28 (s, 3H).

NHS-TCO-Doxorubicin (axial isomer 4). Doxorubicin hydrochloride (53.7 mg; 0.093 mmol) and 3 (39.1 mg; 0.093 mmol) were dissolved in DMF (2.0 mL), and DIPEA (60.1 mg; 0.465 mmol) was added. The solution was stirred under an atmosphere of argon at room temperature for 22 h. HPLC analysis indicated about 60% of the desired product with double peaks. The crude product was split into two portions.
One portion was treated with morpholine (4.0 mg, 0.047 mmol, 5 eq) at room temperature for 24 h. Starting material was still present and the reaction was allowed to stir at room temperature for another 20 h. The conversion rate was about 71%. The product was also shown double peaks. The product was purified by Preparative HPLC to afford a fairly pure product. The product was confirmed by LCMS with m/z 935.9 (M+114-1)
Another portion was treated with 1-methylpiperazine (4.7 mg, 0.047 mmol, 5 eq) at room temperature for 24 h. Starting material was still present and the reaction was allowed to stir at room temperature for another 20 h. The conversion rate was about 64%. The product was also shown double peaks. The product was purified by Preparative HPLC to afford a fairly pure product. The product was confirmed by LCMS with m/z 948.9 (M+114-1)

NHS-TCO-Doxorubicin (axial isomer 4). Doxorubicin hydrochloride (1.05 g; 1.8 mmol) and 3 (761 mg; 1.8 mmol) were dissolved in DMF (18 mL), and DIPEA (1.16 g; 9.0 mmol) was added. The solution was stirred under an atmosphere of nitrogen at room temperature for 22 h. HPLC analysis indicated the reaction went well and the product has a single peak. The rest of the crude product was concentrated to dryness on rotavapor to remove DMF. The residue was purified by FCC (iPrOH/DCM: 0%-23%) to afford a pure product 4 (1.015 g, 66%) as a red solid. ¹H-NMR (CDCl₃): δ=13.97 (s, 1H), 13.22 (s, 1H), 8.03 (d, J=7.9 Hz, 1H), 7.78 (t, J=8.0 Hz, 1H), 7.38 (d, J=8.6 Hz, 1H), 5.85 (m, 1H), 5.59 (m, 1H), 5.51 (s, 1H), 5.29 (s, 1H). 5.16 (d, J=8.4 Hz, 1H), 5.12 (s, 1H), 4.75 (d, J=4.8 Hz, 2H), 4.52 (d, J=5.8 Hz, 1H), 4.15 (q, J=6.5 Hz, 1H), 4.08 (d, J=3.6 Hz, 3H), 3.87 (m, 1H), 3.69 (m, 1H), 3.26 (d, J=18.8 Hz, 1H), 3.00 (m, 2H), 2.81 (s, 4H), 2.4-1.7 (br. m, 13H), 1.62 (s, 2H), 1.30 (d, J=6.5 Hz, 3H), 1.23 (s, 3H) ppm.

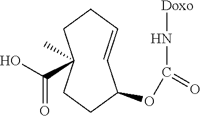

### Acid-TCO-Doxorubicin (axial isomer)

The intermediate 4 (˜2.4 mg) in DMF (0.10 mL) could be treated with saturated sodium bicarbonate (0.10 mL) at room temperature. After 18 h, the starting material was nearly consumed and the reaction was still complicated. The crude product could be purified by Prep HPLC to get a fairly pure product.

### Example A2

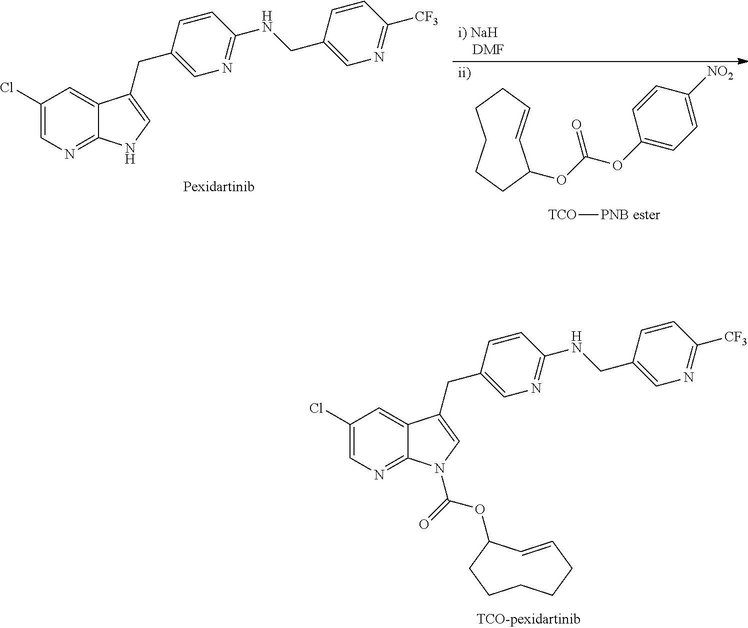

General Procedure for the Preparation of TCO-pexidartinib To a solution of Pexidartinib (PLX3397) (373 mg, 0.89 mmol) in DMF (4.0 mL) 0° C. was added sodium hydride (ca. 60%, 39 mg, ca. 0.96 mmol); and reaction mixture was stirred under nitrogen for 1 h before TCO-PNB ester (200 mg, 0.68 mmol) was added. The resulting mixture was stirred at rt overnight and evaporated in vacuo. The reaction mixture was diluted with water (30 mL) and extracted with ethyl acetate (2×30 mL). The combined organic layers were washed with brine, dried (Na₂SO₄) and evaporated in vacuo. The residue was purified by flash chromatography on silica gel eluting with dichloromethane followed by MeOH—CH₂Cl₂ (0-5%) to give TCO-pexidartinib (145 mg, 37%). LC-MS: 571 [M+H]^{+ 1}H NMR (300 MHz, CDCl₃) δ 8.72 (s, 1H), 8.41 (s, 1H), 8.05 (s, 1H), 7.85 (d, J=6.9 Hz, 1H), 7.66 (s, 1H), 7.62 (d, J=7.8 Hz, 1H), 7.56 (s, 1H), 7.29 (d, J=2.4 Hz, 1H), 6.37 (d, J=8.4 Hz, 1H), 6.15 (m, 1H), 5.74 (s, 1H), 5.60 (d, J=6.0 Hz, 1H), 4.88 (t, J=6.0 Hz, 1H), 4.67 (d, J=6.0 Hz, 2H), 3.87 (s, 1H), 2.50 (m 1H), 2.30 (m, 1H), 2.10-0.80 (m, 8H).

### Example A3

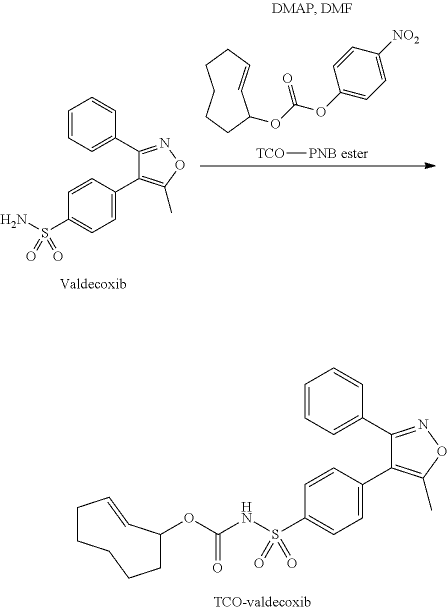

General Procedure for the Preparation of TCO-valdecoxib. To a solution of Valdecoxib (157 mg, 0.5 mmol) in DMF (4 mL) was added TCO-PNB ester (129 mg, 0.44 mmol), DMAP (106 mg, 0.88 mmol). The mixture was stirred at rt for 40 h, and diluted with ethyl acetate (100 mL), washed with brine (40 mL), dried over sodium sulfate, and evaporated in vacuo. The product was purified by flash chromatography on silica gel eluting with DCM followed by MeOH-DCM (5%) to give compound TCO-valdecoxib (201 mg, 97%) as white solid. LC-MS: 467 [M+H]⁺. ¹H NMR (300 MHz, CDCl₃) δ 8.03 (d, J=8.7 Hz, 2H), 7.65 (m, 1H), 7.43-7.32 (m, 7H), 5.73 (m, 1H), 5.64 (d, J=16.5 Hz, 1H), 5.33 (s, 1H), 2.50 (s, 3H), 2.43 (m, 1H), 2.09-0.77 (m, 9H).

### Example A4

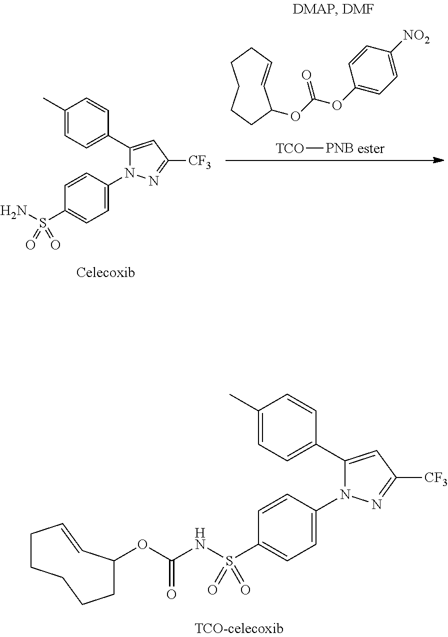

General Procedure for the Preparation of TCO-celecoxib. To a solution of Celecoxib (141 mg, 0.37 mmol) in DMF (4 mL) was added TCO-PNB ester (100 mg, 0.34 mmol), DMAP (106 mg, 0.88 mmol). The mixture was stirred for 40 h and diluted with ethyl acetate (100 mL), and washed with water (30 mL) and brine (30 mL), dried over sodium sulfate, and concentrated in vacuo. The product was purified by flash chromatography on silica gel eluting with methanol (5%) in DCM to afford the product TCO-celecoxib (162 mg, 88%). LC-MS: 534 [M+H]

¹H NMR (300 MHz, CDCl₃) δ 8.01 (d, J=8.7 Hz, 2H), 7.60 (br, 1H), 7.50 (d, J=8.7 Hz, 2H), 7.18 (d, J=8.1 Hz, 2H), 7.14 (d, J=8.1 Hz, 2H), 6.74 (s, 1H), 5.69 (m, 1H), 5.45 (d, J=12.0 Hz, 1H), 5.30 (s, 1H), 2.44 (m, 1H), 2.38 (s 3H), 2.03-0.76 (m, 9H).

### Example A5

### Synthesis of TCO-monomethyl auristatin E (TCO-MMAE) conjugate

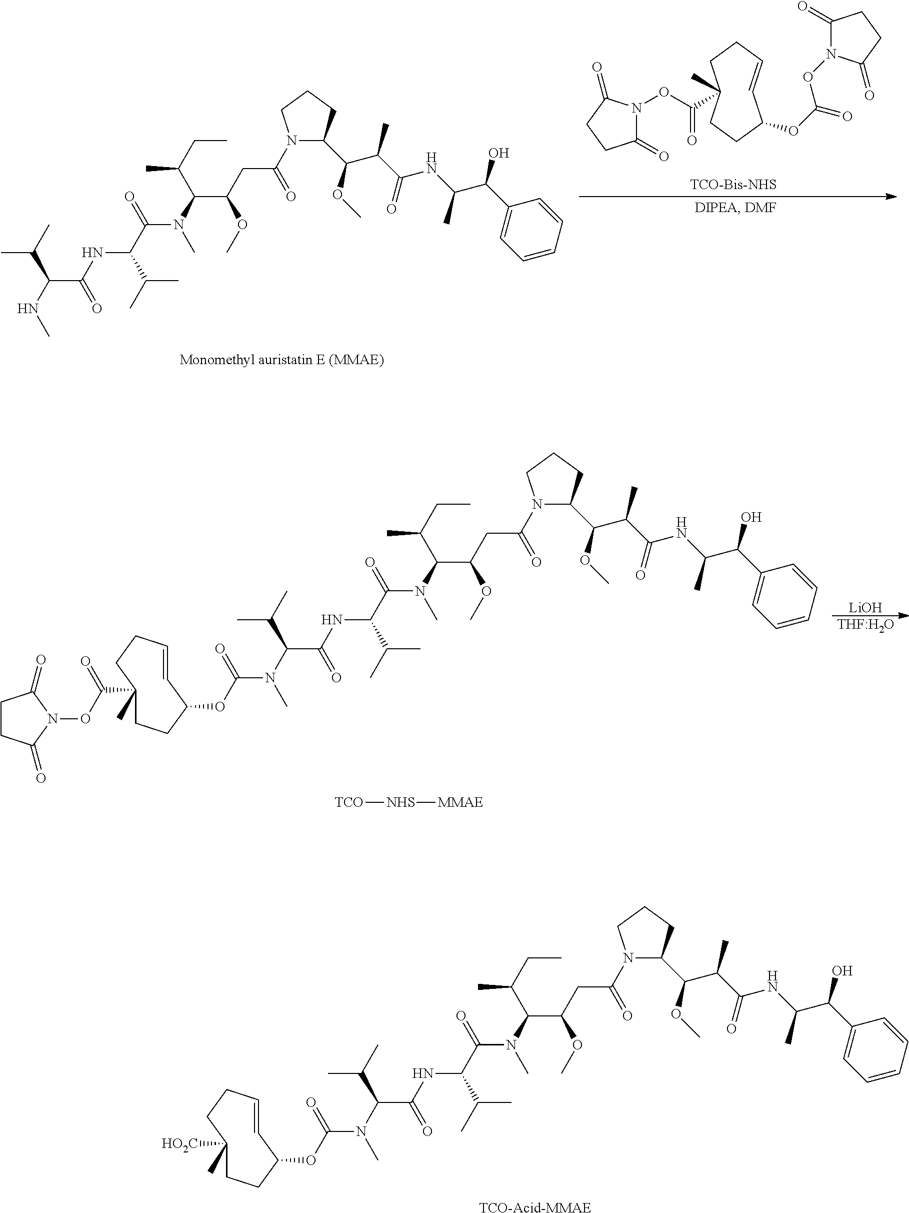

Preparation of TCO-MMAE conjugate. To monomethyl auristatin E (170 mg, 0.24 mmol) in DMF (2 mL) at rt, TCO-Bis-NHS (100 mg, 0.24 mmol) and DIPEA (93 mg, 0.72 mmol) were added. The solution was stirred at rt for 20 h, acetonitrile (ACN, 8 mL) was added and the mixture was purified by prep-HPLC (ACN/water from 0 to 100%, formic acid 0.1%) to give TCO-NHS-MMAE (88 mg, 36%). To TCO-NHS-MMAE (78 mg, 0.076 mmol) in THF (2 mL) and H₂O (2 mL) at rt was added LiOH (9.2 mg, 0.38 mmol). The solution was stirred at rt for 20 h. After removal of solvent, HCl (aq, 0.5 N) was added to pH-3. The mixture was purified by prep-HPLC (ACN/water from 0 to 100%, formic acid 0.1%) to give TCO-Acid-MMAE (54 mg, 76%, two isomers). LCMS: (ESI+) 928 [M+H].

### Example A6

### Synthesis of trans-cyclooctene(TCO)-glycine-doxorubicin conjugate

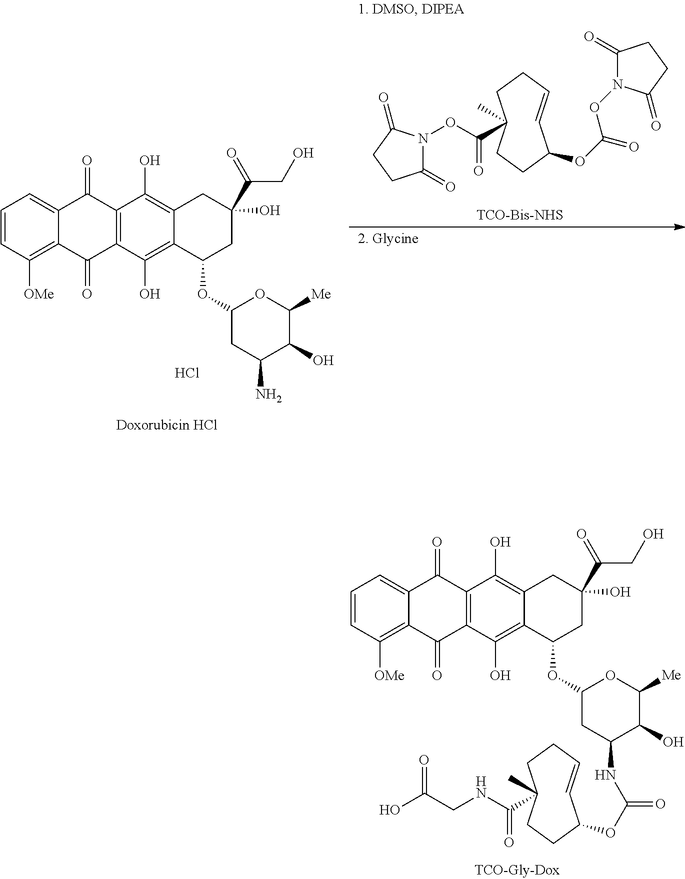

Preparation of TCO-glycine-doxorubicin conjugate. To a solution of doxorubicin hydrochloride (100 mg) in 1 mL DMSO, TCO-Bis-NHS (75 mg) was added. DIPEA (148 μL) was added by injection. The mixture was stirred overnight and then glycine (51 mg) was added to the reaction in one portion, and the reaction was stirred for 24 h. The mixture was diluted with 2 mL H₂O and purified by HPLC to yield TCO-Gly-Dox. MS: (ESI+) 833 [M+Na].

### Example A7

### Antibiotic-TCO Conjugates

### Example A7A

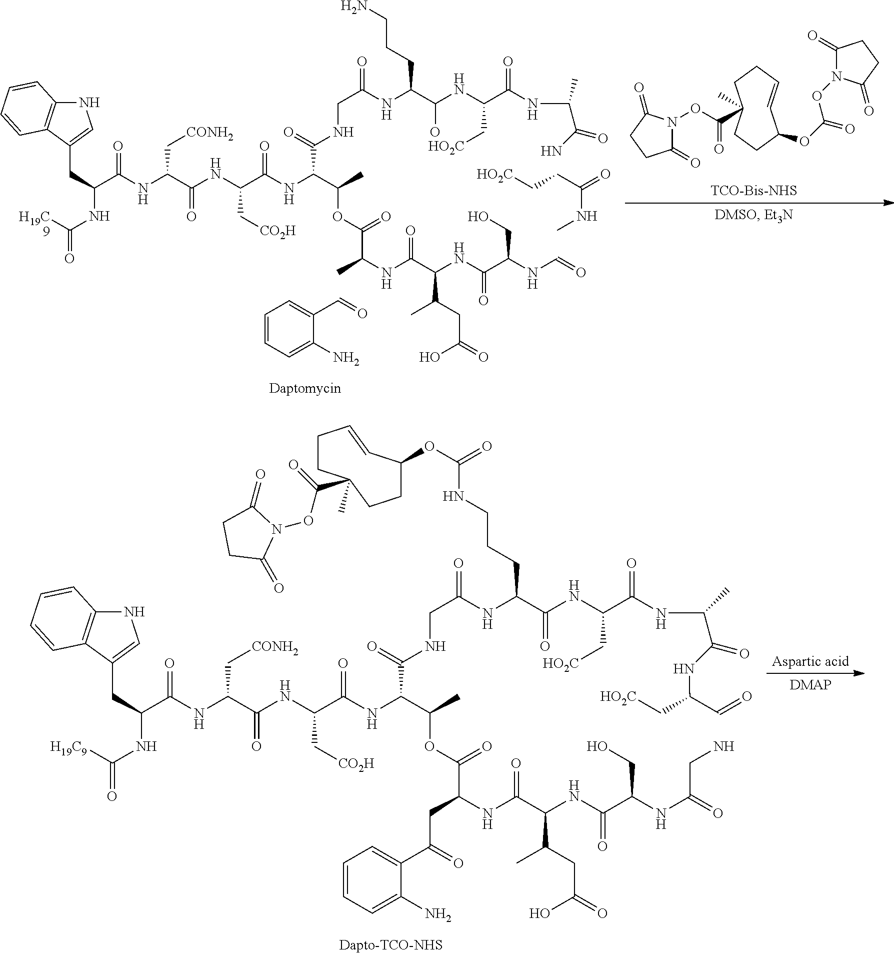

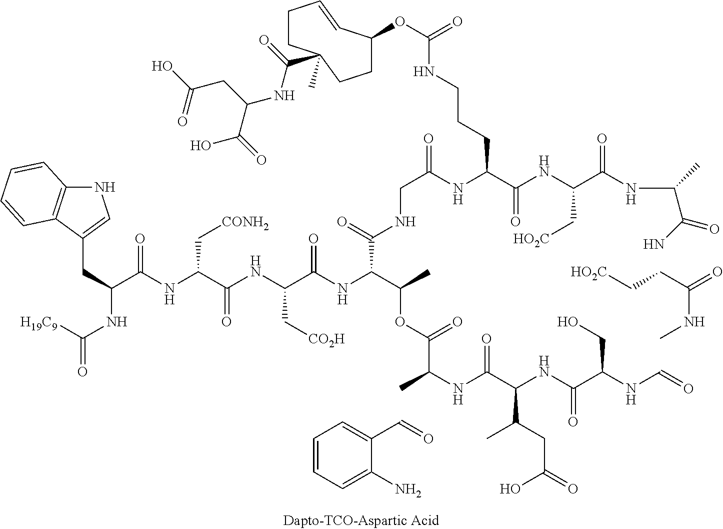

Example protocol: Add daptomycin (100 mg, 0.062 mmol), TCO-Bis-NHS (62.5 mg, 0.149 mmol), and triethylamine (62.5 μL, 45.3 mg, 0.448 mmol) to DMSO and stir at RT overnight to produce Dapto-TCO-NHS. LCMS: (ESI−) 1926.8 [M−H]. To Dapto-TCO-NHS (126.1 mg, 0.0654 mmol), add aspartic acid (104.5 mg, 0.785 mmol) and 4-dimethylaminopyridine (150.9 mg, 1.235 mmol), and stir for 18 h at 37° C. Purify by HPLC to obtain Dapto-TCO-Aspartic Acid. Yield: 100 mg, 0.0514 mmol. LCMS: (ESI−) 1944.8 [M−H].

This approach has been used to produce glycine and aspartic acid-modified TCO-prodrugs, and can be generally applied to for the incorporation of other amino acid cargos as well.

### Example A7B

### daptomycin-TCO-glycine conjugate

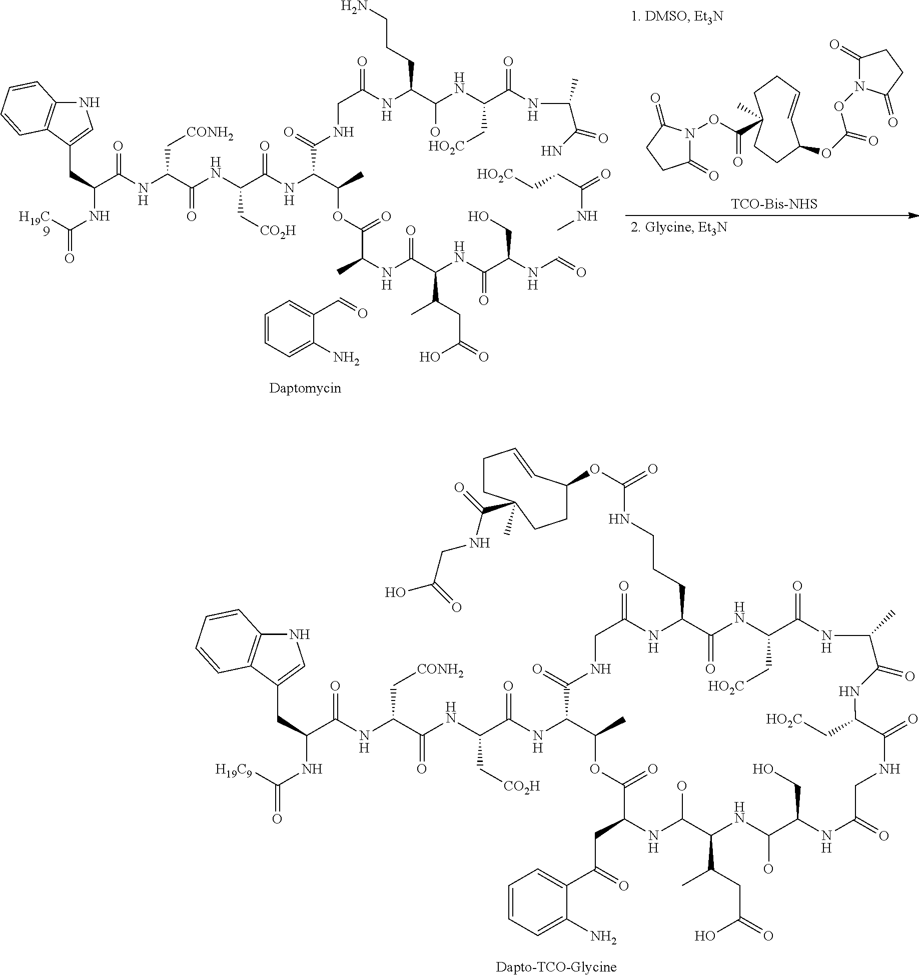

Daptomycin (537 mg, 0.33 mmol), TCO-Bis-NHS (350 mg, 0.83 mmol), and triethylamine (0.350 mL, 2.51 mmol) in DMSO (11 mL). Stir at RT overnight. Then heat to 37° C. Add glycine (300 mg, 4.00 mmol) and triethylamine (1.8 mL, 13 mmol), and stir for 18 h. Add 8 mL water and purify by HPLC. Yield: Dapto-TCO-Glycine-373 mg, 0.20 mmol, 59.6%.

### Example A7C

### Vanco-Bis-TCO-glycine conjugate

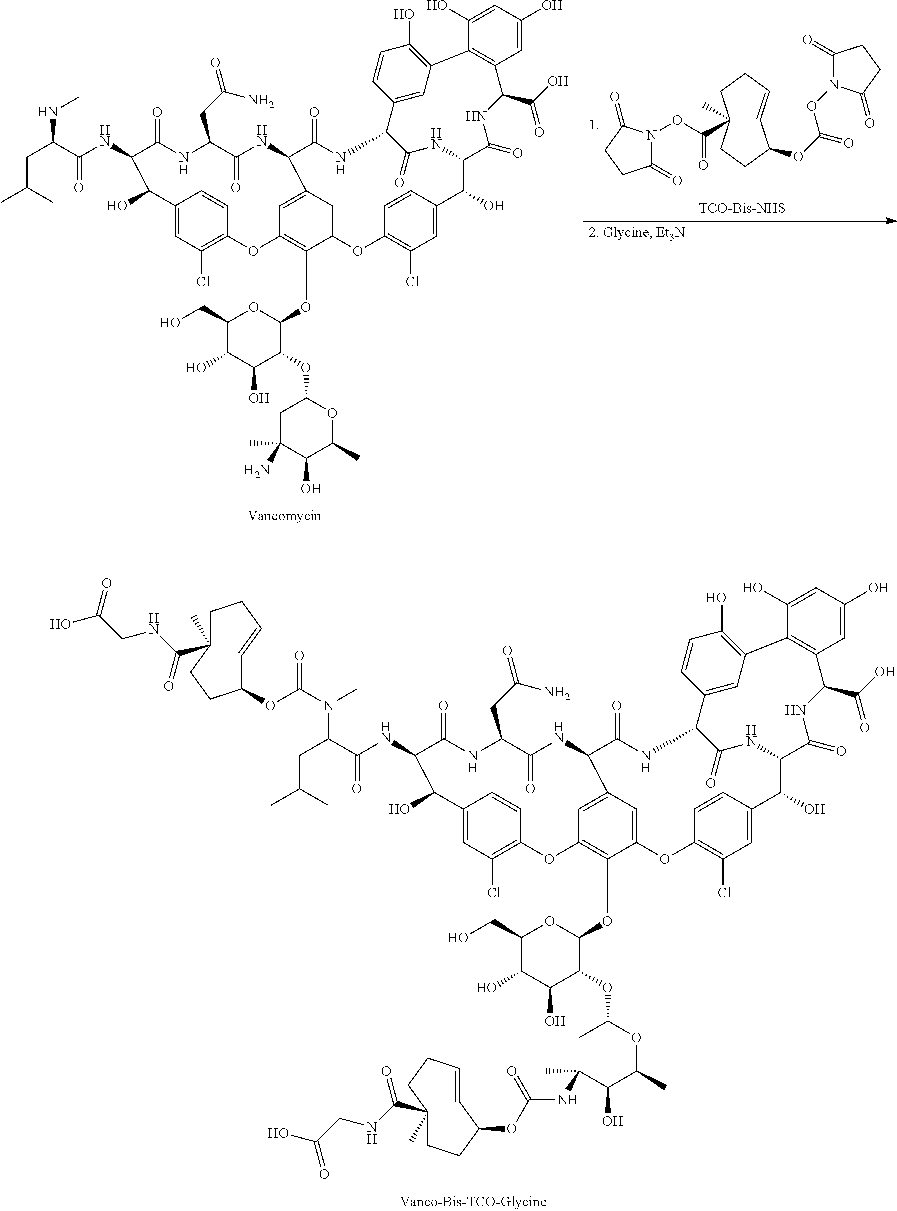

Example A7C can be synthesized using a protocol analogous to Example A7B. Vanco-Bis-TCO-Glycine tested up to 64 μg/ml (32 μM) shows no activity against bacteria as measured by microcalorimetry, indicating the drug deactivation after modification.

General HPLC purification conditions for TCO amino acid conjugates are as follows:

### Column: Higgins Cat #PS-253C-C185, 250×30 mm, Phalanx C18 5 μm

Solvent A: water (0.1% formic acid)
Solvent B: acetonitrile (0.1% formic acid)

| Min | % B |
|---|---|
| 0.01 | 10 |
| 2.00 | 10 |
| 3.00 | 30 |
| 30.00 | 90 |
| 31.00 | 10 |
| 34.00 | 10 |

### Example A8

### Hyaluronic acid modified tetrazine

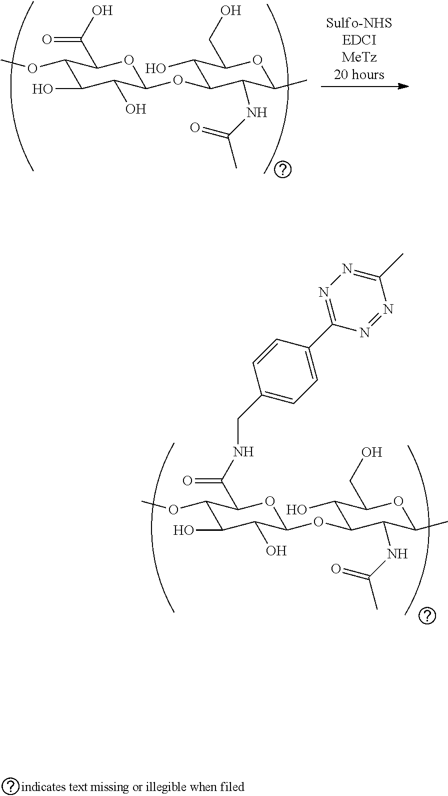

To 5 mL of MES buffer (0.1 M MES, 0.3 M NaCl, pH=6.5) was added 0.0500 grams of Sodium Hyaluronate (200 kDa) and stirred until it dissolved (4 hours). To this, was added N-hydroxysulfosuccinimide (23.3 mg, 0.107 mmols), N, N′-dicyclohexylcarbodiimide (42.0 mg, 0.219 mmols), and (4-(6-methyl-1,2,4,5-tetrazin-3-yl)phenyl) methanamine hydrochloride (15.9 mg, 0.066 mmols). The reaction mixture was stirred for 20 hours in the absence of light for after which time it was quenched with hydroxylamine (66.2 mg, 0.953 mmols). The hyaluronic acid product was purified in the absence of light against deionized water containing a decreasing salt concentration (NaCl, 0.13 M-0.0 M) over 5 days. The hyaluronic acid product was filtered (0.22 μm) and lyophilized for 5 days.

To 5 mL of MES buffer (0.1 M MES, 0.3 M NaCl, pH=6.5) was added 0.0500 grams of Sodium Hyaluronate (100 kDa) and stirred until it dissolved (4 hours). To this, was added N-hydroxysulfosuccinimide (40.6 mg, 0.19 mmols), N,N′-dicyclohexylcarbodiimide (72.1 mg, 0.38 mmols), and (4-(6-methyl-1,2,4,5-tetrazin-3-yl)phenyl) methanamine hydrochloride (28.4 mg, 0.12 mmols). The reaction mixture was stirred for 20 hours in the absence of light for after which time it was quenched with hydroxylamine (117.1 mg, 1.69 mmols). The hyaluronic acid product was purified in the absence of light against deionized water containing a decreasing salt concentration (NaCl, 0.13 M-0.0 M) over 5 days. The hyaluronic acid product was filtered (0.22 μm) and lyophilized for 5 days.

To 5 mL of MES buffer (0.1 M MES, 0.3 M NaCl, pH=6.5) was added 0.0500 grams of Sodium Hyaluronate (60 kDa) and stirred until it dissolved (4 hours). To this, was added N-hydroxysulfosuccinimide (58.2 mg, 0.27 mmols), N,N′-dicyclohexylcarbodiimide (103.9 mg, 0.54 mmols), and (4-(6-methyl-1,2,4,5-tetrazin-3-yl)phenyl) methanamine hydrochloride (40.4 mg, 0.17 mmols). The reaction mixture was stirred for 20 hours in the absence of light for after which time it was quenched with hydroxylamine (165.7 mg, 2.38 mmols). The hyaluronic acid product was purified in the absence of light against deionized water containing a decreasing salt concentration (NaCl, 0.13 M-0.0 M) over 5 days. The hyaluronic acid product was filtered (0.22 μm) and lyophilized for 5 days.

To 5 mL of MES buffer (0.1 M MES, 0.3 M NaCl, pH=6.5) was added 0.0500 grams of Sodium Hyaluronate (5 kDa) and stirred until it dissolved (4 hours). To this, was added N-hydroxysulfosuccinimide (145.9 mg, 0.670 mmols), N,N′-dicyclohexylcarbodiimide (257.3 mg, 1.34 mmols), and (4-(6-methyl-1,2,4,5-tetrazin-3-yl)phenyl) methanamine hydrochloride (100.3 mg, 0.42 mmols). The reaction mixture was stirred for 20 hours in the absence of light for after which time it was quenched with hydroxylamine (413.4 mg, 5.95 mmols). The hyaluronic acid product was purified in the absence of light against deionized water containing a decreasing salt concentration (NaCl, 0.13 M-0.0 M) over 5 days. The hyaluronic acid product was filtered (0.22 μm) and lyophilized for 5 days.

### Example A9

### Hyaluronic Acid Modified Tetrazine

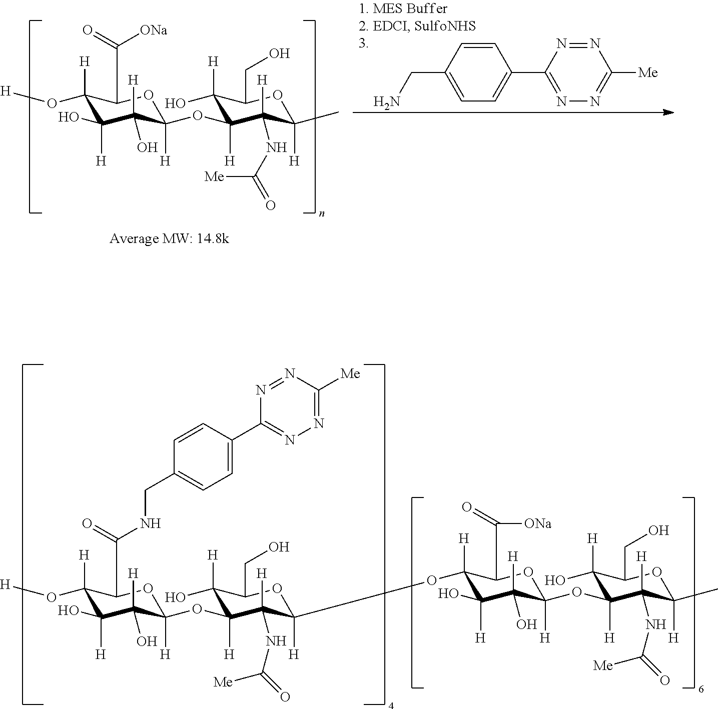

To 5 mL of MES buffer (0.1 M MES, 0.3 M NaCl, pH=4.5) was added 0.5000 grams of Sodium Hyaluronate (14.8 kDa) and stirred until it dissolved. To this, was added N-hydroxysulfosuccinimide (14.2 mg, 0.0625 mmols), N,N′-dicyclohexylcarbodiimide (125.7 mg, 0.625 mmols), and (4-(6-methyl-1,2,4,5-tetrazin-3-yl)phenyl) methanamine hydrochloride (151.2 mg, 0.625 mmols). The reaction mixture was stirred for 4 hours in the absence of light for after which time it is diluted to 1% (w/w) and filtered through a 0.45 μm filter. The hyaluronic acid product was then purified by Tangential flow filtration (TFF), prior to the final sterile filtration (0.22 μm) and lyophilized for 3 days. By elemental analysis, the tetrazine incorporation into the Sodium Hyaluronate starting material is 40%.

### Example A10

### Tetrazine Modified Alginate Gel

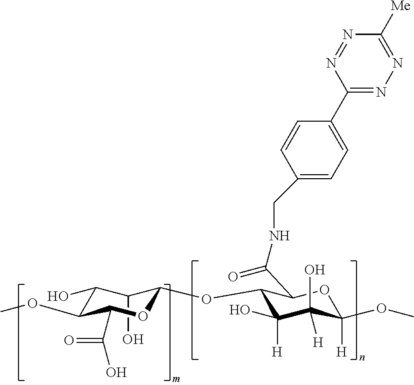

To 5 mL of MES buffer (0.1 M MES, 0.3 M NaCl, pH=6.5) was added 50 mg of UP LVG alginate (75-200 kDa) and stirred until it dissolved (4 hours). To this, was added N-hydroxysulfosuccinimide (34.7 mg, 0.16 mmols), N,N′-dicyclohexylcarbodiimide (61.8 mg, 0.32 mmols), and (4-(6-methyl-1,2,4,5-tetrazin-3-yl)phenyl)methanamine hydrochloride (24.1 mg, 0.10 mmols). The reaction mixture was stirred for 20 hours in the absence of light for after which time it was quenched with hydroxylamine (99.3 g, 1.44 mmols). The alginate product was purified in the absence of light against deionized water containing a decreasing salt concentration (NaCl, 0.13 M-0.0 M) over 4 days. The alginate was filtered (0.22 μm) and lyophilized for 5 days.

### Example C1

### In Vivo Test of Anti-Tumor Efficacy Study of Drug in MC-38 Subcutaneous Syngeneic Model in C57BL/6 mice

### 1. Introduction

BIOMATERIAL 1/PRODRUG 1 treatment is a combination therapy of PRODRUG 1 (a trans-cyclooctene-modified prodrug of doxorubicin) and BIOMATERIAL 1 (a tetrazine-modified hyaluronic acid biomaterial). PRODRUG 1 is attenuated in activity and can be systemically administered with minimal risk of spontaneous conversion and exposure to systemic doxorubicin (Dox). PRODRUG 1 prodrug will only become activated after reacting with BIOMATERIAL 1, which will be injected at a local site.

### 2. Study Objective

This study aimed to evaluate the in vivo therapeutic efficacy of BIOMATERIAL 1/PRODRUG 1 treatment in an MC38 colorectal cancer model in immunocompetent C₅₇BL/6 mice, compared to conventional Dox and in combination with a TLR agonist (TLRa).

### 3. Study Design

5 mice/group for 3 groups, 10 mice/group for 7 groups, 10 groups total.

### 3.1 Treatment Group and Dosing

NOTE: When tumors reached the size around 100 mm³, treatments were begun. The first day of treatment was labeled “Day 1” as below.

TABLE 1
Study design

| | | Tumor | Drug type (1 h | | PRODRUG 1 or Dox |
|---|---|---|---|---|---|
| Group | # of | model | after | Drug Dose | Dose I.V. (mg/kg/ |
| # | animals | (MC38) | BIOMATERIAL 1) | duration | dose) |
| 1 | 5 | 1 injected | Saline | 5 doses, once daily, | — |
| | | tumor (5 × | | 5 days | |
| | | 10⁵ cells) | | | |
| 2 | 10 | 1 injected | PRODRUG 1 | 5 doses, once daily, | 40 |
| | | tumor (5 × | | 5 days | |
| | | 10⁵ cells) | | | |
| 3 | 10 | 1 injected | PRODRUG 1 + | 5 doses, once daily, | 40 |
| | | tumor (5 × | TLR agonist | 5 days; TLR agonist | |
| | | 10⁵ cells) | | | |
| 4 | 5 | 1 injected | Saline | 5 doses, once daily, | — |
| | | tumor (5 × | | 5 days | |
| | | 10⁵ cells) | | | |
| | | 1 non- | | | |
| | | injected | | | |
| | | tumor (1 × | | | |
| | | 10⁵ cells) | | | |
| 5 | 5 | 1 injected | Dox | 3 doses every 4 | 8.1 |
| | | tumor (5 × | | days. | |
| | | 10⁵ cells) | | | |
| | | 1 non- | | | |
| | | injected | | | |
| | | tumor (1 × | | | |
| | | 10⁵ cells) | | | |
| 6 | 10 | 1 injected | Dox + TLR | 3 doses every 4 | 8.1 |
| | | tumor (5 × | agonist | days. TLR agonist | |
| | | 10⁵ cells) | | | |
| | | 1 non- | | | |
| | | injected | | | |
| | | tumor (1 × | | | |
| | | 10⁵ cells) | | | |
| 7 | 10 | 1 injected | PRODRUG 1 + | 5 doses, once daily, | 40 |
| | | tumor (5 × | TLR agonist | 5 days; TLR agonist | |
| | | 10⁵ cells) | | | |
| | | 1 non- | | | |
| | | injected | | | |
| | | tumor (1 × | | | |
| | | 10⁵ cells) | | | |
| 8 | 10 | 1 injected | PRODRUG 1 | 5 doses, once daily, | 40 |
| | | tumor (5 × | | 5 days | |
| | | 10⁵ cells) | | | |
| | | 1 non- | | | |
| | | injected | | | |
| | | tumor (1 × | | | |
| | | 10⁵ cells) | | | |
| 9 | 10 | 1 injected | PRODRUG 1 | 5 doses, once daily, | 40 |
| | | tumor (5 × | | 5 days | |
| | | 10⁵ cells) | | | |
| | | 1 non- | | | |
| | | injected | | | |
| | | tumor (1 × | | | |
| | | 10⁵ cells) | | | |
| 10 | 10 | 1 injected | Saline | 5 doses, once daily, | — |
| | | tumor (5 × | | 5 days | |
| | | 10⁵ cells) | | | |
| | | 1 non- | | | |
| | | injected | | | |
| | | tumor (1 × | | | |
| | | 10⁵ cells) | | | |

For groups 1-3 (single tumor model) Immunocompetent male C₅₇BL/6 mice were inoculated SC in the right flank with 5×10⁵5 MC38 tumor cells.

For groups 4-10 (dual tumor model), Immunocompetent male C₅₇BL/6 mice were inoculated SC in the right flank with 5×10⁵ MC38 tumor cells (large injected tumor) and in the left flank with 1×10⁵ MC38 tumor cells (small non-injected tumor).

Prior to injection, the MC38 cells were suspended in 0.1 mL DMEM media mixed with 50% Matrigel for tumor development. When the average tumor volume of the large tumor reached approximately 100 mm³, animals were randomly grouped according to body weight and tumor volume into 10 treatment groups with 5-10 mice per group. All groups received peritumoral biomaterial injections (100 μL/mouse) near the large tumor (hereafter, the injected tumor). One hour later, groups 1, 4 and 10 were IV administered saline control (QD×5 days); groups 2, 8 and 9 were IV administered PRODRUG 1 prodrug (28.6 mg/kg Dox Eq QD×5 days; cumulative dose of 143 mg/kg Dox Eq) and group 5 was IV administered Dox HCl control (MTD; 8.1 mg/kg Q4D×3 doses; cumulative dose of 24.3 mg/kg). Tumor volumes for both tumors were measured three times weekly in two dimensions using a caliper, and the volume expressed in mm³. 8 groups were used to assess the tumor growth inhibition, while groups 9 and 10 (PRODRUG 1, saline n=10/group) were used to determine the immune cell infiltration using flow cytometry. Groups 3, 6 and 7 (n=10) were used to test Dox+ TLR9 agonist (SL-01) and PRODRUG 1+ TLR9 agonist. The TLR9 agonist was given as an intratumoral injection in the primary tumors alone after the last PRODRUG 1 or Dox dose at 25 μg per mouse. For the complete responder mice, tumor re-challenge was done with 5×10⁵ cells per mouse. Flow cytometric analysis was carried out with the animal groups 9 and 10 (PRODRUG 1, saline n=10/group) to determine the immune cell infiltration using flow cytometry. Tumor collection was performed on the second and fourth subgroups at 1 and 2 weeks, respectively. Followed by RBC lysis and Fc-blocking, the tumor-derived cells were analyzed for cell-surface (CD45, CD3, CD4, CD8, CD25, PD-1) or intracellular (FoxP3) markers using flow cytometry. Cells were also marked with a live/dead stain (Fixable Viability Stain) to discriminate non-viable from viable cells. CD45 is a cell-surface (transmembrane) molecule available on most cells of hematopoietic origin (i.e. from blood). It is used to distinguish infiltrated cells from native cells of the MC38 tumor, which is a colon carcinoma line and lacks CD45. CD3 is a pan T-cell marker, while CD4 and CD8 are present on Helper T cell (T_{H} cells) and cytotoxic T lymphocyte (CTL) subsets, respectively. FoxP3 is an intracellular marker found on CD4+CD25+ cells and are typically identified as the most common type of T_{reg}s. PD-1 is an immune-checkpoint protein and a programmed cell-death receptor found on cells (commonly T-cells). When bound by its cognate ligand(s), PD-1 can trigger apoptosis of antigen-specific (CD4+ or CD8+) T cells.

TABLE 2
Supplementary Treatment Instructions

| Supplementary | | | |
|---|---|---|---|
| treatment | Group | Route | Dose/Frequency/Treatment start |
| TLR9 agonist (ODN D- | 3 | Intratumoral | 25 μg per mouse(volume: 50 μL); Once; One hour after |
| SL01 | | | PRODRUG 1 Dosing completion on Day 5; One hour |
| | | | after Dox Dosing completion on Day 9 |

### 3.2 Re-Treatment Group and Dosing

Re-treatment was administered starting at 38 days post-inoculation (dpi).

Group 2—ALL Remaining Mice (n=8)

1. One 100 μL peritumoral injection of BIOMATERIAL 1 (across 5 poles) 2. After 1 h, 5 daily doses of PRODRUG 1 at 16.6 mg/kg/day IV (lower than before)

Group 3—ALL Remaining Mice (n=10)

Split Group 3 in 2 subgroups.

Each subgroup contained n=5: n=3 with tumor >100 mm³, and n=2 with tumor <100 mm³.

Treated one subgroup only with BIOMATERIAL 1+ TLRa co-administered (peritumoral) and saline IV.

Treated second subgroup with BIOMATERIAL 1+ TLRa co-administered (peritumoral) and PRODRUG 1 5 daily doses IV.

### BIOMATERIAL 1+ TLRa Co-Administration

1. Mixed 100 μL of BIOMATERIAL 1 with 25 μg of TLRa (for 1 mouse; lower TLRa amounts than before)

2. One peritumoral injection with BIOMATERIAL 1 mixed with TLRa

3. After 1 h, 5 daily doses of PRODRUG 1 at 16.6 mg/kg/day IV

### 4.2 Test Articles

### 4.2.1 Biomaterial 1

A tetrazine-modified sodium hyaluronate modified as in the formula

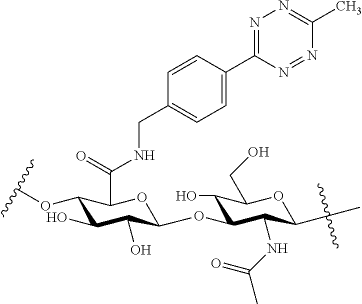

having ˜10-15 kD MW and ˜30% modification
Appearance: Pink liquid filled in syringes
Storage: −20° C.
Formulation: not required

### 4.2.2 Prodrug 1

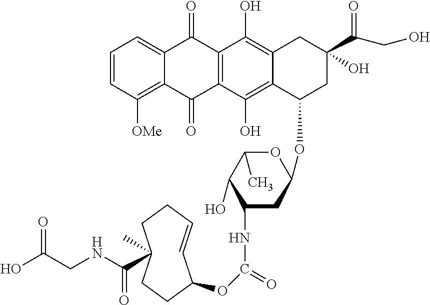 [0581] Appearance: dry red powder
Storage: −20° C.
Formulation: Dissolved PRODRUG 1 powder in sterile phosphate buffered saline (PBS). Adjust pH with 1M NaOH until pH 7.2. Filtered through a 0.2 μm membrane under aseptic conditions. Prepared formulations fresh daily before IV injection.

### 4.2.3 Tlra

ODN D-SL01 from Invivogen
Appearance: dry white solid
Storage: −20° C.
Formulation: Dissolved 200 μg TLRa in 400 μL endotoxin-free water. Prepared formulations fresh daily before IT injection.

### 4.3 FACS Antibodies and Reagents

| marker | Fluorochrome | Cat No | Name | vendor |
|---|---|---|---|---|
| Live/dead | APC-CY7 | 565388 | Fixable Viability Stain 780 200 μg | BD |
| CD45 | AF700 | 560510 | Anti-CD45 Alexa 700 30-F11 50 μg | BD |
| CD3 | PE-CY7 | 552774 | Anti-CD3e PE-Cy7 145-2C11 100 μg | BD |
| CD4 | FITC | 557307 | Anti-CD4 FITC GK1.5 100 μg | BD |
| CD8 | BV510 | 563068 | Anti-CD8a BV510 53-6.7 50 μg | BD |
| CD25 | BV421 | 564370 | Anti-CD25 BV421 3C7 50 μg | BD |
| Foxp3 | PE | 12-4774-42 | ANTI-FOXP3 (150D/E4) PE 100 Test | Thermo Fisher |
| PD-1 | APC | 562671 | Anti-CD279 APC J43 50 μg | BD |

Mouse Fc block (#553141), BD Horizon Brilliant Stain Buffer (#563794) and compensation beads (#554825) were obtained from BD.
Miltenyi Tumor Dissociation Kit (Miltenyibiotec, #130-096-730)
RBC lysis buffer (BD, #555899)
70 μm cell strainers (Miltenyibiotec, #130-098-462)

### 5. Experimental Methods and Procedures

### 5.1 Cell Culture

MC-38 cells were cultured with DMEM supplemented with 10% heat inactivated fetal bovine serum (FBS) at 37° C. in 5% CO₂ incubator. Cells were passaged 2 times a week. Cells were harvested, counted, passaged, and inoculated when around 70% confluent.

### 5.2 Tumor Inoculation and Grouping

Eighty-five animals were enrolled into the efficacy study. When tumor volume reached an average volume of approximately 100 mm³, animals were randomized as below using block randomization by Excel based upon their tumor size. This ensured that all the groups were comparable at the baseline. 5×10⁵ MC-38 cells suspended in 100 μL PBS mixed with 50% matrix gel were inoculated subcutaneously into the right flank. For Groups 4 through 10, 1×10⁵MC-38 cells suspended in 100 μL PBS mixed with 50% matrix gel were inoculated subcutaneously into the left flank.

### 5.3 Observations

At the time of routine monitoring, the animals were checked for any adverse effects of tumor growth and/or treatment on normal behavior such as effects on mobility, food and water consumption (by observation only), and body weight gain/loss (body weights had measured twice weekly in the pre-dosing phase and daily in the dosing phase), eye/hair matting and any other abnormal effect, including tumor ulceration. The sponsor was informed if the body weight loss of any animal reached 10%. Unexpected deaths and observed clinical signs were recorded based on the numbers of animals within each subset. Animals were not allowed to become moribund.

### 5.4 Tumor Measurements and Endpoints

Tumor volume was measured thrice weekly in two dimensions using a caliper, and the volume was expressed in mm³ using the formula: V=0.5 a×b², where a and b are the long and short diameters of the tumor, respectively. The tumor volume was then used for calculations of both T-C and T/C values. T-C was calculated with T as the median time (in days) required for the treatment group tumors to reach a predetermined size, and C as the median time (in days) for the control group tumors to reach the same size. The T/C value (in percent) was an indication of antitumor effectiveness; T and C were the mean volume of the treated and control groups, respectively, on a given day. The T-C value was calculated according to TV. T-C was calculated with T as the median time (in days) required for the treatment group tumors to reach a predetermined size, and C as the median time (in days) for the control group tumors to reach the same size.

### 5.5 Flow Cytometry Analysis (Fc/Facs)

In the same study, 2 groups (n=10) of mice with dual MC38 tumors were used for tumor immune cell profiling and were treated with either BIOMATERIAL 1/PRODRUG 1 TREATMENT or saline. Each group was further divided into 2 subgroups of 5 mice each. Tumor collection was performed on the first and second subgroups at 1 and 2 weeks, respectively. Followed by red blood cell (RBC) lysis and Fc-blocking, the tumor-derived cells were analyzed for cell-surface (CD45, CD3, CD4, CD8, CD25, PD-1) or intracellular (FoxP3) markers using flow cytometry. Cells were also marked with a live/dead stain (Fixable Viability Stain) to discriminate non-viable from viable cells. The cell percentage of each population of interest was identified (Table 6). CD45 is a cell-surface (transmembrane) molecule available on most cells of hematopoietic origin (i.e. from blood). It is used to distinguish infiltrated cells from native cells of the MC38 tumor, which is a colon carcinoma line and lacks CD45. CD3 is a pan T-cell marker, whereas CD4 and CD8 are present on helper T cell and cytotoxic T lymphocyte (CTL) subsets, respectively. FoxP3 is an intracellular marker found on CD4+CD25+ cells and is typically identified as the most common type of regulatory T cells (T_{reg}s). PD-1 is an immune-checkpoint protein and a programmed cell-death receptor found on cells (commonly T cells). When bound by its cognate ligand(s), PD-1 can trigger apoptosis of antigen-specific (CD4+ or CD8+) T cells.

### 5.5.1 Tissue Processing

For tumor samples, tumor tissues were homogenized using the Miltenyi Tumor Dissociation kit. Single cell suspension from tumor samples were RBC lysed, centrifuged to pellet cells, washed with cold PBS and resuspend cell pellets in the staining buffer and store on ice. Cells were then ready for FACS antibody staining.

### 5.5.2 FACS Antibody Staining

1. For each sample, added cells (<10×10⁶ cells/tube) in the staining buffer, into an Eppendorf tube labeled with correct sample name.
Panel 1: Live-dead/CD45/CD3/CD4/CD8/CD25/PD-1
Panel 2: Live-dead/CD45/CD3/CD4/CD25/Foxp3

2. For each tissue type (tumor), prepared two extra tubes (see below) and added cells (<10×10⁶ cells/tube) in the staining buffer into these two tubes.
Tube: no-color
Tube: live-dead only***
Tube: no-color (fix/perm)
Tube: live-dead only (fix/perm)
Note: These two tubes served as gating control for each tissue type.
*** The live-dead only tube was also used for live-dead compensation. Compensation for 8 fluorochrome-conjugated antibodies was prepared separately, using compensation beads from BD and following vendor's manual.

3. Prepared FcR blocking solution (100 μL per tube×tube numbers) by diluting FcR blocking antibody at 1/100 in staining buffer
Note: Since two Brilliant Violet dyes were used in the FACS panel, BD Horizon Brilliant Stain Buffer were added at 5 μL per 100 μL FcR blocking solution

4. Resuspended cell pellets in 100 μL FcR blocking solution and incubated at RT in dark for 3 min

5. For panel 1, added FACS antibodies to desired concentrations (2 μg/ml for each antibody). Incubated at 4° C. in dark for 30 min. For panel 2, added FACS antibodies (except foxp3) to desired concentrations (2 μg/ml for each antibody), incubated at 4° C. in dark for 30 min, then fixated/permeabilized the cells at 4° C. for 40-50 min, and added foxp3 antibody to desired concentrations (2 μg/ml) at 4° C. in dark for 40 min.

6. Added 1 ml staining buffer, centrifuged at 350 g for 4 min at 4° C. to wash and pellet cells

7. Resuspended cells in “Tube: no color” with 500 μL staining buffer (no live-dead added)

8. Resuspended cells in all other tubes with 500 μL staining buffer with 1 μg/ml live-dead

9. Cells were ready for FACS analysis and were subjected to analysis by the Attune Nxt Flow Cytomter (ThermoFisher)

### 5.6 Statistical Analysis

A two-way ANOVA was performed to compare body weight and tumor volume. All the data was analyzed using GraphPad Prism 5. For our analysis, p<0.05 was considered to be statistically significant.

### 5.7 Endpoint Tissue Collection

For Group 9 and Group 10, 5 and 5 mice were sacrificed at 1 and 2 weeks respectively after dosing completion. Tumors were collected for FACS.

### 6. Results and Discussion

### 6.1 Body Weights

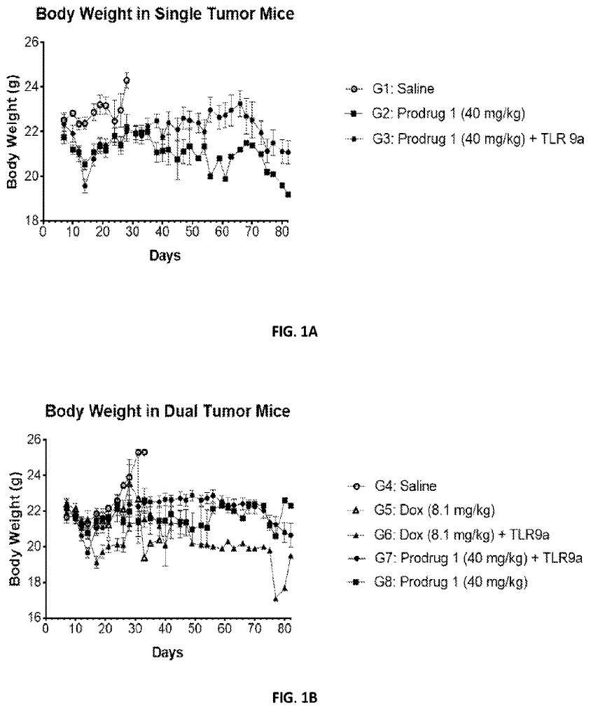

Results of the body weight changes in the tumor bearing mice are shown in FIG. 1A and FIG. 1B for Groups 1-8.

### 6.2 Tumor Volumes

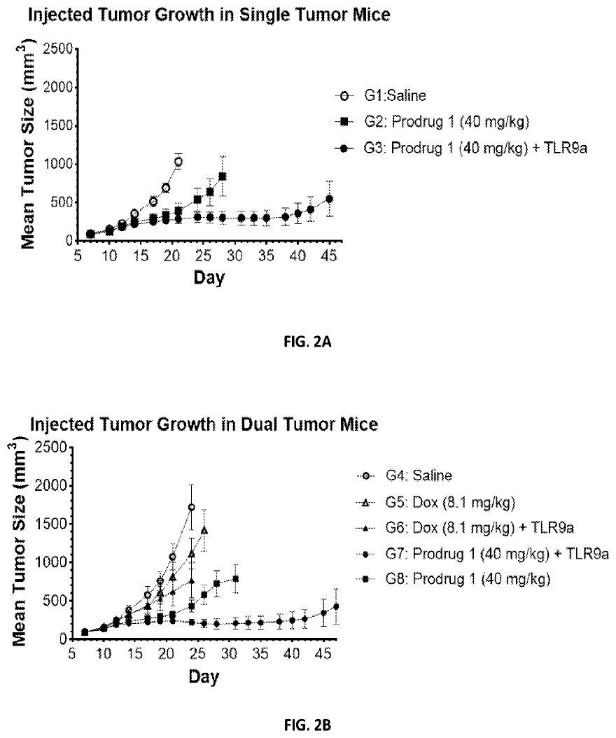

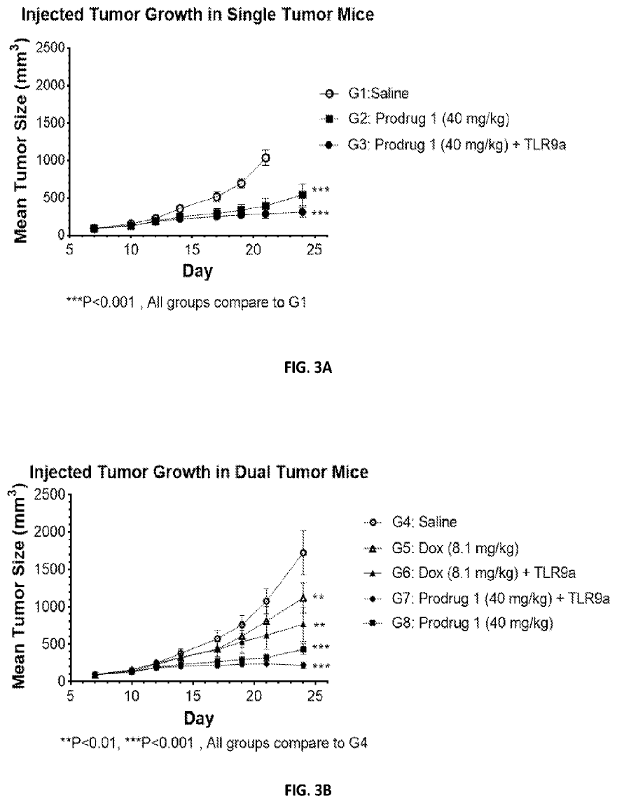

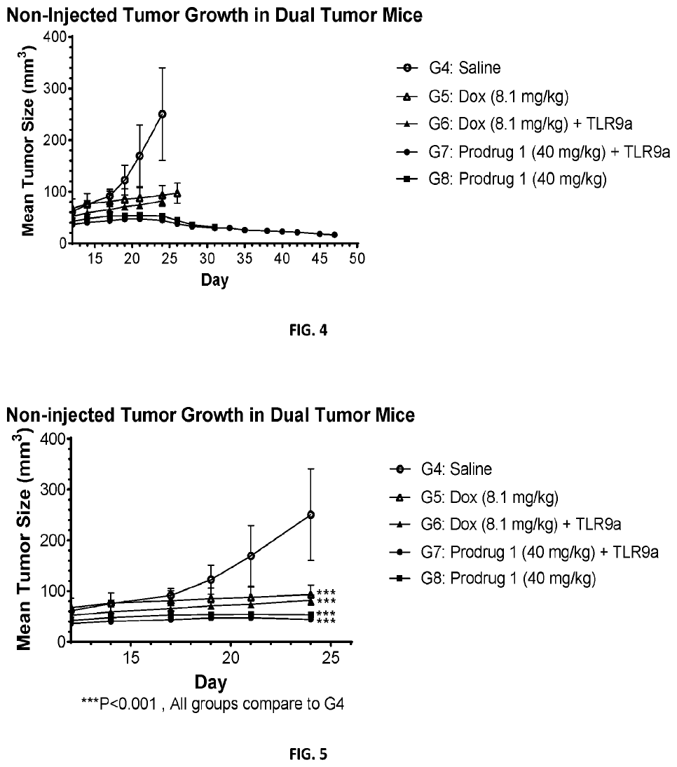

Injected tumor volumes of all treatment groups at different time points are shown in FIG. 2A and FIG. 2B for Groups 1-8. Injected tumor volumes of all treatment groups for analysis are shown in FIG. 3A and FIG. 3B for Groups 1-8. Non-injected tumor volumes of all treatment groups at different time points are shown in FIG. 4 for Groups 4-8. Non-injected tumor volumes of all treatment groups for analysis are shown in FIG. 5 for Groups 4-8.

### 6.3 Tumor Growth Inhibition Efficacy

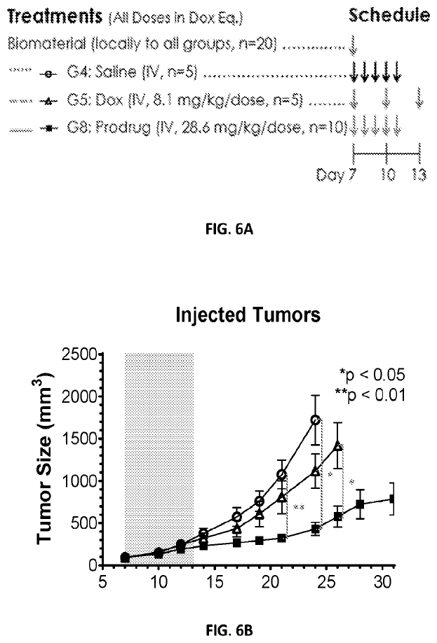

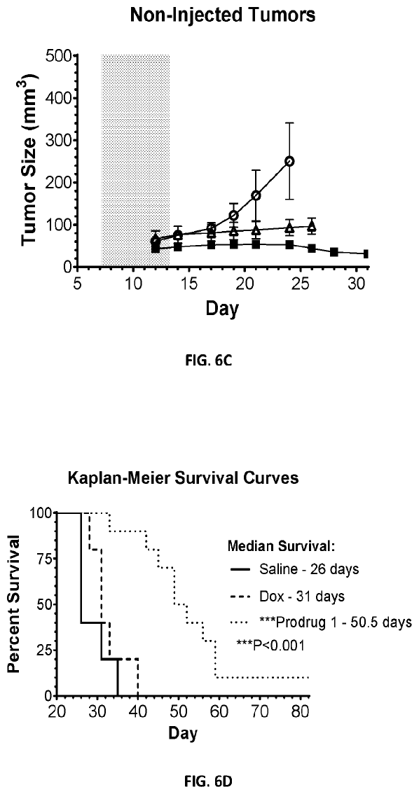

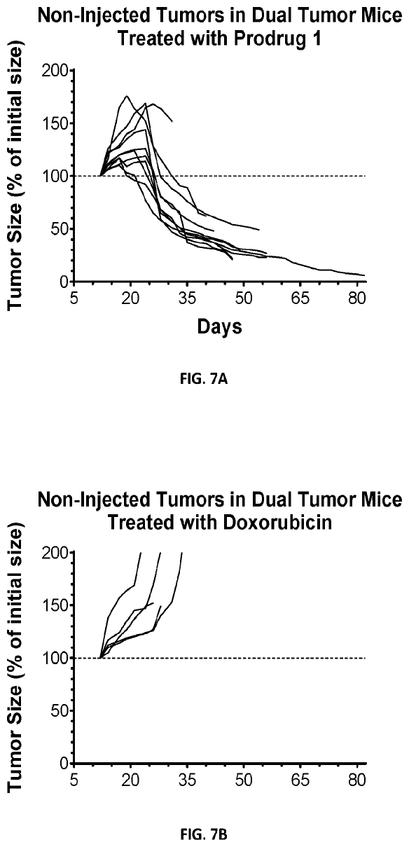

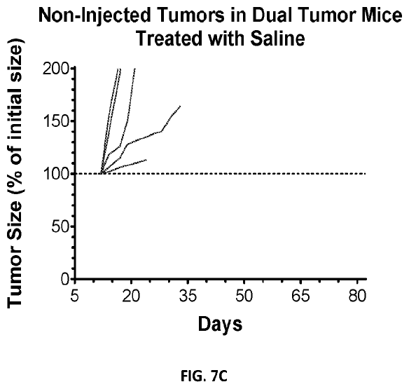

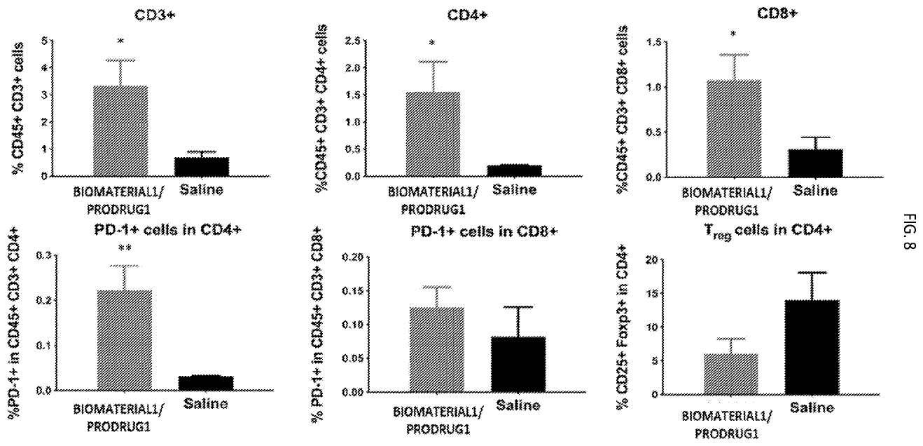

Treatment with BIOMATERIAL 1/PRODRUG 1 treatment resulted in significantly improved antitumor response (p<0.05) and overall survival (p<0.001) compared with that shown with conventional Dox treatment (FIG. 6A-6B and FIG. 6D). Furthermore, the majority of non-injected tumors in the BIOMATERIAL 1/PRODRUG 1 treatment group showed sustained anti-tumor responses, while in the conventional Dox treatment group progressive growth was observed in all non-injected tumors (FIG. 6C).

The tumor growth inhibition efficacy is summarized in Table 3, Table 4, and Table 5.

TABLE 3
Antitumor Activity (Injected tumor) in the Single Tumor Mice

| | | | D21 | | Significant |
|---|---|---|---|---|---|
| Treatment | TV (mm³)^{a} | TV (mm³)^{b} | T/C (%) | 1-T/C (%) | TV^{c} |
| G1 | 94 ± 9.3 | 1036 ± 103.6 | 100 | 0 | — |
| G2 | 94 ± 6.3 | 393 ± 100.5 | 38 | 62 | *** |
| G3 | 93 ± 5.9 | 289 ± 56.2 | 28 | 72 | *** |

^{a}Tumor volume at Day 7;
^{b}Tumor volume at Day 21;
^{c}All groups compare to G1 at Day 21.

TABLE 4
Antitumor Activity (Injected tumor) in Dual Tumor Mice

| | | | D21 | | Significant |
|---|---|---|---|---|---|
| Treatment | TV (mm³)^{a} | TV (mm³)^{b} | T/C (%) | 1-T/C (%) | TV^{c} |
| G4 | 94 ± 10.0 | 1076 ± 170.2 | 100 | 0 | — |
| G5 | 93 ± 8.5 | 810 ± 196.4 | 66 | 34 | ** |
| G6 | 93 ± 5.8 | 621 ± 187.9 | 45 | 55 | *** |
| G7 | 93 ± 5.7 | 238 ± 29.5 | 13 | 87 | *** |
| G8 | 93 ± 5.6 | 322 ± 45.9 | 25 | 75 | *** |

^{a}Tumor volume at Day 7;
^{b}Tumor volume at Day 21;
^{c}All groups compare to G4 at Day 21.

TABLE 5
Antitumor Activity (Non-injected tumor) in the Dual Tumor Mice

| | | | D24 | | |
|---|---|---|---|---|---|
| | TV | TV | T/C | 1-T/C | Significant |
| Treatment | (mm³)^{a} | (mm³)^{b} | (%) | (%) | TV^{c} |
| G4 | 61 ± 3.8 | 251 ± 90.0 | 100 | 0 | — |
| G5 | 67 ± 18.8 | 93 ± 19.2 | 34 | 66 | *** |
| G6 | 52 ± 4.8 | 82 ± 9.9 | 38 | 62 | *** |
| G7 | 36 ± 3.6 | 44 ± 4.9 | 30 | 70 | *** |
| G8 | 42 ± 3.8 | 53 ± 5.2 | 30 | 70 | *** |

^{a}Tumor volume at Day 12;
^{b}Tumor volume at Day 24;
^{c}All groups compare to G4 at Day 24.

c. All groups compare to G4 at Day24.

Immunocompetent C₅₇BL/6 mice were inoculated with mouse MC38 tumors. All tumor cells were implanted at Day 0. Treatments started at Day 7 with local injection of biomaterial at “injected” tumor, followed by systemic therapies. Large (injected) tumor was initiated with 5×10⁵ cells. Small (non-injected) tumor was initiated with 1×10⁵ cells. Tumor growth curves show mean±SEM; data points without errors bars occurred when the standard error was smaller than the symbol used to represent the treatment condition. Curves stopped after 1 or more mice in that group died or were sacrificed when tumor volume reached 2000 mm³.

*, ** Statistical significance in tumor growth curves was determined by unpaired t test with Welch's correction for each day. Saline (group 4) and Dox HCl (group 5) treatments were not significantly different on any day. BIOMATERIAL 1/PRODRUG 1 (group 8) treatment was significantly different from Saline, Dox HCl, or both Saline and Dox HCl treatments on days that are indicated with asterisks and brackets.

*** Statistical significance in survival was determined by log-rank (Mantel-Cox) test; BIOMATERIAL 1/PRODRUG 1 treatment was significantly different from Dox HCl or Saline, while Dox HCl and Saline were not significantly different from each other.

Immunocompetent C₅₇BL/6 mice were inoculated with mouse MC38 tumors. All tumor cells were implanted at Day 0. Treatments started at Day 7 with local injection of biomaterial at “injected” tumor, followed by systemic therapies. Large (injected) tumor was initiated with 5×10⁵ cells. Small (non-injected) tumor was initiated with 1×10⁵ cells. Tumor growths of individual non-injected tumors are displayed as a percentage of the initial volume of each tumor (measurement from day 12 post-inoculation).

### 6.5 Flow Cytometric Analysis

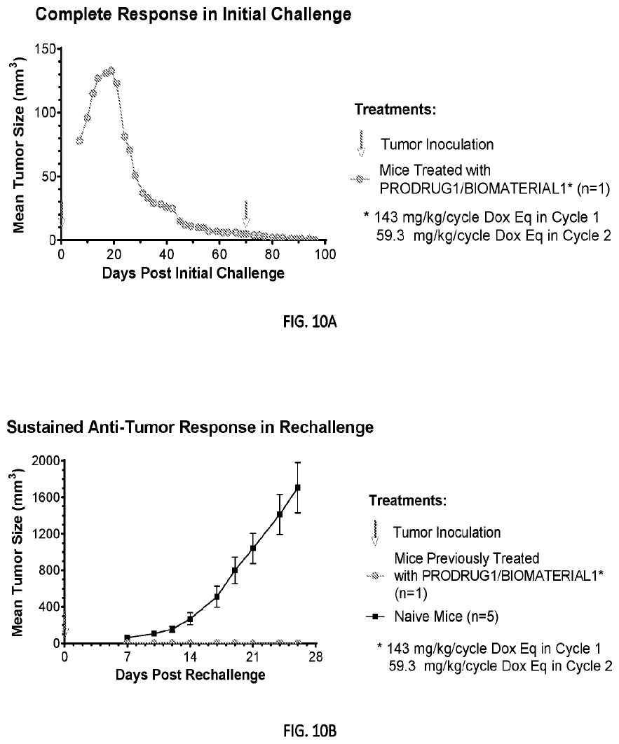

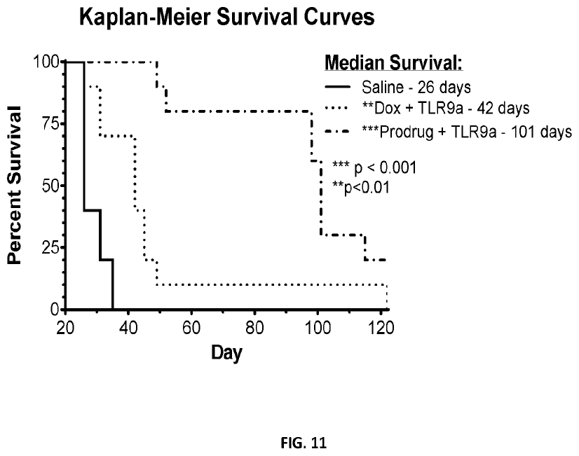

Tumor samples were collected at 1 week or 2 weeks after treatment for immune profiling. Table 6 indicates immune cell populations and the corresponding markers used for detection. FIG. 8 and FIG. 9 show quantification results of immune cell frequency and phenotype in tumor samples at 2 weeks.

At 1-week after completion of treatment, no differences were observed between the treatment and saline groups. At 2 weeks after treatment completion, a significant difference in the T-cell profile was identified between the BIOMATERIAL 1/PRODRUG 1-treated group and the saline group for both injected and non-injected tumors. For both injected (“injected”) (FIG. 8) and non-injected (“non-injected”) (FIG. 9) tumors, the overall % CD45+CD3+ cells increased in BIOMATERIAL 1/PRODRUG 1-treated mice compared with that shown in saline-treated mice. This suggested an increase in total tumor-infiltrating lymphocytes (TILs) in BIOMATERIAL 1/PRODRUG 1-treated mice. Among these cells, the percentages of CD8+ and CD4+ cells were significantly higher in the injected tumor (FIG. 8) with BIOMATERIAL 1/PRODRUG 1 treatment versus saline. In the non-injected tumor, only the CD4+ cell percentage was significantly higher (FIG. 9) in the BIOMATERIAL 1/PRODRUG 1-treated group versus the saline-treated group. In the non-injected tumors (FIG. 9), there were no CD4+CD25+ FoxP3+ cells observed in the BIOMATERIAL 1/PRODRUG 1-treated group.

Taken together, these results suggested an overall increase in helper T cell and CTL effectors and a decrease in T_{reg} effectors infiltrating into the tumors of the BIOMATERIAL 1/PRODRUG 1 TREATMENT-treated group. Although the cell percentage of CD8+ cells in non-injected tumors (FIG. 9) appeared higher and the percentage of FoxP3+ cells in injected tumors (FIG. 8) was lower than in the saline group, a greater number of animals per group may have been required to obtain statistical significance.

Interestingly, in the injected tumors (FIG. 8) of the BIOMATERIAL 1/PRODRUG 1-treated group, there was a higher percentage of PD-1+CD4+ T cells compared with that shown in the saline group. Although elevated PD-1 indicates T-cell exhaustion, the true functional significance of these cells needs to be explored further. This difference was not observed in the non-injected tumors (FIG. 9). This finding also provides a firm basis for applying combination therapies of BIOMATERIAL 1/PRODRUG 1 with anti-PD-1 checkpoint blockers in future studies. Further, there was no difference in the PD-1 expression in CD8+ cells in the injected or non-injected tumors in saline or BIOMATERIAL 1/PRODRUG 1 treatment groups.

Collectively, the flow cytometry data from this study indicated that BIOMATERIAL 1/PRODRUG 1 treatment was capable of immune activation and increased the total number of TILs in both injected and non-injected tumors. These effects were present at 2 weeks, but not after 1 week of treatment, suggesting a temporal response. Solid tumors suppress the immune response by increasing infiltration of T_{reg} cells or engaging checkpoint molecules. This study suggests that BIOMATERIAL 1/PRODRUG 1 treatment may decrease T_{reg} cells in non-injected tumors and may have potential benefits when combined with anti-PD-1 antibodies.

TABLE 6
Immune Cell Populations and Corresponding Markers

| Markers | Cell populations |
|---|---|
| % CD45+CD3+ | % of T lymphocytes in total cells |
| % CD45+CD3+CD4+ | % of CD4+T lymphocytes in total cells |
| % CD45+CD3+CD8+ | % of CD8+T lymphocytes in total cells |
| % PD-1+cells in gated CD45+ | % of exhausted cells in CD4+T cell |
| CD3+CD4+ | population |
| % PD-1+cells in gated CD45+ | % of exhausted cells in CD8+T cell |
| CD3+CD8+ | population |
| % CD25+Foxp3+in gated | % of T_{reg}s in CD4+T cells population |
| CD45+CD3+CD4+ | |
| % CD45+CD3+CD4+ | % of T_{reg}s in total cells |
| CD25+ Foxp3+ | |

T_{reg} = regulatory T cell

### 7. Results of Rechallenge Study

Aside from the dual-tumor groups, this study also investigated a tumor re-implantation in mice inoculated with only one MC38 tumor. On Day 38, all animals in group G2 (Table 1)(n=8) were treated with a second cycle of BIOMATERIAL 1/PRODRUG 1-100-μL intratumoral BIOMATERIAL 1 injection followed by 5 daily doses (1 dose per day) of PRODRUG 1—this time at 11.9 mg/kg/dose Dox Eq (59.3 mg/kg/cycle Dox Eq).

On Day 70, one mouse trending toward complete response from group G2 (FIG. 10A) was re-challenged with 5×10⁵ MC38 tumor cells inoculated SC at the left flank. A control group of 5 naïve mice were also inoculated with 5×10⁵ MC38 tumor cells on the same day. Tumor growth curves for the treated and untreated animals are presented in FIG. 10B.

In addition, mice from group G3 (Table 1) (n=5), and mice from dual tumor groups G6, G7, and G8 (n=10) were rechallenged. Tumors in all naïve mice grew rapidly, while tumor growth in the BIOMATERIAL 1/PRODRUG 1-pretreated mouse was suppressed, suggesting that BIOMATERIAL 1/PRODRUG 1 treatment may trigger an antitumor memory immune response.

### 8. Summary and Conclusion

In this study, the therapeutic efficacy of PRODRUG 1, PRODRUG 1+ TLR agonist, Dox and Dox+ TLRa in the subcutaneous MC-38 cancer model is evaluated.

Treatment with PRODRUG 1, PRODRUG 1+ TLR agonist produced significant antitumor activity, with P<0.001, P<0.001 respectively compared with the control group G1. Treatment of injected tumor with PRODRUG 1, PRODRUG 1+ TLR agonist, Dox and Dox+ TLR agonist produced significant antitumor activity, with P<0.01, P<0.001, P<0.001, P<0.001 respectively compared with the control group G4. Treatment of non-injected tumor with PRODRUG 1, PRODRUG 1+ TLR agonist, Dox and Dox+ TLR agonist produced significant antitumor activity, with P<0.001, P<0.001, P<0.001, P<0.001 respectively compared with the G4. All treatments were well-tolerated in the MC-38 tumor bearing C57BL/6 mice.

Collectively, the flow cytometry data from this study indicated that BIOMATERIAL 1/PRODRUG 1 treatment was capable of immune activation and increased the total number of TILs in both injected and non-injected tumors. These effects were present at 2 weeks, but not after 1 week of treatment, suggesting a temporal response. Solid tumors suppress the immune response by increasing infiltration of T_{reg} cells or engaging checkpoint molecules. This study suggests that BIOMATERIAL 1/PRODRUG 1 treatment may decrease T_{reg} cells in non-injected tumors and may have potential benefits when combined with anti-PD-1 antibodies.

Sixteen mice were re-challenged with MC-38 cells and the tumors of all mice still showed significant change when compared to the vehicle group at the end of the re-implantation study. In comparison, all ten of ten naïve mice that were implanted with MC-38 cells grew tumors normally. This data suggests that treatment may induce an anti-tumor immune memory response.

In conclusion, BIOMATERIAL 1/PRODRUG 1 treatment showed improved tumor growth inhibition and overall survival when compared with conventional Dox treatment. In addition, it showed complete remission in 10% of mice treated, followed by sustained anti-tumor response upon re-challenged. Furthermore, BIOMATERIAL 1/PRODRUG 1 treatment induced immune activation and lead to increased total TILs 2 weeks after starting therapy. Overall, BIOMATERIAL 1/PRODRUG 1 treatment demonstrated a sustained anti-cancer and immunomodulatory effect in both injected and non-injected tumors, suggesting it may be useful in treating localized tumors and metastatic disease.

For reasons of completeness, various aspects of the invention are set out in the following numbered clauses:

Clause A1. A compound of formula (I-A), or a pharmaceutically acceptable salt thereof

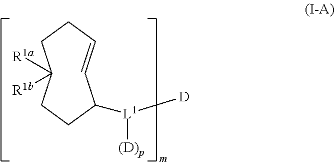

wherein
R^{1a}, at each occurrence, is independently selected from the group consisting of hydrogen, C₁₋₄alkyl, and C₁₋₄haloalkyl;
R^{1b}, at each occurrence, is independently selected from the group consisting of hydrogen, C₁₋₄alkyl, C₁₋₄haloalkyl, C(O)OH, C(O)OC₁₋₄ alkyl, C(O)N(R^{1c})CHR^{1e}CO₂H, C(O)N(R^{1c})CHR^{1e}C(O)OC₁₋₄ alkyl, C(O)N(R^{1c})—C₁₋₆alkylene-CO₂H, and C(O)N(R^{1c})—C₁₋₆ alkylene-C(O)OC₁₋₄ alkyl;
R^{1c}, at each occurrence, is independently hydrogen or C₁₋₄alkyl;
R^{1e}, at each occurrence, is independently —C₁₋₄alkylene-CO₂H, —C₁₋₄alkylene-CONH₂, or —C₁₋₄ alkylene-OH;
D, at each occurrence, is independently a payload selected from the group consisting of a toll-like receptor (TLR) agonist and a stimulator of interferon genes (STING) agonist;
L¹, at each occurrence, is independently a linker;
m, at each occurrence, is independently 1, 2, or 3; and
p, at each occurrence, is independently 0, 1, or 2.
   Clause A2. The compound of clause A1, or a pharmaceutically acceptable salt thereof, wherein
R^{1a} is hydrogen; and
R^{1b} is hydrogen.
   Clause A3. The compound of clause A1, or a pharmaceutically acceptable salt thereof, wherein
R^{1a} is C₁₋₄alkyl; and
R^{1b} is selected from the group consisting of C(O)OH, C(O)OC₁₋₄alkyl, C(O)N(R^{1c})CHR^{1e}CO₂H, C(O)N(R^{1c})CHR^{1e}C(O)OC₁₋₄ alkyl, C(O)N(R^{1c})—C₁₋₆alkylene-CO₂H, and C(O)N(R^{1c})—C₁₋₆ alkylene-C(O)OC₁₋₄ alkyl.
   Clause A4. The compound of clause A3, or a pharmaceutically acceptable salt thereof, wherein
   R^{1b} is selected from the group consisting of C(O)OH, C(O)N(R^{1c})CHR^{1e}CO₂H, and C(O)N(R^{1c})CH₂CO₂H.
   Clause A5. The compound of clause A3 or A4, or a pharmaceutically acceptable salt thereof, wherein R^{e}e is —CH₂CO₂H, —CH₂CH₂CO₂H, —CH₂CONH₂, —CH₂CH₂CONH₂, —CH₂OH, or —CH(CH₃)OH.
   Clause A6. The compound of clause A3 or A4, or a pharmaceutically acceptable salt thereof, wherein R^{1e} is —C₁₋₄alkylene-CO₂H.
   Clause A7. The compound of clause A3 or A4, or a pharmaceutically acceptable salt thereof, wherein R^{1e} is —CH₂CO₂H.
   Clause A8. The compound of any of clauses A3-A7, or a pharmaceutically acceptable salt thereof, wherein R^{1a} is CH₃.
   Clause A9. The compound of any of clauses A3-A8, or a pharmaceutically acceptable salt thereof, wherein R^{1c} is hydrogen.
   Clause A10. The compound of any of clauses A1-A9, or a pharmaceutically acceptable salt thereof, wherein:
L¹ is

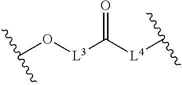

### or —O—;

L³ is a bond or C₁₋₆alkylene;
L⁴ is a bond, —NHN; —N(R¹⁰)—C₂₋₆alkylene-N(R¹¹)—, —N(R¹²)—C₂₋₃alkylene-N(R¹³)C(O)—, —N(R¹⁰)—C₁₋₆alkylene-C(O)NHN; —NHNHC(O)C₁₋₆alkylene-C(O)NHN; —CH(NHC(O)R¹⁴)C₁₋₄alkylene-S—S—C₁₋₄alkylene-OC(O)—, —NHNHC(O)CH(NHC(O)R¹⁵)CH₂C(O)—, —C₁₋₆alkylene-CH(G^{x})OC(O)—,

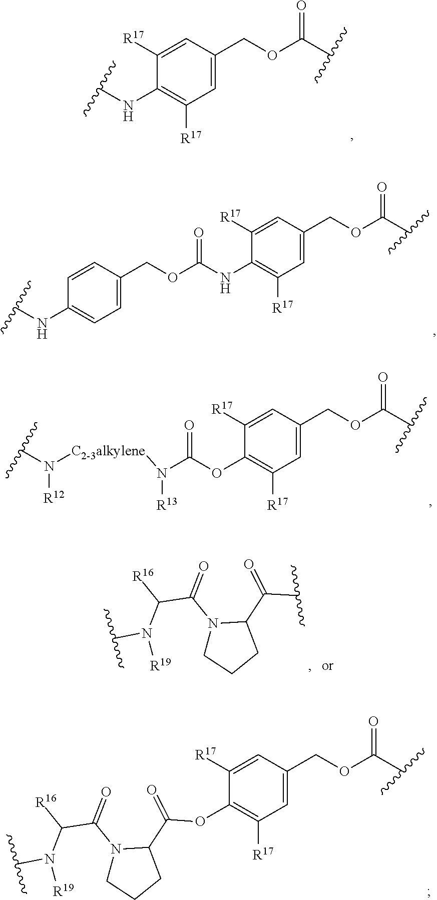

R¹⁰, R¹¹, R¹², R¹³, R¹⁴, R¹⁵ and R¹⁹ are each independently hydrogen or C₁₋₄ alkyl;
R¹⁶ is hydrogen, C₁₋₄ alkyl, —C₁₋₄alkylene-OH, —C₁₋₄alkylene-OC₁₋₄ alkyl, —C₁₋₄alkylene-CO₂H, or —C₁₋₄alkylene-CONH₂;
R¹⁷, at each occurrence, is independently hydrogen or —CH₂OC(O)—; and
G^{x} is phenyl optionally substituted with 1-5 substituents independently selected from the group consisting of halogen, C₁₋₄alkyl, C₁₋₄haloalkyl, C₁₋₄alkoxy, cyano, and nitro.
   Clause A11. The compound of any of clauses A1-A10, or a pharmaceutically acceptable salt thereof, wherein m is 1.
   Clause A12. The compound of clause A11, or a pharmaceutically acceptable salt thereof, wherein:

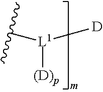

is

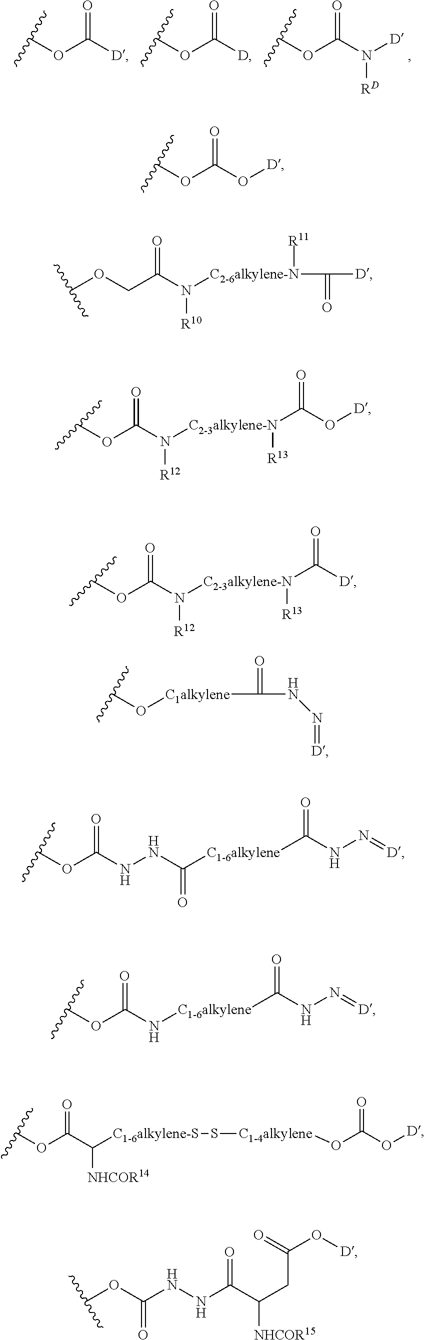

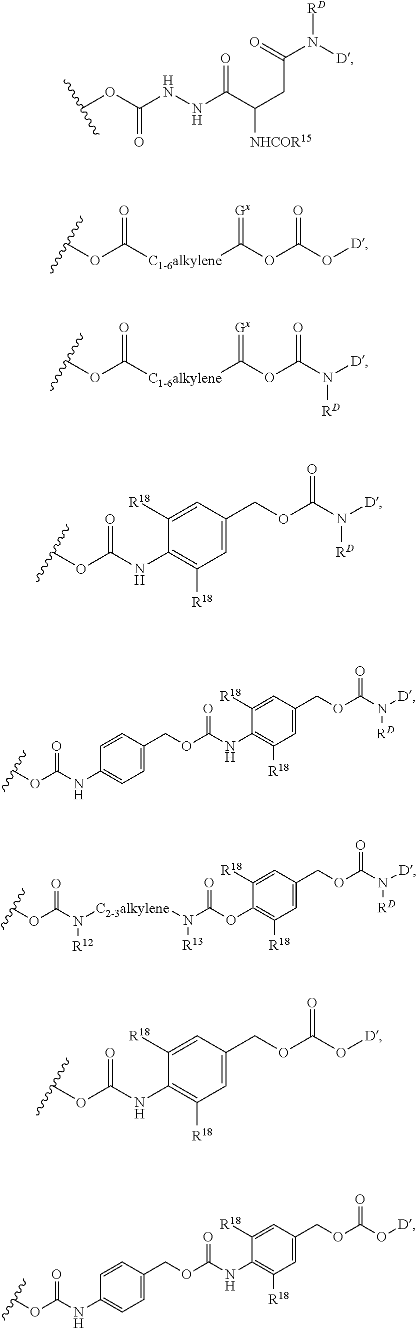

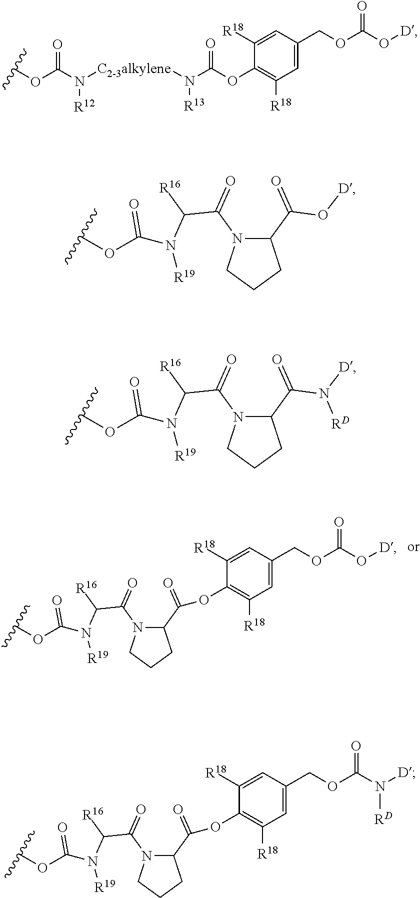

R¹⁸, at each occurrence, is independently hydrogen or —CH₂OC(O)NHD′;
R^{D} is hydrogen or C₁₋₄alkyl on a nitrogen atom of the payload; and
D′ is a payload moiety.
   Clause A13. The compound of any of clauses A1-A12, or a pharmaceutically acceptable salt thereof, wherein p is 0.
   Clause A14. The compound of clause A13, or a pharmaceutically acceptable salt thereof, wherein m is 2 or 3.
   Clause A15. The compound of clause A14, or a pharmaceutically acceptable salt thereof, wherein

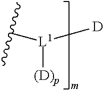

is

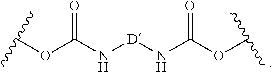

Clause A16. The compound of any of clauses A1-A15, or a pharmaceutically acceptable salt thereof, wherein the payload D is selected from the group consisting of

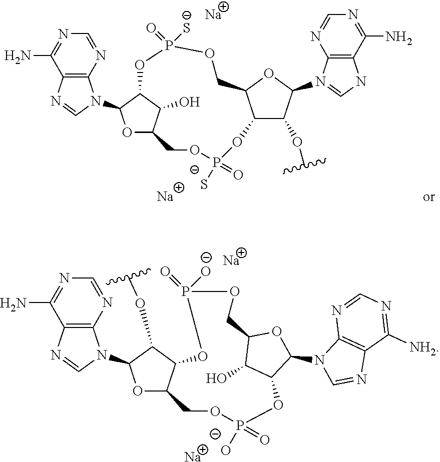

Clause A17. The compound of clause A1, or a pharmaceutically acceptable salt thereof, selected from the group consisting of

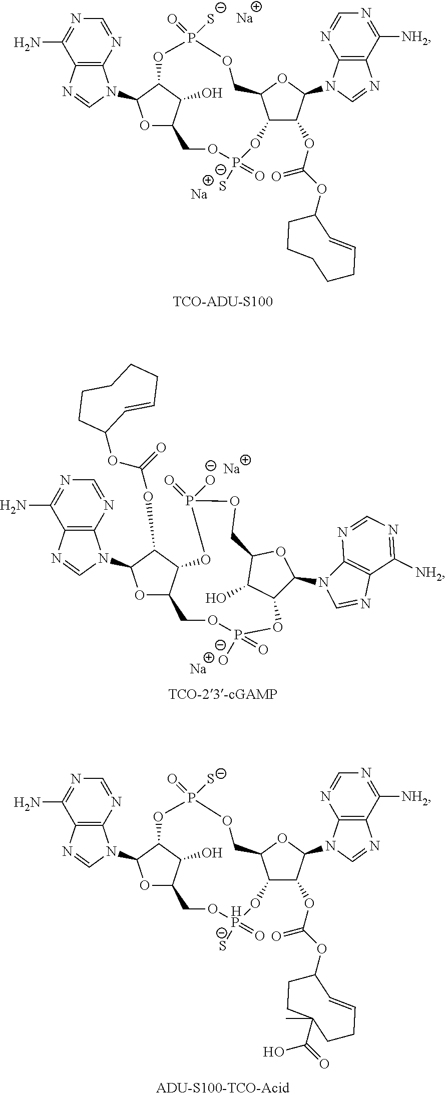

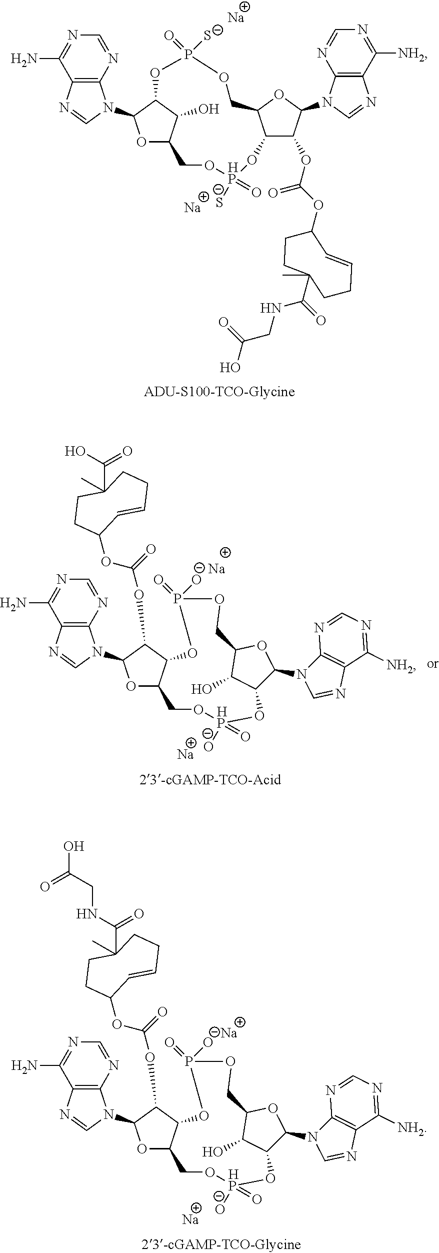

Clause A18. A pharmaceutical composition comprising the compound of any of clauses A1-A17, or a pharmaceutically acceptable salt thereof, and a pharmaceutically acceptable carrier.
Clause A19. A method of treating or preventing a condition or disorder or enhancing or eliciting an immune response, the method comprising administering to a subject in need thereof, a therapeutically effective amount of the compound of any of clauses A1-A17, or a pharmaceutically acceptable salt thereof, or the pharmaceutical composition of clause A18, and a therapeutic support composition, the therapeutic support composition comprising a biocompatible support and a tetrazine-containing group of formula

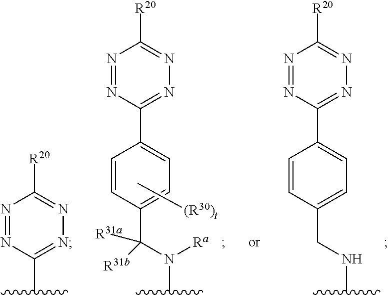

wherein

R²⁰ is selected from the group consisting of hydrogen, halogen, cyano, nitro, alkyl, alkenyl, alkynyl, heteroalkyl, aryl, heteroaryl, heterocycle, cycloalkyl, cycloalkenyl, CF₃, CF₂—R′, NO₂, OR′, SR′, C(═O)R′, C(═S)R′, OC(═O)R″, SC(═O)R′″, OC(═S)R′″, SC(═S)R′″, S(═O)R′, S(═O)₂R, S(═O)₂NR′ R″, C(═O)O—R′, C(═O)S—R′, C(═S)O—R′, C(═S)S—R′, C(═O)NR′R″, C(═S)NR′ R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S) R′R′″, SC(═S)R′R″, NR′C(═O)NR″R″, and NR′C(═S)NR″R″;

R′ and R″ at each occurrence are independently selected from hydrogen, aryl and alkyl;

R′″ at each occurrence is independently selected from aryl and alkyl;

R³⁰ is halogen, cyano, nitro, hydroxy, alkyl, haloalkyl; alkenyl, alkynyl, alkoxy; halalkoxy; heteroalkyl, aryl, heteroaryl, heterocycle, cycloalkyl, or cycloalkenyl;

R^{a}, R^{31a} and R^{31b} are each independently hydrogen, C₁-C₆-alkyl, or C₁-C₆-haloalkyl; and

t is 0, 1, 2, 3, or 4.

Clause A20. The method of clause A19, wherein the tetrazine-containing group is linked or directly bonded to a hyaluronic acid biocompatible support.
Clause A21. The method of clause A20, wherein the therapeutic support composition comprises substituted hyaluronic acid units of formula (II),

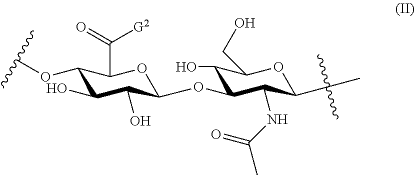

wherein G² is

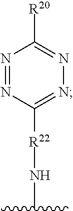

and
R²² is a linker of 1 to 100 linking atoms.
Clause A22. The method of clause A21, wherein:

### G² is

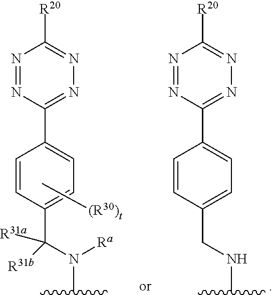

Clause A23. The method of clause A21, wherein

### G² is

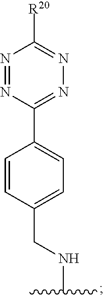

and
R²⁰ is hydrogen or C₁₋₄alkyl.
Clause A24. The method of any of clauses A19-A23, wherein the method is a method of treating or preventing a cancer.
Clause A25. The method of clause A24, wherein the cancer is a melanoma, renal cancer, prostate cancer, ovarian cancer, breast cancer, glioma, lung cancer, soft tissue carcinoma, soft tissue sarcoma, osteosarcoma, or pancreatic cancer.
Clause A26. The method of clause A24 or A25, wherein the cancer is a solid tumor.
Clause A27. The method of clause A24 or A25, wherein the cancer is a soft tissue sarcoma.
Clause A28. The method of clause A27, wherein the soft tissue sarcoma is a fibrosarcoma, rhabdomyosarcoma, or Ewing's sarcoma.
Clause A29. The method of any of clauses A19-A23, wherein the method is a method of enhancing or eliciting an immune response.
Clause A30. The method of clause A29, wherein the immune response is an increase in one or more of leukocytes, lymphocytes, monocytes, and eosinophils.
Clause A31. The method of any of clauses A19-A30, further comprising administering a therapeutically effective amount of an additional therapeutic agent selected from the group consisting of an anticancer agent, an immune checkpoint inhibitor, or a compound of formula (I-B), or a pharmaceutically acceptable salt thereof,

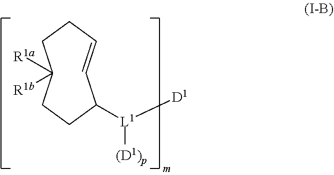

wherein
D¹, at each occurrence, is independently a payload selected from an anticancer drug payload, a microbial immunosuppressive drug payload, an anti-restenosis drug payload, antibiotic drug payload, antifungal drug payload, antiviral drug payload, anti-inflammatory/anti-arthritic drug payload, a corticosteroid drug payload, and an immunosuppressant drug payload; and R^{1a}, R^{1b}, L¹, and m are as defined in any of claims 1-11.
Clause A32. The method of clause A31, wherein p is 0; m is 1; and -L¹- is

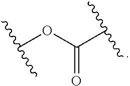 [SMILES: *OC(*)=O]

Clause A33. The method of clause A31 or A32, wherein the anticancer drug is doxorubicin.
Clause A34. A kit comprising the compound of any of clauses A1-A17, or a pharmaceutically acceptable salt thereof, or the pharmaceutical composition of clause A18, and instructions for use thereof.
Clause A35. The kit of clause A34, further comprising the therapeutic support composition as defined in any of clauses A19-A23.
Clause A36. The kit of clause A34 or A35, further comprising the compound of formula (I-B), as defined in any of clauses A31-A33.
Clause B1. A method of treating cancer or enhancing or eliciting an immune response comprising administering to a subject in need thereof:

a) a therapeutically effective amount of a compound of formula (II-A), or a pharmaceutically acceptable salt thereof,

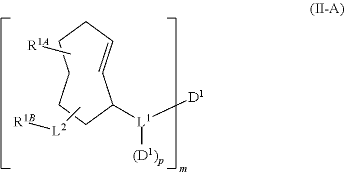

wherein
R^{1A}, at each occurrence, is independently selected from the group consisting of C₁₋₄alkyl, C₁₋₄haloalkyl, and C₁₋₄alkoxy;
R^{1B}, at each occurrence, is independently selected from the group consisting of G¹, OH, —NR^{1C}—C₁₋₄alkylene-G¹,
   —NR^{1c}—C₁₋₄alkylene-N(R^{1d})₂, —N(R^{1c})CHR^{1e}CO₂H, —N(R^{1c})—C₁₋₆alkylene-CO₂H, —N(R^{1f})—C₂₋₄alkylene-(N(C₁₋₄alkylene-CO₂H)—C₂₋₄alkylene)ₙ-N(C₁₋₄alkylene-CO₂H)₂, —N(R^{1c})CHR^{1e}(O)OC₁₋₆alkyl, —N(R^{1c})—C₁₋₆alkylene-C(O)OC₁₋₆alkyl, and —N(R^{1f})—C₂₋₄alkylene-(N(C₁₋₄alkylene-C(O)OC₁₋₆alkyl)-C₂₋₄alkylene)ₙ-N(C₁₋₄alkylene-C(O)OC₁₋₆ alkyl)₂;
R^{1c} and R^{1d}, at each occurrence, are independently hydrogen or C₁₋₄alkyl;
R^{1e}, at each occurrence, is independently —C₁₋₄alkylene-CO₂H, —C₁₋₄alkylene-CONH₂, or —C₁₋₄ alkylene-OH;
R^{1f}, at each occurrence, is independently hydrogen, C₁₋₆ alkyl, or C₁₋₄ alkylene-CO₂H;
D¹, at each occurrence, is independently an anticancer agent payload;
L¹, at each occurrence, is independently a linker;
L², at each occurrence, is independently selected from the group consisting of —C(O)— and C₁₋₃ alkylene;
G¹, at each occurrence, is independently an optionally substituted heterocyclyl;
m is 1, 2, or 3
n, at each occurrence, is independently 0, 1, 2, or 3; and
p, at each occurrence, is independently 0, 1, or 2;
   b) a therapeutic support composition comprising a support and a tetrazine-containing group of formula

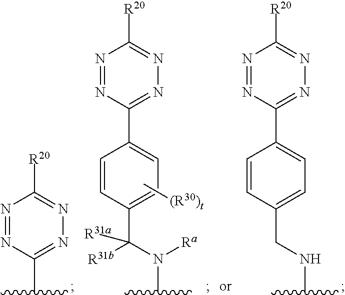

wherein R²⁰ is selected from the group consisting of hydrogen, halogen, cyano, nitro, alkyl, alkenyl, alkynyl, heteroalkyl, aryl, heteroaryl, heterocycle, cycloalkyl, cycloalkenyl, CF₃, CF₂—R′, NO₂, OR′, SR′, C(═O)R′, C(═S)R′, OC(═O)R″, SC(═O)R′″, OC(═S)R′″, SC(═S)R′″, S(═O)R′, S(═O)₂R″, S(═O)₂NR′ R″, C(═O)O—R′, C(═O)S—R′, C(═S)O—R′, C(═S)S—R′, C(═O)NR′R″, C(═S)NR′ R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S) R′R′″, SC(═S)R′R″, NR′C(═O)NR″R″, and NR′C(═S)NR″R″; R′ and R″ at each occurrence are independently selected from hydrogen, aryl and alkyl; and R′″ at each occurrence is independently selected from aryl and alkyl; R³⁰ is halogen, cyano, nitro, hydroxy, alkyl, haloalkyl; alkenyl, alkynyl, alkoxy; halalkoxy; heteroalkyl, aryl, heteroaryl, heterocycle, cycloalkyl, or cycloalkenyl; R^{a}, R^{31a} and R^{31b} are each independently hydrogen, C₁-C₆-alkyl, or C₁-C₆-haloalkyl; and t is 0, 1, 2, 3, or 4;

wherein the tetrazine-containing group is linked or directly bonded to the support; and
c) a therapeutically effective amount one or more immunomodulatory agents, or a pharmaceutically acceptable salt thereof.
Clause B2. The method of clause B1, wherein the method is the method of enhancing or eliciting an immune response wherein the administration of a), b), and c) enhances or elicits an immune response against a cancer in the subject.
Clause B3. The method of clause B1 or B2, wherein the immune response is an increase or decrease in one or more of innate and adaptive immune cells including but not limited to leukocytes, lymphocytes, monocytes, eosinophils, and antibodies.
Clause B4. The method of clause B1, wherein the method is the method of treating cancer.
Clause B5. The method of any of clauses B1-B4, wherein the cancer is a melanoma, renal cancer, prostate cancer, ovarian cancer, breast cancer, glioma, lung cancer, soft tissue carcinoma, soft tissue sarcoma, osteosarcoma, rhabdomyosarcoma, colon cancer or pancreatic cancer.
Clause B6. The method of any of clauses B1-B5, wherein the cancer is a solid tumor.
Clause B7. The method of any of clauses B1-B5, wherein the cancer is a soft tissue sarcoma.
Clause B8. The method of clause B7, wherein the soft tissue sarcoma is a fibrosarcoma, rhabdomyosarcoma, or Ewing's sarcoma.
Clause B9. The method of any of clauses B1-B5, wherein the cancer is a diffuse intrinsic pontine glioma.
Clause B10. The method of any of clauses B1-B9, further comprising administering a therapeutically effective amount of an immune checkpoint inhibitor.
Clause B11. The method of any of clauses B1-B10, wherein the immunomodulatory agent(s) is a toll-like receptor (TLR) agonist.
Clause B12. The method of clause B11, wherein the toll-like receptor (TLR) agonist is Bacillus Calmette-Guerin (BCG), lipopolysaccharide, peptidoglycan, polyriboinosinic-polyribocytidylic acid (Poly I:C), Imiquimod, Coley's toxin, Polyadenylic-polyuridylic acid (Poly A:U), Monophosphoryl lipid A, single- and double-stranded RNA, or a CpG oligodeoxynucleotide (ODN).
Clause B13. The method of any of clauses B1-B10, wherein the immunomodulatory agent(s) is a stimulator of interferon genes (STING) agonist.
Clause B14. The method of any of clauses B1-B10, wherein the immunomodulatory agent(s) is a cytokine, cytokine inhibitor, cytokine receptor agonist, or cytokine receptor antagonist.
Clause B15. The method of any of clauses B1-B10, wherein the immunomodulatory agent(s) is a chemokine, chemokine inhibitor, chemokine receptor agonist, or chemokine receptor antagonist.
Clause B16. The method of any of clauses B1-B15, wherein a), b), and c) are administered simultaneously, separately, or sequentially, and in any order.
Clause B17. The method of any of clauses B1-B16, wherein the immunomodulatory agent(s) is administered simultaneously with the therapeutic support composition.
Clause B18. The method of clause B16 or B17, wherein the simultaneous administration is by coinjection, coimplantation, or coformulation.
Clause B19. The method of any of clauses B1-B18, wherein
R^{1B} is selected from the group consisting of G¹, OH, —NR^{1c}_C₁₋₄alkylene-G¹⁻, —NR^{1c}—C₁₋₄alkylene-N(R^{1d})₂, —N(R^{1c})CHR^{1e}CO₂H, —N(R^{1c})CH₂CO₂H, and —N(R^{1f})—CH₂CH₂—(N(CH₂CO₂H)CH₂CH₂)ₙ—N(CH₂CO₂H)₂;
R^{1e} is —CH₂CO₂H, —CH₂CH₂CO₂H, —CH₂CONH₂, —CH₂CH₂CONH₂, —CH₂OH, or —CH(CH₃)OH; and
R^{1f} is hydrogen or CH₂CO₂H.
   Clause B20. The method of any of clauses B1-B18, wherein
R^{1A} is C₁₋₄alkyl;
R^{1B} is selected from the group consisting of G¹, OH, —NR^{1c}—C₁₋₄alkylene-G¹, —NR^{1c}—C₁₋₄ alkylene-N(R^{1d})₂, —N(R^{1c})CHR^{1e}CO₂H, —N(R^{1c})CH₂CO₂H, and —N(R^{1f})—CH₂CH₂—(N(CH₂CO₂H)CH₂CH₂)ₙ—N(CH₂CO₂H)₂;
R^{1e} is —C₁₋₄alkylene-CO₂H;
R^{1f} is hydrogen or C₁₋₄alkylene-CO₂H;
G¹ is a 4- to 8-membered monocyclic heterocyclyl containing a first nitrogen and optionally one additional heteroatom selected from nitrogen, oxygen, and sulfur, G¹ being attached at the first nitrogen and optionally substituted with 1-4 substituents independently selected from the group consisting of C₁₋₄ alkyl, C₁₋₄ haloalkyl, halo, cyano, OH, —OC₁₋₄alkyl, and oxo; and
n is 0, 1, or 2.
   Clause B21. The method of clause B20, wherein
R^{1A} is CH₃;
R^{1e} is —CH₂CO₂H;
R^{1f} is hydrogen or CH₂CO₂H; and
G¹ is a piperazinyl, morpholinyl, piperidinyl, azepanyl, or pyrrolidinyl, attached through a ring nitrogen atom and optionally substituted with 1-4 substituents independently selected from the group consisting of C₁₋₄ alkyl, C₁₋₄ haloalkyl, halo, cyano, OH, —OC₁₋₄alkyl, and oxo.
   Clause B22. The method of any of clauses B1-B21, wherein L² is —C(O)—.
   Clause B23. The method of clause B22, wherein
   R^{1B} is selected from the group consisting of OH, N(H)CH₂CO₂H, —N(H)CHR^{1e}CO₂H, —N(H)—CH₂CH₂—(N(CH₂CO₂H)CH₂CH₂)ₙ—N(CH₂CO₂H)₂, and —N(CH₂CO₂H)—CH₂CH₂—N(CH₂CO₂H)₂; and
   R^{1e} is —CH₂CO₂H.
   Clause B24. The method of any of clauses B1-B23, wherein:
L¹ is

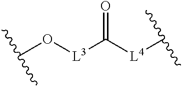

### or —O—;

L³ is a bond or C₁₋₆alkylene;
L⁴ is a bond, —NHN; —N(R¹⁰)—C₂₋₆alkylene-N(R¹¹)—, —N(R¹²)—C₂₋₃alkylene-N(R¹³)C(O)—, —N(R¹⁰)—C₁₋₆alkylene-C(O)NHN; —NHNHC(O)C₁₋₆alkylene-C(O)NHN; —CH(NHC(O)R¹⁴)C₁₋₄alkylene-S—S—C₁₋₄alkylene-OC(O)—, —NHNHC(O)CH(NHC(O)R¹⁵)CH₂C(O)—, —C₁₋₆alkylene-CH(G^{x})OC(O)—,

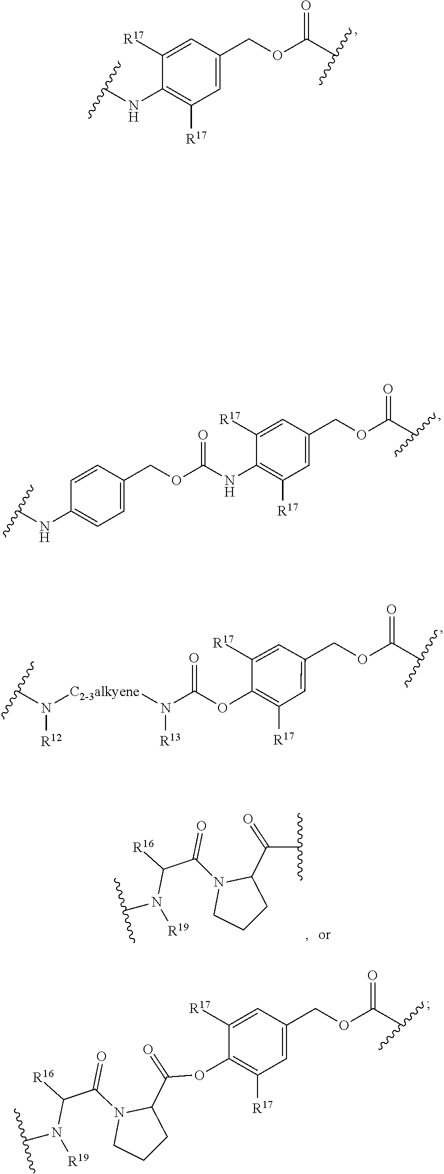

R¹⁰, R¹², R¹³, R¹⁴, R¹⁵, and R¹⁹ are each independently hydrogen or C₁₋₄alkyl;
R¹⁶ is hydrogen, C₁₋₄alkyl, —C₁₋₄alkylene-OH, —C₁₋₄alkylene-OC₁₋₄alkyl, —C₁₋₄alkylene-CO₂H, or —C₁₋₄alkylene-CONH₂;
R¹⁷, at each occurrence, is independently hydrogen or —CH₂OC(O)—; and
G^{x} is phenyl optionally substituted with 1-5 substituents independently selected from the group consisting of halogen, C₁₋₄alkyl, C₁₋₄haloalkyl, C₁₋₄alkoxy, cyano, and nitro.
   Clause B25. The method of any of clauses B1-B24, wherein m is 1.
   Clause B26. The method of clause B25, wherein:

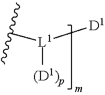

is

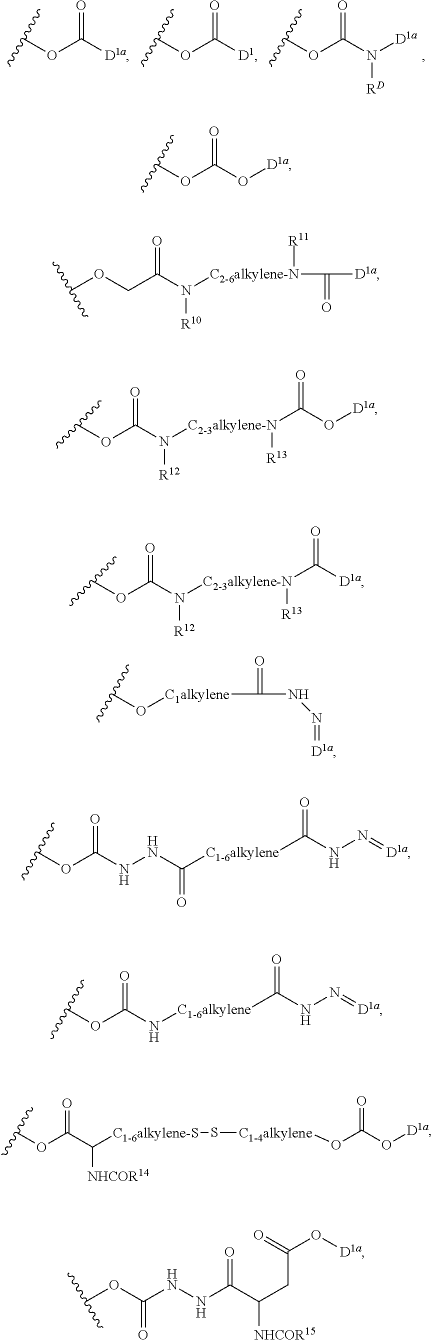

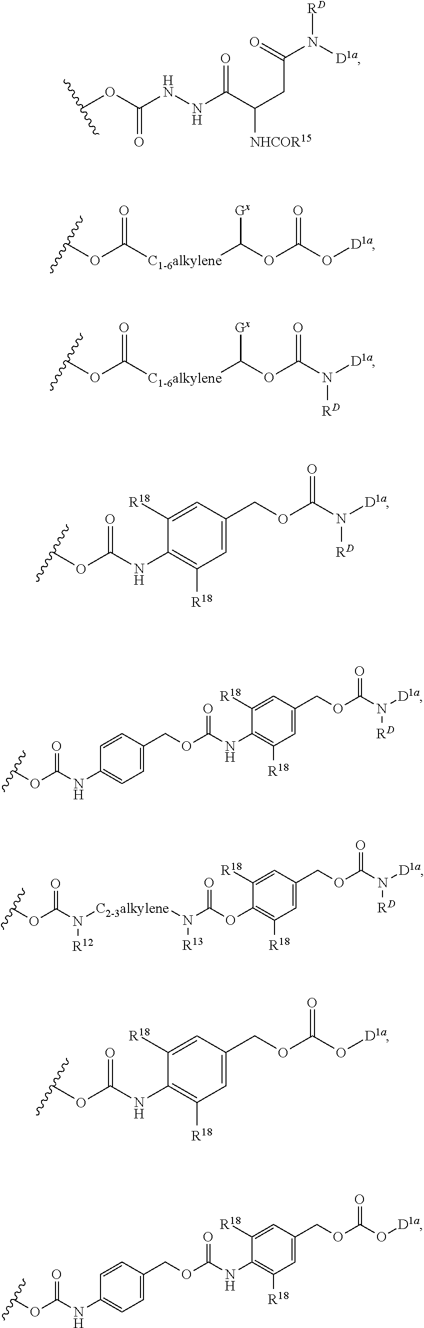

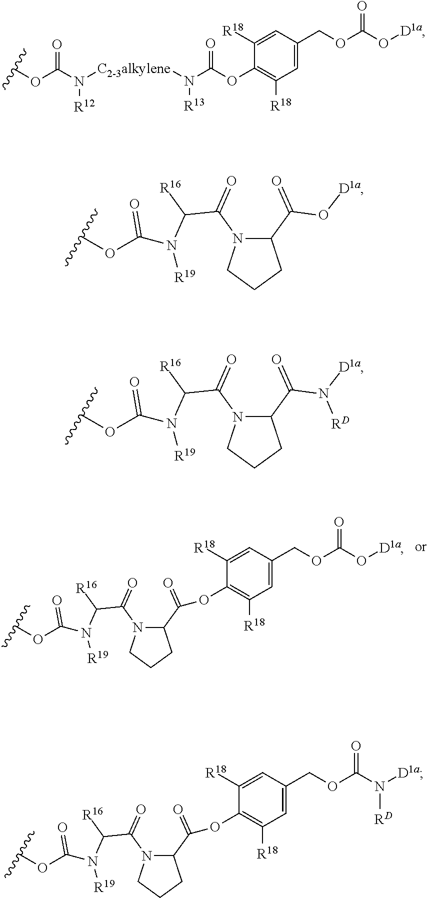

R¹⁸, at each occurrence, is independently hydrogen or —CH₂OC(O)NHD^{1a};
R^{D} is hydrogen or C₁₋₄alkyl on a nitrogen atom of the payload; and
D^{1a} is a payload moiety.
   Clause B27. The method of any of clauses B1-B26, wherein p is 0.
   Clause B28. The method of clause B27, wherein m is 2 or 3.
   Clause B29. The method of clause B28, wherein

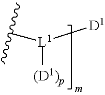

is

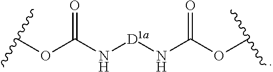

Clause B30. The method of any of clauses B1-B29, wherein the therapeutic support composition comprises substituted hyaluronic acid units of formula (II),

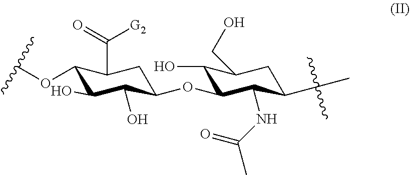

wherein G² is

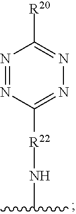

and R²² is a linker of 1 to 100 linking atoms.
Clause B31. The method of clause B30, wherein:

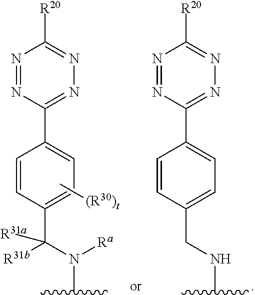

G² is

Clause B32. The method of clause B31, wherein

### G² is

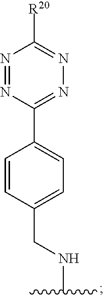

and
R²⁰ is hydrogen or C₁₋₄alkyl.
Clause B33. A kit comprising
a) the compound of formula (I-A), as described in any of clause B1 or B19-B29, or a pharmaceutically acceptable salt or composition thereof;
b) one or more immunomodulatory agents, or a pharmaceutically acceptable salt or composition thereof; and
c) instructions for use.
Clause B34. The kit of clause B33 further comprising the therapeutic support composition, as described in any of clauses B1 or B30-B32.
Clause B35. A kit comprising
a) the therapeutic support composition, as described in any of clauses B1 or B30-B32;
b) one or more immunomodulatory agents, or a pharmaceutically acceptable salt or composition thereof; and
c) instructions for use.
Clause B36. A pharmaceutical composition comprising
a) the compound of formula (I-A), as described in any of clauses B1 or B19-B29, or a pharmaceutically acceptable salt thereof;
b) one or more immunomodulatory agents, or a pharmaceutically acceptable salt thereof; and
c) a pharmaceutically acceptable carrier.
Clause B37. A pharmaceutical composition comprising
a) the therapeutic support composition, as described in any of clauses B1 or B30-B32;
b) one or more immunomodulatory agents, or a pharmaceutically acceptable salt thereof; and
c) a pharmaceutically acceptable carrier.
Clause B38. The method, composition, or kit of any preceding claim, wherein the support is polysaccharide hydrogel, alginate, agarose, cellulose, hyaluronic acid, chitosan, chitin, chondroitin sulfate, heparan sulfate, heparin, gelatin, collagen, polymer matrix, a metal, a ceramic, or a plastic, each of which may be optionally modified.
Clause C1. A method of treating cancer comprising:
a) administering to a subject in need thereof, a therapeutically effective amount of a compound of formula (II-A), or a pharmaceutically acceptable salt thereof,

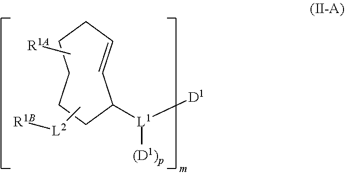

wherein
R^{1A} is selected from the group consisting of C₁₋₄ alkyl, C₁₋₄ haloalkyl, and C₁₋₄ alkoxy;
R^{1B} is selected from the group consisting of G¹, OH, —NR^{1c}_C₁₋₄alkylene-G¹, —NR^{1c}—C₁₋₄ alkylene-N(R^{1d})₂, —N(R^{1c})CHR^{1e}CO₂, —N(R^{1c})—C₁₋₆alkylene-CO₂H, —N(R^{1f})—C₂₋₄alkylene-(N(C₁₋₄alkylene-CO₂H)—C₂₋₄alkylene)ₙ-N(C₁₋₄alkylene-CO₂H)₂, —N(R^{1c})CHR^{1e}—C(O)OC₁₋₆alkyl, —N(R^{1c})—C₁₋₆alkylene-C(O)OC₁₋₆alkyl, and —N(R^{1f})—C₂₋₄alkylene-(N(C₁₋₄alkylene-C(O)OC₁₋₆alkyl)-C₂₋₄alkylene)ₙ-N(C₁₋₄alkylene-C(O)OC₁₋₆alkyl)₂;
R^{1c} and R^{1d}, at each occurrence, are independently hydrogen or C₁₋₄alkyl;
R^{1e} is —C₁₋₄alkylene-CO₂H, —C₁₋₄alkylene-CONH₂, or —C₁₋₄alkylene-OH;
R^{1f} is hydrogen, C₁₋₆alkyl, or C₁₋₄alkylene-CO₂H;
D¹, at each occurrence, is independently a payload;
-L¹-is a linker;
-L²-is selected from the group consisting of —C(O)— and C₁₋₃ alkylene;
G¹ is an optionally substituted heterocyclyl;
m is 1, 2, or 3
n is 0, 1, 2, or 3; and
p is 0, 1, or 2; and
   b) locally administering, at a first tumor in the subject, a therapeutic support composition comprising a support and a tetrazine-containing group of formula

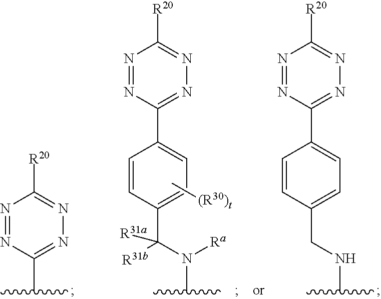

wherein R²⁰ is selected from the group consisting of hydrogen, halogen, cyano, nitro, alkyl, alkenyl, alkynyl, heteroalkyl, aryl, heteroaryl, heterocycle, cycloalkyl, cycloalkenyl, CF₃, CF₂—R′, NO₂, OR′, SR′, C(═O)R′, C(═S)R′, OC(═O)R″, SC(═O)R′″, OC(═S)R′″, SC(═S)R′″, S(═O)R′, S(═O)₂R″, S(═O)₂NR′ R″, C(═O)O—R′, C(═O)S—R′, C(═S)O—R′, C(═S)S—R′, C(═O)NR′R″, C(═S)NR′ R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S) R′R′″, SC(═S)R′R″, NR′C(═O)NR″R″, and NR′C(═S)NR″R″; R′ and R″ at each occurrence are independently selected from hydrogen, aryl and alkyl; and R′″ at each occurrence is independently selected from aryl and alkyl; R³⁰ is halogen, cyano, nitro, hydroxy, alkyl, haloalkyl; alkenyl, alkynyl, alkoxy; halalkoxy; heteroalkyl, aryl, heteroaryl, heterocycle, cycloalkyl, or cycloalkenyl; R^{a}, R^{31a} and R^{31b} are each independently hydrogen, C₁-C₆-alkyl, or C₁-C₆-haloalkyl; and t is 0, 1, 2, 3, or 4;

wherein the tetrazine-containing group is linked or directly bonded to the support; wherein the subject has a second tumor and the administration of a) and the administration of b) inhibits growth of the second tumor.
Clause C2. A method of enhancing or eliciting an immune response against a second tumor in a subject comprising
a) administering a compound of formula (I-A), or a pharmaceutically acceptable salt thereof to the subject; and
b) locally administering a therapeutic support composition to the subject at a first tumor;
wherein the compound of formula (I-A) and the therapeutic support composition are as defined in clause C1;
wherein the administration of a) and the administration of b) enhances or elicits an immune response against the second tumor.
Clause C3. The method of clause C1 or C₂, wherein the therapeutic support composition is not locally administered at the second tumor.
Clause C4. A method of inhibiting tumor metastasis in a subject at risk of tumor metastasis comprising
a) administering a compound of formula (I-A), or a pharmaceutically acceptable salt thereof to the subject; and
b) locally administering a therapeutic support composition to the subject at a first tumor;
wherein the compound of formula (I-A) and the therapeutic support composition are as defined in clause C1.
Clause C5. The method of clause C4, wherein the administration of a) and the administration of b) enhances or elicits an immune response that inhibits the metastasis.
Clause C6. The method of clause C5 or C6, wherein the inhibiting of tumor metastasis comprises inhibiting development of a second tumor in the subject.
Clause C7. The method of any of clauses C4-C6, further comprising identifying the subject at risk of tumor metastasis.
Clause C8. The method of any of clauses C4-C7, further comprising selecting the subject at risk of tumor metastasis.
Clause C9. The method of any of clauses C4-C8, wherein the subject at risk of metastasis suffers from a first tumor characterized as a solid cancer of stage II-III or later, or a high grade tumor.
Clause C10. The method of any of clauses C4-C9, wherein first tumor cells are separated from the first tumor.
Clause C11. The method of clause C10, wherein the first tumor cells are present in tissue surrounding the first tumor, present in tumor cell-platelet aggregates, present in systemic circulation of the subject, and/or present at a second tissue location in the subject.
Clause C12. The method of any of clauses C1-C11, wherein the subject displays a biomarker for tumor metastasis.
Clause C13. The method of clause C12, wherein the biomarker is one or more of CCR7, CXCR4, E-cadherin, EpCAM, VCAM1, Integrin-alpha10, N-cadherin, vimentin, fibronectin.
Clause C14. The method of any of clause C1-C13, further comprising administering a therapeutically effective amount of one or more immunomodulatory agents.
Clause C15. The method of clause C14, wherein the one or more immunomodulatory agents is one or more of an immune checkpoint inhibitor, a toll-like receptor (TLR) agonist, a stimulator of interferon genes (STING) agonist, a cytokine, a cytokine inhibitor, a cytokine receptor agonist, a cytokine receptor antagonist, a chemokine, a chemokine inhibitor, a chemokine receptor agonist, or a chemokine receptor antagonist.
Clause C16. The method of clause C14 or C15, wherein the one or more immunomodulatory agents comprises one or more TLR agonists selected from the group consisting of Bacillus Calmette-Guerin (BCG), lipopolysaccharide, peptidoglycan, polyriboinosinic-polyribocytidylic acid (Poly I:C), Imiquimod, Coley's toxin, Polyadenylic-polyuridylic acid (Poly A:U), Monophosphoryl lipid A, single- and double-stranded RNA, or a CpG oligodeoxynucleotide (ODN).
Clause C17. The method of any of clauses C1-C16, wherein the administrations a), b), and/or of the one or more immunomodulatory agents c) are simultaneous, separate, or sequential, and in any order.
Clause C18. The method of any of clauses C14-C17, the immunomodulatory agent(s) is administered simultaneously with the therapeutic support composition.
Clause C19. The method of clause C17 or 18, wherein the simultaneous administration is by coinjection, coimplantation, or coformulation.
Clause C20. The method of any of clauses C2-C3 or C5-C19, wherein the immune response is an increase or decrease in one or more of innate and adaptive immune cells.
Clause C21. The method of any of clauses C2-C3 or C5-C19, wherein the immune response is an increase or decrease of one or more of leukocytes, lymphocytes, monocytes, eosinophils, and antibodies.
Clause C22. The method of any of clauses C2-C3 or C5-C19, wherein the immune response is an increase in CD3, CD4, CD8, and/or PD-1 positive tumor-infiltrating lymphocytes, in the first tumor and/or second tumor.
Clause C23. The method of any of clauses C2-C3 or C5-C19, wherein the immune response is a decrease in regulatory T-cells in the first tumor and/or second tumor.
Clause C24. The method of any of clauses C1-C23, wherein the support is polysaccharide hydrogel, alginate, agarose, cellulose, hyaluronic acid, chitosan, chitin, chondroitin sulfate, heparan sulfate, heparin, gelatin, collagen, polymer matrix, a metal, a ceramic, or a plastic, each of which may be optionally modified.

1. A compound of formula (I), or a pharmaceutically acceptable salt thereof 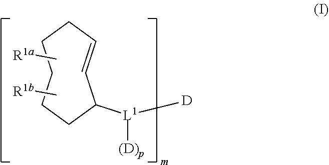
wherein
R^{1a}, at each occurrence, is independently selected from the group consisting of C₁₋₄alkyl, hydrogen, and C₁₋₄haloalkyl;
R^{1b}, at each occurrence, is independently selected from the group consisting of C(O)N(R^{1c})—C₁₋₆alkylene-CO₂H, C(O)OH, C(O)N(R^{1c})CHR^{1e}CO₂H, C(O)N(R^{1c})—C₁₋₆alkylene-C(O)OC₁₋₄alkyl, C(O)OC₁₋₄alkyl, C(O)N(R^{1c})CHR^{1e}C(O)OC₁₋₄alkyl, hydrogen, C₁₋₄alkyl, and C₁₋₄ haloalkyl;
R^{1c}, at each occurrence, is independently hydrogen or C₁₋₄alkyl;
R^{1e}, at each occurrence, is independently —C₁₋₄alkylene-CO₂H, —C₁₋₄alkylene-CONH₂, or —C₁₋₄ alkylene-OH;
D, at each occurrence, is independently a cyclic dinucleotide;
L¹, at each occurrence, is independently a linker;
m, at each occurrence, is independently 1, 2, or 3; and
p, at each occurrence, is independently 0, 1, or 2.

2. The compound of claim 1, or a pharmaceutically acceptable salt thereof, of formula (I-A)

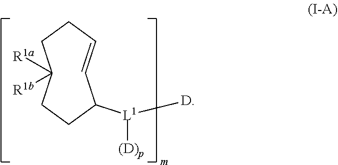

3. The compound of claim 1 or 2, or a pharmaceutically acceptable salt thereof, wherein R^{1a} is hydrogen.

4. The compound of claim 1 or 2, or a pharmaceutically acceptable salt thereof, wherein R^{1a} is C₁₋₄ alkyl.

5. The compound of claim 1 or 2, or a pharmaceutically acceptable salt thereof, wherein R^{1a} is CH₃.

6. The compound of any of claims 1-5, or a pharmaceutically acceptable salt thereof, wherein R^{1b} is hydrogen.

7. The compound of any of claims 1-5, or a pharmaceutically acceptable salt thereof, wherein R^{1b} is C(O)N(R^{1c})—C₁₋₆alkylene-CO₂H.

8. The compound of claim 7, or a pharmaceutically acceptable salt thereof, wherein R^{1b} is C(O)N(R^{1c})CH₂CO₂H.

9. The compound of any of claim 1-5 or 7-8, or a pharmaceutically acceptable salt thereof, wherein R^{1c} is hydrogen.

10. The compound of any of claims 1-5, or a pharmaceutically acceptable salt thereof, wherein R^{1b} is C(O)OH.

11. The compound of any of claims 1-10, or a pharmaceutically acceptable salt thereof, wherein D, at each occurrence, is independently 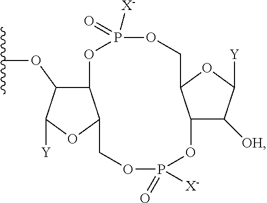
wherein Y is a nucleobase and X is O or S.

12. The compound of any of claims 1-11, or a pharmaceutically acceptable salt thereof, wherein: 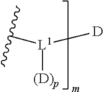
is 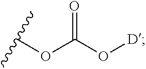
and
D′ is a cyclic dinucleotide payload moiety.

13. The compound of claim 12, or a pharmaceutically acceptable salt thereof, wherein the cyclic dinucleotide payload moiety is

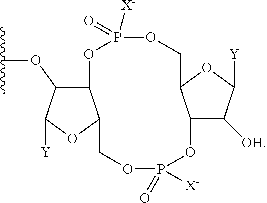

14. The compound of claim 12, or a pharmaceutically acceptable salt thereof, wherein the cyclic dinucleotide payload moiety is

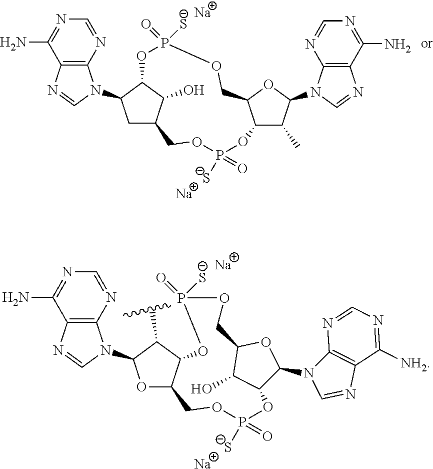

15. The compound of claim 1, or a pharmaceutically acceptable salt thereof, selected from the group consisting of

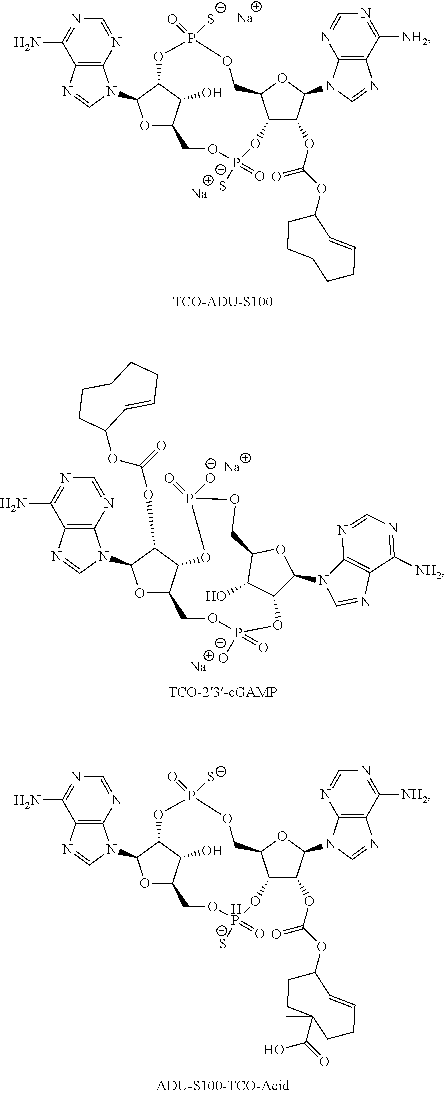

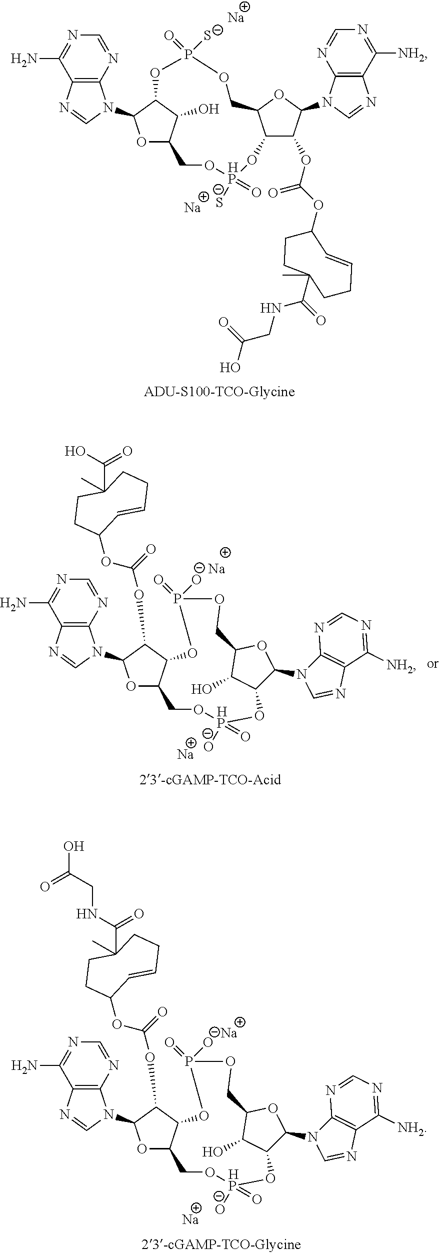

16. A pharmaceutical composition comprising the compound of any of claims 1-15, or a pharmaceutically acceptable salt thereof, and a pharmaceutically acceptable carrier.

17. A pharmaceutical combination comprising a compound of any of claims 1-15, or a pharmaceutically acceptable salt thereof, or the pharmaceutical composition of claim 16, and a therapeutic support composition for use in the treatment of cancer; or for use in enhancing or eliciting an immune response, the therapeutic support composition comprising a biocompatible support and a tetrazine-containing group of formula 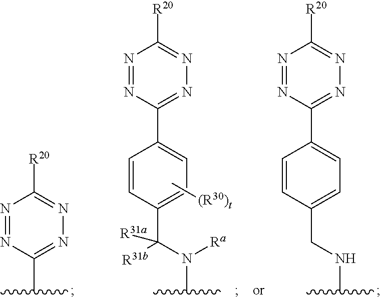
wherein
R²⁰ is selected from the group consisting of hydrogen, halogen, cyano, nitro, alkyl, alkenyl, alkynyl, heteroalkyl, aryl, heteroaryl, heterocycle, cycloalkyl, cycloalkenyl, CF₃, CF₂—R′, NO₂, OR′, SR′, C(═O)R′, C(═S)R′, OC(═O)R″, SC(═O)R′″, OC(═S)R′″, SC(═S)R′″, S(═O)R′, S(═O)₂′″, S(═O)₂NR′ R″, C(═O)O—R′, C(═O)S—R′, C(═S)O—R′, C(═S)S—R′, C(═O)NR′R″, C(═S)NR′ R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S) R′R′″, SC(═S)R′R″, NR′C(═O)NR″R″, and NR′C(═S)NR″R″;
R′ and R″ at each occurrence are independently selected from hydrogen, aryl and alkyl;
R′″ at each occurrence is independently selected from aryl and alkyl;
R³⁰ is halogen, cyano, nitro, hydroxy, alkyl, haloalkyl; alkenyl, alkynyl, alkoxy; halalkoxy; heteroalkyl, aryl, heteroaryl, heterocycle, cycloalkyl, or cycloalkenyl;
R^{a}, R^{31a} and R^{31b} are each independently hydrogen, C₁-C₆-alkyl, or C₁-C₆-haloalkyl; and
t is 0, 1, 2, 3, or 4.

18. The pharmaceutical combination of claim 17, wherein the tetrazine-containing group is linked or directly bonded to a hyaluronic acid biocompatible support.

19. The pharmaceutical combination of claim 17, wherein the therapeutic support composition comprises substituted hyaluronic acid units of formula (II), 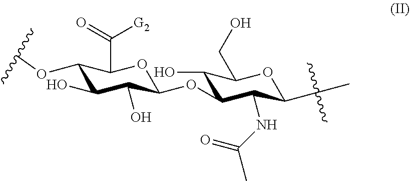
wherein G² is 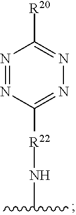
and
R²² is a linker of 1 to 100 linking atoms.

20. The pharmaceutical combination of claim 19, wherein:
G² is

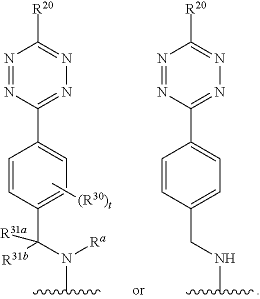

21. The pharmaceutical combination of claim 19, wherein G² is 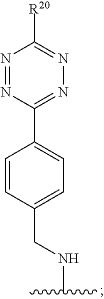
and R²⁰ is hydrogen or C₁₋₄alkyl.

22. The pharmaceutical combination of any of claims 17-21, wherein the use is for treating or preventing a cancer.

23. The pharmaceutical combination of claim 22, wherein the cancer is a melanoma, renal cancer, prostate cancer, ovarian cancer, breast cancer, glioma, lung cancer, soft tissue carcinoma, soft tissue sarcoma, osteosarcoma, or pancreatic cancer.

24. The pharmaceutical combination of claim 22 or 23, wherein the cancer is a solid tumor.

25. The pharmaceutical combination of claim 22 or 23, wherein the cancer is a soft tissue sarcoma.

26. The pharmaceutical combination of claim 25, wherein the soft tissue sarcoma is a fibrosarcoma, rhabdomyosarcoma, or Ewing's sarcoma.

27. The pharmaceutical combination of any of claims 17-21, wherein the use is for enhancing or eliciting an immune response.

28. The pharmaceutical combination of claim 27, wherein the immune response is an increase in one or more of leukocytes, lymphocytes, monocytes, and eosinophils.

29. The pharmaceutical combination of any of claims 17-28, further comprising an additional therapeutic agent selected from the group consisting of an anticancer agent, an immune checkpoint inhibitor, or a compound of formula (I-B), or a pharmaceutically acceptable salt thereof, 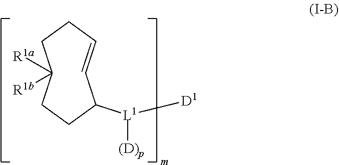
wherein
D¹, at each occurrence, is independently a payload selected from an anticancer drug payload, a microbial immunosuppressive drug payload, an anti-restenosis drug payload, antibiotic drug payload, antifungal drug payload, antiviral drug payload, anti-inflammatory/anti-arthritic drug payload, a corticosteroid drug payload, and an immunosuppressant drug payload; and
R^{1a}, R^{1b}, L¹, and m are as defined in any of claims 1-11.

30. The pharmaceutical combination of claim 29, wherein p is 0; m is 1; and -L¹-is

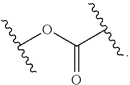 [SMILES: *OC(*)=O]

31. The pharmaceutical combination of claim 29 or 30, wherein the anticancer drug is doxorubicin.

32. A kit comprising the compound of any of claims 1-15, or a pharmaceutically acceptable salt thereof, or the pharmaceutical composition of claim 16, and instructions for use thereof.

33. The kit of claim 32, further comprising the therapeutic support composition as defined in any of claims 19-23.

34. The kit of claim 32 or 33, further comprising the compound of formula (I-B), as defined in any of claims 29-31.
